(19) United States
(12) Patent Application Publication
(10) Pub. No.: US 2016/0319301 A1
(43) Pub. Date: Nov. 3, 2016

(54) Methods for Increasing Resistance to Soybean Cyst Nematode in Soybean Plants

(71) Applicants: Syngenta Participations AG, Basel (CH); The United States of America, as Represented by the Secretary of Agriculture, Washington, DC (US)

(72) Inventors: Xiang Huang, Cary, NC (US); Benjamin Matthews, Beltsville, MD (US)

(21) Appl. No.: 15/209,965

(22) Filed: Jul. 14, 2016

Related U.S. Application Data
(63) Continuation of application No. 14/038,168, filed on Sep. 26, 2013.
(60) Provisional application No. 61/706,044, filed on Sep. 26, 2012.

(51) Int. Cl. C12N 15/82; A01H 1/02; A01H 5/10

(52) U.S. Cl. CPC C12N 15/8285; A01H 5/10; A01H 1/02

(57) ABSTRACT

The invention relates to methods and compositions for increasing resistance to infection by soybean cyst nematode on a soybean plant, plant part or plant cell. Nucleotide sequences that confer resistance to soybean cyst nematode when expressed in soybean are provided.

### STATEMENT OF PRIORITY

This application claims the benefit, under 35 U.S.C. §119(e), of U.S. Provisional Application No. 61/706,044; filed Sep. 26, 2012, the entire contents of which is incorporated by reference herein.

### STATEMENT REGARDING ELECTRONIC FILING OF A SEQUENCE LISTING

A Sequence Listing in ASCII text format, submitted under 37 C.F.R. §1.821, entitled 9207-84TS_ST25.txt, 439,617 bytes in size, generated on Sep. 12, 2013 and filed via EFS-Web, is provided in lieu of a paper copy. This Sequence Listing is hereby incorporated by reference into the specification for its disclosures.

### FIELD OF THE INVENTION

The invention relates to methods for controlling nematode parasitism by overexpression of a recombinant plant polynucleotide, RNAi and/or antisense.

### BACKGROUND OF THE INVENTION

Nematodes are obligate, sedentary endoparasites that feed on the roots, leaves and stems of more than 2,000 vegetables, fruits, and ornamental plants, causing an estimated $100 billion crop loss worldwide. Nematodes are present throughout the United States, but are mostly a problem in warm, humid areas of the south and west, as well as in sandy soils. Soybean cyst nematode (SCN), Heterodera glycines, was first discovered in North Carolina in 1954. It is the most serious pest of soybean plants. Once SCN is present in a field, it cannot feasibly be eradicated using known methods. Although soybean is the major economic crop attacked by SCN, SCN parasitizes some fifty hosts in total, including field crops, vegetables, ornamentals, and weeds.

The interaction of a plant parasitic nematode with the root of a host plant is especially interesting because the nematode injects fluid containing numerous effector proteins into a selected root cell to commandeer its metabolic machinery forcing it to form a complex, metabolically active feeding site. Cyst nematodes, such as SCN, form a feeding site called a “syncytium.” Some proteins injected by the nematode into the host cell may be targeted to the nucleus of the host cell to reorganize transcription, while others subvert the host cell and make it more accommodating to the nematode (Opperman and Bird (1998) Curr Opin Plant Biol 1:342-346; Davis et al. (2000) Ann Rev Phytopathol 38:365-96; Gheysen and Mitchum (2011) Curr Opinion Plant Biol 14:415-421; Niblack et al. (2006) Annu. Rev. Phytopath 44:283-303; Schmitt et al. eds., (2004) Biology and Management of the Soybean Cyst Nematode. Marceline, Mo.: Schmitt & Assoc. 2^{nd} ed.). The secretions of SCN originate from one dorsal and two subventral esophageal secretory glands and contain numerous proteins (Gao et al. (2003) Mol. Plant Microbe Interact 16:720-726). These secretions are injected into a host soybean (Glycine max) cell adjacent to the vascular system of the root to form the feeding site. Numerous effector proteins are present within the secretion and their identity varies according to the plant parasitic nematode. Many of these proteins are thought to be effector molecules that inhibit the host plant defense response or promote changes in the host to promote the development of the nematode. For example, a cDNA encoding the cellulase, β-1,4-endoglucanase, was isolated from G. rostochiensis and Hederodera glycines (SCN; Smant et al. (1998) Proc Nat Acad Sci USA 95:4906-11; Yan et al. (1998) Gene 220:61-70). Many other proteins are also present in these nematode secretions, some with unknown functions. Plant parasitic nematode secretions and parasitism genes have been reviewed in-depth by numerous authors (Caillaud et al. (2008) J Plant Physiol 165:104-113; Davis et al. (2000) Ann Rev Phytopatho 38:365-96; Gao et al. (2003) Mol Plant-Microbe Interactions 16:720-726; Haegeman et al. (2012) Gene 492:19-31; Hogenhout et al. (2009) Mol Plant-Microbe Interactions 22:115-122).

Traditional practices for managing nematodes include maintaining proper fertility and soil pH levels in nematode-infested land; controlling other plant diseases, as well as insect and weed pests; using sanitation practices such as plowing, planting, and cultivating of nematode-infested fields only after working non-infested fields; cleaning equipment thoroughly after working in infested fields; not using seed from plants grown on infested land for planting non-infested fields unless the seed has been properly cleaned; rotating infested fields and alternating host crops with non-host crops, such as, corn, oat and alfalfa; using pesticides or fumigants (e.g., nematicides); and planting resistant soybean varieties. While many of these can be effective, in addition to being time consuming and costly to implement, some of these approaches are no longer feasible, such as the application of nematicides, due to their toxicity and negative environmental impact. Thus, there is currently no efficient and effective approach to control nematode infection in plants. Therefore, there is a need for compositions and methods for preventing, controlling, and reducing nematode parasitism in plants.

Accordingly, the present invention overcomes the deficiencies in the art by providing compositions and methods comprising overexpression of recombinant plant polynucleotides, RNAi and/or antisense for control of soybean cyst nematode.

### SUMMARY OF THE INVENTION

One aspect of the invention is a method of increasing resistance of a soybean plant cell to infection by a soybean cyst nematode, comprising: introducing into a soybean plant cell a recombinant nucleic acid molecule comprising one or more nucleotide sequences selected from the group consisting of: (a) a nucleotide sequence of any of SEQ ID NOs:1-97; (b) a nucleotide sequence encoding a double stranded RNA molecule comprising at least 18 consecutive nucleotides of a nucleotide sequence of any of SEQ ID NOs:1-97 and the reverse-complement thereof; (c) a nucleotide sequence encoding a portion of a nucleotide sequence of any of SEQ ID NOs: 1-97, which when expressed produces an antisense nucleotide sequence; and (d) any combination of (a)-(c), to produce a transgenic soybean plant cell, thereby increasing resistance of the soybean plant cell to infection by a soybean cyst nematode as compared to a control.

Another aspect of the invention provides a method of increasing resistance of a soybean plant or plant part to infection by a soybean cyst nematode, comprising: (a) introducing into a soybean plant cell a recombinant nucleic acid molecule comprising one or more nucleotide sequences selected from the group consisting of: (i) a nucleotide sequence of any of SEQ ID NOs:1-97; (ii) a nucleotide sequence encoding double stranded RNA molecule comprising at least 18 consecutive nucleotides of a nucleotide sequence of any of SEQ ID NOs:1-97, and the reverse-complement thereof; (iii) a nucleotide sequence encoding a portion of a nucleotide sequence of any of SEQ ID NOs: 1-97, which when expressed produces an antisense nucleotide sequence; and (iv) any combination of (i)-(iii), to produce a transgenic soybean plant cell, wherein the recombinant nucleic acid molecule is expressed in the transgenic soybean plant cell; and (b) regenerating a transgenic soybean plant and/or soybean plant part from the transgenic soybean plant cell of (a), wherein the transgenic soybean plant and/or soybean plant part comprises in its genome the recombinant nucleic acid molecule and has increased resistance to infection by the soybean cyst nematode as compared to a control.

A further aspect of the invention provides a method of reducing soybean cyst nematode cyst formation on a soybean plant cell, comprising: introducing into a soybean plant cell a recombinant nucleic acid molecule comprising one or more nucleotide sequences selected from the group consisting of: (a) a nucleotide sequence of any of SEQ ID NOs:1-97; (b) a nucleotide sequence encoding a double stranded RNA molecule comprising at least 18 consecutive nucleotides of a nucleotide sequence of any of SEQ ID NOs: 1-97, and the reverse-complement thereof; (c) a nucleotide sequence encoding a portion of a nucleotide sequence of any of SEQ ID NOs: 1-97, which when expressed produces an antisense nucleotide sequence; and (d) any combination of (a)-(c), to produce a transgenic soybean plant cell, thereby reducing soybean cyst nematode cyst formation on a soybean plant, soybean plant part, or soybean plant cell as compared to a control.

An additional aspect of the invention provides a method of reducing soybean cyst nematode cyst formation on a soybean plant and/or soybean plant part, comprising: (a) introducing into a soybean plant cell a recombinant nucleic acid molecule comprising one or more nucleotide sequences selected from the group consisting of: (i) a nucleotide sequence of any of SEQ ID NOs:1-97, or a fragment thereof; (ii) a nucleotide sequence encoding a double stranded RNA molecule comprising at least 18 consecutive nucleotides of a nucleotide sequence of any of SEQ ID NOs: 1-97, and the reverse-complement thereof; (iii) a nucleotide sequence encoding a portion of a nucleotide sequence of any of SEQ ID NOs: 1-97, which when expressed produces an antisense nucleotide sequence; and (iv) any combination of (i)-(iii), to produce a transgenic soybean plant cell, wherein the recombinant nucleic acid molecule is expressed in the transgenic soybean plant cell; and (b) regenerating a transgenic soybean plant and/or soybean plant part from the transgenic soybean plant cell of (a), wherein the transgenic soybean plant and/or soybean plant part comprises in its genome the recombinant nucleic acid molecule and has reduced soybean cyst nematode cyst formation as compared to a control.

A further aspect of the invention provides a method of reducing soybean cyst nematode cyst development on roots of a soybean plant infected by a nematode, comprising (a) introducing into a soybean plant cell a recombinant nucleic acid molecule comprising one or more nucleotide sequences selected from the group consisting of: (i) a nucleotide sequence of any of SEQ ID NOs:1-97, or a fragment thereof; (ii) a nucleotide sequence encoding a double stranded RNA molecule comprising at least 18 consecutive nucleotides of a nucleotide sequence of any of SEQ ID NOs:1-97, and the reverse-complement thereof; (iii) a nucleotide sequence encoding a portion of a nucleotide sequence of any of SEQ ID NOs:1-97, which when expressed produces an antisense nucleotide sequence; and (iv) any combination of (i)-(iii), to produce a transgenic soybean plant cell, wherein the recombinant nucleic acid molecule is expressed in the transgenic soybean plant cell; and (b) regenerating a transgenic soybean plant and/or soybean plant part from the transgenic soybean plant cell of (a), wherein the transgenic soybean plant and/or soybean plant part comprises in its genome the recombinant nucleic acid molecule and has reduced soybean cyst nematode cyst development on roots of the soybean plant as compared to a control.

In some aspects of the invention, the recombinant nucleic acid sequence comprises one or more nucleotide sequences of any of SEQ ID NOs:1-53, or a fragment thereof, or any combination thereof, and said nucleotide sequence is overexpressed in the plant, plant part and/or plant cell. In other aspects, the recombinant nucleic acid sequence comprises one or more nucleotide sequences of: a nucleotide sequence encoding a double stranded RNA molecule comprising at least 18 consecutive nucleotides of a nucleotide sequence of any of SEQ ID NOs:43-97, and the reverse-complement thereof; a nucleotide sequence encoding a portion of a nucleotide sequence of any of SEQ ID NOs:43-97, which when expressed produces an antisense nucleotide sequence; or any combination thereof.

Additional aspects of the invention provide compositions including nucleic acid constructs for transforming a plant, plant part and/or plant cell. Also provided herein are transformed plant cells, plants and/or plant parts as well as progeny plants, harvested and processed products produced from said transformed plant cell, plant, plant parts, and/or progeny plants.

These and other aspects of the invention are set forth in more detail in the description of the invention below.

### BRIEF DESCRIPTION OF THE DRAWINGS

FIG. 1 shows the gene expression vector pRAP15 for over-expressing genes using the figwort mosaic virus (FMV) promoter. The vector contains as a selectable marker a nucleotide sequence encoding enhance green fluorescent protein (eGFP) driven by the Agrobacterium rhizogenes rolD promoter, and the attR1 and attR2 sites for Gateway® cloning. The vector also contains a nucleotide sequence encoding tetracycline resistance (TetR) for bacterial selection and a nucleotide sequence encoding bar for selection of transformed plant cells.

FIG. 2 shows transformed soybean roots on a composite plant. The transformed roots display green fluorescence under light from a Dark Reader® lamp. (A) Roots after approximately 3 weeks. (B) Roots after second trim.

FIG. 3 shows expression of transcript levels of genes encoded by C45 and C49 as measured by qRT-PCR in transformed roots. The x-axis represents the experimental roots and the y-axis represents the fold in expression levels based on the qRT-PCR analysis of the three replicates of each gene.

FIG. 4 shows the Percent Female Index calculated from mature female cysts found on transformed soybean roots over-expressing a gene as compared to that calculated from mature female cysts found on control plants. (A) Female index of 45 genes supporting nematode development less than the empty vector control when over-expressed; (B) Female index of 57 genes supporting nematode development more than the empty vector control when over-expressed.

FIG. 5 Simplified version of phenylpropanoid biosynthesis showing the location of tested genes encoding enzymes in the pathway. Genes tested included two encoding phenylalanine ammonia lyase (PAL, EC 4.3.1.24; A45, A53) and single genes encoding chalcone synthase (ChS, EC 2.3.1.74; A52), 4-coumarate CoA ligase (4CL, EC 6.2.1.12, A48), cinnamate-4-hydroxylase (C4H, EC 1.14.13.11; A11) and cinnamoyl CoA reductase (CCR, EC 1.2.1.44; A46). The Female Index (FI) obtained when these genes were overexpressed in soybean roots is indicated.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, +5%, ±1%, +0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The invention is directed in part to the discovery that by expressing the recombinant nucleic acid molecules of the invention in a plant cell, plant and/or plant part, the plant cell, plant and/or plant part can be made to have increased resistance to soybean cyst nematode infection, reduced soybean cyst nematode cyst formation and/or reduced soybean cyst nematode cyst development on roots of the soybean plant.

Accordingly, in one embodiment, the invention provides a method of increasing resistance of a soybean plant cell to infection by a soybean cyst nematode, comprising: introducing into a soybean plant cell a recombinant nucleic acid molecule comprising, consisting essentially of, or consisting of one or more nucleotide sequences of: (a) a nucleotide sequence of any of SEQ ID NOs:1-97, or a fragment thereof; (b) a nucleotide sequence encoding a double stranded RNA molecule comprising at least 18 consecutive nucleotides of a nucleotide sequence of any of SEQ ID NOs: 1-97 and the reverse-complement thereof; (c) a nucleotide sequence encoding a portion (e.g., consecutive nucleotides) of a nucleotide sequence of any of SEQ ID NOs: 1-97, which when expressed produces an antisense nucleotide sequence; or (d) any combination of (a)-(c), to produce a transgenic soybean plant cell, thereby increasing resistance of the soybean plant cell to infection by a soybean cyst nematode as compared to a control soybean plant cell that does not comprise (i.e., is not transformed with) said recombinant nucleic acid molecule. In some embodiments of the invention, the method further comprises regenerating a transgenic soybean plant and/or soybean plant part from the transgenic soybean plant cell, wherein the regenerated transgenic soybean plant and/or soybean plant part comprises in its genome the recombinant nucleic acid molecule and has increased resistance to infection by soybean cyst nematode as compared to a control soybean plant and/or soybean plant part that does not comprise (i.e., is not transformed with) said recombinant nucleic acid molecule. In still further embodiments, the method further comprises obtaining a progeny soybean plant from the transgenic soybean plant, wherein said progeny plant comprises in its genome the recombinant nucleic acid molecule and has increased resistance to infection by soybean cyst nematode as compared to a control.

In an additional aspect of the invention, a method of reducing soybean cyst nematode cyst formation on a soybean plant cell is provided, the method comprising: introducing into a soybean plant cell a recombinant nucleic acid molecule comprising, consisting essentially of, or consisting of one or more nucleotide sequences of: (a) a nucleotide sequence of any of SEQ ID NOs:1-97, or a fragment thereof; (b) a nucleotide sequence encoding a double stranded RNA molecule comprising, consisting essentially of, or consisting of at least 18 consecutive nucleotides of a nucleotide sequence of any of SEQ ID NOs:1-97, and the reverse-complement thereof; (c) a nucleotide sequence encoding a portion (e.g., consecutive nucleotides) of a nucleotide sequence of any of SEQ ID NOs:1-97, which when expressed produces an antisense nucleotide sequence; or (d) any combination of (a)-(c), to produce a transgenic soybean plant cell, thereby reducing soybean cyst nematode cyst formation on a soybean plant, soybean plant part, or soybean plant cell as compared to a control soybean plant, soybean plant part, or soybean plant cell that does not comprise (i.e., is not transformed with) said recombinant nucleic acid molecule. In some embodiments, the method further comprises regenerating a transgenic soybean plant and/or soybean plant part from the transgenic soybean cell, wherein the regenerated transgenic soybean plant and/or soybean plant part comprises in its genome the recombinant nucleic acid molecule and has reduced soybean cyst nematode cyst formation as compared to a control soybean plant and/or soybean plant part. In still other embodiments, the method further comprises obtaining a progeny soybean plant from the transgenic soybean plant, wherein said progeny plant comprises in its genome the recombinant nucleic acid molecule and has reduced soybean cyst nematode cyst formation as compared to a control soybean plant (e.g., a soybean plant that does not comprise in its genome the recombinant nucleic acid molecule of this invention).

In further aspects of the invention, a method is provided for reducing the number of mature female soybean cyst nematodes on roots of a soybean plant infected by a nematode, comprising (a) introducing into a soybean plant cell a recombinant nucleic acid molecule comprising one or more nucleotide sequences selected from the group consisting of: (i) a nucleotide sequence of any of SEQ ID NOs: 1-97, or a fragment thereof; (ii) a nucleotide sequence encoding a double stranded RNA molecule comprising at least 18 consecutive nucleotides of a nucleotide sequence of any of SEQ ID NOs:1-97, and the reverse-complement thereof; (iii) a nucleotide sequence encoding a portion of a nucleotide sequence of any of SEQ ID NOs:1-97, which when expressed produces an antisense nucleotide sequence; and (iv) any combination of (i)-(iii), to produce a transgenic soybean plant cell, wherein the recombinant nucleic acid molecule is expressed in the transgenic soybean plant cell; and (b) regenerating a transgenic soybean plant and/or soybean plant part from the transgenic soybean plant cell of (a), wherein the transgenic soybean plant and/or soybean plant part comprises in its genome the recombinant nucleic acid molecule and has a reduced number of mature female soybean cyst nematodes on roots of the soybean plant as compared to a control. In some embodiments, the method further comprises obtaining a progeny soybean plant from the transgenic soybean plant, wherein said progeny plant comprises in its genome the recombinant nucleic acid molecule and has a reduced number of mature female soybean cyst nematodes on its roots as compared to a control.

In additional embodiments of the invention, a method is provided for reducing soybean cyst nematode cyst development on roots of a soybean plant infected by a nematode, comprising (a) introducing into a soybean plant cell a recombinant nucleic acid molecule comprising, consisting essentially of, or consisting of one or more nucleotide sequences of: (i) a nucleotide sequence of any of SEQ ID NOs:1-97, or a fragment thereof; (ii) a nucleotide sequence encoding a double stranded RNA molecule comprising, consisting essentially of, or consisting of at least 18 consecutive nucleotides of a nucleotide sequence of any of SEQ ID NOs:1-97, and the reverse-complement thereof; (iii) a nucleotide sequence encoding a portion (e.g., consecutive nucleotides) of any of SEQ ID NOs:1-97, which when expressed produces an antisense nucleotide sequence; or (iv) any combination of (i)-(iii), to produce a transgenic soybean plant cell, wherein the recombinant nucleic acid molecule is expressed in the transgenic soybean plant cell; and (b) regenerating a transgenic soybean plant and/or soybean plant part from the transgenic soybean plant cell of (a), wherein the transgenic soybean plant and/or soybean plant part comprises in its genome the recombinant nucleic acid molecule and has reduced soybean cyst nematode cyst development on roots of the soybean plant as compared to a control soybean plant and/or soybean plant part that does not comprise (i.e., is not transformed with) said recombinant nucleic acid molecule. In some embodiments, the method further comprises obtaining a progeny soybean plant from the transgenic soybean plant, wherein said progeny plant comprises in its genome the recombinant nucleic acid molecule and has reduced soybean cyst nematode cyst development on roots of the soybean plant as compared to a control soybean plant.

As used herein, “reduced cyst development” refers to a reduction in the continued development of the cyst after it first forms as compared to a control. Thus, “reduced cyst development refers to cysts that are of a smaller size, contain fewer eggs per cyst and are white or cream-colored as compared to mature cysts, which are larger and brown-colored.

As used herein, “reduced cyst formation” means that the numbers of cysts formed are reduced as compared to a control. Further, as used herein, a reduced number of mature females can be equivalent to a reduction in the number of cysts (e.g., a reduction in the number of mature females that can produce cysts will result in a reduced number of cysts formed).

In representative embodiments, a recombinant nucleic acid molecule of the invention can comprise, consist essentially of, or consist of one or more nucleotide sequences of SEQ ID NOs:1-97 and/or SEQ ID NOs:1-53, or a fragment thereof. In particular aspects of the invention, the one or more nucleotide sequences of SEQ ID NOs:1-97 and/or SEQ ID NOs:1-53, or fragments thereof, can be overexpressed in a plant cell, plant or plant part. In some embodiments, the polypeptides encoded by the nucleotide sequences of SEQ ID NOs:1-97, and/or a fragment thereof, can be the amino acid sequences of SEQ ID NOs:98-194.

In other embodiments of the invention, a recombinant nucleic acid molecule of the invention can comprise, consist essentially of, or consist of one or more nucleotide sequences encoding a double stranded RNA molecule comprising, consisting essentially of, or consisting of at least 18 consecutive nucleotides of a nucleotide sequence of any of SEQ ID NOs:43-97, and the reverse-complement thereof.

In still other embodiments, a recombinant nucleic acid molecule of the invention can comprise, consist essentially of, or consist of one or more nucleotide sequences encoding a portion (e.g., consecutive nucleotides) of a nucleotide sequences of any of SEQ ID NOs:43-97, which when expressed produces an antisense nucleotide sequence.

In additional embodiments, a recombinant nucleic acid molecule of the invention can comprise, consist essentially of, or consist of one or more nucleotide sequences encoding a double stranded RNA molecule comprising, consisting essentially of, or consisting of at least 18 consecutive nucleotides of a nucleotide sequence of any of SEQ ID NOs:43-97, and the reverse-complement thereof; and one or more nucleotide sequences comprising, consisting essentially of, or consisting of a nucleotide sequence that encodes a portion (e.g., consecutive nucleotides) of a nucleotide sequence of any of SEQ ID NOs:43-97, which when expressed produces an antisense nucleotide sequence.

In other embodiments, a recombinant nucleic acid molecule of the invention can comprise, consist essentially of, or consist of one or more nucleotide sequences of a nucleotide sequence of any of SEQ ID NOs:1-53, or a fragment thereof, wherein the nucleotide sequences of any of SEQ ID NOs:1-53, or a fragment thereof, can be overexpressed in a plant, plant part and/or plant cell; and one or more nucleotide sequences of a nucleotide sequence that encodes a portion (e.g., consecutive nucleotides) of a nucleotide sequence of any of SEQ ID NOs:43-97, which when expressed produces an antisense nucleotide sequence.

In other embodiments, a recombinant nucleic acid molecule of the invention can comprise, consist essentially of, or consist of one or more nucleotide sequences of a nucleotide sequence of any of SEQ ID NOs:1-53, or a fragment thereof, wherein the nucleotide sequences of any of SEQ ID NOs: 1-53, or a fragment thereof, can be overexpressed in a plant, plant part and/or plant cell; and one or more nucleotide sequences that encodes a double stranded RNA molecule comprising, consisting essentially of, or consisting of at least 18 consecutive nucleotides of a nucleotide sequence of any of SEQ ID NOs:43-97 and the reverse-complement thereof.

In further embodiments, a recombinant nucleic acid molecule of the invention can comprise, consist essentially of, or consist of one or more nucleotide sequences of a nucleotide sequence of SEQ ID NOs:1-53, or a fragment thereof, wherein the one or more nucleotide sequences can be overexpressed in a plant, plant part and/or plant cell; one or more nucleotide sequences encoding a double stranded RNA molecule comprising, consisting essentially of, or consisting of at least 18 consecutive nucleotides of a nucleotide sequence of any of SEQ ID NOs:43-97 and the reverse-complement thereof; and one or more nucleotide sequences comprising, consisting essentially of, or consisting of a nucleotide sequence encoding a portion (e.g., consecutive nucleotides) of any of SEQ ID NOs:43-97, which when expressed produces an antisense nucleotide sequence

The terms “increase,” “increasing,” “increased,” “enhance,” “enhanced,” “enhancing,” and “enhancement” (and grammatical variations thereof), as used herein, describe an elevation in the resistance of a plant to a nematode plant pest (e.g., a soybean plant having increased resistance to infection by a soybean cyst nematode) by the introduction of a recombinant nucleic acid molecule of the invention into the plant cell, plant and/or plant part, thereby producing a transgenic plant cell, plant and/or plant part having increased resistance to the pest. This increase in resistance can be observed by comparing the resistance of the plant transformed with the recombinant nucleic acid molecule of the invention to the resistance of a plant lacking (i.e., not transformed with) the recombinant nucleic acid molecule of the invention. Thus, as used herein, the terms “increase,” “increasing,” “increased,” “enhance,” “enhanced,” “enhancing,” and “enhancement” (and grammatical variations thereof), and similar terms indicate an elevation of at least about 5%, 10%, 15%, 20%, 25%, 30%, 35%, 40%, 45%, 50%, 55%, 60%, 75%, 80%, 85%, 90%, 95%, 100%, 150%, 200%, 300%, 400%, 500% or more as compared to a control (e.g., a plant, plant part, plant cell that does not comprise at least one recombinant nucleic acid molecule of the invention).

As used herein, the terms “reduce,” “reduced,” “reducing,” “reduction,” “diminish,” “suppress,” and “decrease” (and grammatical variations thereof), describe, for example, a decrease in the growth of a nematode plant pest (e.g., soybean cyst nematode), a decrease in soybean cyst formation, a decrease in the number of mature female cyst nematodes, and/or a decrease in soybean cyst nematode cyst development on roots, a decrease in the ability of the nematode to survive, grow, feed, and/or reproduce, a decrease in the infectivity of a nematode plant pest, and/or a decrease in the infestation of a plant by a nematode plant pest, as compared to a control as described herein. Thus, as used herein, the terms “reduce,” “reduces,” “reduced,” “reduction,” “diminish,” “suppress,” and “decrease” and similar terms mean a decrease of at least about 5%, 10%, 15%, 20%, 25%, 30%, 35%, 40%, 45%, 50%, 55%, 60%, 75%, 80%, 85%, 90%, 95%, or 100% or more as compared to a control (e.g., a soybean plant that does not comprise at least one recombinant nucleic acid molecule of the invention). In particular embodiments, the reduction results in no or essentially no (i.e., an insignificant amount, e.g., less than about 10%, less than about 5% or even less than about 1%) detectable infection, cyst formation and/or cyst development.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or a functional untranslated RNA. A “functional” RNA includes any untranslated RNA that has a biological function in a cell, e.g., regulation of gene expression. Such functional RNAs include but are not limited to RNAi (e.g., siRNA, shRNA), miRNA, antisense RNA, anti-microRNA antisense oligodeoxyribonucleotide (AMO; see e.g., Lu et al. Nucleic Acids Res. 37(3):e24: 10.1093/nar/gkn1053), ribozymes, RNA aptamers and the like.

As used herein, “overexpress,” “overexpressed,” “overexpression” and the like, in reference to a polynucleotide means that the expression level of said polynucleotide is greater than that for the same polynucleotide in its native or wild type genetic context (e.g., in the same position in the genome and/or associated with the native/endogenous regulatory sequences). A nucleotide sequence can be overexpressed by inserting it into an overexpression vector. Such vectors are known in the art.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

Also as used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

A “portion” or “fragment” of a nucleotide sequence of the invention will be understood to mean a nucleotide sequence of reduced length relative to a reference nucleic acid or nucleotide sequence and comprising, consisting essentially of and/or consisting of a nucleotide sequence of contiguous nucleotides identical or almost identical (e.g., 80%, 81%, 82%, 83%, 84%, 85%, 86%, 87%, 88%, 89%, 90%, 91%, 92%, 93%, 94%, 95%, 96%, 98%, 99% identical) to the reference nucleic acid or nucleotide sequence. Such a nucleic acid fragment or portion according to the invention may be, where appropriate, included in a larger polynucleotide of which it is a constituent.

Thus, in some embodiments, a recombinant nucleic acid molecule of the invention can comprise, consist essentially of, or consist of a portion or fragment of a nucleotide sequence of the invention (e.g., SEQ ID NOs:1-97) for use, for example, in the overexpression of said nucleotide sequence on a soybean plant cell, soybean plant or soybean plant part. In some embodiments, a fragment of a nucleotide sequence of the invention comprises, consists essentially of, or consists of the nucleotide sequence between and encompassing the start and stop codons of any of the nucleotide sequences of SEQ ID NOs:1-97.

Thus, in some embodiments, exemplary fragments of nucleotide sequences of the invention can comprise, consist essentially of, consist of a fragment of the nucleotide sequence of SEQ ID NO:85 (A1) from nucleotide 160 to nucleotide 636; a fragment of the nucleotide sequence of SEQ ID NO:86 (A2) from nucleotide 231 to nucleotide 1193; a fragment of the nucleotide sequence of SEQ ID NO:79 (A3) from nucleotide 214 to nucleotide 960; a fragment of the nucleotide sequence of SEQ ID NO:92 (A4) from nucleotide 214 to nucleotide 1176; a fragment of the nucleotide sequence of SEQ ID NO:78 (A5) from nucleotide 193 to nucleotide 1011; a fragment of the nucleotide sequence of SEQ ID NO:56 (A6) from nucleotide 161 to nucleotide 835; a fragment of the nucleotide sequence of SEQ ID NO:12 (A7) from nucleotide 14 to nucleotide 688; a fragment of the nucleotide sequence of SEQ ID NO:6 (A8) from nucleotide 90 to nucleotide 704; a fragment of the nucleotide sequence of SEQ ID NO:37 (A9) from nucleotide 3 to nucleotide 962; a fragment of the nucleotide sequence of SEQ ID NO:24 (A10) from nucleotide 115 to nucleotide 1671; a fragment of the nucleotide sequence of SEQ ID NO:18 (A11) from nucleotide 54 to nucleotide 1673; a fragment of the nucleotide sequence of SEQ ID NO:1 (A12) from nucleotide 132 to nucleotide 2009; a fragment of the nucleotide sequence of SEQ ID NO:72 (A13) from nucleotide 43 to nucleotide 1572; a fragment of the nucleotide sequence of SEQ ID NO:31 (A15) from nucleotide 34 to nucleotide 594; a fragment of the nucleotide sequence of SEQ ID NO:87 (A18) from nucleotide 101 to nucleotide 1521; a fragment of the nucleotide sequence of SEQ ID NO:58 (A20) from nucleotide 159 to nucleotide 755; a fragment of the nucleotide sequence of SEQ ID NO:94 (A21) from nucleotide 162 to nucleotide 566; a fragment of the nucleotide sequence of SEQ ID NO:62 (A22) from nucleotide 130 to nucleotide 495; a fragment of the nucleotide sequence of SEQ ID NO:74 (A23) from nucleotide 131 to nucleotide 1120; a fragment of the nucleotide sequence of SEQ ID NO:45 (A24) from nucleotide 151 to nucleotide 987; a fragment of the nucleotide sequence of SEQ ID NO:3 (A25) from nucleotide 17 to nucleotide 587; a fragment of the nucleotide sequence of SEQ ID NO:73 (A26) from nucleotide 156 to nucleotide 424; and/or a fragment of the nucleotide sequence of SEQ ID NO:28 (A27) from nucleotide 39 to nucleotide 278.

In further embodiments of the invention, exemplary fragments of nucleotide sequences of the invention can comprise, consist essentially of, consist of a fragment of the nucleotide sequence of SEQ ID NO:5 (A30) from nucleotide 159 to nucleotide 1169; a fragment of the nucleotide sequence of SEQ ID NO:70 (A31) from nucleotide 187 to nucleotide 1821; a fragment of the nucleotide sequence of SEQ ID NO:96 (A32) from nucleotide 229 to nucleotide 1449; a fragment of the nucleotide sequence of SEQ ID NO:55 (A33) from nucleotide 194 to nucleotide 871; a fragment of the nucleotide sequence of SEQ ID NO:82 (A34) from nucleotide 184 to nucleotide 1245; a fragment of the nucleotide sequence of SEQ ID NO:11 (A35) from nucleotide 191 to nucleotide 1699; a fragment of the nucleotide sequence of SEQ ID NO:81 (A36) from nucleotide 189 to nucleotide 716; a fragment of the nucleotide sequence of SEQ ID NO:40 (A37) from nucleotide 218 to nucleotide 1825; a fragment of the nucleotide sequence of SEQ ID NO:2 (A38) from nucleotide 239 to nucleotide 790; a fragment of the nucleotide sequence of SEQ ID NO:59 (A39) from nucleotide 79 to nucleotide 717; a fragment of the nucleotide sequence of SEQ ID NO:36 (A42) from nucleotide 163 to nucleotide 1260; a fragment of the nucleotide sequence of SEQ ID NO:17 (A43) from nucleotide 260 to nucleotide 1081; a fragment of the nucleotide sequence of SEQ ID NO:42 (A44) from nucleotide 51 to nucleotide 392; a fragment of the nucleotide sequence of SEQ ID NO:53 (A45) from nucleotide 69 to nucleotide 2180; a fragment of the nucleotide sequence of SEQ ID NO:77 (A46) from nucleotide 94 to nucleotide 1110; a fragment of the nucleotide sequence of SEQ ID NO:68 (A48) from nucleotide 40 to nucleotide 1680; a fragment of the nucleotide sequence of SEQ ID NO:48 (A49) from nucleotide 30 to nucleotide 1328; a fragment of the nucleotide sequence of SEQ ID NO:83 (A50) from nucleotide 66 to nucleotide 743; a fragment of the nucleotide sequence of SEQ ID NO:39 (A51) from nucleotide 154 to nucleotide 762; a fragment of the nucleotide sequence of SEQ ID NO:27 (A52) from nucleotide 248 to nucleotide 1414; a fragment of the nucleotide sequence of SEQ ID NO:47 (A53) from nucleotide 245 to nucleotide 2383; a fragment of the nucleotide sequence of SEQ ID NO:41 (A60) from nucleotide 198 to nucleotide 1142; a fragment of the nucleotide sequence of SEQ ID NO:91 (A61) from nucleotide 169 to nucleotide 834; a fragment of the nucleotide sequence of SEQ ID NO:71 (A64) from nucleotide 171 to nucleotide 950; a fragment of the nucleotide sequence of SEQ ID NO:16 (A65) from nucleotide 140 to nucleotide 700; a fragment of the nucleotide sequence of SEQ ID NO:66 (A66) from nucleotide 186 to nucleotide 1247; a fragment of the nucleotide sequence of SEQ ID NO:35 (A67) from nucleotide 441 to nucleotide 1652; and/or a fragment of the nucleotide sequence of SEQ ID NO:57 (A68) from nucleotide 244 to nucleotide 1779.

In still further embodiments of the invention, exemplary fragments of nucleotide sequences of the invention can comprise, consist essentially of, consist of a fragment of the nucleotide sequence of SEQ ID NO:64 (C3) from nucleotide 3 to nucleotide 967, from nucleotide 15 to nucleotide 407, and/or from nucleotide 198 to nucleotide 733; a fragment of the nucleotide sequence of SEQ ID NO:76 (C6) from nucleotide 10 to nucleotide 489; a fragment of the nucleotide sequence of SEQ ID NO:38 (C7) from nucleotide 1 to nucleotide 744; a fragment of the nucleotide sequence of SEQ ID NO:84 (C8) from nucleotide 57 to nucleotide 758; a fragment of the nucleotide sequence of SEQ ID NO:13 (C9) from nucleotide 1 to nucleotide 723; a fragment of the nucleotide sequence of SEQ ID NO:20 (C12) from nucleotide 48 to nucleotide 989; a fragment of the nucleotide sequence of SEQ ID NO:29 (C13) from nucleotide 37 to nucleotide 1557; a fragment of the nucleotide sequence of SEQ ID NO:14 (C14) from nucleotide 95 to nucleotide 2056; a fragment of the nucleotide sequence of SEQ ID NO:67 (C15) from nucleotide 144 to nucleotide 590; a fragment of the nucleotide sequence of SEQ ID NO:23 (C16) from nucleotide 65 to nucleotide 1306; a fragment of the nucleotide sequence of SEQ ID NO:30 (C17) from nucleotide 80 to nucleotide 376; a fragment of the nucleotide sequence of SEQ ID NO:4 (C19) from nucleotide 85 to nucleotide 513; a fragment of the nucleotide sequence of SEQ ID NO:49 (C20) from nucleotide 22 to nucleotide 816 and/or from nucleotide 149 to nucleotide 848; a fragment of the nucleotide sequence of SEQ ID NO:10 (C21) from nucleotide 92 to nucleotide 842; a fragment of the nucleotide sequence of SEQ ID NO:93 (C22) from nucleotide 19 to nucleotide 1623; a fragment of the nucleotide sequence of SEQ ID NO:52 (C23) from nucleotide 65 to nucleotide 1666; a fragment of the nucleotide sequence of SEQ ID NO:34 (C27) from nucleotide 20 to nucleotide 538; a fragment of the nucleotide sequence of SEQ ID NO:15 (C28) from nucleotide 1 to nucleotide 954; a fragment of the nucleotide sequence of SEQ ID NO:7 (C29) from nucleotide 114 to nucleotide 923; and/or a fragment of the nucleotide sequence of SEQ ID NO:95 (C32) from nucleotide 51 to nucleotide 1346.

In other embodiments of the invention, exemplary fragments of nucleotide sequences of the invention can comprise, consist essentially of, consist of a fragment of the nucleotide sequence of SEQ ID NO:60 (C34) from nucleotide 235 to nucleotide 1098; a fragment of the nucleotide sequence of SEQ ID NO:75 (C36) from nucleotide 63 to nucleotide 1418; a fragment of the nucleotide sequence of SEQ ID NO:22 (C37) from nucleotide 287 to nucleotide 976; a fragment of the nucleotide sequence of SEQ ID NO:61 (C39) from nucleotide 1 to nucleotide 321; a fragment of the nucleotide sequence of SEQ ID NO:33 (C40) from nucleotide 71 to nucleotide 901; a fragment of the nucleotide sequence of SEQ ID NO:26 (C42) from nucleotide 1 to nucleotide 381; a fragment of the nucleotide sequence of SEQ ID NO:21 (C43) from nucleotide 61 to nucleotide 1806; a fragment of the nucleotide sequence of SEQ ID NO:51 (C45) from nucleotide 99 to nucleotide 869; a fragment of the nucleotide sequence of SEQ ID NO:46 (C49) from nucleotide 149 to nucleotide 796; a fragment of the nucleotide sequence of SEQ ID NO:32 (C52) from nucleotide 100 to nucleotide 1038; a fragment of the nucleotide sequence of SEQ ID NO:54 (C53) from nucleotide 75 to nucleotide 698; a fragment of the nucleotide sequence of SEQ ID NO:88 (C55) from nucleotide 20 to nucleotide 694; a fragment of the nucleotide sequence of SEQ ID NO:50 (K1) from nucleotide 1 to nucleotide 1110; a fragment of the nucleotide sequence of SEQ ID NO:63 (R3) from nucleotide 186 to nucleotide 536; a fragment of the nucleotide sequence of SEQ ID NO:80 (R4) from nucleotide 23 to nucleotide 745; a fragment of the nucleotide sequence of SEQ ID NO:43 (R5) from nucleotide 1 to nucleotide 351; a fragment of the nucleotide sequence of SEQ ID NO:65 (R7) from nucleotide 178 to nucleotide 921 and/or from nucleotide 1 to nucleotide 921; a fragment of the nucleotide sequence of SEQ ID NO:9 (R8) from nucleotide 248 to nucleotide 871; a fragment of the nucleotide sequence of SEQ ID NO:25 (R24) from nucleotide 143 to nucleotide 826; a fragment of the nucleotide sequence of SEQ ID NO:90 (R25) from nucleotide 79 to nucleotide 345; a fragment of the nucleotide sequence of SEQ ID NO:69 (R27) from nucleotide 60 to nucleotide 1094; a fragment of the nucleotide sequence of SEQ ID NO:89 (R28) from nucleotide 65 to nucleotide 988; a fragment of the nucleotide sequence of SEQ ID NO:44 (R29) from nucleotide 61 to nucleotide 1464; a fragment of the nucleotide sequence of SEQ ID NO:97 (R30) from nucleotide 41 to nucleotide 2149; a fragment of the nucleotide sequence of SEQ ID NO:19 (R48) from nucleotide 318 to nucleotide 1419, from nucleotide 217 to nucleotide 1419, or from nucleotide 1 to nucleotide 1419; and/or a fragment of the nucleotide sequence of SEQ ID NO:8 (Si) from nucleotide 41 to nucleotide 490.

Accordingly, in particular aspects of the invention, a recombinant nucleic acid molecule comprising, consisting essentially of or consisting of one or more nucleotide sequences of SEQ ID NOs:1-97, or a fragment or portion thereof as described herein, can be introduced into a soybean plant cell, soybean plant and soybean plant part and overexpressed in said soybean cell, soybean plant and soybean plant part, thereby producing a soybean cell, soybean plant and soybean plant part having increased resistance to infection by a soybean cyst nematode, having reduced soybean cyst formation and/or having reduced soybean cyst nematode cyst development on roots as compared to a soybean plant, plant part or plan cell that does not comprise said recombinant nucleic acid molecule.

In other embodiments, the invention provides one or more double stranded RNA (dsRNA) molecules that comprise, consist essentially of, or consist of at least 18 consecutive nucleotides of a nucleotide sequence of any of the nucleotide sequences of SEQ ID NO: 1-97, and the reverse complement thereof. Thus, in some embodiments, a dsRNA molecule comprises, consists of, or consists essentially of a fragment or a portion of a nucleotide sequence of this invention (e.g., any of SEQ ID NOs:1-97) that is at least about 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 325, 350, 375, 400, 425, 450, 475, 500, 525, 550, 575 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500 consecutive nucleotides in length, and the like, and any range therein, of any of SEQ ID NOs:1-97, and the reverse complement thereof. Thus, in some embodiments of the invention, a portion of a nucleotide sequence of this invention can be at least about 18 nucleotides in length. In further embodiments, the dsRNA can comprise the full length cDNA of any of the nucleotide sequences of the invention (e.g., SEQ ID NOs: 1 to 97) plus the untranslated regions at both the 5′ and 3′ ends.

Thus, dsRNA molecules can be designed using one or more of the nucleotide sequences of the invention for specific silencing of the expression of the nucleotide sequence from which each dsRNA is designed. Methods for designing dsRNA molecules are well known in the art. See, for example, March et al. Methods in Molecular Biology 388:427-433 (2007); RNAi Technology, Enfield and Gaur, eds. CRC Press (2011); and RNA Interference Techniques, S. Harper, ed., Humana Press, New York (2011).

Thus, in some aspects of the invention, a recombinant nucleic acid molecule comprising one or more nucleotide sequences encoding one or more dsRNA molecules, and the reverse complements thereof, can be introduced into a soybean plant cell, plant or plant part, thereby producing a soybean cell, soybean plant and soybean plant part having increased resistance to infection by a soybean cyst nematode, having reduced soybean cyst formation and/or having reduced soybean cyst nematode cyst development on roots as compared to a soybean plant, plant part or plan cell that does not comprise said recombinant nucleic acid molecule

The invention further provides a nucleotide sequence that encodes a portion of (e.g., 18 or more consecutive nucleotides) of a nucleotide sequence of any of SEQ ID NOs:1-97, which when expressed produces an antisense nucleotide sequence. Any antisense nucleotide sequence as known in the art useful with this invention can be employed in the methods described herein. Thus, for example, the nucleotide sequences of any of SEQ ID NOs:1-97 or fragments thereof, also can be used to make antisense nucleotide sequences such as microRNAs and/or anti-microRNA antisense oligodeoxyribonucleotide (AMO) inhibitors as described in, for example, Lu et al. (Nucleic Acids Res. 37(3):e24: 10.1093/nar/gkn1053).

Accordingly, in representative embodiments, a nucleotide sequence encoding a portion or fragment (e.g., consecutive nucleotides) of a nucleotide sequence of this invention, which when expressed produces an antisense nucleotide sequence, can comprise, consist essentially of, or consist of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 325, 350, 375, 400, 425, 450, 475, 500, 525, 550, 575 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500 or more consecutive nucleotides of any of SEQ ID NOs:1-97.

It is to be understood that additional nucleotides can be added at the 3′ end, the 5′ end or both the 3′ and 5′ ends to facilitate manipulation of the antisense nucleotide sequence but that do not materially affect the basic characteristics or function of the antisense nucleotide sequence molecule in RNA interference (RNAi). Such additional nucleotides can be nucleotides that extend the complementarity of the antisense nucleotide sequence along the target sequence and/or such nucleotides can be nucleotides that facilitate manipulation of the antisense nucleotide sequence or a nucleic acid molecule encoding the antisense nucleotide sequence, as would be known to one of ordinary skill in the art. For example, a TT overhang at the 3′ end can be present, which is used to stabilize a siRNA duplex and does not affect the specificity of the siRNA.

Thus, in some aspects of the invention, a recombinant nucleic acid molecule comprising one or more nucleotide sequences encoding a portion of consecutive nucleotides (e.g., 18 nucleotides or more) of a nucleotide sequence of any of SEQ ID NOs:1-97, which when expressed produces an antisense nucleotide sequence, can be introduced into a soybean plant cell, plant or plant part, thereby producing a soybean cell, soybean plant and soybean plant part having increased resistance to infection by a soybean cyst nematode, having reduced soybean cyst formation and/or having reduced soybean cyst nematode cyst development on roots as compared to a soybean plant, plant part or plan cell that does not comprise said recombinant nucleic acid molecule

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to said nucleotide sequence of the invention.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 70%, least about 75%, at least about 80%, least about 85%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 150 residues in length. Thus, in some embodiments of the invention, the substantial identity exists over a region of the sequences that is at least about 16, at least about 18, at least about 22, at least about 25, at least about 30, at least about 40, at least about 50, about 60, about 70, about 80, about 90, about 100, about 110, about 120, about 130, about 140, about 150, or more residues in length, and any range therein. In representative embodiments, the sequences can be substantially identical over at least about 22 nucleotides. In some particular embodiments, the sequences are substantially identical over at least about 150 residues. In some embodiments, sequences of the invention can be about 85% to about 100% identical over at least about 16 nucleotides to about 22 nucleotides. In other embodiments, the sequences can be about 85% identical over about 22 nucleotides. In still other embodiments, the sequences can be 100% homologous over about 16 nucleotides. In a further embodiment, the sequences are substantially identical over the entire length of the coding regions. Furthermore, in representative embodiments, substantially identical nucleotide or protein sequences perform substantially the same function (e.g., conferring increased resistance to a nematode plant pest, reducing the growth of a nematode plant pest, reducing nematode cyst development).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially identical when the two sequences hybridize to each other under stringent conditions. In some representative embodiments, two nucleotide sequences considered to be substantially identical hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology—Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

In particular embodiments, a further indication that two nucleotide sequences or two polypeptide sequences are substantially identical can be that the protein encoded by the first nucleic acid is immunologically cross reactive with, or specifically binds to, the protein encoded by the second nucleic acid. Thus, in some embodiments, a polypeptide can be substantially identical to a second polypeptide, for example, where the two polypeptides differ only by conservative substitutions.

In some embodiments, the recombinant nucleic acids molecules, nucleotide sequences and polypeptides of the invention are “isolated.” An “isolated” nucleic acid molecule, an “isolated” nucleotide sequence or an “isolated” polypeptide is a nucleic acid molecule, nucleotide sequence or polypeptide that, by the hand of man, exists apart from its native environment and is therefore not a product of nature. An isolated nucleic acid molecule, nucleotide sequence or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

In other embodiments, an isolated nucleic acid molecule, nucleotide sequence or polypeptide may exist in a non-native environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the recombinant nucleic acid molecules, nucleotide sequences and their encoded polypeptides are “isolated” in that, by the hand of man, they exist apart from their native environment and therefore are not products of nature, however, in some embodiments, they can be introduced into and exist in a recombinant host cell.

In some embodiments, the nucleotide sequences and/or recombinant nucleic acid molecules of the invention can be operatively associated with a variety of promoters for expression in soybean plant cells. Thus, in representative embodiments, a recombinant nucleic acid of this invention can further comprise one or more promoters operably linked to one or more nucleotide sequences.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence, means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

A “promoter” is a nucleotide sequence that controls or regulates the transcription of a nucleotide sequence (i.e., a coding sequence) that is operably associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” In particular aspects, a “promoter” useful with the invention is a promoter capable of initiating transcription of a nucleotide sequence in a cell of a soybean plant.

The choice of promoter will vary depending on the temporal and spatial requirements for expression, and also depending on the host cell to be transformed. Thus, for example, expression of the nucleotide sequences of the invention can be in any plant and/or plant part, (e.g., in leaves, in stalks or stems, in ears, in inflorescences (e.g. spikes, panicles, cobs, etc.), in roots, seeds and/or seedlings, and the like). For example, where expression in a specific tissue or organ is desired, a tissue-specific or tissue preferred promoter can be used (e.g., a root specific/preferred promoter). In contrast, where expression in response to a stimulus is desired a promoter inducible by stimuli or chemicals can be used. Where continuous expression at a relatively constant level is desired throughout the cells of a plant a constitutive promoter can be chosen. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

Promoters useful with the invention include, but are not limited to, those that drive expression of a nucleotide sequence constitutively, those that drive expression when induced, and those that drive expression in a tissue- or developmentally-specific manner. These various types of promoters are known in the art.

Examples of constitutive promoters include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087, all incorporated by reference

Additional examples of tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). In some particular embodiments, the nucleotide sequences of the invention are operably associated with a root-preferred promoter.

Particularly useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in a plant through the application of an exogenous chemical regulator. Regulation of the expression of nucleotide sequences of the invention via promoters that are chemically regulated enables the polypeptides of the invention to be synthesized only when the crop plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid (e.g., the PR1a system), steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

In further aspects, the nucleotide sequences of the invention can be operably associated with a promoter that is wound inducible or inducible by pest or pathogen infection (e.g., a nematode plant pest). Numerous promoters have been described which are expressed at wound sites and/or at the sites of pest attack (e.g., insect/nematode feeding) or phytopathogen infection. Ideally, such a promoter should be active only locally at or adjacent to the sites of attack, and in this way expression of the nucleotide sequences of the invention will be focused in the cells that are being invaded. Such promoters include, but are not limited to, those described by Stanford et al., Mol. Gen. Genet. 215:200-208 (1989), Xu et al. Plant Molec. Biol. 22:573-588 (1993), Logemann et al. Plant Cell 1:151-158 (1989), Rohrmeier and Lehle, Plant Molec. Biol. 22:783-792 (1993), Firek et al. Plant Molec. Biol. 22:129-142 (1993), Warner et al. Plant J. 3:191-201 (1993), U.S. Pat. No. 5,750,386, U.S. Pat. No. 5,955,646, U.S. Pat. No. 6,262,344, U.S. Pat. No. 6,395,963, U.S. Pat. No. 6,703,541, U.S. Pat. No. 7,078,589, U.S. Pat. No. 7,196,247, U.S. Pat. No. 7,223,901, and U.S. Patent Application Publication 2010043102.

In some embodiments, a recombinant nucleic acid molecule of the invention can be an “expression cassette” or can be comprised within an expression cassette. As used herein, “expression cassette” means a recombinant nucleic acid molecule comprising a nucleotide sequence of interest (e.g., the nucleotide sequences of the invention), wherein said nucleotide sequence is operably associated with at least a control sequence (e.g., a promoter). Thus, some embodiments of the invention provide expression cassettes designed to express the nucleotides sequences of the invention. In this manner, for example, one or more plant promoters operably associated with one or more nucleotide sequences of the invention (e.g., SEQ ID NOs:1-97, and/or portions or fragments thereof) are provided in expression cassettes for expression in a soybean plant, plant part and/or plant cell.

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used.

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette of the invention.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac,” pp. 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin, which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in an expression cassette of the invention.

An expression cassette of the invention also can include polynucleotides that encode other desired traits. Such desired traits can be other polynucleotides which confer nematode resistance, or which confer insect resistance, or other agriculturally desirable traits. Such polynucleotides can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

Thus, an expression cassette can include a coding sequence for one or more polypeptides for agronomic traits that primarily are of benefit to a seed company, grower or grain processor. A polypeptide of interest can be any polypeptide encoded by a polynucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431. Thus, in some embodiments, the expression cassette or expression vector of the invention can comprise one or more polynucleotide sequences that confer insect resistance and/or additional nematode resistance. Polynucleotides that confer insect resistance include, but are not limited to, polynucleotides coding for Bacillus thuringiensis (Bt) toxins, for example, the various delta-endotoxin genes such as Cry1Aa, Cry1Ab, Cry1Ac, Cry1B, Cry1C, Cry1D, Cry1Ea, Cry1Fa, Cry3A, Cry9A, Cry9C and Cry9B; as well as genes encoding vegetative insecticidal proteins such as Vip1, Vip2 and Vip3). An extensive list of Bt toxins can be found on the worldwide web at Bacillus thuringiensis Toxin Nomenclature Database maintained by the University of Sussex (see also, Crickmore et al. (1998) Microbiol. Mol. Biol. Rev. 62:807-813).

In other embodiments, a polypeptide of interest also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956. See also, on the World Wide Web at lifesci.sussex.ac.uk/home/Neil_Crickmore/Bt/.

In addition to expression cassettes, the nucleic acid molecules and nucleotide sequences described herein can be used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Vectors for use in transformation of plants and other organisms are well known in the art. Non-limiting examples of general classes of vectors include a viral vector including but not limited to a plasmid vector, a phage vector, a phagemid vector, a cosmid vector, a fosmid vector, a bacteriophage, an artificial chromosome, or an Agrobacterium binary vector in double or single stranded linear or circular form which may or may not be self transmissible or mobilizable. A vector as defined herein can transform prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some representative embodiments, the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

A non-limiting example of a vector is the plasmid pBI101 derived from the Agrobacterium tumefaciens binary vector pBIN19 allows cloning and testing of promoters using .beta.-glucuronidase (GUS) expression signal (Jefferson et al, 1987, EMBO J. 6: 3901-3907). The size of the vector is 12.2 kb. It has a low-copy RK2 origin of replication and confers kanamycine resistance in both bacteria and plants. There are numerous other expression vectors known to the person skilled in the art that can be used according to the invention. Further non-limiting examples of vectors include pBIN19 (Bevan, Nucl. Acids Res. (1984)), the binary vectors pCIB200 and pCIB2001 for use with Agrobacterium, the construction of which is disclosed, for example, in WO 95133818 (example 35) (see also EP 0 332 104, example 19), the binary vector pCIB10, which contains a gene encoding kanamycin resistance for selection, the wide host-range plasmid pRK252, the construction of which is described by Rothstein et al. (Gene 53: 153-161 (1987)). Various derivatives of pCIB10 have been constructed which incorporate the gene for hygromycin B phosphotransferase are described by Gritzret al. (Gene 25:179-188 (1983)). These derivatives enable selection of transgenic plant cells on hygromycin only (pCIB743), or hygromycin and kanamycin (pCIB715, pCIB717).

An additional example of a vector useful for direct gene transfer techniques in combination with selection by the herbicide Basta (or phosphinothricin) is pCIB3064. This vector is based on the plasmid pCIB246, which comprises the CaMV 35S promoter in operational fusion to the E. coli GUS gene and the CaMV 35S transcriptional terminator and is described in the PCT published application WO 93/07278. An additional transformation vector is pSOG35 which utilizes the E. coli gene dihydrofolate reductase (DHFR) as a selectable marker conferring resistance to methotrexate and the construction of which is described, for example, in WO 95/33818. Another transformation vector is the vector pGL2 (Shimamoto et al. Nature 338, 274-276 (1989)) which contains the Streptomyces hygromycin phosphotransferase gene (hpt) operably linked to the 35S promoter and 35S terminator sequences.

Thus, numerous transformation vectors are available for plant transformation, and the expression cassettes of this invention can be used in conjunction with any such vectors. The selection of vector will depend upon the preferred transformation technique and the target species for transformation. Accordingly, in further embodiments, a recombinant nucleic acid molecule of the invention can be comprised within a recombinant vector. The size of a vector can vary considerably depending on whether the vector comprises one or multiple expression cassettes (e.g., for molecular stacking). Thus, a vector size can range from about 3 kb to about 30 kb. Thus, in some embodiments, a vector is about 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, or any range therein, in size. In some particular embodiments, a vector can be about 3 kb to about 10 kb in size.

In additional embodiments of the invention, a method of producing a transgenic plant cell is provided, said method comprising introducing into a plant cell a recombinant nucleic acid molecule/nucleotide sequence of the invention (e.g., SEQ ID NOs:1-97, and/or portions or fragments thereof), thereby producing a transgenic plant cell that can regenerate a transgenic plant or plant part having increased resistance to infection by a soybean cyst nematode, having reduced soybean cyst formation and/or having reduced soybean cyst nematode cyst development on roots as compared to a plant regenerated from a plant cell that does not comprise said recombinant nucleic acid molecule. In some embodiments, the transgenic plant cell comprises more than one (e.g., 2, 3, 4, 5, 6, 7, 8, 9, 10, etc.) recombinant nucleic acid molecule/nucleotide sequence of the invention. Thus, in some aspects of the invention, the transgenic plants, or parts thereof, comprise and express one or more nucleic acid molecules/nucleotide sequences of the invention, thereby producing one or more polypeptides and/or dsRNAs and/or antisense molecules of the invention.

In representative embodiments, a method of producing a transgenic plant cell is provided, said method comprising introducing into a soybean plant cell a recombinant nucleic acid molecule of the invention, said recombinant nucleic acid molecule comprising, consisting essentially of, or consisting of a nucleotide sequence of the invention operably linked to a heterologous promoter, which when expressed in a plant confers increased resistance to infection by a soybean cyst nematode, reduced soybean cyst formation and/or reduced soybean cyst nematode cyst development on roots, the nucleotide sequence comprising, consisting essentially of, or consisting of: (a) a nucleotide sequence of any of SEQ ID NOs:1-97, or a fragment thereof; (b) a nucleotide sequence encoding a double stranded RNA molecule comprising, consisting essentially of, or consisting of at least 18 consecutive nucleotides of a nucleotide sequence of any of SEQ ID NOs: 1-97 and the reverse-complement thereof; (c) a nucleotide sequence encoding a portion of any of SEQ ID NOs:1-97, which when expressed produces an antisense nucleotide sequence; (d) a nucleotide sequence having substantial sequence identity (e.g., 80%, 85%, 90%, 95%, 96%, 97%, 98%, 99%) to a nucleotide sequence of (a), (b) or (c) above; (e) a nucleotide sequence which anneals under stringent hybridization conditions to the nucleotide sequence of (a), (b), (c) or (d) above; (e) a nucleotide sequence that differs from the nucleotide sequences of (a), (b), (c), (d) or (e) above due to the degeneracy of the genetic code; or (f) any combination of the nucleotide sequences of (a)-(e), thereby producing a transgenic plant cell that can regenerate a plant having increased resistance to infection by a soybean cyst nematode, having reduced soybean cyst formation and/or having reduced soybean cyst nematode cyst development on roots as compared to a soybean plant regenerated from a soybean plant cell that does not comprise said recombinant nucleic acid molecule.

Thus, in some embodiments, the invention provides a transgenic soybean plant or plant part that is regenerated from the transgenic plant cell of the invention, wherein said transgenic plant or plant part comprises in its genome one or more recombinant nucleic acid molecules/nucleotide sequences of the invention and has increased resistance to infection by a soybean cyst nematode, having reduced soybean cyst formation and/or having reduced soybean cyst nematode cyst development on roots as compared to a control plant or plant part that is regenerated from a plant cell that does not comprise said recombinant nucleic acid molecule.

Thus, in some representative embodiments, the invention provides a transgenic soybean plant, soybean plant part or soybean plant cell comprising a recombinant nucleic acid molecule comprising one or more nucleotide sequences selected from the group consisting of: (a) a nucleotide sequence of any of SEQ ID NOs:1-97, or a fragment thereof; (b) a nucleotide sequence encoding a double stranded RNA molecule comprising at least 18 consecutive nucleotides of a nucleotide sequence of any of SEQ ID NOs:1-97 and the reverse-complement thereof; (c) a nucleotide sequence encoding a portion of a nucleotide sequence of any of SEQ ID NOs:1-97, which when expressed produces an antisense nucleotide sequence; and (d) any combination of (a)-(c), wherein said transgenic soybean plant, soybean plant part or soybean plant cell has increased resistance to infection by a soybean cyst nematode, reduced soybean cyst nematode cyst formation and/or reduced soybean cyst nematode cyst development on roots. In some embodiments, the one or more nucleotide sequences of any of SEQ ID NOs:1-97, or a fragment thereof, are overexpressed in said transgenic soybean plant, plant part and/or plant cell.

As used herein, the term “plant part” includes but is not limited to embryos, pollen, ovules, seeds, leaves, flowers, branches, fruit, stalks, roots, root tips, anthers, and/or plant cells including plant cells that are intact in plants and/or parts of plants, plant protoplasts, plant tissues, plant cell tissue cultures, plant calli, plant clumps, and the like. Further, as used herein, “plant cell” refers to a structural and physiological unit of the plant, which comprises a cell wall and also may refer to a protoplast. A plant cell of the invention can be in the form of an isolated single cell or can be a cultured cell or can be a part of a higher-organized unit such as, for example, a plant tissue or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like.

In some particular embodiments, the invention provides a transgenic seed produced from a transgenic plant of the invention, wherein the transgenic seed comprises a nucleic acid molecule/nucleotide sequence of the invention (e.g., SEQ ID NOs:1-97, and/or portions or fragments thereof).

“Plant cell culture” means cultures of plant units such as, for example, protoplasts, cell culture cells, cells in plant tissues, pollen, pollen tubes, ovules, embryo sacs, zygotes and embryos at various stages of development. In some embodiments of the invention, a transgenic tissue culture or transgenic plant cell culture is provided, wherein the transgenic tissue or cell culture comprises a nucleic acid molecule/nucleotide sequence of the invention.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

Additional aspects of the invention include a harvested product produced from the transgenic soybean cells, soybean plants and/or soybean plant parts of the invention, as well as a processed product produced from said harvested product. A harvested product can be a whole soybean plant or any soybean plant part, as described herein, wherein said harvested product comprises a recombinant nucleic acid molecule/nucleotide sequence of the invention. Thus, in some embodiments, a non-limiting example of a harvested product includes a seed, a fruit, a flower or part thereof (e.g., an anther, a stigma, and the like), a leaf, a stem, and the like. In other embodiments, a processed product includes, but is not limited to, a flour, meal, oil, starch, cereal, and the like produced from a harvested seed or other part of a transformed soybean plant of the invention, wherein said transformed seed, plant or plant part comprises in its genome a recombinant nucleic acid molecule/nucleotide sequence of the invention.

“Introducing,” in the context of a polynucleotide of interest (e.g., the nucleotide sequences and recombinant nucleic acid molecules of the invention; e.g., SEQ ID NOs:1-97, and/or portions or fragments thereof), means presenting the nucleotide sequence of interest to the plant, plant part, and/or plant cell in such a manner that the nucleotide sequence gains access to the interior of a cell. Where more than one nucleotide sequence is to be introduced these nucleotide sequences can be assembled as part of a single polynucleotide or nucleic acid construct, or as separate polynucleotide or nucleic acid constructs, and can be located on the same or different expression constructs or transformation vectors. Accordingly, these polynucleotides can be introduced into plant cells in a single transformation event, in separate transformation events, or, for example, they can be incorporated into a plant as part of a breeding protocol.

The term “transformation” as used herein refers to the introduction of a heterologous nucleic acid into a cell. Transformation of a cell may be stable or transient. Thus, in some embodiments, a plant cell of the invention is stably transformed with a nucleic acid molecule of the invention. In other embodiments, a plant of the invention is transiently transformed with a recombinant nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

A polynucleotide of the invention (e.g., any one of SEQ ID NOs:1-97, and/or portions or fragments thereof, and/or any combination thereof) can be introduced into a cell by any method known to those of skill in the art. In some embodiments of the invention, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hofgen & Willmitzer (1988) Nucleic Acids Res. 16:9877).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and/or plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more polynucleotides sought to be introduced) also can be propelled into plant tissue.

Thus, in particular embodiments of the invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

Likewise, the genetic properties engineered into the transgenic seeds and plants, plant parts, and/or plant cells of the invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the plant, plant part and/or plant cell in any number of ways that are well known in the art. The methods of the invention do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, or in separate transformation events, or, alternatively, a nucleotide sequence can be incorporated into a plant, as part of a breeding protocol.

Thus, in additional embodiments, the invention provides a method of producing a soybean plant having increased resistance to infection by a soybean cyst nematode, having reduced soybean cyst formation and/or having reduced soybean cyst nematode cyst development on roots, the method comprising the steps of (a) crossing a transgenic plant of the invention with itself or another plant to produce seed comprising a recombinant nucleic acid molecule of the invention; and (b) growing a progeny plant from said seed to produce a plant having increased resistance to infection by a soybean cyst nematode. In some embodiments, the method further comprises (c) crossing the progeny plant of (b) with itself or another plant and (d) repeating steps (b) and (c) for an additional 0-7 (e.g., 0, 1, 2, 3, 4, 5, 6, 7) generations to produce a plant having increased resistance to infection by a soybean cyst nematode, having reduced soybean cyst formation and/or having reduced soybean cyst nematode cyst development on roots.

In further embodiments, a method of producing a soybean plant having increased resistance to infection by a soybean cyst nematode, having reduced soybean cyst formation and/or having reduced soybean cyst nematode cyst development on roots, is provided, the method comprising the steps of (a) crossing a transgenic soybean plant of the invention with itself or another soybean plant to produce soybean seed comprising a recombinant nucleic acid molecule of the invention; and (b) growing a progeny soybean plant from said seed to produce a soybean plant having increased resistance to infection by a soybean cyst nematode. In some embodiments, the method further comprises (c) crossing the progeny soybean plant of (b) with itself or another soybean plant and (d) repeating steps (b) and (c) for an additional 0-7 (e.g., 0, 1, 2, 3, 4, 5, 6, 7) generations to produce a soybean plant having increased resistance to infection by a soybean cyst nematode, having reduced soybean cyst formation and/or having reduced soybean cyst nematode cyst development on roots as compared to a control soybean plant.

The invention further provides a plant crop comprising a plurality of transgenic soybean plants of the invention planted together in an agricultural field.

In addition, the invention provides a method of improving the yield of a soybean plant crop when said soybean plant crop is contacted with a soybean cyst nematode plant pest, the method comprising cultivating a plurality of soybean plants comprising a recombinant nucleic acid molecule of the invention as the soybean plant crop, wherein the plurality of plants of said soybean plant crop have increased resistance to soybean cyst nematode infection, have reduced soybean cyst formation and/or have reduced soybean cyst nematode cyst development on roots, thereby improving the yield of said soybean crop when contacted with a soybean cyst nematode plant pest as compared to a control soybean crop contacted with said soybean cyst nematode plant pest, wherein the control soybean crop is produced from a plurality of soybean plants lacking said nucleic acid molecule.

Thus, in some embodiments, the invention provides a method of controlling a soybean cyst nematode, comprising contacting the soybean cyst nematode with a transgenic plant and/or a part thereof comprising a recombinant nucleic acid molecule of the invention, thereby controlling the soybean cyst nematode as compared to the control of a soybean cyst nematode contacted with a control plant or plant part, said control plant lacking said recombinant nucleic acid molecule.

To “contact” a nematode plant pest with a polypeptide or polynucleotide of the invention and/or composition thereof or to “deliver” to a nematode plant pest a polypeptide or polynucleotide of the invention and/or composition thereof means that the nematode plant pest comes into contact with, is exposed to, the polypeptides and/or polynucleotides of this invention, resulting in a toxic effect on and control of the soybean cyst nematode (e.g., control, increase resistance, reduced infectivity, reduced infestation, reduced cyst formation, reduced growth, and the like). A soybean cyst nematode can be contacted with a polypeptide of the invention or nematicidal composition of the invention using any art known method. For example, contacting includes providing the polypeptide(s)/polynucleotides of the invention in a transgenic plant, wherein the nematode eats (ingests) one or more parts of the transgenic plant, any other art-recognized delivery system.

“Effective amount” refers to that concentration or amount of a polypeptide, polynucleotide, or nematicidal composition that inhibits or reduces the ability of a nematode plant pest to survive, grow, feed and/or reproduce, or that limits nematode-related damage or loss in crop plants. Thus, in some embodiments of the invention, an “effective amount” can mean killing the nematode. In other embodiments, an “effective amount” does not mean killing the nematode. In some embodiments, the nematode does not come into contact with the polypeptide, polynucleotide, or nematicidal composition. Instead, the polypeptide, polynucleotide, or nematicidal composition may stop cell changes that allow the nematode to feed. For example, the polypeptide, polynucleotide, or nematicidal composition may stop nutrient flow to the nematode, kill the feeding cell, or modify the soybean cell in such a way as to delay or stop soybean cyst nematode growth and development. Thus, in some embodiments, “effective amount” can refer to a concentration or amount of the polypeptide, polynucleotide, or nematicidal composition that can alter the host (soybean) cell to delay or reduce nematode development or kill the nematode, due to modifications to the host cell by said polypeptide, polynucleotide, or nematicidal composition.

The term “control” in the context of an effect on a soybean cyst nematode means to inhibit or reduce, through a toxic effect, the ability of the organism to survive, grow, feed, and/or reproduce, or to limit damage or loss in crop plants that is related to the activity of the soybean cyst nematode. To “control” a soybean cyst nematode may or may not mean killing the soybean cyst nematode, although in some embodiments “control” means killing the nematode.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### Examples

### Example 1

### Bioinformatics

Genes were selected from published gene expression studies of the interaction of soybean roots with SCN over time using the Affymetrix microarray platform (Klink et al. (2007) Planta 226: 1389-1409; Klink et al. (2007) Planta 226: 1423-1447; Klink et al. (2009) Plant Molecular Biology 71:525-567; Klink et al. (2009) Plant Physiol. 151:1017-1022) and Ithal et al. (Molec Plant Path Interact 20:293-305 (2007)). The GenBank number associated with the gene expression data was used to obtain full length open reading frames of the genes either by building contigs from expressed sequence tags (ESTs) found in GenBank or by blasting the DNA or predicted protein sequence against soybean genome database found at Phytozome.net (Joint Genome Institute, U.S.D.O.E.; Center for Integrative Genomics, U.C. Berkeley). Primers for PCR amplification of the open reading frame were designed using Primer 3 (biotools.umassmed.edu/bioapps/primer3_www.cgi; See, Table 1) and OligoAnalyzer 3.1 (Integrated DNA Technologies, Coralville, Iowa). DNA sequences within 2000 nt of the ATG start site of genes were obtained from the Glycine max genome database found at Phytosome.net.

TABLE 1
Primers used in PCR amplification and sequencing.

| | | Amplicon |
|---|---|---|
| Primer | Sequence | size |
| M13-F | 5′-GTAAAACGACGGCCAG-3′ (SEQ ID NO: 195) | — |
| M13-R | 5′-CAGGAAACAGCTATGAC-3′ (SEQ ID NO: 196) | |
| FMV-F | 5′-AAGAAGCCCTCCAGCTTCAAAG-3′ (SEQ ID NO: 197) | |
| eGFP-F | 5′-ATGGTGAGCAAGGGCGAGGAGC-3′ (SEQ ID NO: 198) | 706 bp |
| eGFP-R | 5′-TCGTCCATGCCGAGAGTGATCCCG-3′ (SEQ ID NO: 199) | |
| Rᵢ-F | 5′-TCAGCCTCCCCGCCGGATG-3′ (SEQ ID NO: 200) | 812 bp |
| Rᵢ-R | 5′-ATGCAAAAGACAGGATTGATCGCA-3′ (SEQ ID NO: 201) | |

### Example 2

### Amplification and Cloning of Open Reading Frames (ORFs)

The ORFs of target genes were cloned using the Gateway® (Invitrogen, Carlsbad, Calif.) system. ORFs were amplified from template cDNA using cDNA libraries previously reported (Heinz et al. 1998; Khan et al. 2004), representing RNA from the SCN-resistant soybean cultivar ‘Peking’ 3 days after infection (dai) with SCN NH1-RHp (also known as race 3). The Heinz UniZap Library was made from roots and shoots of resistant soybean cultivar Glycine max ‘Peking’ 2-3 dai with SCN race 3. The Khan TriplExZ library was made from roots only of the resistant soybean cultivar Glycine max ‘Peking’ 2-4 dai with SCN race 3. ORFs were amplified using gene-specific primers containing CACC at the 5′end of the forward primer, which is necessary for directional cloning using the Gateway® (Invitrogen) system. The 50-μL PCR reaction used 2 μL of cDNA library template and 1 unit of Platinum® Taq Polymerase High Fidelity (Invitrogen) according to the manufacturer's instructions. Cycling conditions were as follows: an initial denaturation step of 94° C. for 2 min; 35 cycles of 94° C. for 45 sec, gene-specific primer Tₘ for 30 sec, and 68° C. for 1 min per kb of amplicon; and a final extension step of 68° C. for 25 min.

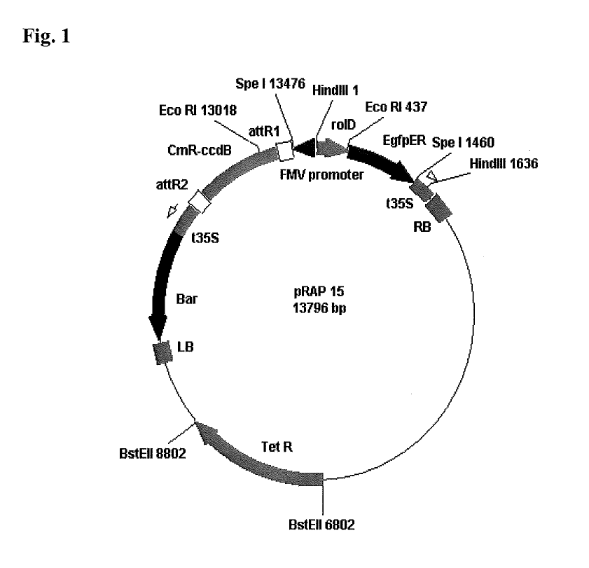

The PCR amplicons were gel-purified on 0.8% agarose gels stained with Syber® Safe DNA gel stain using the E-Gel system (Invitrogen) and cloned into pENTR using a pENTR™ Directional TOPO® Cloning Kit (Invitrogen) and transformed into competent Escherichia coli cells using One Shot® Mach1™ T-1 chemically competent cells (Invitrogen). Transformed cells were grown on LB plates containing 50 μg mL⁻¹ kanamycin; pENTR plasmids were harvested using a QIAprep® Miniprep kit (Qiagen, Valencia, Calif.); and the sequence of each insert was confirmed by DNA sequencing using the vector-specific primers M13-F and M13-R (Table 1). The inserts were transferred to the gene expression vector pRAP15 (FIG. 1), which contains the enhanced green fluorescent protein gene (eGFP; Haseloff et al. (1997) Proc Natl Acad Sci USA 94:2122-2127) for visualization of transformed roots as well as attR1 and attR2 sites for directional cloning using Invitrogen's Gateway® technology mediated by LR Clonase™ II Enzyme Mix (Invitrogen). The Clonase II reaction product was used to transform E. coli cells as described above, and transformed cells were grown on LB plates containing 10 μg mL⁻¹ tetracycline. The pRAP15 plasmids were harvested and presence of the insert in the correct orientation downstream from the figwort mosaic virus (FMV) promoter was confirmed by PCR using the FMV-specific primer FMV-F (Table 1) and the G. max gene-specific reverse primer. Taq DNA Polymerase Recombinant (Invitrogen) was used in the PCR reaction according to the manufacturer's instructions with cycling conditions as follows: an initial denaturation step of 94° C. for 3 min; 35 cycles of 94° C. for 45 sec, gene-specific primer Tₘ for 30 sec, and 72° C. for 1 min per kb of amplicon; and a final extension step of 72° C. for 10 min. The pRAP15 vector bearing the inserted gene of interest was used to transform chemically competent Agrobacterium rhizogenes ‘K599’ cells (Haas et al. (1995) Appl Environ Microbiol 61:2879-2884) using the freeze-thaw method (Hofgen et al. (1988) Nucleic Acids Res 16:9877) with selection on LB plates containing 5 μg mL⁻¹ tetracycline. Plasmids were harvested and presence of the insert in pRAP15 was confirmed as described above. Presence of eGFP was confirmed by PCR using eGFP-F and eGFP-R primers (Table 1). Presence of the A. rhizogenes Rᵢ plasmid, which is necessary for root transformation, was confirmed by PCR using Rᵢ-F and Rᵢ-R primers (Table 1).

### Example 3

### Formation of Composite Soybean Plants

A. rhizogenes clones containing the genes of interest were grown as described previously (Ibrahim et al. BMC Genomics 12:220 (2011), www.biomedcentral.com/1471-2164/12/220). Briefly, clones were grown individually in 5 ml Terrific Broth (Research Products International Corp., Mt. Prospect, Ill.) medium containing 5 ug/ml tetracycline on a rotary shaker at 250 rpm at 23-25 C. Cells were collected by centrifugation at 5000 rpm for 30 min at 4 C and resuspended in Murashige and Skoog medium (Murashige and Skoog (1962) Physiol Plant. 15, 473-497) as described by Klink et al (Plant Physiol. 151:1017-1022 (2009)) for root transformation. Cells containing pRAP15 with no gene of interest was grown to transform roots serving as controls.

Composite plants were prepared as described previously (Klink et al. Plant Physiol. 151:1017-1022 (2009)), as modified by Ibrahim et al. (BMC Genomics 12:220 (2011), www.biomedcentral.com/1471-2164/12/220). Briefly, for each gene tested, one hundred soybean plants, cv. Williams 82, were grown in Promix in the greenhouse. At approximately seven days, the plantlets were cut at the soil line and the base of each plant was submerged in a co-cultivation solution containing A. rhizogenes. The cocultivation solution was comprised of MS salts (4.40519 mg/ml Duchefa Biochemie; product #M0222.0050) and 3% sucrose at pH 5.7. After vacuum infiltration for 30 min, the plantlets were co-cultivated on a rotary shaker overnight at 23° C. at 65 rpm. The stems were rinsed with water, placed in a beaker of water, and incubated for approximately 48 hr at 23° C. under growing lights. The plantlets were planted in pre-wetted Promix in the greenhouse. Four weeks later, non-transformed roots were excised, while transformed roots were recognized by the presence of eGFP and retained. Fluorescence of eGFP was perceived using a Dark Reader Spot lamp (Clare Chemical Research, Dolores, Colo.). Non-transformed roots were removed again two weeks later during a second trimming. Approximately 12 to 20 healthy plants with the large, healthy roots were selected and planted in sand for inoculation with SCN.

### Example 4

### Nematode Preparation

SCN population NL1-RHg was grown in a greenhouse at the United States Department of Agriculture, Beltsville, Md. as described previously (Klink et al. (2007) Planta 226: 1389-1409). Briefly, mature SCN females and cysts were washed from roots of susceptible soybean plants three months after inoculation and purified by sucrose flotation (Jenkins, W R. Plant Dis. Rep 48:692 (1964)

The purified females and cysts were placed on a three inch diameter, 150 um sieve (Newark Wire Cloth Co, Clifton, N.J.), partially submerged in a small tray of water. Females and cysts were gently crushed with a rubber stopper against the sieve, allowing eggs to be collected in the tray below. The eggs were further purified by passing the solution through a 61 um sieve and collected in a 25 um sieve that retained the eggs, but allowed small particles to pass. To reduce microbial contamination, a 0.5% sodium hypochlorite solution was poured into the sieve and slowly drained out for 1.5 minutes before washing the sieve with one liter of sterile double distilled H₂O. Eggs were placed in 120 ml of sterile 3 mM ZnSO₄ in a small, covered tray and allowed to hatch on a rotary shaker at 25 rpm at 26° C. Four days later, SCN at the J2 stage were separated from unhatched eggs by passing the solution through a 30 um mesh nylon cloth (Spectrum Labs Inc, Rancho Dominguez, Calif.). J2s were concentrated by placing 200 ml of the solution in one liter glass beakers on a rotary shaker at 100 rpm. J2s quickly gathered to the center bottom of the beaker and were collected with a Pasteur pipette and placed in a bottle. Sterile water was added to a final volume of 100 ml and three samples of five ml of J2 were counted under a dissecting microscope. Volume of the solution was adjusted to achieve a concentration of 1,000 J2/ml for inoculation of transgenic roots of composite plants.

### Example 5

### Inoculation of Test Plants and Female Index

Twelve to twenty transformed composite plants were used in each assay. Two holes, 4 cm deep were made in the sand on either side of the plant. One ml of nematode inoculum was added to each hole to provide 2,000 juveniles per plant. After 35 days, test plants were placed in water and the roots were gently rubbed to dislodge the female nematodes. These were collected between nested 850 um and 250 um sieves and washed onto lined filter paper in a Buchner funnel (Krusberg et al. (1994) J Nematol 26:599). Wash effluent from nematode harvests was treated by diverting the waste stream through a soil trap and then through a Norweco Tablet Feeder [Stock #MD-45061 Model: XT2000], which dosed the waste water with Norweco Blue Crystal Disinfecting tablets [Calcium Hypochlorite EPA Registration 63243-4]. The treated waste water was then held in covered polyethylene tanks for an hour before being released as normal sewage. Wash effluent from nematode harvests was treated by diverting the waste stream through a soil trap and then through a Norweco Tablet Feeder [Stock #MD-45061 Model: XT2000], which dosed the waste water with Norweco Blue Crystal Disinfecting tablets [Calcium Hypochlorite EPA Registration 63243-4]. The treated waste water was then held in covered polyethylene tanks for an hour before being released as normal sewage. Females were counted under a dissecting microscope. Numbers of females were compared to vector plant controls to determine significant change in infectivity. Root weights after washing were taken to normalize the data.

### Example 6

### Quantitative Real Time PCR (qRT-PCR)

The expression of selected genes transformed into soybean roots was confirmed by qRT-PCR as described previously (Ibrahim et al. BMC Genomics 12:220 (2011), www.biomedcentral.com/1471-2164/12/220; Tremblay et al. (2009) Physiol. Molec Plant Pathol 73:163-174). Three soybean roots per construct (pRAP15, pRAP15-C45 (GDSL esterase/lipase CPRD49; phytozome ID: Glyma0466s00200.1) and pRAP15-C49 (possible lysine decarboxylase (carboxy-lyase); phytozome ID: Glyma17g37660.1)) were harvested, and RNA was extracted using a QIAGen RNeasy Mini Kit according to the manufacturer's instructions. After the RNA was isolated using an RNeasy Mini kit (QIAgen), contaminating DNA was removed by DNase digestion using a TURBO DNA-free kit (Invitrogen) according to manufacturer's instructions. The RNA concentration was determined using a Nanodrop (ND 1000 Spectrophotometer) and purity was confirmed by gel electrophoresis. The RNA was precipitated with ethanol (2.5 vol of 96-100%) and 1/10 vol of NaOAC (3M) and resuspended in 50 ul RNAse Free water (Qiagen). RNA was converted into cDNA library using Superscript III First Strand-Synthesis system for RT-PCR (Invitrogen) according to manufacturer's instruction. PCR was performed to determine the primers reliability and to confirm the size of the amplicon and that only one product was produced for each primer pair.

Soybean roots transformed with pRAP15 served as controls to measure endogenous gene expression. Primers were designed using Primer3 software to produce an amplicon between 100 and 200 bp (Table II) and Tm's ranging from 58 to 62° C. (C45: Forward: 58° C.; Reverse: 57° C.; C49: Forward: 58° C.; Reverse: 62° C.) (Table 2). The gene encoding rs-21 served as control (Klink et al. (2005) Plant Molecular Biology 59: 969-983). Llambda phage DNA served as the standard. Reactions containing no RNA or template processed with no reverse transcriptase were used as negative control.

TABLE 2
Primers for qRT-PCR

| | Phytozome | | Tm | | Tm | Amplicon |
|---|---|---|---|---|---|---|
| Gene | number | Forward primer | ° C. | Reverse primer | ° C. | (bp) |
| Rs-21 | Glyma09g00210.1 | CTAAGATGCAGAA | 54° C. | GAGAGCAAAAGTG | 55° C. | 168 |
| | | CGAGGAAGG | | GAGAAATGG | | |
| | | (SEQ ID NO: 202) | | (SEQ ID NO: 203) | | |
| C45 | Glyma0466s00200.1 | GCAGATGGGTTAA | 58° C. | GACATCCAATGCA | 57° C. | 203 |
| | | TGGAGCTTTGTG | | GACTAGGTTTCC | | |
| | | (SEQ ID NO: 204) | | (SEQ ID NO: 205) | | |
| C49 | Glyma17g37660.1 | CGTGGATGGGTAC | 58° C. | TGGTTCATCTCCCA | 62° C. | 186 |
| | | TACAACTCGTTG | | ACTTTGCTTTG | | |
| | | (SEQ ID NO: 206) | | (SEQ ID NO: 207) | | |

qRT-PCR reactions were conducted in triplicate for each root cDNA sample using Brilliant II Syber Green Master Mix qPCR Kit (Strategene, La Jolla, Calif.) according to the manufacturer's instructions. Reactions were incubated for 10 min at 95° C., then for forty cycles at 30 s at 95° C., 1 min at 55° C. and 0.5 min at 72° C., then incubated for 3 min at 72° C. Relative levels of gene expression were determined using the Stratagene Mx3000P Real-Time PCR system (Stratagene) as described by the manufacturer. DNA accumulation during the reaction was measured with SYBR Green. The Ct (cycle at which there is the first clearly detectable increase in fluorescence) values were calculated using software supplied with the Stratagene Mx3000P Real-Time PCR system. The SYBR green dissociation curve of the amplified products demonstrated the production of only one product per reaction. Data analysis was performed according to the sigmoidal model to get absolute quantification as described in Tremblay et al. (Physiol. Molec Plant Pathol 73:163-174(2009)).

### Example 7

### Gene Selection and Assay System

More than 100 genes were selected to be over-expressed in soybean roots to determine their effect on SCN development. The genes were chosen from gene expression data derived from microarray experiments reported previously (Klink et al. (2007a) Planta 226: 1389-1409; Klink et al. (2007b) Planta 226: 1423-1447; Klink et al. (2009a) Plant Molecular Biology 71:525-567; Klink et al. (2009b) Plant Physiol. 151:1017-1022; Ithal et al. (2007) Molec Plant Path Interact 20:293-305). Genes were chosen in this study which were increased, decreased or had no change in transcript abundance in the host during nematode infection. Sequences of the gene probes on the microarrays were obtained from Affimetrix GeneChip (www.affymetrix.com/analysis/index.affx). The sequences were used to build contigs using soybean ESTS found in the NCBI GenBank database, and they were also used to identify highly related genes of soybean found in the Phytozome Glycine max database. The contigs and soybeans genes found in Phytozome were used to design primers using Primer3 to clone the full-length open reading frame (ORF). The ORF of each gene was cloned into pRAP15 (FIG. 1) for overexpression in soybean roots of composite plants.

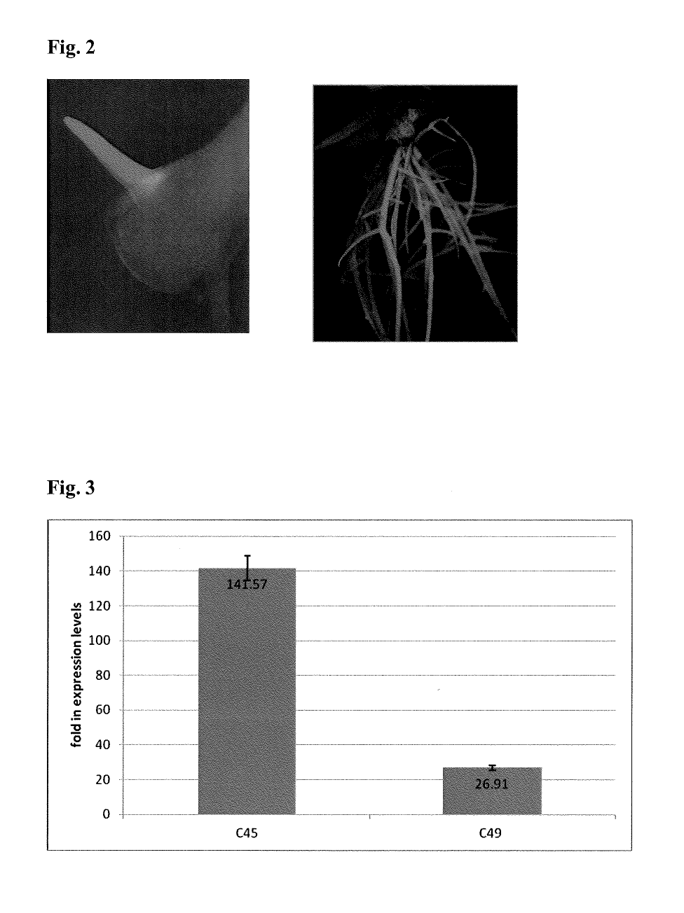

An assay system was devised using composite soybean plants having roots that were transformed with the gene construct, because assays could be conducted more quickly than if transgenic soybean plants were used. Transformed roots were visualized by the presence of eGFP (FIG. 2). Non-transformed roots were excised from the composite plant at approximately 4 weeks after transformation and again two weeks later to remove any remaining non-transformed roots. Transgenic roots were inoculated with 2000 J2 juveniles of SCN per root. After 32 to 35 dai, the plants were harvested and mature SCN females were collected and counted. The female index was calculated using twelve to twenty composite plants with roots recognized as transgenic due to the presence of eGFP

To confirm that transcripts of the genes were over-expressed, transcript levels from three roots for each of two constructs, C45 and C49, were measured using qRT-PCR. The transcript levels of genes encoded by C45 and C49 were increased 141 and 27-fold, respectively, as compared to control roots transformed with empty pRAP15 as measured by qRT-PCR (FIG. 3)

TABLE 3
Effect of overexpression of the nucleotide sequences of SEQ ID NOs: 1-97.

| SEQ ID | | | | Mean # | pRAP15 | % of | |
|---|---|---|---|---|---|---|---|
| NO: | Phytozome ID No | Putative Function | Gene | females | control | control | P-value |
| 1 | Glyma08g02610.1 | b-glucanase | A12 | 49 | 142 | 35 | 0.0001 |
| 2 | Glyma08g41040.1 | unknown | A38 | 13 | 37 | 35 | 0.01 |
| 3 | Glyma08g14550.1 | lipase | A25 | 42 | 103 | 41 | 0.008 |
| 4 | Glyma07g05830.2 | cytoch b5 | C19 | 24 | 58 | 41 | 0.002 |
| 5 | Glyma13g22650.1 | diacyanin | A30 | 18 | 37 | 49 | 0.03 |
| 6 | Glyma10g30340.1 | unknown | A8 | 62 | 127 | 49 | 0.003 |
| 7 | Glyma17g35360.1 | lipase | C29 | 29 | 58 | 50 | 0.006 |
| 8 | Glyma19g19680.1 | calmodulin SCaM-3 | S1 | 64 | 127 | 50 | 0.003 |
| 9 | Glyma15g16560.1 | DREPP membrane | R8 | 21 | 41 | 51 | 0.008 |
| 10 | Glyma12g07780.1 | asc perox | C21 | 55 | 103 | 53 | 0.03 |
| 11 | Glyma08g11490.1 | Serinehydroxymethyl- | A35 | 70 | 127 | 55 | 0.007 |
| | | transferase 2 | | | | | |
| 12 | Glyma05g38130.1 | Thaumatin | A7 | 71 | 127 | 56 | 0.007 |
| 13 | Glyma01g39810.1 | arabinogalactan | C9 | 101 | 165 | 61 | 0.056 |
| 14 | Glyma03g32850.1 | HSP70 | C14 | 36 | 58 | 62 | 0.03 |
| 15 | Glyma04g10880.1 | Phosphate responsive | C28 | 100 | 159 | 63 | 0.005 |
| 16 | Glyma03g30380.1 | Dirigent-like protein | A65 | 99 | 157 | 63 | 0.28 |
| 17 | Glyma13g29690.1 | aquaporin | A43 | 53 | 82 | 65 | 0.031 |
| 18 | Glyma20g24810.1 | Cinnamate 4- | A11 | 83 | 127 | 65 | 0.036 |
| | | hydroxylase | | | | | |
| 19 | Glyma04g08520.1 | transporter | R48 | 27 | 41 | 66 | 0.09 |
| 20 | Glyma13g27020.1 | Annexin | C12 | 110 | 167 | 66 | 0.25 |
| 21 | Glyma13g23680.1 | nitrate/oligopeptide | C43 | 43 | 63 | 68 | 0.10 |
| | | transporter | | | | | |
| 22 | Glyma20g27950.1 | polyubiquitin | C37 | 109 | 159 | 69 | 0.018 |
| 23 | Glyma08g05710.1 | HMG I/Y | C16 | 132 | 192 | 69 | 0.01 |
| 24 | Glyma07g30880.1 | monosaccharide | A10 | 89 | 127 | 70 | 0.076 |
| | | transporter | | | | | |
| 25 | Glyma20g36790.1 | auxin repressor | R24 | 36 | 51 | 71 | 0.09 |
| 26 | Glyma06g41610.1 | thioredoxin-related | C42 | 143 | 201 | 71 | 0.011 |
| 27 | Glyma08g11520.1 | chalcone synthase | A52 | 47 | 66 | 71 | 0.23 |
| 28 | Glyma14g09990.1 | Phytosulfokine | A27 | 75 | 103 | 73 | 0.19 |
| | | precursor protein | | | | | |
| 29 | Glyma19g02180.1 | AAA+-type ATPase | C13 | 126 | 167 | 75 | 0.46 |
| 30 | Glyma20g24280.1 | NADH: ubiquinone | C17 | 126 | 167 | 75 | 0.45 |
| | | oxidoreductase | | | | | |
| 31 | Glyma09g33140.1 | Dirigent-like protein | A15 | 28 | 37 | 76 | 0.30 |
| 32 | Glyma16g07830.1 | 2OG-Fe(II) oxygenase | C52 | 46 | 58 | 79 | 0.28 |
| 33 | Glyma14g00640.1 | Chlorophyll A-B | C40 | 132 | 159 | 83 | 0.38 |
| | | binding protein | | | | | |
| 34 | Glyma11g10240.4 | expansin; rare | C27 | 140 | 167 | 84 | 0.52 |
| | (variant) | lipoprotein A | | | | | |
| 35 | Giyma14g35370.1 | Class-II DAHP | A67 | 69 | 82 | 84 | 0.47 |
| | | synthetase | | | | | |
| 36 | Glyma04g40580 | O-methyltransferase | A42 | 107 | 127 | 84 | 0.37 |
| 37 | Glyma17g03130.1 | epoxide hydrolase | A9 | 108 | 127 | 85 | 0.41 |
| 38 | Glyma06g14960.1 | superoxide dismutase | C7 + his | 54 | 63 | 86 | 0.44 |
| 39 | Glyma20g36700.1 | unknown | A51 | 109 | 127 | 86 | 0.39 |
| 40 | Glyma17g06220.1 | cytokinin | A37 | 32 | 37 | 86 | 0.58 |
| | | dehydrogenase | | | | | |
| 41 | Glyma13g26960.1 | annexin | A60 | 136 | 157 | 87 | 0.55 |
| 42 | Glyma10g00970.1 | unknown | A44 | 58 | 66 | 88 | 0.63 |
| 43 | Glyma14g38220.2 | DAD1 | R5 | 37 | 41 | 90 | 0.63 |
| 44 | Glyma12g08530.1 | Fragile fiber 8 | R29 | 37 | 41 | 90 | 0.77 |
| 45 | Glyma19g38390.1 | Short chain | A24 | 53 | 58 | 91 | 0.75 |
| | | dehydrogenase | | | | | |
| 46 | Glyma17g37660.1 | lysine decarboxylase | C49 | 146 | 159 | 92 | 0.61 |
| 47 | Glyma19g36620.1 | Phenylalanine | A53 | 120 | 127 | 94 | 0.75 |
| | | ammonia-lyase | | | | | |
| 48 | Glyma13g16620.1 | nuclease HARBI1-like | A49 | 63 | 66 | 95 | 0.83 |
| 49 | Glyma08g21420.1 | acid phosphatase | C20 | 195 | 201 | 97 | 0.86 |
| 50 | Glyma15g41840.1 | lipase | K1 | 118 | 121 | 98 | 0.89 |
| 51 | Glyma0466s00200.1 | GDSL esterase/lipase | C45 | 156 | 159 | 98 | 0.91 |
| 52 | Glyma08g25950.1 | cytochrome P450 | C23 | 57 | 58 | 98 | 0.94 |
| 53 | Glyma02g47940.1 | Phenylalanine | A45 | 65 | 66 | 98 | 0.92 |
| | | ammonia-lyase | | | | | |
| 54 | Glyma09g05230.1 | DREPP membrane | C53 | 160 | 159 | 101 | 0.97 |
| | | polypeptide | | | | | |
| 55 | Glyma02g33780.1 | glutathione S- | A33 | 162 | 157 | 103 | 0.89 |
| | | transferase | | | | | |
| 56 | Glyma04g04310.1 | WOX TF | A6 | 107 | 103 | 104 | 0.88 |
| 57 | Glyma16g27350.1 | sucrose transport | A68 | 88 | 82 | 107 | 0.80 |
| 58 | Glyma06g47740.1 | pectin esterase | A20 | 65 | 59 | 110 | 0.46 |
| | | inhibitor | | | | | |
| 59 | Glyma12g01580.1 | heat shock protein | A39 | 143 | 127 | 113 | 0.46 |
| 60 | Glyma03g37940.1 | WRKY TF | C34 | 67 | 59 | 114 | 0.40 |
| 61 | Glyma02g38030.1 | TFIIA | C39 | 230 | 201 | 114 | 0.41 |
| 62 | Glyma05g30380.1 | Cu binding | A22 | 78 | 65 | 120 | 0.30 |
| 63 | Glyma20g30720.1 | abscissic stress | R3 | 49 | 41 | 120 | 0.36 |
| 64 | Glyma08g04740.1 | unknown | C3 | 244 | 201 | 121 | 0.19 |
| 65 | Glyma20g35270.1 | auxin-responsive | R7 | 50 | 41 | 122 | 0.29 |
| 66 | Glyma14g10200.1 | IgA FC receptor-like | A66 | 101 | 82 | 123 | 0.42 |
| 67 | Glyma04g41750.1 | ubiquitin conjugating | C15 | 206 | 167 | 123 | 0.50 |
| | | enzyme | | | | | |
| 68 | Glyma17g07190.1 | 4-coumarate: coenzyme | A48 | 79 | 63 | 125 | 0.35 |
| | | A ligase | | | | | |
| 69 | Glyma02g16480.1 | kelch repeat, F-box | R27 | 53 | 41 | 129 | 0.18 |
| 70 | Glyma15g14040.1 | berberine-like | A31 | 48 | 37 | 130 | 0.31 |
| 71 | Glyma11g10240.1 | pollen allergen | A64 | 204 | 157 | 130 | 0.27 |
| 72 | Glyma03g27740.1 | cytochrome P450 | A13 | 66 | 50 | 132 | 0.04 |
| 73 | Glyma11g37370.1 | B12D protein | A26 | 77 | 58 | 133 | 0.11 |
| 74 | Glyma17g01230.1 | BAF60 TF | A23 | 78 | 58 | 134 | 0.13 |
| 75 | Glyma20g23080.1 | calreticulin | C36 | 81 | 59 | 137 | 0.07 |
| 76 | Glyma13g23400.1 | ribosomal protein S11 | C6 | 228 | 165 | 138 | 0.053 |
| 77 | Glyma13g44700.1 | cinnamoyl CoA | A46 | 221 | 157 | 141 | 0.11 |
| | | reductase | | | | | |
| 78 | Glyma13g30950.1 | unknown | A5 | 84 | 59 | 142 | 0.04 |
| 79 | Glyma01g39460.1 | O-methyltransferase | A3 | 84 | 59 | 142 | 0.058 |
| 80 | Glyma01g42670.1 | Thaumatin PR5b | R4 | 289 | 201 | 144 | 0.06 |
| 81 | Glyma13g01230.1 | PR1-like | A36 | 119 | 82 | 145 | 0.026 |
| 82 | Glyma08g27590.1 | membrane type III | A34 | 120 | 82 | 146 | 0.037 |
| 83 | Glyma20g26610.1 | secretory protein | A50 | 96 | 63 | 152 | 0.035 |
| 84 | Glyma04g08200.1 | endopeptidase | C8 | 255 | 165 | 155 | 0.036 |
| 85 | Glyma17g03360.1 | SAM22 PR10 | A1 | 94 | 59 | 159 | 0.001 |
| 86 | Glyma02g40000.1 | cationic peroxidase | A2 | 94 | 59 | 159 | 0.006 |
| 87 | Glyma02g42290.1 | auxin permease | A18 | 95 | 59 | 161 | 0.005 |
| 88 | Glyma09g31110.1 | metal ion transport | C55 | 97 | 59 | 164 | 0.0004 |
| 89 | Glyma06g12340.1 | ACC oxidase | R28 | 68 | 41 | 166 | 0.061 |
| 90 | Glyma07g16420.1 | unknown | R25 | 69 | 41 | 168 | 0.007 |
| 91 | Glyma19g09810.1 | cupin domain | A61 | 291 | 157 | 185 | 0.004 |
| 92 | Glyma04g39860.1 | peroxidase III | A4 | 115 | 59 | 195 | 0.0001 |
| 93 | Glyma03g29450.1 | Ca-depend kinase | C22 | 124 | 63 | 197 | 0.005 |
| 94 | Glyma15g03390.1 | unknown | A21 | 135 | 59 | 229 | 0.0001 |
| 95 | Glyma02g37020.1 | NAD dehydratase | C32 | 146 | 63 | 232 | 0.0001 |
| 96 | Glyma17g13550.1 | pectate lyase | A32 | 92 | 37 | 249 | 0.02 |
| 97 | Glyma16g33840.1 | oligopeptide | R30 | 104 | 41 | 254 | <0.0001 |
| | | transporter | | | | | |

### Example 8

### Nucleotide Sequences Decreasing the Female Index 50% or More

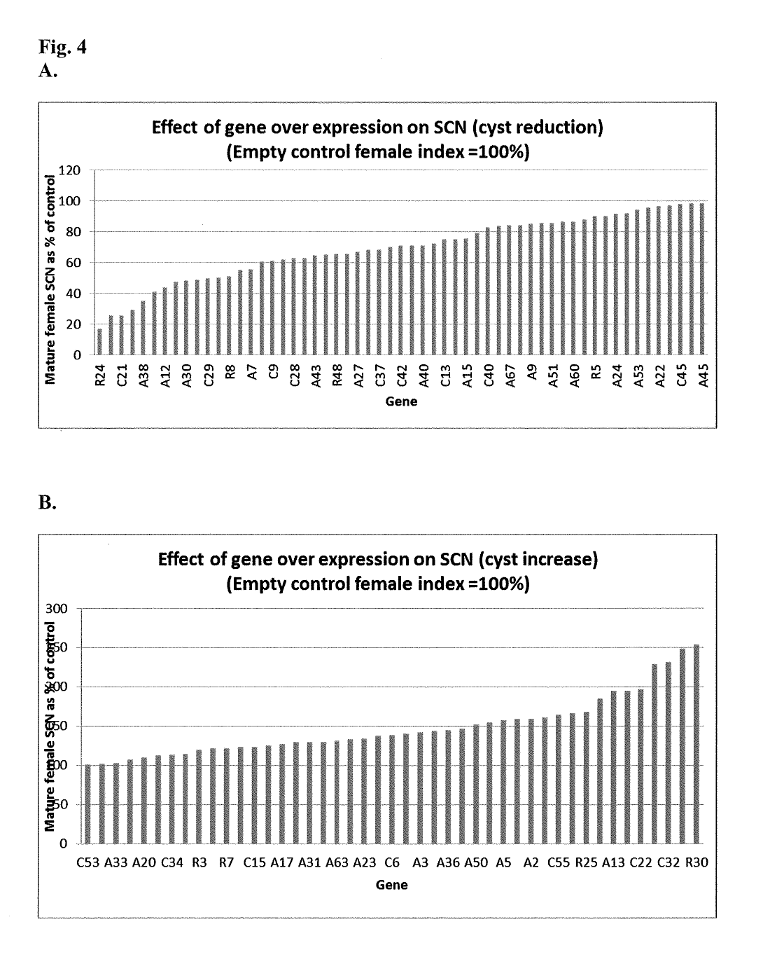

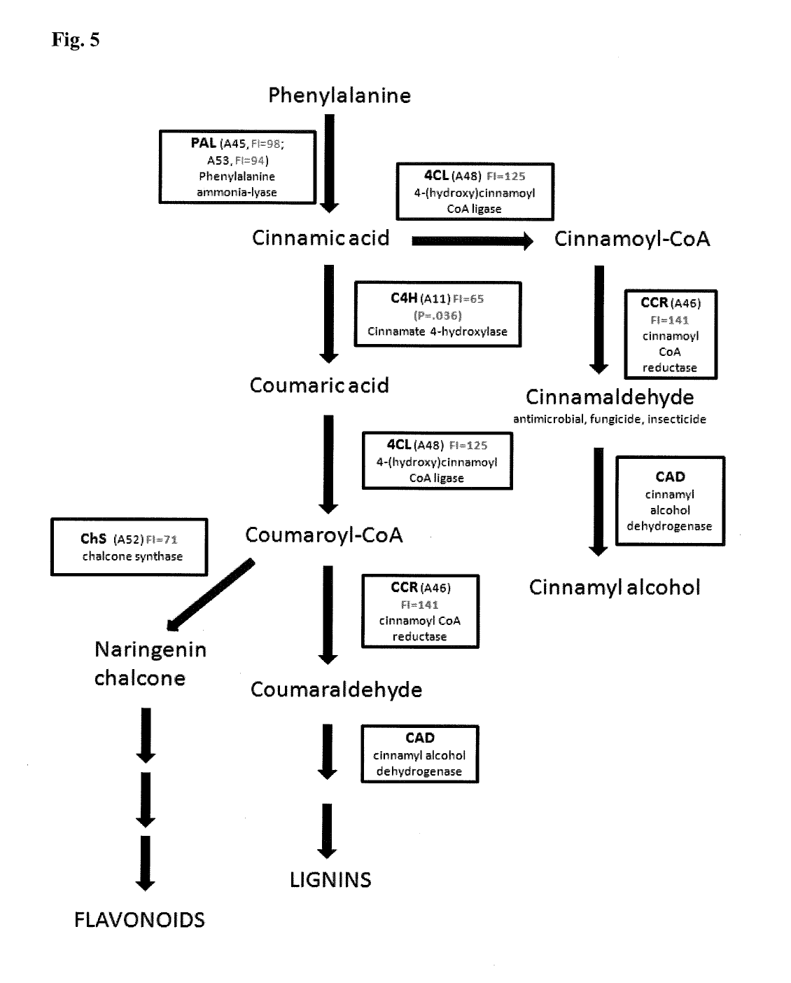

Nine genes decreased the Female Index (FI) by 50% or more when over-expressed in transgenic roots (FIG. 4) These leads include A38, A12, A25, C19, C21, A08, A30, C29, SO1, and R08 (Table 3).

TABLE 4
Genes identified as reducing the Female Index of SCN by about 25% or more when
overexpressed.

| | | | Percent | |
|---|---|---|---|---|
| Phytozome ID No. | Putative Function | Gene | Reduction | GenBank ID |
| Glyma08g41040.1 | unknown-probably TF | A38 | 35 | CD395088 |
| Glyma08g02610.1 | b-glucanase (PR2) | A12 | 36 | BI943300 |
| Glyma08g14550.1 | lipoxygenase LH2; lipase | A25 | 36 | CD409280 |
| Glyma07g05830.2 | cytoch b5 | C19 | 41 | CF807399 |
| Glyma12g07780.1 | asc perox | C21 | 47 | CD412482 |
| Glyma10g30340.1 | unknown | A08 | 49 | CF805971 |
| Glyma13g22650 | diacyanin-cu containing protein | A30 | 49 | BE659015 |
| Glyma17g35360.1 | Regulator protein | C29 | 50 | CD391061 |
| Glyma19g19680.1 | calmodulin SCaM-3 | S01 | 50 | L01432 |
| Glyma15g16560.1 | DREPP membrane | R08 | 51 | BG509247 |
| Glyma20g36790.1 | auxin repressor | R24 | 53 | CF806954 |
| Glyma08g11490.1 | serinehydroxymethyltransferase 2 | A35 | 55 | CD400862 |
| Glyma05g38130.1 | thaumatin | A07 | 56 | BQ628525 |
| Glyma01g39810 | arabinogalactan | C09 | 61 | BQ612879 |
| Glyma03g32850.1 | HSP70 BiP | C14 | 62 | AW350100 |
| Glyma03g30380.1 | dirigent-like protein | A65 | 63 | CF807760 |
| Glyma04g10880.1 | phosphate responsive | C28 | 63 | BE059056 |
| Glyma20g24810.1 | cinnamate_4-hydroxylase | A11 | 65 | CF808939 |
| Glyma13g29690 | aquaporin | A43 | 65 | CD403744 |
| Glyma13g27020.1 | annexin | C12 | 66 | AW100836 |
| Glyma04g08520.1 | transporter | R48 | 66 | AW309763 |
| Glyma08g05710.1 | HMG I/Y | C16 | 67 | AJ319868 |
| Glyma13g23680.1 | nitrate/oligopeptide transporter | C43 | 68 | BI969343 |
| Glyma14g09990.1 | Phytosulfokine precursor protein | A27 | 69 | BK000119 |
| Glyma20g27950.1 | polyubiquitin | C37 | 69 | CK606562 |
| Glyma07g30880.1 | monosaccharide transporter | A10 | 70 | AJ563365 |
| Glyma08g11520.1 | chalcone synthase 2 | A52 | 71 | BQ081473 |
| Glyma06g41610.1 | thioredoxin-related | C42 | 71 | CF808262 |
| Glyma04g04310.1 | WOX TF | A06 | 75 | BE058056 |
| Glyma19g02180.1 | AAA+-type ATPase | C13 | 75 | AW100836 |
| Glyma20g24280.1 | NADH:ubiquinone oxidoreductase | C17 | 75 | CA800345 |

One gene, A38 encodes a possible transcription factor, while three genes may be involve in signaling, a lipoxygenase A25, and calmodulin SCaM-3 S01. Other genes in this group include a β-glucanase (A12) and a peroxidase (C21). Four genes are of unknown function, including a gene C29 encoding a protein containing a hydrolase domain and a gene encoding a protein similar to cytochrome b5 proteins.

Gene A38 (Glyma08g41040; CD395088) decreased the female index approximately 65%. It encodes a protein containing amino acid sequence similarity to ParB and STAT proteins as supported by a search of its predicted amino acid sequence against Pfam (pfam.sanger.ac.uk/search). ParB recognizes specific DNA motifs, the A-box and B-box ParB is involved in the partitioning of DNA during cell division (Funnell (1988) J Bacteriol 170:954-960; Mohl et al. 1997 Cell 88:675-684). The STAT domain (Signal transducer and activator of transcription) is found in a family of transcription factors involved in cell growth and differentiation (Ihle (1996) Cell 84:331-334). This ParB-STAT protein is closely-related to Glyma18g15530.1, Glyma01g05350.1, Glyma02g11750.1, with 68.3, 66.1 and 65% similarity, respectively. Transcripts of gene A38 were elevated 40-, 22-, and 48-fold in syncytia formed by SCN in soybean cv. Peking in an incompatible interaction at 3, 6 and 9 dai, respectively (Klink et al. (2007a) Planta 226: 1389-1409; Klink et al. (2007b) Planta 226: 1423-1447; Klink et al. (2009a) Plant Molecular Biology 71:525-567; Klink et al. (2009b) Plant Physiol. 151:1017-1022). It was also elevated at 2 dai in syncytia formed during the compatible interaction of SCN with soybean cv. Williams 82 at 2 dai, but transcript levels were unchanged in syncytia at 5 and 10 dai (Ithal 2007). In the susceptible interaction of root-knot nematode with soybean, transcript levels of this ParB-STAT protein were elevated 7- and 12-fold in galls 12 dai and 2 months after infection (mai; Ibrahim et al. (2011) BMC Genomics 12:220 www.biomedcentral.com/1471-2164/12/220).

Overexpression of gene A12 decreased the female index to 36% of controls. It encodes a β-1,4-endoglucanase (Glyma08g02610.1; BI943300) that catalyzes the hydrolysis of cellulose. It is encoded by a 1875 bp ORF encoding a protein of 625 aa. There are fifteen homologues (>2e-34) of this β-glucanase in soybean with Glyma05g36930 (4,0e-64) being most closely related. Transcripts of this gene were elevated over 300-fold in syncytia formed by SCN in Peking 3dai, over 200-fold at 6 dai and over 100-fold at 9 dai (Klink et al. 2007) and were unchanged in syncytia analyzed from the compatible interaction of SCN with Williams 82. The first protein identified as a secreted protein from the esophageal glands of SCN was a β-1,4-endo glucanase (Smant et al. (1998) Proc Nat Acad Sci USA 95:4906-11; Yan et al. (1998) Gene 220:61-70).

When gene A25 was over-expressed in transgenic soybean roots, the female index was also 36% of controls. A25 encodes a lipoxygenase (Glyma08g14550.1; CD409280), a member of the PLAT domain family. It has 92% similarity with Glyma05g31310.1 and 86.3% similarity with Glyma11g38220.1. Transcripts of this lipoxygenase were elevated over 250-, 100-, and 60-fold in syncytia of Peking at 3, 6, and 9 dai, respectively (Klink et al. 2007, 2009).

Overexpression of gene C19 (Glyma07g05830.2; CF807399) decreased the female index to 41% as compared to controls. It is a member of the cytochrome b5 superfamily, and its function is unknown. It has numerous homologues with high similarity at the amino acid level. It has 97.2% similarity at the amino acid level with Glyma16g02410.1; 76.8% similarity with Glyma04g41010; 76.1% similarity with Glyma06g13840.1; 71.8% similarity with Glyma03g42070; and 71.1% similarity with Glyma19g44780. It was approximately 2-fold induced in synctytia from the incompatible interaction of SCN with Peking at 3, 6, and 9 dai (Klink et al. (2007a) Planta 226: 1389-1409; Klink et al. (2007b) Planta 226: 1423-1447; Klink et al. (2009a) Plant Molecular Biology 71:525-567; Klink et al. (2009b) Plant Physiol. 151:1017-1022) and in syncytia from the compatible interaction at 2, 5 and 10 dai (Ithal et al. (2007) Molec Plant Path Interact 20:293-305).

Cytosolic ascorbate peroxidase 2, represented by C21, (Glyma12g07780.1; CD412482) detoxifies hydrogen peroxide and is induced in response to stress. When soybean ascorbate peroxidase is over-expressed soybean roots, it decreased the female index of SCN to 47% of controls. When ascorbate peroxidase is over-expressed in yeast, it decreases the accumulation of reactive oxygen species and suppresses plant cell death (Moon et al. 2002). Glyma11g15680.1 is 93.2% similar to it at the amino acid level, while Glyma11g15680.3 is 75.2% similar. Other homologues were less than 63% similar. Soybean ascorbate peroxidase (C21) transcript levels were unchanged in synctia from the compatible and incompatible interactions (Klink et al. (2007) Planta 226: 1389-1409; Klink et al. (2007) Planta 226: 1423-1447; Klink et al. (2009) Plant Molecular Biology 71:525-567; Klink et al. (2009) Plant Physiol. 151:1017-1022; Ithal et al. (2007) Molec Plant Path Interact 20:293-305).

Gene A30 (Glyma13g22650; BE659015) overexpression reduced the female index 51%. Gene A30 encodes a 336 amino acid, blue copper protein, plastocyanin-like of unknown function. Its closest homologue is Glyma17g12150.1 at 42.3% amino acid similarity. Transcripts of gene A30 were elevated approximately 63-, 120-, and 57-fold in synctytia from the incompatible interaction of SCN with Peking at 3, 6, and 9 dai (Klink et al. (2007) Planta 226: 1389-1409; Klink et al. (2007) Planta 226: 1423-1447), while its transcripts were elevated 10-6- and 5-fold in synctytia from the compatible interaction of SCN with Williams 82 at 2, 5 and 10 dai, respectively (Ithal et al. (2007) Molec Plant Path Interact 20:293-305).

Over expression of gene A08 (Glyma10g30340.1; CF805971) reduced the female index to 49% of controls. Gene A08 has no known function and no significant matches in Pfam, but it has been identified as an uncharacterized protein in other plants, such as Ricinus communis, Populus trichocarpa, and Medicago truncatula, according to our blastp results. Transcript levels were unchanged in syncytia from incompatible and compatible interactions of SCN with Peking and Williams 82, respectively (Klink et al. (2007) Planta 226: 1389-1409; Klink et al. (2007) Planta 226: 1423-1447; Ithal et al. (2007) Molec Plant Path Interact 20:293-305).

Gene C29 (Glyma17g35360.1; CD391061) also has no known function, but it contains an alpha/beta hydrolase domain common to proteases, lipases peroxidases and other hydrolytic enzymes. When it is over-expressed in soybean roots, the female index is reduced by 50%. It had high similarity (98.9%) with Glyma0092s00240.1. Other related genes encode proteins with similarity below 76%. Transcript levels were unchanged in syncytia from incompatible and compatible interactions of SCN with Peking and Williams 82, respectively (Klink et al. (2007) Planta 226: 1389-1409; Klink et al. (2007) Planta 226: 1423-1447; Ithal et al. (2007) Molec Plant Path Interact 20:293-305).

Similarly, when the gene R08 encoding calmodulin SCaM-3 (Glyma19g19680.1; L01432) was over-expressed, the female index of SCN was reduced by 50%. Several soybean genes encode closely-related proteins, including Glyma02g44350.1, Glyma14g04460.1, Glyma14g04460.1 and Glyma05g12900.1; all had 100% similarity at the amino acid level. Transcript levels of this gene were unchanged in syncytia from incompatible and compatible interactions of SCN with Peking (Klink et al. (2007) Planta 226: 1389-1409; Klink et al. (2007) Planta 226: 1423-1447). However, transcript levels were elevated approximately 2-fold in syncytia from the compatible reaction of SCN with Williams 82 at 2 and 5 dai, and they were 5-fold elevated at 10 dai (Ithal et al. (2007) Molec Plant Path Interact 20:293-305).

Genes Increasing the Female Index More than Two-Fold.

Overexpression of several genes, including R30, A32, C32, and A21, appeared to enhance susceptibility (Table 5).

TABLE 5
Genes increasing the Female Index of SCN when over-expressed.

| | | | % of | |
|---|---|---|---|---|
| Gene | Function | ID no | control | GenBank No |
| Glyma16g33840.1 | oligopeptide | R30 | 254 | BI972216 |
| | transporter | | | |
| Glyma17g13550.1 | pectate lyase | A32 | 249 | CD397515 |
| Glyma02g37020.1 | NAD dehydratase | C32 | 232 | CF806679 |
| Glyma15g03390.1 | unknown | A21 | 229 | AW307334 |
| Glyma03g29450.1 | Ca-depend kinase | C22 | 197 | BU765503 |
| Glyma04g39860.1 | peroxidase III | A04 | 195 | CF809087 |
| Glyma19g09810.1 | cupin domain | A61 | 185 | U21722 |
| Glyma07g16420.1 | unknown | R25 | 168 | CF808812 |
| Glyma06g12340.1 | ACC oxidase | R28 | 166 | AW349263 |
| Glyma09g31110.1 | metal ion transport | C55 | 164 | CA852377 |
| Glyma02g42290.1 | auxin permease | A18 | 161 | CA820051 |
| Glyma17g03360.1 | SAM22 PR10 | A01 | 159 | CF921432 |
| Glyma02g40000.1 | cationic peroxidase | A02 | 159 | BU548599 |
| Glyma04g08200.1 | endopeptidase | C08 | 155 | BG509166 |
| Glyma20g26610.1 | secretory protein | A50 | 152 | CA852440 |
| Glyma03g27740.1 | cytochrome P450 | A13 | 148 | AW310572 |
| Glyma08g27590.1 | membrane type III | A34 | 146 | BG839541 |
| Glyma13g01230.1 | PR1-like | A36 | 145 | AW278629 |
| Glyma01g42670.1 | Thaumatin PR5b | R04 | 144 | CF807955 |
| Glyma01g39460.1 | O-methyltransferase | A03 | 142 | AW349604 |
| Glyma13g30950.1 | unknown | A05 | 142 | BQ628412 |
| Glyma13g44700.1 | cinnamoyl CoA | A46 | 141 | BQ454241 |
| | reductase | | | |

Gene R30 (Glyma16g33840.1; BI972216) encodes an OPT oligotransporter that increased the SCN female index 2.5-fold when over-expressed in soybean roots. It has 96.7% similarity at the amino acid level with Glyma09g29410.1 Transcripts of this gene were not altered in abundance on microarrays during the incompatible reaction, but were slightly down regulated at −1.2 and −3.3-fold in the compatible interaction of SCN with soybean roots at 2 and 10 dai, respectively (Ithal et al. (2007) Molec Plant Path Interact 20:293-305).

Overexpression of the gene A32 (Glyma17g13550.1; CD397515) encoding pectate lyase also increased the female index of SCN approximately 2.5-fold. It has 93.6% aa similarity with Glyma05g02890.1 93.6%, and is has more than 80% aa similarity with six other soybean genes. It was over-expressed in syncytia from both resistant and susceptible interactions ((Klink et al. (2007) Planta 226: 1389-1409; Klink et al. (2007) Planta 226: 1423-1447; Klink et al. (2009) Plant Molecular Biology 71:525-567; Klink et al. (2009) Plant Physiol. 151:1017-1022; Ithal et al. (2007) Molec Plant Path Interact 20:293-305)

The gene C13 (Glyma02g37020; CF806679) encoding UDP-glucuronate 4-epimerase (EC:5.1.3.6) increased the female index 2.3-fold when over-expressed. Its closest relative at the amino acid level is Glyma17g07740.1 at 97.2%, with Glyma01g33650.1 and Glyma03g03180.1 having 77% similarity. The transcripts of this gene were only slightly less abundant than controls in the incompatible reaction of SCN with Peking at 6 and 9 dai at −1.8 and −1.6-fold, respectively. Interestingly, this gene is increased 28.7-fold in galls formed by RKN in soybean roots two mai (Ibrahim et al. (2011) BMC Genomics 12:220 www.biomedcentral.com/1471-2164/12/220).

Similarly, overexpression of gene A21 (Glyma15g03390.1; AW307334) of unknown function yielded a 2.3-fold increase in the female index. This gene encodes a peptide of 134 amino acids and possesses no domains similar to those in Pfam. It has similarity to Glyma13g41990.1 at 89.6% and Glyma11g13460.1 at only 48.5%. Transcript levels of A21 were increased 16-, 31.3- and 73.5-fold at 3, 6, and 9 dai in syncytia from the incompatible interaction of SCN with Peking ((Klink et al. (2007) Planta 226: 1389-1409; Klink et al. (2007) Planta 226: 1423-1447; Klink et al. (2009) Plant Molecular Biology 71:525-567; Klink et al. (2009) Plant Physiol. 151:1017-1022).

We examined the effect of overexpression of six genes related to flavinoid production, specifically, two genes encoding phenylalanine ammonia lyase (PAL, EC 4.3.1.24; A45, A53), and one gene encoding chalcone synthase (ChS, EC 2.3.1.74; A52), 4-coumerate CoA ligase (4CL, EC 6.2.1.12, A48), cinnamate-4-hydroxylase (C4H, EC 1.14.13.11; A11) and cinnamoyl CoA reductase (CAD, EC 1.2.1.44; A46), respectively (FIG. 5). Overexpression of genes encoding PAL had no effect on SCN development even though transcripts of both PAL genes (Glyma19g36620 and Glyma02g47940 showed a 25-fold increased in abundance at 3 6 and 9 dai in syncytia formed in the resistant interaction of SCN with Peking (Klink et al. (2007) Planta 226: 1423-1447; Klink et al. (2009) Plant Molecular Biology 71:525-567; Klink et al. (2009) Plant Physiol. 151:1017-1022). Both PAL genes were also over-expressed in the resistant reaction of SCN with PI 88788 (Klink et al. (2011) Plant Molec Biol 75:141-165), but there was no change in transcript abundance in the susceptible interaction at 2, 5 and 10 dai (Ithal et al. (2007) Molec Plant Path Interact 20:293-305). Overexpression of the gene encoding ChS decreased the female index (FI) of SCN to 71% of control. Overexpression of the gene encoding cinnamate 4 hydroxylase decreased the FI of SCN to 65% of the control, while overexpression of the gene encoding cinnamoyl CoA reductase (CAD) increased the FI to 141% of the control.

Three genes related to auxin were over expressed and tested. When the gene encoding auxin repressor (R24) was over-expressed, the FI was 53% of control. When the gene encoding auxin permease (A18) was over expressed, the FI was 161% of control. The FI was 75% of control when the gene encoding the WOX transcription factor (A06) was over-expressed. Because these three auxin related genes appeared to have an effect on SCN development. The promoter region 2000 bp upstream of the ATG start site was selected for each of ten genes decreasing and increasing the FI the most and the sequences were analyzed for the presence of the auxin ARF binding sequence TGTCTC. None of the ten sequences of genes decreasing the FI the most contained the ARF binding sequence, however five of the top six genes that increased the FI the most contained the ARF binding sequence (Table 5).

TABLE 6
Auxin response element TGTCTC in promoter of six genes producing
the highest Female Index of SCN when over-expressed in soybean roots.

| | | Location | Reverse | |
|---|---|---|---|---|
| Gene | Function | (nt) | sequence | FI |
| Glyma16g33840.1 | Oligopeptide | 1720, 253 | | 254 |
| | transporter | | | |
| Glyma17g13550.1 | Pectate lyase | 1989 | | 249 |
| Glyma02g37020.1 | NAD | 1582 | | 232 |
| | dehydratase | | | |
| Glyma15g03390.1 | Unknown | 1426 | | 229 |
| Glyma03g29450.1 | Ca-depend | — | | 197 |
| | kinase | | | |
| Glyma04g39860.1 | Peroxidase III | 1685 | 148, 134 | 195 |
| Glyma19g09810.1 | Cupin domain | | 1392, 326 | 185 |

At times, the cysts of SCN varied in size and color. The variation in color correlated with size such that smaller, less mature cysts were creamy, while larger, more mature cysts appeared brown. The average number of mature cysts and small cysts were counted for four gene trials, wherein genes A12, A25, A40 and A61 were over-expressed (Table 6). The average number of eggs were counted in each type of cyst. Fewer eggs were produced by cysts on the roots with low FI as compared to high FI. Thus, genes that decreased the FI greatly when over-expressed also produced cysts that produced fewer eggs.

TABLE 7
Effect of overexpression of genes on egg production.

| | | Avg no. | Avg no | Avg no | Avg no | |
|---|---|---|---|---|---|---|
| Con- | | mature | small | eggs/mature | eggs/small | Number |
| struct | FI | cysts | cysts | cysts | cyst | of eggs |
| pRAP | 100 | 157 | 0 | 205 | 0 | 32.201 |
| control | | | | | | |
| A12 | 36 | 37.4 | 5.6 | 138.4 | 12.2 | 5.244 |
| A25 | 46 | 53.2 | 18.8 | 75.3 | 13.5 | 4.260 |
| A40 | 71 | 89.2 | 22.4 | 94.1 | 18.4 | 8.806 |
| A61 | 185 | 291 | 0 | 285.6 | 0 | 83.110 |

### Example 9

### Analysis of Overexpression in Soybean Composite Plants

The interaction of pests and pathogens with their host is complex. The pathogen often attempts to co-opt the cellular machinery of the host to its own benefit. Plant parasitic nematodes accomplish this through effector proteins that the nematode injects into the host cell it selects so it can establish a feeding site (Haegeman et al. 2012; Williamson and Kumar 2006; Gheysen and Mitchum 2011; Caillaud et al. 2008; Gao et al. 2003). Analysis of these effector proteins, their localization within the host cell after injection, and their interaction with host proteins provides insights into mechanisms involve in the formation of nematode feeding sites within a plant and provide clues to the molecular strategies used by the nematode to co-opt host cell functions. As shown herein, some host genes deter the development of SCN when over-expressed, while other genes encourage the development of SCN. The nematode injects proteins into the host cell to commandeer the cellular machinery to form a feeding site. T hus, although a host gene transcript or protein may be in greater abundance at some point during pathogen attack, the role of the gene or protein in the host-pathogen interaction may be unclear.

Thus, we found that some genes having a large increase in transcript abundance decreased the female index of SCN by half. For example, the transcript levels of genes encoding lipoxygenase (A25) and endo-β-1,4-glucanase (A12) were increased over 100-fold in syncytia during the incompatible interaction in Peking at 3 and 6 dai. Both lipoxygenase (A25) and endo-β-1,4-glucanase (A12) decreased the female index of SCN by almost two-thirds. Transcript levels of genes encoding peroxidase (A04), SAM22 (A01), and cationic peroxidase (A02) were also increased 100-fold or more, yet their effect was to increase the female index of SCN 1.5- to 2.5-fold.

The amount of transcript for two genes, one encoding ascorbate peroxidase (C21) and another encoding a protein of unknown function (A08), decreased or did not change upon nematode infection, yet they both decreased the female index by 50% or more when over-expressed. In contrast, genes R30 and C22 increased the female index by almost 2-fold or more, but had little or no change in transcript abundance. Thus, it appears that although gene expression analysis provides insights into host-pathogen interaction, the over expression of individual genes in the host, as shown here, can provide further insights into the host-pathogen interaction.

Several genes of unknown function decreased the FI of SCN when over-expressed in roots. For example, a gene A38, Glyma08g41040, decreased the FI by almost two-thirds. The protein contains sequences with similarity to ParB and STAT protein motifs. However, its function is unknown. Other genes of unknown function C19, A30 and A08 also reduced the FI by 50% or more when over-expressed.

Overexpression of a gene encoding a protein containing a PLAT domain representative of lipoxygenase LH2 (A25) decreased the FI of SCN to 26% of control levels. Lipoxygenase LH2 is associated with jasmonic acid signaling. Sigma factor sig B positive regulator, required for activation of the sigma-B transcription factor lipase (C29) and a member of the AB hydrolase superfamily, decreased the FI index by 50% or more.

Overexpression of endo-β-1,4-glucanase-6 (A12; cellulase) in soybean roots decreased the FI to 36% of control values. Using yeast two-hybrid assays, Hamamouch et al. J. Expt Bot 63:3683-3695 (2012) showed that an Arabidopsis β-1,3-glucanase is the target of the cyst nematode effector protein 30C02 from H. schachtii. When the β-1,3-glucanase At4g16260 gene was over-expressed in Arabidopsis, the number of cysts per plant was decreased by approximately 22- to 38-percent. When the effector protein 30C02 was over-expressed in Arabidopsis, the number of cysts of H. schachtii per plant doubled. Furthermore, RNAi silencing of effector protein 30C02 decreased the average number of cyst per plant by approximately 75%.

Another nematode effector protein was recently described from H schachtii that is similar to annexins (Patel et al. J Exp Bot 61:235-248 (2010)). This effector is a homolog of the H. glycines effector Hg4F01 gene. The protein encoded by Hs4F01 had 33% identity with annexin-1(annAt1) from Arabidopsis. We over-expressed two genes encoding soybean annexin, Glyma13g27020 (C12) and Glyma13g27020 (A60) in soybean roots. Neither had a very significant effect on the FI of SCN when over-expressed, although Glyma13g27020 decreased the FI of SCN by approximately 33% (P=0.25). When the H schachtii effector Hg4F01 was over-expressed in Arabidopsis, the plants, the plants were more susceptible to H. schachtii and had approximately 25% more cysts than wild-type plants (Patel et al. J Exp Bot 61:235-248 (2010)). But, when Arabidopsis plants transformed with Hg4F01 were infected with Meloidogyne incognita, there was no significant change in the number of cysts formed.

When genes encoding several membrane proteins, some functioning as transporters, were over expressed, the female index of SCN increased dramatically. These include genes encoding an oligopeptide transporter (R30), a metal ion transporter (C55), an auxin permease (A18), a membrane type III protein, and a thaumatin-related protein. The syncytium serves as a nutrient sink, providing resources for nematode growth and development. Several of these proteins may be involved in transport of nutrients to aid in the function of the syncytium.

Flavonoids are produced in developing galls of RKN and in syncytia induced by the cyst nematode H. schachtii when infecting Arabidopsis (Jones et al. (2007). Mutant lines of Arabidopsis defective in portions of the pathway for flavonoid production supported nematode development and there was no indication that flavonoids were required for syncytium development. Thus, flavonoids do not appear to support nematode development, but are produced by the host as part of its defense response. We examined the effect of overexpression of six genes related to flavonoid production (see, FIG. 5). Overexpression of the gene encoding cinnamoyl CoA reductase (CAD) increased the FI to 141% of the control. C4H converts cinnamic acid into p-coumeric acid, which is a precursor of both flavonoids and lignin. Thus, an increase in C4H could increase availability of p-coumaric acid for increased flavonoid production supporting the host defense response. CAD catalyzes reactions leading to guaiacyl and syringyl lignin production. It is uncertain why an increase in CAD expression leads to an increase in mature female nematodes, as the phenylpropanoid biosynthetic pathway leading to the synthesis of lignins, as well as many other compounds, is quite complex. Ferulic acid 5-hydroxylase (F5H) catalyzes the hydroxylation of guaiacyl lignin towards syringyl lignin precursors. When F5H is over-expressed in Arabidopsis, the amount of syringyl lignin is elevated 3.5-fold and the number of RKN egg masses and juveniles is greatly reduced (Wuyts et al. 2006 J. Exp. Bot. 57: 2825-2835). In the same study, tobacco producing less syringyl lignin supported increased production of RKN egg masses and juveniles. We also showed that overexpression of either of two genes encoding the first enzyme of this pathway, phenylalanine ammonia lyase (PAL), had no effect on the FI of SCN. This is in agreement with Wuyts et al. (Id), who showed that overexpression of PAL in Arabidopsis had no effect on M. incognita reproduction.

The level of indole-3-acetic acid or auxin in plant tissue is controlled though a variety of mechanisms, including synthesis, conjugation to amino acids (Ding et al. (2008) Plant Cell 20:228-240), transcriptional repressors, influx, and efflux transporters, and other means (Woodward et al (2005) Ann Bot 95:707-735). A role for auxin in the interactions of plant parasitic nematodes was suggested as long ago as in 1948 by Goodey when describing galls formed by Anguillulina balsamophila on Wyethia amplexicaulis Nutt. leaves (Goodey J B (1948) J Helminthol 22:109-16; for review see Gutierrez et al. (2009) Russian J Plant Physiol 56:1-5). Balasubramanian et al. (Nature 194: 774-775 (1962)) identified indole precursors of auxin in nematode galls of Meloidogyne javanica. When auxin (NAA) is applied to peach resistant to M. javanica, they became susceptible (Kochba et al. (1971) J Amer Soc Hort Sci 96:458-461). IAA applied to tomato roots increased the size of galls formed by M. javanica (Glazer et al. 1986 Physiol Molec Plant Pathol. 28:171-179).

Several lines of evidence from our data support a role for auxin in allowing SCN development in overexpression of auxin repressor (R24) decreased the FI to 53% of controls. The protein encoded by R24 has 78% amino acid identity to IAA16 [AT3G04730] from Arabidopsis thaliana, a repressor of auxin-responsive genes. Overexpression of a WOX transcription factor similar to WOX4 (WUSCHEL-RELATED HOMEOBOX4) mildly decreases the number female soybean cyst nematode reaching maturity to 75% of controls. In Arabidopsis, the WOX4 transcription factor is required for auxin-dependent growth of cambium cells (Suer et al (2011) Pl Cell 23:3247-3259). The third gene that was over-expressed encodes an auxin-permease auxin influx-transporter protein1-like LAX 4. The role of auxin in the enhancement of susceptibility within the host is emphasized further by the presence of the auxin transcription factor binding element ARF in five of the six sequences within 2000 nt upstream of the start site of genes greatly increasing the FI. Combined, these facts implicate auxin as playing a role in plant susceptibility to nematodes

### Example 10

### Control of Soybean Cyst Nematode in Whole Transgenic Soybean Plants

### Transformation of Soybean Plants.

Transformation of soybean to produce transgenic soybean plants is accomplished using mature seed targets of variety Williams 82 via A. tumefaciens-mediated transformation using explant materials and media recipes as described in Hwang et al. (WO008112044) and Que et al. (WO008112267) except where noted below. Using this method, genetic elements within the left and right border regions of the transformation plasmid are efficiently transferred and integrated into the genome of the plant cell, while genetic elements outside these border regions are generally not transferred. Mature seeds are sterilized by chlorine gas which is generated by reaction of Clorox and concentrated HCl in a desiccator. Explants are prepared from sterilized mature seeds as described in Hwang et al. (WO008112044) and infected with A. tumefaciens strain EHA101 harboring the transformation binary vector and allowed to incubate for an additional 30 to 240 minutes. Excess A. tumefaciens suspension is then removed and the explants are moved to plates containing a non-selective co-culture medium. The explants are co-cultured with the remaining A. tumefaciens at 23° C. for 4 days in the dark and then transferred to recovery medium supplemented with an 300 mg/L antibiotics mixture consisting of Ticarcillin:Potassium Clavulanate (T/C, 15:1) where they are incubated in the dark for seven days. The explants are then transferred to regeneration medium containing glyphosate (75 mM) and a mixture of 150 mg/L of antibiotics T/C to inhibit and kill A. tumefaciens. Shoot regeneration and elongation is carried out in elongation media containing 50 mM of glyphosate. The 5-enolpyruvylshikimate-3-phosphate synthase (EPSPS) gene is used as a selectable marker during the transformation process. Regenerated plantlets are transplanted to soil as described (WO08112267) and tested for the presence of EPSPS and 35S promoter/the junction sequence of eFMV-03 and 35S promoter/enhancer eFMV-07 sequences by TaqMan PCR analysis (Ingham et al., 2001). This screen allows for the selection of transgenic events that carry the T-DNA. Plants positive for the two tested sequences and negative for the two tested sequences were transferred to the greenhouse for analysis of miRNA expression seed setting.

(1) Overexpression of any one or more of the nucleotide sequences of SEQ ID NOs:1-97, and fragments of SEQ ID NOs 1-97, as described herein, in whole soybean plants.
(2) Expression of dsRNA molecules comprising a portion of any one or more of the nucleotide sequences of SEQ ID NOs:1-97 in whole soybean plants.
(3) Expression of antisense nucleotide sequences encoding a portion of any one or more of the nucleotide sequences of SEQ ID NOs:1-97, in whole soybean plants.

### Evaluation of Cyst Formation in the Transformed Plants.

Plants transformed with an expression cassette comprising the sequences of the invention are inoculated with J2 stage soybean cyst nematodes (SCN J2). Briefly, 3-week old seedling of the transgenic T1 generation soybean seedlings grown in pots are inoculated with SCN J2 suspension at the level of 3000 J2 per plant. One month after nematode inoculation, the number of cysts is determined for both the transgenic plants comprising the expression cassette comprising the sequences of the invention and for the null segregants from the same T0 parents.

The foregoing is illustrative of the present invention, and is not to be construed as limiting thereof. The invention is defined by the following claims, with equivalents of the claims to be included therein.

1-16. (canceled)

17. A method of increasing resistance of a soybean plant cell to infection by a soybean cyst nematode, comprising:
(a) introducing into a soybean plant cell a recombinant nucleic acid molecule comprising one or more nucleotide sequences selected from the group consisting of:
(i) a nucleotide sequence having at least 70% identity to any of SEQ ID NOs:1-10 or 45, or a fragment thereof;
(ii) a nucleotide sequence encoding a polypeptide having at least 70% identity to an amino acid sequence of any of SEQ ID NOs:98-107 or 142; and
(iii) any combination of (i) and/or (ii), to produce a transgenic soybean plant cell; and
(b) overexpressing the recombinant nucleic acid molecule in the soybean plant cell, thereby increasing resistance of the soybean plant cell to infection by a soybean cyst nematode as compared to a control.

18. The method of claim 17, further comprising regenerating a transgenic soybean plant and/or soybean plant part from the transgenic soybean plant cell, wherein the regenerated transgenic soybean plant and/or soybean plant part comprises in its genome the recombinant nucleic acid molecule and has increased resistance to infection by soybean cyst nematode as compared to a control.

19. A method of reducing soybean cyst nematode cyst formation on a soybean plant cell, comprising:
(a) introducing into a soybean plant cell a recombinant nucleic acid molecule comprising one or more nucleotide sequences selected from the group consisting of:
(i) a nucleotide sequence having at least 70% identity to any of SEQ ID NOs: 1-10 or 45, or a fragment thereof;
(ii) a nucleotide sequence encoding a polypeptide having at least 70% identity to an amino acid sequence of any of SEQ ID NOs: 98-107 or 142; and
(iii) any combination of (i) and/or (ii), to produce a transgenic soybean plant cell; and
(b) overexpressing the recombinant nucleic acid molecule in the soybean plant cell, thereby reducing soybean cyst nematode cyst formation on a soybean plant, soybean plant part, or soybean plant cell as compared to a control.

20. The method of claim 19, further comprising regenerating a transgenic soybean plant and/or soybean plant part from the transgenic soybean cell, wherein the regenerated transgenic soybean plant and/or soybean plant part comprises in its genome the recombinant nucleic acid molecule and has reduced soybean cyst nematode formation as compared to a control.

21. The method of claim 20, further comprising obtaining a progeny soybean plant from the transgenic soybean plant, wherein said progeny plant comprises in its genome the recombinant nucleic acid molecule and has reduced soybean cyst nematode cyst formation as compared to a control.

22. The method of claim 17, wherein at least one of the one or more nucleotide sequences of the recombinant nucleic acid is operably linked to a promoter.

23. A transgenic soybean plant, soybean plant part or soybean plant cell having increased resistance to infection by a soybean cyst nematode produced by the method of claim 17.

24. A transgenic soybean plant, soybean plant part or soybean plant cell having reduced soybean cyst nematode formation produced by the method of claim 19.

25. A crop comprising a plurality of the plants of claim 23 planted together in an agricultural field.

26. A soybean seed produced by the soybean plant of claim 23, wherein the seed comprises the recombinant nucleic acid molecule in its genome.

27. A processed product produced from the harvested product of claim 24.

28. A soybean seed meal produced from the soybean seed of claim 26.

29. A product harvested from the crop of claim 25.

30. A processed product produced from the harvested product of claim 29.

31. A transgenic soybean plant, soybean plant part or soybean plant cell comprising a recombinant nucleic acid molecule comprising one or more nucleotide sequences selected from the group consisting of:
(a) a nucleotide sequence having at least 70% identity to any of SEQ ID NOs: 1-10 or 45, or a fragment thereof;
(b) a nucleotide sequence encoding a polypeptide having at least 70% identity to an amino acid sequence of any of SEQ ID NOs: 98-107 or 142; and
(c) any combination of (a) and/or (b).

32. The transgenic soybean plant, plant part or plant cell of claim 31, wherein at least one of the one or more nucleotide sequences of the recombinant nucleic acid is operably linked to a promoter.

33. The transgenic soybean plant, plant part or plant cell of claim 31, wherein the transgenic plant, plant part or plant cell has increased resistance to infection by a nematode.

34. A crop comprising a plurality of the plants of claim 31 planted together in an agricultural field.

35. A seed produced by the transgenic soybean plant of claim 31, wherein the seed comprises the recombinant nucleic acid molecule in its genome.

36. A product harvested from the plant of claim 31.

37. A product harvested from the crop of claim 34.

38. A processed product produced from the harvested product of claim 36.

39. A seed meal produced from the seed of claim 38.

40. A method of producing a soybean plant having increased resistance to infection by a nematode, having reduced soybean cyst nematode cyst formation and/or having reduced soybean cyst nematode cyst development on roots, comprising;
(a) crossing a transgenic plant of claim 31 with itself or another plant to produce seed comprising a recombinant nucleic acid molecule of the invention; and
(b) growing a progeny plant from said seed to produce a plant having increased resistance to infection by a soybean cyst nematode, reduced soybean cyst nematode cyst formation and/or reduced soybean cyst nematode cyst development on roots.

41. The method of claim 40, further comprising
(c) crossing the progeny plant of (b) with itself or another plant and
(d) repeating steps (b) and (c) for an additional 0-7 generations to produce a plant having increased resistance to infection by a soybean cyst nematode, reduced soybean cyst nematode cyst formation and/or reduced soybean cyst nematode cyst development on roots.


SEQUENCE LISTING (207 sequences)

SEQ ID NO: 1 - DNA, 2182 bp, Glycine max
aatccccgtactctctctctctctcccttttcttgaaacttccctctttcctttaaggattccctcctaatttggtgggattatccctcatcagcattgccattccctgtttcactcaccacattgaagacatggaaaagcttattcgcatgttccctttgctccttgttcttcttctgtgccccaattttgcctttgctggccatgactacggccaagctcttagcaagagccttctgttctttgaagctcagagatctggttaccttccccacaaccagagggtcacatggagagcacattctggtttgcaagatggcaaagcaagcggggtggatctagttggagggtactatgatgcaggggacaatgtgaagttcgggttacccatggcgtttaccgttacgatgatgtcgtggagcatcattgaatacggcaaacaaatggctgctagtggtgaacttggtcacgccatggaagccgttaagtggggtacggactacttcatcaaagcgcaccctcaagccaatgttctctatggagaggtgggagatgggaacactgaccattactgttggcaaagaccagaggacatgacaaccgaccgtcatgcctacaaggtcgatccgagcaatcccggttccgatctcgccggagaaaccgccgccgcaatggccgccgcatccatagttttcagacgctctaaccctgcctatgccggcgaacttctccgccatgcctaccagctatttgattttgctgacaaatacagaggcaaatatgacagcagcatcacggttgcccaaaagtattacagatccatcagtggttacaatgatgaattgctctgggctgctgcttggttgtatcaagcatctaacaaccagtattacttggattaccttggaagaaatggtgattccatgggtggaactggttggagcatgactgaatttggttgggatgtcaagtatgctggtgttcagacattagttgccaagttcctgatgcaaggcaaatctgggcatcatgctccagttttcgagagataccagcagaaggctgaaactttcatgtgttcatgccttgggaagagtaatcgcaatgttcagaagactcctggtggcctcatcttccgccagagatggaacaacatgcagtttgtcacaagtgcctcatttttagccactgtctactctgactaccttgcttcatctggtagaaacctgagatgcagttcagggaatgtcccccctgctgagcttctctcccttgcaaagtctcaggtggactaccttcttggggacaacccaagagctactagttacatggtgggctatggaagcaacttcccacaaagggttcatcacagaggttcatccattgtttccatcaaggttaacccttcatttgtcagctgccgtggaggttatgctacatggtttagcagcaaaaggagcgaccccaacctgctcactggtgctcttgttggtggacctgatgcatatgatgactttgctgatgaaagagataactatgagcagacagagccagcaacatataacaatgctcctcttattggtattctggcaagactaggtggaggtcatggtggctataaccagcttcttccagttgttgttccagctcctaagcctgctgttaccaagcctcaaccaacccccagtcccaagacaactccttctccagcttcatggtcaggcccaatttccattgaacagaaaaggaccacctcatgggttgccaatggaaaaacttattacagatactcaacagtcgtgactaacaaatctaacaagagtctcaattctctcaatctttcaatatccaagctttatggtccaatctggggtgtcacaaagtcaggtgattcatacacattcccatcatggctcagctcattatcagctggcaaaagccttgaatttgtctacatccattctgcttctccagcagatgtctccgtggcgaactacgtgttggcctgaaagtggcatgggaatggtttactgctttcttgtgtaagactgtgtagccatacaaatgtgcagcgaggggagtgtctttacgtttttccttctataatttggtgtcaaatcttcttcttcttcttcccttgtgtattttgcatttcagtaggttagatcaaaataggtagatt

SEQ ID NO: 2 - DNA, 1109 bp, Glycine max
atcatatattcaatcacgcactttacaaagtttctccggtaacatttcctaattgctttctcttttccacctattgaccacaaactctggaacttgctctctcccctcccctcactctatatatacctttccaccccactctctctcttcacccttgttccttctcacttcacaacacctcacccattttccactctttcccttctaccttgttccacctcttaatttttctaacaaaatggaggaaagtgtgctaacaaagaggccaagagaagaagaacccctagagaacaaagatagtacttatgaattactagagtccttatcaaagaggcacaggtcatacaaccacatactctccctccttgaatcagaggaagatgactccacacaagacctatcttctctcatcacttccctccaacaagaaatcaccaattgtgcctctgattcggacacccttttgaaccaacacagcctcaccaacaccaccaccaccaccacaacaaatagtaatttagaggattgttcatcctcaacaactaaatattctagtgacatgatggaggaacatgatgacaaagaaggggtcatgagacaccttcttgaagcttctgatgatgaacttgggattccaaataaagaggatgaatcactcgatcttggtgaagatgggttcaagttcaacggtggagacatgttttcttcaatttgtgatgggttgttgtgggagcttgaagatgaagctgctaactactatgatcttttgcagtctcaactctttctctagttttttttttttggtgggggatagaaagtgaaaaaccatagttttcatgcgttcaagatgaaatccaaggaggaaaaaaatgaaaaatttactagtttttattttttcatttttttgtcccctctttttgtaaattaaaatcatgggacgtttatgtgtggttggttactacttggcaatatgtcgatctgtagaaaaagcagctaggcttatatattcacattacacaaagtaattaattagctagtaatgatcttctaaactatttcatatgttaattttattgacttttaagggtaaatgaaaattacttcttttt

SEQ ID NO: 3 - DNA, 755 bp, Glycine max
accacttggacgcaaaaatggcacctgcaacactgtctctgtctctcctcctcctgttctcactcagcatcgccgtaaccgttagatcgagcgactacgactacgattgtgtgtacactgcatacgttagaaccggttcggtgttgaagggcggaacggactctaagatcgggctgaagctgtacgacaagtacggctactacatctacattaaaaacctcgaagcatggggcggcttgatgggcaagggctacgactacttcgagcgcggcaacctcgacatcttcagcggaaggggaccctgtcttgacggacccgtctgtgccgttaacgttacttctgacggatcgggttctcaccacggctggtaccttaactacgtccaggtcacctccactgggccccacctttcctgcgctcaggatcagtacgaggtggagcagtggctcgctctcgacacttcgccttaccagctttgggccgttaggaaccactgccgctacagtttggaccgggcccagcccgtttcggagcgccccggatccggatccggatctgcgttctctattttgaatgcacgagcctgaggaaggtttttgagtcgctggtgttttatgtgtgcttcgtgtatttatatttattaatattgagaaacagatcgccatagtacttgaattggcatcgggtttgggttaacaacccgttttgtctatgtagtcttcaataataattggccctatctgtatggcctcgat

SEQ ID NO: 4 - DNA, 667 bp, Glycine max
gacaaaccacatcacgggtaaaagtgttgtcccaaagaaagagcatctttctcctttctttcatcaccgttggaagagactgagatgggttcaaaaaccaagacttttacctttgaggaggtggctaagcacaatcacaggaaggattgctggattatagtcaaagggaaggtgtatgatgtcaccccatttttggatgatcatccaggaggtgatgaagttttagtgactgcaacagagaaggatgccaccactgattttgaagatattgggcacagtgattcagcaacagagatgatggaaaaatactttgttggtgaggttgacaccaacactcttccagcacaagttaccagcagcagcagtgtacgcccaccaacacaagcacctgtctataacaatcaatcttctggatttgttgtgaagatcttgcagtacatagtgccattgctgatattggcctttgcatttggcctgcagtactatggcaaaaaaagcaagtcagaaaattgaacttgagttgtgaccattgttttagtacttctattattcataagtctctaacaacagtgtcctagtggcaatagactggtgccattgttgtttcttttctttttggggcatcatttaaaagcacttcactatctcaaatatcacgcggggatta

SEQ ID NO: 5 - DNA, 1464 bp, Glycine max
cttatcccccttttccctataaatgcaaacaaaaggcattggccttcaacgtcacaagttctttcactggaaggagaacaagaagaaaaaaccgacacaaagatctagtcctagctagaatcaaacaggggcgatctcaccttcaagaacacatcgccatggcaaggaacttgcttcttgtgctttttgcagtagccaccctcctccatggctcagcagcccaaaccagacacatggtcggtgatgccacgggctggatcatccctgctggcggcgctgccacctacaccgcctgggcttccaacaaaaccttcaccgtaaacgacactctcgtgttcaacttcgccaccggccaacacaacgtggccaaggtaacgaaatcggctttcgacgcgtgcaacggcgggagcgccgttttcaccctgacctccggtccagcgaccgtgaccctgaacgaaacaggggagcaatactacatttgctccgttggatcacactgttccgctggccagaaactggcaattaacgttaacagagcttcttctaccggtccttcccctgcacctcaacccagaggaagtggttctccaccaagggctagccctgtccccacacaagcaccacaagccagtagccccactcctccaccaaggtctgcaccagcacctgcttttggtccatcctctgaaccagctactttcattgttggagaaaccgctggctggattgttcctggcaacgcttccttttacacagcttgggcctctggcaaaaacttcagggttggagacgtccttgtgttcaactacgcatcaaatacacacaacgtagaggaggttacaaaggccaacttcgatgcttgcagctcagcctctcctattgccactttcaccactccacctgcgagagtgaccctcaacaaatctggtcaacatttcttcatctgtggaattccagggcactgtttgggaggtcaaaagcttgccatcaacgtcaccggcagcagcaccgccaccccaccttccgccgcggccccccccaccactccttctagcccttctccagccggtgctgtcactcctcccccacagaactctggtgctgcatctcttggagttgttggagtatttgccaccctgctttcagttgccgccacttttttctattagatactccacgagattattctagtcaggagcttgcgagtcatcatcatcgctctttgattgtgtttcatgaattcagtttttttcttatttttttgaggatgacaataaagttagctttaatttaatttctcttcatcggctacagttttggttttttgttatgtttttgagtaaaaataaattggtacggtgtgaatatgttaacaagtgtatttctttgatagttaacatcgttgtattacagtgcatatatttggaattggagataaaaaatatttgtgacacctttcgcaat

SEQ ID NO: 6 - DNA, 800 bp, Glycine max
gatcacacactttccaagttttaattcttcttctcttactcctcccaccttcaagtctctaataattggttatattatatccctaacaaatgggttcctccaagttattgggtattatggccatgcttttcatagtgcttctacccatggctgccaaaggggataatattactgatttctttgataaggtttgtgaagaagtggaatgtggtaagggaagctgcgtagtaaacacaagttacccattaaacttcgtttgtgaatgcgattctggctggaagcgaacccaagatgacgatgatgaatatgccactagctttcttccatgtgtcattcccgaatgtagcttgaactatggttgtcagccagcaccaccgccagttccagagaagagttttccacataacttctcagcttttgatccttgctattgggcgtactgtggggaaggtacatgcaccaagaacagaacacatacacacagatgcgaatgccaacccaattactataatcttctcaacatctcagtttttccttgttacagtgaatgtactcttggatctgattgttcgagactcggaatcaaagttgcaaattcatccactgatagtggcagtcaagatagctcagcctcaatcttcacaggaaggtttcattggatggttatgttgttgatgtccacgggtatggttatgtggagctagtacgcaaagggatagtcaaaattattttggcgaattattgatttgattgttgttcatcaattggcgtttgatgactactgtgtgatgtactgtata

SEQ ID NO: 7 - DNA, 1176 bp, Glycine max
gagtgaatgatggaataagtggtggtagcggttataaatagcagccaaagagaagaaaggctcggcaaaaaagaaaaatataaaataaaaccgccacaaatttctccatagttatgggcacttccattctcgacgccctgaacgttcgcgtggaaggttccggcgacaagtaccttgtcctggcccacggcttcggcacggaccaatccgcgtggcagcgcgtgctcccttacttcacccgcaactacagcgtcattctctacgacctcgtctgcgccggcagcgtcaaccccgaccacttcgactaccgccgttataccaccctcgacgcctacgtcgacgacctcctcaacatcctcgacgccctccgcgtcccccgctgcgcctacgtcggccactccatctccgccatgatcggcatgctcgcctccatccgccgcccggacctcttctccaaactcatcctcatcggcgcctcccctaggtacaacaaattcctgaacgacaaggactaccacgggggatttgagcagggagaaatcgagcaggtgttttcggcaatggaggcgaactacgaggcgtgggtcaacggattcgctccgctggcggtcggggcggatgtaccggcggcggtgagggaattttcccggacgctgttcaacatgcgccctgacatctcgctcttcgtgtcgcggacggtgttcaacagcgacctgagggggattctgggcctggtgaacgtcccctgctgtattatgcagacggcacgtgacatgtccgtgccggccagcgtggctacctacatgagggaccacatcgccggaaagagcaccatccagtggctcgacacggaggggcacctcccccaccttagtgctccttcatacctagctcgccagctagagattgcgctctcgcagtagcgcgtgtaagtctaggcgcgtggctctggtggttaattaagtaattaactctactagtactattactaccagattaggctagaagctactgctacagaacaacaacaacaacaacaacaagtgtacgaaaggctccgaaactggacacctggcgaataacgtgtgggtgttggttctttgactgggatttttatttaaggataagaatctgcacgtggatttcacgtgatgcgtggccatttttagtattatt

SEQ ID NO: 8 - DNA, 856 bp, Glycine max
aaaagagcgattttcccctcaccaaagaagaaccaaaacaatggcggatcaactcaccgatgaacagatctccgagttcaaggaagccttcagcttgttcgataaggacggcgatggttgcatcacaaccaaggagcttggaactgttatgcgttcattggggcaaaacccaactgaggcagaactccaggacatgatcaatgaagtggatgctgatgggaatggtaccattgacttccctgagttcctaaacctcatggctaggaagatgaaggacactgattctgaggaggagctgaaagaggcattccgggttttcgacaaggatcagaatgggttcatctctgctgctgagctccgtcatgtgatgaccaacctcggggagaaactcaccgatgaagaggtcgatgagatgattcgtgaggctgatgttgatggagatggccaaataaactacgaggagttcgttaaggtgatgatggccaagtaagtaatcatcacattatggccacaagaacctaaataaagaagtgttgattaaaaggaaatgaccaaaaaaacagaggtaaggggaaggacaagggtttgggctgtataacagtgatgtagtagtccttgtcctttcttcttcctacatttatatttgacggtttttgaatccaaccttaaaatgtcaattagtggcaattcctgctactaatggatgtagtggaactgaaccttcttagtttgtctgtaattttttccaagaaataaagggttgtaatgttaatgtcacttcgcattctaccgctcattttttttttttgaaagatgacgaatattgtgatatttgacatgcatcgtagtttatttg

SEQ ID NO: 9 - DNA, 1230 bp, Glycine max
tttgttttgttgtaacttggaagtttgtatggtggtgtgtgtgctggattggggagtgtgtgtgtgccttcaaagtagtgccttcaaagtattagatacttaatatttatatatcatgtgtgtgtgttgatttggaaggacaagtggctataacactacttgcattcagttagactttgatagtttgattagctctaataaggttaaggaccagagagagaaagagaaataagaagaaaaacacaagatgggttattggaagtctaaggttcttcccaagatcaagaaggttttcgagaagaatagcaccaagaaagctgctgctgctgaggccaccaagtcctttgatgagtcaaaggaggaatacaacaaagcctttgaagaaaagaagactgaacttcaaaccaaagttgttgaaatatatgaggcttcatcaactgaaatcaagagtttggttaaagaacccaaggaagctggtttgaagaagaactccacagaagtccagaagttcctagaagagctggttaaaattgatttccctggatcaaaggcggcatctgaagcatcttcaaagtttggaccagccttggcttcaggttcagttttctttgtgtttgagaaggtgtccactttcattgttacagaagaaaaagaagttgaagcccctcctgcagtagaaactaaaacagaagaagaaacaagtagtgttgtcaaagagagggagacagtggttgaagaagaaaaaaaggaagaggaaaaaccacaagcagacgagacaagtgatgagaaaaaagtggaagaaaaacaagctgagactgctgctaaagaggaagagaaacctgctgaaccagcagaaccaccaaagccttgaaaatttcgttctgaacagaagctatatatatgtagctgcttcatgaagatgcagaagatcatatacgtgcattcttagtttgcatatagtccaaaattgtcacataccattcccttattcttttattcctgtgtaaaattggttttgtgctgcttagtttgatattgttgcagatttggtgattgatatgcatgtcaattaatgtttttatgatgaatcgttttgtaatattattcatttttcagtcaaggaattgtgacgtttagtttggttcatcgaatccttcccagctacctgttgtttcatttaaaagttatcagtgccattatggaagtcctgtttggcagggttttacattt

SEQ ID NO: 10 - DNA, 963 bp, Glycine max
cattgttggcttgtgctctgctcgtgcctctagggttatcatatctccatttccatttcctctctctcagtttccaagaatcttaagctatgggaaagtcttacccaaccgtgagcgctgattaccagaaggccgttgagaaggcgaagaagaagctcagaggcttcatcgctgagaagagatgcgctcctctaatgctccgtttggcatggcactctgctggaacttacgacgtgagctcgaagaccggtggtcccttcggaaccataaagcacccctccgaactcgctcacggcgctaacaacggtcttgacatcgctgttaggcttttggagccactcaaagcggagtttcctattttgagctacgccgatttctaccagttggctggcgttgttgccgttgaggtcaccggtggacctgaagttcccttccatcctggaagagaggacaaacctgagccaccaccagagggtcgcttgcccgatgccactaagggttctgaccatttgagagatgtgtttggcaaagctatggggcttagtgaccgagatatcgttgctctgtctgggggtcacactattggagctgcgcacaaggagcgttctggattcgagggtccctggacctctaatcctcttattttcgacaactcatacttcaaggagttgttgagtggtgagaaagaaggcctccttcagctaccttctgacaaggcacttttgtctgaccctgtgttccgccctcttgttgagaaatatgcatcggacgaagatgccttcttcgctgattacgctgaggctcaccaaaagctttctgagcttgggtttgctgaagcctaagcaacgttttggggattgttggagaggcacggtgcccataatatttctctcataaattctatggtcatggtttctaatgcgccactgtgggttcctcaaatttttagatagcttttgaatg

SEQ ID NO: 11 - DNA, 2092 bp, Glycine max
acctacaagtaactaaccaaccggaagcacaggctacaacggtcctttcacacccggtctcaaagcttttaaaaacgaacacatacgcactcacatttccattccacctcaacaaacacaacaacactctctcttctcgctcttggcttttcgctcttcactcactctcattcattcatttccaccgttcatggatccagtaagcgtgtggggtaacacgcccttggcgacggtggatcccgagatccatgacctcatcgagaaggagaagcgccgtcaatgccgcggaatcgagctcatcgcctccgagaacttcacctccttcgccgtcatcgaggccctcggcagcgctctcacgaacaaatactccgagggcatgccgggcaaccgctactacggcggcaatgaatacatcgaccagatcgaaaacctctgccgctcacgcgccctccaagccttccacctcgacgcccaatcctggggcgtcaacgtccagccctactccggctccccggccaacttcgccgcctacaccgccgtcctcaacccccacgaccgcatcatggggctagatctcccctccggcggccacctcacccacggctactacacctccggcggaaagaagatctccgccacctccatttacttcgagagtctcccttacaaggtaaactccaccaccggctacatcgactacgaccgcttggaagaaaaagccctagacttcaggccaaaactcataatctgcggtggcagcgcgtaccctcgcgattgggactacaaacgtttcagggaagtcgctgataagtgcggagcattgcttctctgcgacatggcgcacactagcggccttgtggccgcgcaggaagtgaacagccccttcgagtattgcgacattgtgaccaccacgactcacaagagcttgcggggcccacgtgcggggatgatcttttaccggaagggccccaagccgccgaagaaggggcagccggagaacgcggtttatgatttcgaggacaagattaacttcgcggtgttcccttcgctgcagggtgggccccacaaccaccagatcggtgctctcgccgtggcgctgaagcaggccgcgtcgcccgggtttaaggcctacgcgaagcaggttaaggcgaacgccgttgcgcttggaaaatacttgatggggaaagggtacagccttgtcactggcggaacggagaaccatcttgttttgtgggatctgagacctcttggattgactggtaatatatataggattggatctctaccttctggttttgatttgttacaaatgtctataaatctgacttgttcgttgtgtgattgttttgcagggaataaggtggagaaactctgtgatctctgtaacattactgttaacaagaacgctgtttttggtgatagcagtgccttggcccctggtggagtgcgaattggtgcccctgccatgacttctaggggtttggttgaaaaagactttgagcagattggtgagttccttcaccgtgctgtgactctcacactggagatccagaaggagcatggcaaacttctcaaggatttcaacaagggtctcgtcaacaacaaggctattgaagatctcaaagctgatgttgagaagttctctgccttgtttgacatgcctggcttcctggtatctgaaatgaagtacaaggattaggttcaaccataccactttctactaaattgtgtcactcaagttcgacacaaagtgcagaaatggagaaaaaggaaatatgtgtcttcctttcctgggagtgatagggtttatcgccatggtgtttcaattcaaaagtttgaagtttctttgtctttgatttcatgtttaattttgttagcctgattgatatcatattttttttcttatttaacaattgaaataatacgtgctgcctttctttctttttttttcctcgctagctagtagtatgtttcatgatttcatcttctaatattgctcaacagaacatcttaattcttaacaaccatgagttttagtggagttaagcaaaagaaaaagttattctaataaatctatcgtctttcttatgcc

SEQ ID NO: 12 - DNA, 869 bp, Glycine max
aaacactctctaaatggcggctctcagaaaagccgtgttctttgtaatcgctcaatgcttcaccttttccgcatatgctgcaaggtttgaaatcacaaaccgatgcacatacactgtctgggctgcgtctgtgcctgttggcggtggcgtgcaattaaacccgggccagtcatggtccgtggacgtgcctgcaggaacgaaaggggcccgcgtttgggcccgaaccggctgcaacttcgacggttcgggccgcggtggatgccagaccggtgactgcgggggtgtcctcgactgcaaagcttacggtgcgcctcccaacaccctggctgaatacggcctgaacgggttcaacaatttggacttcttcgacatctccctcgtcgacggttttaacgtgcccatggactttagtccaacctcgaatggatgcacacgtggcataagctgcactgcggacattaacggacagtgccctagtgagctaaagactcaaggaggttgcaacaacccttgcactgtcttcaaaaccgaccagtactgttgcaattccggtagctgtgggcccactgattattccagattcttcaagcaaaggtgccccgatgcttatagttaccccaaggatgatccaactagcaccttcacttgtaatggtgggactgactatagggttgtcttttgtccttgaacccaataaccttacataatatataatatatgcactaaaataaagatcaataaaattacataaggcgtgtgcactttgcgtgcccctcctcgtctatgatgtgtgtacttctattctaaatgaaatggggtacttatgaataatatattaaactctttgggcaatgaaattccatttcaat

SEQ ID NO: 13 - DNA, 723 bp, Glycine max
atggatcggaaaagcctcctctccctcgcaatcatcgccattgtcgtcgccgccgtcggaggacagtctccagcggcgtcgcccaccacttctcctccggctgccaccaccactccctttgcatccccagccacagccccttctaagccaaaatcaccggcgccagttgcttctcccacgtcatcatctccaccagcttcgtcccccaatgctgcaaccgcaaccccacccgcttcatctcccacagtagcatctcctccgtctaaagctgccgctccagctccggtggctacacctcccgcagccactcctcctgcggctacaccaccggcagccacacctcctgccgtgactccagtgagttcgccgccagcgccagttccggtgagttctccacctgctcctgttccggtgagctctccacctgcactcgcaccgacaactcccgcacccgtggtagcacccagcgctgaggttccggcacccgctcccaagtcaaagaagaagacaaagaagagtaagaagcacactgcaccagcaccgtcgccgtcattgcttggccctcccgctcctccggtaggagctccgggatcaagccaggattccatgtctcctggtccagctgtatctgaggacgagagtggagctgaaaccatcaggtgcttgaagaaggtcattggatgcttagccttgagctgggctacccttgttttgttcttctag

SEQ ID NO: 14 - DNA, 2175 bp, Glycine max
cacttgaatcacagtggtgccttatcgctgtggttatcatcttcttcttcctctttcttagatctcttctttctctacgccgcacgccgaaaccatggccggaaaaggagagggtcctgctatcggaatcgatctcggaaccacctactcttgcgtcggtgtgtggcaacatgaccgcgttgaaatcatcgccaacgaccaagggaacagaaccacgccgtcttacgtcggattcactgacaccgagcgtctcatcggtgatgcggccaagaatcaagtcgccatgaaccccatcaacaccgtcttcgatgccaagaggttgattggtcgtagattcagtgattcctctgttcagagtgatatcaaattgtggcctttcaaggtcattcctggtgctgctgacaagccaatgatcgtggttaactacaagggtgaagagaagcaatttgccgcagaagaaatctcttccatggtgctcatcaagatgcgtgagattgctgaggcttacctaggctccacagtgaagaatgctgttgtcaccgtccctgcttacttcaatgattctcagcgtcaagctaccaaagacgctggtgtcattgctggtctaaatgtgatgcgaattatcaatgagcctactgcagctgccattgcatatggtcttgataagaaggccacgagtgttggtgagaagaatgtgttgatttttgaccttggtggtgggacatttgatgtgtctttactcaccattgaggagggtatctttgaggtgaaagccactgctggtgacacccatcttggaggtgaagattttgataacaggatggtgaaccactttgttcaagagttcaagagaaagaacaagaaggacattagtggaaaccccagagcacttagaaggttgaggactgcttgtgagagggccaagagaacactttcatcgactgctcagaccaccattgaaattgattctctatacgagggaattgatttctactccactgttactcgtgccagatttgaggagctcaacatggatctcttcaggaaatgtatggagccggtggagaaatgtttgagggatgctaagatggacaaaagaagtgttgatgatgttgtccttgttggtggttctaccagaattcccaaggttcaacaactgctgcaggacttctttaatggaaaagagctttgcaagagcattaatcccgatgaggctgttgcatatggtgcggctgttcaggctgcaatcttaagtggtgagggcaatgagaaggttcaggatcttctcctcctggatgtcacccctctgtctcttggtttggagactgccggtggtgtgatgactgtcctgatccctaggaacactacaattccaacaaagaaggaacaagttttctcaacatactctgacaaccagcctggtgtgcttatccaggtctttgagggtgaaagagcaaggaccagagataacaatttgttgggcaaatttgagctatctggcattcctcctgcacccaggggtgttcctcagattacagtgtgctttgacattgatgccaatggtatcttgaatgtctctgccgaagataaaaccactggccagaaaaataagatcactatcaccaatgacaagggtagattgtcaaaggaagatattgagaagatggttcaagaggctgagaagtacaagtctgaggatgaagagcacaagaagaaggttgaggccaaaaacgctttggaaaactatgcatacaacatgaggaacaccgtgaaggatgacaagattggtgagaaacttgacccggctgacaagaagaagattgaggatgcaattgagcaagcaatccagtggttagacagcaaccagcttgcagaagcagatgagtttgaggacaaaatgaaggaattggaaagcatctgcaatccaatcattgccaagatgtaccaaggtggtgctggtccagacgtgggtggtgctggtgcagcagaggatgagtatgctgctcctccttctggtggaagtggtgctggccccaagattgaggaagtggactaaatgtagttgctttcctccagatacctgttttaaaaacttattttactttatgcttttttaattggattttaatcgcatgatttttgttggatgggattcaaactatttttcccttctga

SEQ ID NO: 15 - DNA, 1200 bp, Glycine max
atggccggtcttgttgtttcttcccaatgctttctcaaactcttgcttgtagtttcagtctttcatgtttcctttgctgctaggaggttgaatgagctggttcaggaccagtctcagttgctccattaccacaatggtcctcttctatacggcaaaatcgccgtcaacctcatctggtatggtaacttcaaaccatcccaaaaggctatcatcaccgatttcgttacctcactgtcatccccagcgtctcagagcagccaaccctctgttgccacgtggtggaaaaccacggagaagtactaccacctgagtcccaggaaggcttctctctccctctctctcggcgatcagattctcgacgagacatactcgctggggaagtcactaaccggcaagaacctcgtccagctggcttccaaaggcggacagaggaactccatcaacgttgttctgacatccgctgatgtggcggtcgaaggtttctgtatgagccgatgtgggacccacggctcttccgcttctcatctgaagaagaacagcaagagctacaagttcgcctacatctgggtgggaaactccgaaacccaatgcccggggcaatgcgcgtggccattccaccaacccatttatgggccccagagcccacccttggttgctcccaacaacgatgtgggacttgacggaatggtcataaacctcgccagcctccttgccggaaccgccaccaaccccttcggaaacggctacttccagggtcccgcggaggctccgctcgaagccgcgtcggcgtgccctggggtttatgggaaaggggcttaccctggttatgctggggacttgctggttgactctaccactggtgctagctacaatgtaaagggggctaatgggaggaagtaccttgttcctgctctgtatgatccttctacgtcgtcttgctcaacgcccgtgtgatcagaagggttattttggtgatgccgatcgttctgttccatgctgctactacgatgatgttagaaaccacggattagggaggtgtacaaagcttctaataaacatttttgtagaattatttcatttatttgtttgtttatactatcttctggatatgtacgcggattaaggatgttatgtagtagaagcaaatgttcttccctttgctttttaatattaaaaaatttattattattactcttcacc

SEQ ID NO: 16 - DNA, 1053 bp, Glycine max
ttaagtaaccctgcgtattatatatagtttgtcacaacttagtaattctcacatcgagtccgagaagcattcctagctagaagaaggaaatcacttttcactttcgggctatttgctttcttagggcatctctaacaatatggccaaatccactttctttgtctgccttaacctttcattactcttctctctagtcacagccacttactactcaagtttaaccccaacacttttgggttttcgcgaggagaagttcacccacctccacttcttcttccatgatgtcgtgtcgggcccaaagcccagcatggtgtttatcgccgagcccaacgggaaagcgaaggatgcccttccgttcggaaccgttgtggcgatggacgaccctttaaccgtgggccccgaacaggactcaaaacttgtgggaaaagcccaagggatttacacttcgatatcacaggaagagatgggactaatgatggtgatgacgatggcattcaccaatggagatttcaacggcagcaccatcagcgtgttggggaggaacatgatcatgagtgaacctgttagggaaatggctattgttggtggcactggagcttttaggtttgcacgtggctacgctcaagccaggttttactctgttgatttcaccaaaggagatgctatcgtggaatacgatgtattcgtgaaccattattgagtcctacgtattagtttttattggtttataataaaagaacaaaagatagaattattgtagaacaaaaagaatgagtaattaaccagtagtagttttggtccaagggtgatagatgcttagaagtccaccacacacctttgcataagacttgaatttctttgagtctttgtagtaagctcctaatgtcattttcatgacattattgttagtcccgcctcctgatcgatgttaccttcttatctttttcgttgtgaagagtaatgatatactggatctccttaatacagagatttatattcttgaaaattaatgaaattaaattcatgttgttatatataatctaacttgatttt

SEQ ID NO: 17 - DNA, 1337 bp, Glycine max
ggagacattaaaagtggttgtttggtctcaacctcctgtggaaaaaattgataaaagttcatactctgaatatctctctctgtatgctccctgactcttacccttttacgactttttggtgcccataaattgaagttgctccctcgcttctccatcacttatggtctttgaaacaaagctagcatagcatttttcttttacaaagcaagacacccttttccttttcattccatcatctctttagctttgtccaaagaccatgtctgtggtggctgataattctgcaaacaatggaagccaccaagtggttttaaatgtaaacggtgatgcccccaagaagtgtgatgactcagccaaccaagactgtgtacctcttctgcagaagttggtagcagaggtggtggggacgtacttcttgatatttgcagggtgtgcttcggtggtggtgaaccttgacaaggacaaagtggtaacacaacctgggatttcaattgtttggggtctcactgttatggttttggtttactcagttggtcacatctctggtgctcatttcaaccctgctgtcaccattgctcatgctaccaccaaaaggtttccactgaagcaggtaccagcttatgtgatagctcaggtcgttggagccacacttgctagtggaactcttagacttatattcaatggcaagaatgaccattttgcaggaacactcccttccggttctgatttgcagtcttttgtggtcgaattcataatcactttttatctcatgtttgtcatttctggtgttgccaccgataacagagcgattggtgagctggcagggcttgcagttgggtctacggtgctgctaaatgtgatgtttgccgggccaatcacaggagcatcaatgaatccagcaagaagtttagggcctgctattgtgcaccatgaatacagaggaatatggatatatttggtgtcaccaactcttggagctgtggctggtacatgggcctataatttcatcaggtacacaaacaagccagtgcgtgaaatcaccaagagtgcctctttcctcaaaggtagtgaagctgagtgatacaacagcaaatgcatgaatgttccttttgcttcttcatttcttatcttcaagttcaccatgtattagggttaaaatgtataaggaagaatcactggtttagtgtatcgaataaccatgtaatgtttttttggtaatgcataaccatgtaatatagctagaggaagtgaaaaatacaatttctccttatccatgcaacactatatatccttctatatgttttgattctcacacaaaagaaaaaaagagacttact

SEQ ID NO: 18 - DNA, 1800 bp, Glycine max
gtccattaaattaaatcctagctacgtttccctccccaagtgcacacaaaaaaatgggtcttcaaatcaaggaaccgctccttttcactcttgtaacaatatcacttatttcaattacaaaactcttgcattcttatttttctatacctttctctccatccaatctttccattgctattgccaccctcatttttgttctaatctcatacaaattttcctcatcctctataaaacactcttccactactctgcccccaggtcctctatctgttccaatatttggtaactggctacaagttggcaatgaccttaaccaccgtcttctagcatcaatgtctcaaacctatggtcccgtgttcctactcaaactaggttccaaaaacttggtcgtggtctctgaccccgagcttgccacccaagtgctccacgcacaaggcgtagaatttggctctcgcccacggaacgttgtgtttgatatcttcacggggaatggccaagacatggttttcaccgtctacggcgaccactggcgcaaaatgcgaagaataatgacactgccattcttcaccaacaaggttgtccacaattacagcaacatgtgggaggaggagatggacttggtggtgcgtgacctcaacgtgaatgagagggtgaggagcgaagggatagttatcagaaggcggcttcagctgatgctgtacaatatcatgtataggatgatgtttgatgccaagtttgagtctcaagaagaccctttgttcattcaggccaccaggtttaactccgagagaagccgtttggcgcagagttttgaatacaattacggggattttatacccttgctccggccattcttgagagggtacctcaacaagtgcaaggacttgcagtctaggaggttggcatttttcaacacccactacgttgagaaaagaagacaaataatggctgccaatggggagaagcacaagatcagctgtgcaatggatcacatcatagatgctcagatgaagggagaaatcagcgaagagaatgtgatctacatagtagaaaacatcaacgttgcagcaattgagacaacactatggtccatagagtgggcagtagcagagttggtgaaccatccaaccgtccaaagcaagattcgtgatgagatatcaaaagtgctaaaaggggagccagttacagaatccaacctacacgagctaccatacttacaagccacggtgaaagagacactgagacttcacaccccaattcctcttctggtgccccacatgaacctggaagaagcaaagctaggagggcacactgttccaaaagagtcaaaggtggtggtgaatgcttggtggcttgccaacaacccttcatggtggaagaacccagaggagttcaggccagaaaggttcttggaagaggaatgtgcaacagatgcagttgcaggaggaaaagttgactttaggttcgtgccatttggtgtgggaaggaggagttgccctgggatcatacttgcattgccaatactggggcttgtgattgcaaagttggtgaaaagttttcagatgagtgctccagcggggacaaagattgatgtgagtgaaaaaggagggcaattcagcttgcacattgccaaccactccactgtgttgttccatccaattaagacactatgatctgttaggtcataacaaaacaagcacttagcttctgaaaaagtcttgagagttgagtcttccggtgttgctttcttagatgcatttgtttatctttctatttcaattatatacagtatactgcgtg

SEQ ID NO: 19 - DNA, 1419 bp, Glycine max
atggtcgagtttggaacgagaatgggatccatatggacggtgttgttttgttttgcatcggttttcgcgatcgtttctgcagaacgaagcattgtgaaaatggaagtgccgagattcaatgtcaccagccgagagcctcaacagagtttcctcaccaaagctttgaatttcttatggcaatctggagaatcagggtaccaacacgtgtggccggatatggaatttggatgggaaattgtgttgggtacttttgttgggttttgtggtgccgcgtttggaagtgttggtggagtaggcggaggtggcatattcgtccctatgcttagcctaatcattggatttgatccaaaatcatccacagctatctcaaaatgtatgatcatgggtgcagctgtgtcaactgtttactacaaccttaagctaaggcatcctacgttgaatatgcccatcattgactatgatttggcacttctcattcaaccaatgctcatgcttggcatcagcattggagtggtctttaacgttgtatttcctgattggattgtgactatattgcttattgttctcttcttaggcacatcaacaaaagcattctttaaaggggttgaaacatggaaaaaggaaaccataatgaaaaaggaggctgctaaacgacaagaatcaaacggttctggggctgtagtggaatacaaacctcttcccagtggaccagaaaaggacaccaaggaacaagagatgagtattattgaaaatgtatattggaaggagtttggacttctggtgttcgtttgggtttcattccttgcactacagattgccaaggaaaactatacgactacttgttcaacattatattgggtactgaacttgttacaggtcccagtctcagttggagtaactgcatatgaggcagctgccttgttcagtggacgtagagtaattgcttccacgggagaacaagggaaagatttcactgttctccagttaatgatctattgtgtctttggtgttctggctggtgtagttggtggcatgttgggactaggaggaggattcgttatgggtccactttttctggagttgggagtcccacctcaggtgtcaagcgcaacagccacctttgcaatgaccttctcctcatctatgtctgtcatagaatactacctgttgaaacgttttccagttccttatgctctttacttcattcttgtggctactattgctgcctttgttggacagcacattgtgagaaaactaattatcctattcgggagagcatctcttataatcttcattctagcctctacaatatttgtcagtgccgtctcgttaggtggagttggcattgtcaatatggtacacaagatccagaatcatgaatacatgggatttgaggatctttgcaaatacgggtcatag

SEQ ID NO: 20 - DNA, 1250 bp, Glycine max
gatcatttcatagtaagcactaaagaaaaaacgacattaattcatctatggctacccttattgctcccagcaaccactctccccaagaagatgctgaggctctcagaaaggcttttgaaggatgggggactgatgagaaaactgtcatagtaatattgggtcatagaactgtttatcagaggcagcaaatcagaagagtctatgaggaaattttccaggaggatcttgtgaaacgcctagagtctgagatcaaaggagactttgagaaagccgtgtaccgatggctactagaacctgcagatcgtgatgctgttttggccaatgttgccatcaagaatggcaaaaactacaatgtcattgtggaaatcgccactatcctctccccggaagagcttttggccgtgagacgtgcttatctcaaccgctacaagcactccttggaagaagatgttgcagctcatacctctggccatctacgccagcttttagtggggttggtgacctcatataggtatgttggtgatgagatcaatccaaaattggcacaaactgaggctgaaattcttcatgatgctgtgaaagagaagaagggtagctatgaagagaccattagggtcttgaccacaaggagcaagacccaacttgttgcaactttcaaccgctacagagagatccatggcacttccatctctaagaaactggtggatgaaggatctgatgagtttcaaagggcattgtacactgccattcgtggcatcaatgatcctattaagtactatgaaaaggtggtgcggaatgcaatcaaaaaggttggaactgatgaggatgcactcacccgtgtggtcgtgagccgcgctgagaaggacttgaagataatctcagaggtttactacaagagaaacagtgttcttcttgagcatgctattgccaaggaaacctcaggggactacaagaagttccttctcacactgttgggaaaagaagactaaaaacttttcttcgagggcatttgtgattatgagtttaggacactaagctatacttgtgtgtgatggtttcatgtgtgttggttttccttgagtctgttttcagttttccttgatcatgagaacttggagttttatcttatagagtattaatatgatagctgcttggtggcttttaaattgaacaggctcaaaataaatgatatctcagtgcccattttattttaaaaaattgatttgagttaaacaaaaaaaaaaaaaaaa

SEQ ID NO: 21 - DNA, 1905 bp, Glycine max
  misc_feature (851)..(851): n may be G or T
tcccacctaggcctctctattgtagcacttaagtttcttttcttcgtggaacttaagaaaatggaggagaaaatgagttggacagttgcagacgctgtgaactataaaggcttccctgcagacaggtccaaaacaggtggttgggtacccgcagctcttattttagggattgaaatagtggagaggctctccaccatggggattgcagtgaacctggtaacatacatgatctcaatcatgcatctgccaagctcaactgcagccaatactgtgactgactttatgggcacatcatttctcctgtgtttgctgggaggttttctagcagattccttccttggcagatacaagacaattggaatctttgcttcaatacaaacgctgggtactgctacattagcaatctcaacaaaattgccagggctacgtccaccaccttgccatgctaacagtgacagttgcaaacaagccaacggattccaaatgggaatcttgtacttgtcactgtaccttattgcactaggaactggtggcctgaagtccagtgtttcaggatttggttctgaccaattcgatgagaaagatgagaaggagaaatcccaaatggcctatttcttcaacaggtttttcttcttcatcagttttggaactctggcagctgtcacagtacttgtctatctgcaagatgaagtgagtcgcagcttggcgtatggaatatgttctgtttctatgatcatagccattatcgtgttcttatcaggaactaaaagatacagatacaagaggagtttgggaagccccattgtccacattttccaagttatagcagcatcaataaagaaaaggaagangcaactcccatacaatgttggttctttgtatgaggacactcctgaggcttcaagaatagagcacaccgaacagttccgtttcttggagaaagctgccattgtggcagaaggtgattttgagacaaatgtatgtggttctgaatcaaacccatggaaattatgctcactaacaagggtagaggaggtgaaaatgatggtgagacttctgccagtgtgggccacaaccatcatattttggaccatttacgcacagatgatcaccttttcagttgagcaagcctccaccatggaaaggaatattgggagtttccaaatccctgctggctctctcacagtcttttttgtggctgcaatactaatcactctggctgtgtatgaccgactcatcatgcccctttggaaaaagtggaacgggaaaccaggtttcacagacctacaaaggatagcaattgggcttgtattttccatttttggaatggcggctgcttcagtgtgcgagaggaaacggttatccgcggcaaaaagtgtaagtggtggaaaccaagcaacaacactgcctataagcgttttcctcctgatcccacagttcttcttggtgggttctggtgaagcattcatatacacaggccagcttgatttcttcataacaaggtcgccaaaaggaatgaaaaccatgagcacgggtctctttctcacaactttgtctctcggtttcttcatcagcagtttccttgtctcggttgttaagaaagttactgggacaagagacggccaagggtggctagcagacaacataaacaagggcaggctcgacttgttctatgcactgcttaccatacttagtttcattaattttgtagcgtttgcggtatgtgcactttggttcaagcctaagaaacccaaacaaccagctatgcaaatgggtccacagcagaggaaaagtgttgaaggctgaatagtttttaggatttcggaatgttatcatggttttattgctttgtagataggctgttatgtgtatgcatacgaagaatgggttgattttgggtttttt

SEQ ID NO: 22 - DNA, 1135 bp, Glycine max
gagttcaattccaatatattccattctctctgtgtatttccctacctctcccttcaagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggttttgagctcgttgtgtaatgttggatgtgttcccaaaacatttgaagaactttgatgtttaatgggtctgtaataatgtcccttgaaaataagttcggtttgtgttgaactcaattgtgtcccattaataatagtactctaatatcccaaaaaaaaaaaaaaaa

SEQ ID NO: 23 - DNA, 1665 bp, Glycine max
attaatcaaaatcaaatgacctaaccactctctctttttcccaattttcgaatctctctcctccatggacccaacttcgatccctccccctccggccaccaccgtccccttcactgtcgaacccagcaatcacgtgacccccgccgacaacaccaacaccaaccaccctccttacgatgagatgatatacacagcaatcggggctctgaaggagaaagatggttcgagcaagcgagccataggaaagtacatggaacaagtgtacaaggaccttccacccacccactctgctttgttgactcaccatctcaaccgattgaaatccgctggtcttctcatcctcgtcaaaaaatcctacaagctccccggatctgatcctcttcccgtgctccaggcccaaaagccccgcggtcgccccccaaagctcaaatcccagcccaacactgagctcacatggcctgcgctagccctcaacgacaaccctgctcttcaatccgcaaaaagaggcccgggtcgtcccaaaaagattgcaggacccgtaggtgtgagcccgggcccgatggtgccgggcagaaggggtcgtccacctgggaccgggagatctaagctgcccaagaggcctggccgtcctcccaagcccaaatccgtgtcggcgatctccagtgggctcaaacgacgtccgggccgcccacccaaagccgaatccaacgtaaacgtcatccccttcgcagcccctgttgctcccggcctgcccacagtgcagcccatcgttcctactgcttccgtgcccaatggatctcccaggcccagaggaaggcccaaaaagattgttgccggtgcgggggctccggcgctctcttccgttggtggtgcaccacgtggccgcggacggccccgtggggtcttgccgttggtgaggccgggccgccctcagaagctcgccgttggaaggcccaagaatcctgcaagaaggcccgtgggccgccccaagggatcgacagctgctgcaatcacagcacataaggctgctaatgatgatctaagaaggaagcttgaacacttccaatcaaaagtgaaggagtctcttggcacgcttaagccttattttaaccatgaaagtccagtcactgcaattgcggcaattcaagagttggaagtactgtcaactttggaccttaaagcaccattgagggatgagacccatcaacagccacagccacagccacaggtgtatgaacagcaatatccccagccacagccactgctgcagcaattttttcaaccacatacatcagccccaagctaggcctgaacttgatgaaggacaacttgtacaagtgtttttaacaagacacaagagataaaagatctgtaaagcggttaggaagtagtggtactgatttttctgagggtgaagtgactctgtctattttagtttgttgttgtgcaacatttagtttgacccctgaagtaattagcgggtactgttgattctgttgttatttatttgacttttacagtttactttagtcacattaatgtgtatgtttggttcagcctcctttgttgttttatatttttaatggttactgcttgctatttctcatttcccattaatcattattgagtggttgtgtgaaaaaaaaaaaaaaaaaaaaaaaaaaa

SEQ ID NO: 24 - DNA, 1800 bp, Glycine max
ctcttcaccattctctttcttctttgtttcaagtttcaacaacccagattcactctctctctcgtaattttctctcttattgaggctagaaaaagagagcaaaaaaactaaaaaatgcctgcggtcggaggtataaacaccggagggggcaaggagtaccctggaagcctcactctatttgtgacggtaacatgtatagttgcagccatgggtggtttaatcttcggttacgatatcggaatttcaggtggagtgacgtccatggatccgtttctgctcaagttttttccgtcagtgttccggaagaaaaactccgacaaaacggtgaaccagtactgccaatacgacagccagacactgacgatgttcacgtcgtccttgtacctcgccgctttgctgtcgtcgttggttgccgccaccgtcacgcgtaaattcggccggaaactctccatgcttttcggaggcttgcttttcctcgtcggtgccctcatcaacggtttcgcccaacacgtttggatgttaatcgtgggtcggatcttgctcgggttcggtatcggcttcgccaatcagtctgtgccactctacctatctgaaatggctccatacaaatatagaggagcattgaacattggctttcagttgtccatcactgttggtatccttgtggccaatgtgttgaactatttctttgctaaaatcaaaggtggttggggatggaggttgagtttgggaggtgctatggtccctgcccttataatcacagtaggatcactagtccttccagacactcccaattccatgattgaaaggggtgatcgcgagaaggccaaggctcagcttcagagaattcgcggcatcgacaatgttgatgaagagttcaatgaccttgtggcagcaagtgaatcctctagccaagtggagcacccttggaggaacttgttgcaaagaaagtacagaccccacctcaccatggcagtgttgattccattcttccagcaactcactggaatcaatgtcatcatgttttacgcgccggtcctgttcagctccatcgggtttaaggatgatgctgctctaatgtcagctgtgatcaccggcgttgttaatgttgtcgcaacttgtgtctcaatttatggtgttgacaagtggggtaggagagcccttttccttgaaggtggagtccaaatgctcatttgccaggctgtagttgcagctgcaattggagcaaagtttggaactgatgggaacccaggtgatttgccaaagtggtatgcaattgttgtggttctcttcatttgcatttatgtatcagcatttgcctggtcatggggtcccctaggttggttggtgcctagtgagatctttcccttggagattcgttcggctgctcagagtatcaatgtgtcggtgaacatgcttttcactttcttgattgcacaagtcttcttgacaatgctttgccacatgaagttcggcttgttcctcttctttgccttcttcgtgttgatcatgacattcttcgtctacttcttcttgcccgaaacaaagggcattccaattgaagaaatggggcaggtttggcaggcacaccccttctggtccagattcgtggagcatgatgattatggcaatggtgttgagatgggaaagggagctattaaagaagtgtagttagtcctcgtcttggtttatttttctcaatgactagcgttttagcttttgttggtaaatcatactattggtttcaatgtattgaattgttcctaaaaattaaaaagggttatttttcttatgtatccc

SEQ ID NO: 25 - DNA, 1116 bp, Glycine max
gtttccatttcccaaattctcattatctctctctctctctgtttcgctttcaccgaagaataacttgactttcccgaactccacctgagaaaaagatacccttaattcgcaggcgcgtcttgtgttggacaacaaccacaagatgattaacttcgaagaaaccgagttgcgactcggcctccctggcaacgactcagcactcaagggaagtgctgccaagagaggcttctctgaaactgcttctgttgatttgaagcttaatctttcttcttgcattaacgactctgcctcggattcaccctcatctgtgtctacagagaagcccaaagagaacaagactaccactgctgaacctcctccagctaatgatccagcaaaaccacctgcaaaggcacaagtggtgggttggccaccagttaggtcatttagaaagaacatagttcaaaggaacagcaatgaagaagaggcagagaagagcacgaagaatgctttcgtgaaggtgagcatggatggtgcaccgtatctacgaaaggtggacataaagttgtacaagagctaccaggagctgtcagatgcgctggctaagatgttcagttccttcacgattgaaaagtgtgggtcccaagggatgaaggactttatgaatgagaccaatggctctgactatgtacccacatatgaagacaaggacggagactggatgctcgtcggtgatgtgccctgggagatgttcgttgaatcatgcaagcgtcttcgcataatgaaaggctccgaggcaatcggtcttgcgccaagagccgtggaaaagtgcaagaacagaagctgaagaagtgatttcatctgcgtcacaaggaagcaaattgactcgagcaagatcgagtctaatataggaataagagtttaattatcatgtgcctttgttatcttttttgtaattttcttatatgtgtattatatgaatatgagaaaatagttggaaattcgttagggacacgaaataatatagaagatgtttatatgatcccgtttttgtttccaagtaaaaaatgtgactttgtctattccattgccagcttgcttacctaatgtcaatgtttcgggttaatcttaactggg

SEQ ID NO: 26 - DNA, 700 bp, Glycine max
atgaaaggtcaacaacagctaaaaaaatctaaggtggtgaaaattgactcaagaaaatcatgggaacaccacataactaatgcaaccaataaaggctaccctgttatggttcatttctctgcttattggtgcatgccttcaatagctatgaatcatttctttcaacaactggcctccacctatcaaaatgttctctttctgaatgttgatgttgatgaggtcaaggaagttgcttccaagttggaaattaaagcaattcctaccttttgtttgatgaatggaggagctccagtggataaaattgtgggtgcaaaccctgatgaattaaggaaaaggatcaattgttttattcaccagaaacattcacccaagtcagtgtgatgatgcttgaacgaacacattgaacagttgccggagctgtaaatatgacccattatgtttattatcttaataaaataaagtactaaataaaattgattccttgtatgtcacaaattccaagtggttgatcttttgttatttgtataagtttcggtgtgtacatggaatagtgtcggtacgaaagggcaagttgctgtacaataaaagggtatttattgtattcattttcttcagaatgctattttatttttctcttttttatatactttgtggtcgtatatatcaagctatcaatgcaacttgattgccaagtcatatt

SEQ ID NO: 27 - DNA, 1830 bp, Glycine max
cttagacatgcggtacatacaccatatatttgaaagaaaaaaaagcgtagtcagaggaagcatgcgcgcatctacctacccacccttttcaattatgcatgtatatatatatctgagccactttgccacattcattcccaccctcatacccttttctttcgtgcctagctactccttaattactttcattctttaatttgctgcaagctatagcttcattagttcattcacaaaattaattattacaatggtgagtgttgaagagatccgtcaggcacaacgtgcagaaggccctgccactgtcatggctattggcaccgccactcctcccaactgcgtggatcagagtacctatcctgactattatttccgcatcaccaacagcgagcacatgaccgagctcaaagaaaaattcaaacgcatgtgtgataagtcgatgattaagaagcgatacatgtacttaaacgaagagatcctgaaggagaatcccagtgtttgtgcatatatggcaccttcgttggatgcaaggcaagacatggtggttatggaggtaccaaagttgggaaaagaggctgcaactaaggcaatcaaggaatggggtcaacccaagtccaagattacccatctcatcttttgcaccactagtggtgtcgacatgcctggtgctgattatcagctcactaaactattaggccttcgtccctccgtcaagcgttacatgatgtaccaacaaggctgctttgccggtggcacggtgcttcgtttggccaaagacctcgctgaaaacaacaagggtgctcgcgtgcttgtcgtttgttctgagatcaccgcagtcacattccgcggcccaactgacacccatcttgatagccttgtgggtcaagccttgtttggagatggtgcagccgctgtcattgttggatcagaccccttaccagttgaaaagcctttgtttcagcttgtctggactgcccagacaatccttccagacagtgaaggggctattgatggacaccttcgcgaagttggtctcactttccatctcctcaaggatgttcctggactcatctccaagaatattgagaaggccttggttgaagccttccaacccttgggaatctccgattacaattctatcttctggattgcacaccctggtggacccgcaattttggaccaagtggaggctaagttaggcttgaagcctgaaaaaatggaagctactaggcatgtgctcagcgagtatggtaacatgtcaagtgcatgtgtgctattcatcttggatcaaatgcggaagaaatcaatagaaaatggacttggcacaaccggcgaaggccttgactggggtgtgctatttggtttcggtcctggactcactgttgagactgttgtactccgcagtgtcactgtctaatcatatatattgagcaagaacacagatccttcttttcttcttatgtattattgcttttttagtttgaaaaatgtattctttctcttttgctttctcacattcttcttttttgtataccagtaaacactaaacgagaacacatcttattattaatgcaattaagcttatacacaattgatctatactatacagttgcaaagtgaatatcttctattttatttttaccaatttcatctccaacaaaagattcttcgtgattgtgtataaattggactgtagaactgtttggaattatgtggtttgtgtaaagagagttgtgccgattaacttagtccagtttagcttaaaattttctgtcttgggtgagttatttgactgtgatatacaacattatatgaaacagatcaataagtatc

SEQ ID NO: 28 - DNA, 451 bp, Glycine max
caaagaatctgaggttgagtgattaaaagaatagaactatgtctaaagtggtcacactcttcactttggctcttctattaagcttcaatttaatccatgcctctcgtcctaatccttcacttaacgttgtctcttcttcgcatgaggatgttgcagctacaaaggaagagatagatgaagagagttgtgaagaaggcacagaagaatgtttgataagaaggacgctagctgcacatgtcgattatatctacactcaaaagcataaacccaaaccttaatggcatcatcgcaggaccttcttcggggaccaaatatagcaatacattatataaatcaatataccatacgtatatatgtgtacttatttcataaataaatatatgctttgtggttgaatttttggttagttttctcctttggccttggttgaattttgtgtaatttgcatacg

SEQ ID NO: 29 - DNA, 1759 bp, Glycine max
ggtgatcacacacagaagatcgatctggtagaaacgatgaaggaactatgggcacaaatggggtcactaatggccactattgtgttcatgtacacaatctttgagcgcttcttccctccacacttgcgtgaaaaacttcaagcctatactcaaaaactcaccaaccatttcaacccttacatccaaataagcttccctgagttctcaggggaacgactgaagaagagtgaagcatacacagccatccaaacctacctgagtgcaaactcatcccagagagccaaaaggctcaaagctgaagttgtgaatgatagccaaacccctcttgtcctcagcatggatgacaacgaagagataactgacgagtttcatggcatcaaactatggtggagtgcaaacaaagtaagcaacaatccacaaagatacaaccctttttcttattacggttcctcagacgagaaaaggttctacaagctaacattccataagcgccaccgtgatattgtcaccatgtcttacataaagcatgttttggatgagggaaaggatattgagatgagaaacagacagttgaagctttacaccaacaatcctagtagtggttggtatggttacaagcaatcaaagtggagccacattgtgtttgagcaccctgccacttttgaaacactcgctatggatcggagaaagaaggaggatatacttaaagaccttgtgaagtttaagaagggaaaggattattatgcaaagataggtaaagcttggaagcgtggctatcttttgtacggtcctccggggactggtaagtctaccatgattgctgctattgctaatttcatgaactatgatgtgtatgatcttgagttaaccgcggtgaaggacaacactgagctaaggaagctgttgattgagactccgagtaagtctatcacggtgattgaggacattgattgctcactcgatcttaccggccagaggaagaagaaaaaggaagagaatgaggatgaagaacaaaaagatcctatgaggagaaatgaggaggagagtagcaagagtagtaaggtgactctgtctgggttgttgaattttatagatgggatttggtctgcttgtggaggtgagaggatcattgttttcacgacgaattatgtggagaaacttgaccctgctttgattaggagagggagaatggataagcacatagagatgtcttattgttgctatgatgcattcaaggtgctggccaagaactacttggatgttgagtctcatcatttgtttggtgcaattgggggattgttggaagaaacagatatgtccccggctgatgttgctgagaatctgatgccaaagtctgtggatgaagatgtggagatttgcctgcacaaattgattaaggctcttgaagaggccaaggaggaaaaagcaaggaaaaaggcagaagaagaagaagaagcacggttgaaggaggagaaagtgaaggaagagtctactcagatggaggaaaaacacaaaggaaaaactggggaggatgtgaaggaaaatggttttcattgaggggaaatggaaatgtttctaggaaaatagcaatctgttattgagccaaccagttagatttgctcttagttggttgttgctgcattatactatcactactaggatttggtcatttgcaatatgtaacaatttgcccaatgtagtttcttggctaaataaaagttaattaaccttgtagtcttgtgcaaaaaaaaaaaaaaaa

SEQ ID NO: 30 - DNA, 600 bp, Glycine max
cctcgtgccgaattcggcacgagggagaagagagagttgaattggaagaaagagtagatcgatcagagaaagaggggagatggaagttgaagggtcatcgaagaagatgatagcgactcaggaggagatggtggaggccagggttcctctggcctacagggaccagtgcgcccacttgctcatccctctcaacaagtgcaggcaggccgagttctacctcccatggaagtgccaggaccagcgtcactcctacgaaaagtgccagtacgagctcgttatggagagaatgctccagatgcaaaagatccgcgagcaccaacaaaaccccaacgccaaacaaccccttatcgcgcttcccaaacccgccaatgcctgattcatattcactgaacctttgcttgggacagatgttatttcatgttttgctattgtatggatcaaagtgactttggaatgtttgtccaaagactacttcagactttttatattttatttctggctatttctgctgtcatggacccatgtttctccttggaagaaataaaattccttgttaacgtttcttaaagggaaaaaattaactgattgatttcataaaaa

SEQ ID NO: 31 - DNA, 845 bp, Glycine max
gcattctaatcacaacaactaaccaacgtagttatggcttcctctctacacaactatgttcttgctatcttacttttggccataacccaattcaaaaccacatctgcgggctcccatcaccacctccagcacctcaaatcccttcacttctctctgtttcaacacgagaccataaacaaaaccggatacatcatagtggatggtataaaaggaggagcaggggtgactcaaaccacaaccccttttggcaccttatttgcctttcaggaccctttgactgttgcagccaacaggtcctccaaactagttgggattgcagaagggactacagtcacatctagtctcgacgggcttcggagcatttcgatcgccaagctaaccctgcgtttgaagcaccacaagggctccctttccattgttggtgtcacaaacaatgtcaaaccctctgatcttccagtggtaggaggcactgaagatttcatgtttgtgcaaggctatattagtacttctccagttgatctcaagggtcttactgttgtctacaagattgagtttcatctttactggcccccatatgcaactcaagcctcatgaactatgttgcatgggtagaatttatgtatgttgtgaattttaaggtatccctgtatctatggtcatgaaggtcctgaacaagaacagcctaataattttgtttggaaaattaccacctgtagcgttattgtcttcgtttaaatttgttcttagtagtcagtaatttctattgtatttccaagatgttgattcaagtggttaatcttccattttcttgtctgtttgtgctattattattattattttagtac

SEQ ID NO: 32 - DNA, 1280 bp, Glycine max
tacatgtgtcacaagcattgaattttgcagtgataacttgcatcatatcaaaatctttgggcaagatagggagcttaattaaagtcctaattaaggatcatggaatcccaaacacagtctgagctaccagttgtagacttcaccaacaaaaatctgaaaccgggtactgatgcatgggtttcagcttctcaagtagtgcgtggtgcacttgaggatcatggtggtttcttggcactctatgataaggtcagtttggagacatacgactctgtttattctgaaatgatgaatttttttgacctctcaatagaaacaaaacggaggaaaaccactgaaaagcctatattcagctattctgggcaacgacctgggattcctttatatgaatctgtgggtatcatgaacccactcagctttcaggattgccagaaatacacacacgttatgtggccccaagaaaatcatcatttctgtgaaagtgtcaattcctatgcaaaacaactagtggaattggatcatattgtaaagagaatggtgtttgagagctatggactcgagacaaagaaatttgagactttacttgaatcaactgagtatgtgcttcgaggctacaaatacagaattccccgagagggtgagagcaatttgggagttgctcctcattgtgacacagctttcctaactatattgaatcagaaggtagaaggcttgggggtcaaattgaaggatgggaaatggttagaggtcggtgcttcaccctcgctatatttggtaatgggcggcgatgcattgatggtttggagtaatgacaggatacccgcttgtgaacatagagttttaatgaactcaaagatagacagatactccatgggactactttcatatgctgctaagataatggaaccacaagaggagttagttgatgaggaatatcctctacgttataaaccatttgatcattatggatatcttcgtttctttctcactgaagaggccataaaatctgattccaggatcaaagcatattgtggtatttgaaacacatattgtggtgtgaatttagtgtcatgttctaagggtgtaaaaagttgaaaaagttgcatttgctgattgccttatgtggtgtcatatatatatatatatgtgtgtgtgtgtgtgtggttattgccttatgtgtgtgtgtgttttgcctcaatgttaatggtaaaggttacagcttattttgtggatgaatatttgctgagtttgttatattctgtagatgaaatggacataaaatg

SEQ ID NO: 33 - DNA, 1070 bp, Glycine max
ggagtacaacttcacattttagaagaataccccctcttttgctgcatcagaggagttgtaaattaagagcatggctgcacaagctcttgtatcatcatcttctcttaccttctcagcggaggctgcaagacaaagtcttggaccaagatcactccaatctccatttggcttctccagaaaagcctcctttcttgttaaggcagctgctaccccccctgtcaagcaaggatcagacaggcctttgtggtttgcatcaaagcaaagtctttcttacttggatggcagccttccgggtgactatggatttgaccctctgggactttcagaccctgaaggaacaagagggttcattgagccgaaatggctagcatatggtgagataatcaatggtcgttatgcaatgttgggtgcagttggtgcaatagcacctgaaattctaggcaaggctggtctgatccctcaggagacagcactcccatggttcagaacgggtgtgttcccacctgcaggaacctacaactactgggcagactcctacacactgttcgtgtttgagatggcactgatgggatttgcagagcacagaagattccaagactgggccaaaccaggctctatgggcaaacaatacttcctaggactagagaaggggcttggaggttctggtgagccagcttatccaggaggacctttcttcaacccactaggctttggtaaggatgagaagtccttgaaggatttgaagctcaaggaagtgaagaatggaaggttggctatgttggcaatcttgggttactttgttcaagctcttgtcacaggtgttggcccataccaaaacctcctagatcatttggctgaccccgtgcacaacaacatcttgaccagtctcaagttccactgaatgtattatatgttattttacatattctttacgttgccaaaactcaactacctttctacctttctatatcatgtggacattttgttccttgatgtggcgctgtaagaaatgacatgtaaaacaactagaaacattcgacatctttcttgtgtcaaaaaaaaaaaaaaaa

SEQ ID NO: 34 - DNA, 1045 bp, Glycine max
gaccaaacaacaaatattcatggctttctttattctctccttcctcttccttcttcttgtttcatctgccactgcttgtgatcgctgcttgtatcaatccaaggcttcctatttctccaaagcttctgctctttcatctggggcatgtgggtatggctctttggcactagacataagtggtggacaccttgcagctggtgtggattctctcttcaaaaatggagcaggttgtggtgcctgctttcagataagatgcaagaacccaactctatgtagcaaagaaggtaccaaagtggtattgactgatcttaatcacaacaatcaaactgattttgtgctaagcagcagagcctttgcgggcatggctcaaaagggtatgggccaacaaatactgaagcttggcattgctgaaattgaatacaagagagtaccttgtgattacaaaaatcagaatttggccgttcgagttgaagaatcaagcaagaagcctgattacttggcaattaaatttttgtatcaagggggacaacagagatagtagccgttgatgtggctcaggttgggtcttcaaactggagcttcatgagcagaaaccacggggcagtatgggacacaagcagggtaccccaaggcgcattgcaatttaggttagtggtaacggcagggtatgatggcaagtggatttgggcaaagaaggtcctacctgctgattggaaaaatggacttatatatgattctggccttcagattactgacattgcacaagaggcttgttccccctgcgatgatgggacatgattctttgctgaaaaataactcacatatacgcatagatagtaggcatcaattttattaatactatactataggctcacaatgcaacttataataatactaaggaatcatggactagcttaataaatttgaaaagcattcttatttgtaaaccactctccacttcgttactttatattctttctcattgtaagttgtaaccaaccaaaataatatattcttcctttttggagcaaaaaaaaaaaaaaaa

SEQ ID NO: 35 - DNA, 1905 bp, Glycine max
tccctttgtgactccaaacaaagcccacacaccaacttcatttccattcacaatgtcaatttcctccacttccaactccctcattccccccaaatctttaatcccccaatcccaccccctcattcccaacatcaggcccgggctccggcccaagcctggcccatccccttccatcctcgccgtccacgccgccgagcccgccaaaatggtggtttgttgccgacaagaaccaggatcaagaccaacaatcggcatttccggccgcaatcaaagcaaacaagtggggtgtggacagcggtaagtccaagaaggccctgcagctgcccgaataccccaaccaggaggatctcgaggccgtcctccgcaccctcgacgcctcccctcccatcgtcttcgccggcgaggcccggacactcgaggagcacctcgccgaggccgccatgggaaatgccttcctcctccagggcggagactgtgccgagagcttcaaggagttcaatgccaacaacatccgtgacaccttccgcatcatcctccagatgagcgtcgtcatgatgttcggcggccaaatgcccgtcatcaaggtggggagaatggcggggcaatttgcgaagccgaggtcggattcgtttgaggagaagaacggcgtgaagcttccgagttacagaggggacaacattaacggagactcctttgacgagaagtcgaggattccggatccgcagaggatgattagggcttattgccaagccgcggcgacgctgaatcttctcagagctttcgccaccggtggttatgctgctatgcagagggttactcagtggaatttggacttcacggatcacagcgaacagggagataggtaccgagagcttgctaaccgagttgatgaggctcttggattcatggctgctgctgggctcacagtggaccatcccataatgagaacaactgaattctggacatctcatgagtgcttattgttgccttatgaacaatccctcaccaggttggattcaacttctggtctctactatgactgttcagcccatatgctctgggttggggaacgaaccaggcagcttgatggtgcccatgtcgagtttctaagaggagttgctaatcccttgggaattaaggtaagtgacaagatggatccaaatgagcttgttagactcattgagatcttgaatccccaaaacaaaccagggagaataactgtgattacgaggatgggagctgaaaatatgagggtgaagcttccacatctcatcagggcagtgcgcagagcagggcaaattgtcacctgggtcagtgatcctatgcatggaaacaccattaaggctccatgtggtcttaaaactcgccccttcgatttcatcagggctgaagtgagagcattctttgatgtgcacgagcaagaaggaagccacccaggaggggttcatctagagatgacgggtcagaatgtgaccgagtgcattggtgggtcaaggacggtcacatttgatgacttgagctcacgttaccacacacactgtgacccaaggctcaatgcttcacaatctcttgagcttgctttcatcatcgccgagcgtttgagaaagagcaggatcagatcgcagcaacctcttgcccctctaggagtgtaaaagtgccttcaaaaccaacaagagaaagatatttttgttcttttttttttttgtcctacatatttattattaatcgatggttgtcactttgattttgtgttgtgtttgtatgtgtatactacctagtaatgcgtttggactggcaaggacaagagtacttctatggataataaagaaacgaaaacgtttcaattgcgttggctactttctctttggaagtatatgatacaacgaatgcgatagaagaatcaaa

SEQ ID NO: 36 - DNA, 1598 bp, Glycine max
catttcacatggatcatatcatattccatatttgtggctcgggtgtggtggttggggtggggcacaagtgtaggtgggtggaccattaacatcatcattcatttctgaactctatgcatatataagcacttcacttcacagagcaaagacacaatctgaggcatgggttcaacaggtgagactcagattactccaacccatgtatctgatgaagaggcaaaccttttcgccatgcaactagccagtgcctcagtactccctatggttctcaaatcagctcttgagcttgatctgttggaaatcatagccaaggctggccctggtgttcacctttccccctccgacattgcttctcggctcccaacacacaaccctgatgcacccgttatgttggaccgtatattgcgcctcttggcttgctacaatatcctctctttttctcttcgcactctccctcatggcaaggttgagaggctctatggtctcgcccctgttgctaagtacttggtcaggaacgaagatggtgtctccattgctgctctcaacctcatgaaccaggacaaaatcctcatggaaagctggtactatttgaaagatgcagtccttgaagggggtattccatttaacaaagcatatggaatgacagcctttgaataccatggaacggatccaaggtttaacaaggttttcaacaaggggatggctgatcactctaccattacaatgaagaaaattcttgagacctacacaggctttgagggacttaaatccctggttgatgttggtggaggaactggagctgtagtcaacatgattgtctcaaagtatcccactattaagggcattaattttgatttgccccatgtcattgaagatgccccatcttatccaggagtggaacatgttggtggagatatgtttgtcagtgttccaaaagctgatgctatttttatgaagtggatttgccacgattggagtgatgagcactgcttgaagtttttgaagaactgctatgaggcactaccagataatgggaaagtgattgtggcggaatgcattcttccggtggctccagactctagcttggccacaaagggtgtggttcacatcgatgtgatcatgttggctcacaatccaggtgggaaagagagaacagagaaagagtttgaggctctggccaaaggctctggattccaaggtttccaagtcctgtgctgtgctttcaatacctacgtcatggaatttctcaaaaaggtttaagttctttggcgtggattcatatcaagttgcatttggattttgactttgagactctgcttggggtgctacttacgaatgttttcccggaaaaacgtaaatttcttctaatgctttatgaaaagaataacgaacaagttcaatgtataccgcctttataaaataataacaagtttcatattgtggttctggttcatatatagttctagctacgcgttctcttaagtgccaacccttataggagaaatttgttgttgtttttccattctatttctagccatccaaacaacatgtttttttactctattttcctctatttatatggttcatatttatgtccc

SEQ ID NO: 37 - DNA, 1102 bp, Glycine max
taatggaagggatagagcaccaaacactgaacgtgaatggcataaacatgcacatagccgagaagggtgaaggcccattgatcctcttcatccatggcttccccgacctatggtactcctggcgccaccagatcacagctcttgcctccctaggttaccgctgcgtggcgccggaccttcgtgggtacggcgacactgacgtgccggccaaccccacagcctacaccagcctccacgtcgtcggcgacctcgttgggcttctcgacgcgatcgtcggcgacgaggagaaggtgtttgtggtgggtcatgactggggtgcaatgactgcatggtctctttccctctatcgccccgaacgaatcagggctcttgttaacttgagcgttgttttcactccccgaaaccccaagagaaaaccccttgacactttaagagcagtgtacggaaatgactactatatctgcagatttcaggagcctggggaaatagaagctgagtttgcacagatcggcactgcaagagttctgaaggaattcctgacataccgcaaccctggtccactttatctaccaaagggtaaagcgtttgcacatccaactgattctcccatagcgttgccctcatggctatctgaagaagaatgtgactactatgccagtaaatatgacaagactggcttcacagggggattgaactactacagaaatttggatctaaattgggagctgacagcatcgtggactggtgctcaagtgaaagttccagttaagtttatagtgggtgatctagacctgacctataatgcaccaggagccaaggaatacattcacaaaggtgggttcaagagagatgtgccgcttctggaggatgtggttgttcttgaaggggctggtcactttctccatcaagaaaggcctgatgaaatcagcaaccacatttacgatttctttaagaaattctgatatgcctatgctcatgttggatgtgtctccctttgttcttggtttcattgaacggccttgttcttgctggctgagtggctgggtctgtattctcatagtggatgtgcatgcgtccatcgaaaactcttgctgagtgtgtt

SEQ ID NO: 38 - DNA, 1019 bp, Artificial
  Glycine max MnSOD (LOC100101896) with C-terminal 6 x His tag, mRNA
atggccgcgcgagctctgttgaccagaaaaaccctagccaccgtgctccgcaacgacgcgaagcccataatcggagttggcataacagcagcggctactcattcacgcgggttgcacgtgtacacgctacccgatctggattacgactatggcgcactggagccagccatcagcggcgacatcatgcagctgcaccaccagaagcaccaccagacttacatcaccaactacaacaaggccctcgagcagctccaagacgccatcgccaagaaagattcctccgccgtcgttaagctccagggcgccatcaagttcaacggcggaggtcatgtcaaccattctattttctggaaaaatctagctcctgttcgtgaaggaggtggtgaaccacccaagggttcactgggatgggctattgacacacattttggttcttttgaagcattaatacaaaaagttaacgcagaaggtgctgcactacaggggtctggatgggtgtggcttggtctggacaaagagttgaagaggcttgtagttgaaaccactgccaaccaggacccactggttactaagggaccaaatttggttccattgattggtattgatgtttgggagcatgcgtactacttacagtacaagaatgttagaccagactatctgaagaacatttggaaagttattaattggaaatatgccagtgaagtgtatgagaaagagagctctcatcatcaccatcaccactagtctgaaagtgctacttgatatagctcttgagatgacagatttgcagcacggtaagagatgtggaataaaatgatgtgatgtgatgtgataaataacactaactattttatgtagttgtactgaagaacctcgagcattgtatggctgcatttcttgtactaagttcctcttgtcttatggattttctgatgctctgttaagatactattcgtttccagtgatgtatatcttttgcctccagggtttgatccttttaaaaaaaaaaaaaaaaaaaa

SEQ ID NO: 39 - DNA, 1034 bp, Glycine max
aacaatcctaaaacgcgtaacaaatatcactatatataacggctaaacccaaatcctcctttgatcatacatacaccttctaaattctaattcttgttctcttcctcccccgatcttcaagtctctaattggttatattatataccctagcaaatgggttcctccaagttattgggtactatggccatgcttttcgttgtgctgctacccatggctgccaaaggggataatattactgatttccttgataaggtttgtgaagaagtggaatgtgggaagggaagctgcgtagtaaacacaagttacccattaaacttcgtttgtgaatgcgattctggctggaagcgaacccaagatgacgatgataaatatgccgctagctttcttccatgtgtcattcccgaatgtagcttgaactatggttgtcagccagcaccaccgccagttccagagaagagttttccacataacttctcagcttttgatacttgctactgggcgtactgtggggaaggtacatgcaccaagaacaggacacatacacacagatgcgaatgtcaacccaattactataatcttctcaacatctcagtttttccttgttacagtgaatgtactcttggatctgattgttcgagactcggaataaaagttgcaaattcatccactgatagtggcagtcaagatagctcagcctcaatcttcacaggaaagttccattggatcgttatgttgttgatgtccacgggtatggttatgtagagctaggacgcaaagggatgatgaaaactattttggcaaattattgatttgattgttgttcatcagtcattggcgtttgatgactactgtgtggtatactctatatatagatatatataatttttttgtataatatagggagtgagatttgaactttagagcttgtataaactagtcaaattccaccgttagtggattatatatagataatactataactgcagtgttattattttcttgtataataaattaataacatatcttctcccatt

SEQ ID NO: 40 - DNA, 2210 bp, Glycine max
ccaaaaaagtgtgtaaaatcatttttgtatttctaaccctctctttctcatgcaagctagcttcttctttctatttatagcccttctcccttcacccaccaacacaccacaactccaaactttttcctcttgttctcttcttcttcaacacaactagtcataatccagctcatctctctctccctctctctttgttctctctttctctttctccaaaatggctctaaactacccttttttaacctacttcatactactgctagtaaccataacccgtttgatattcactgtgggaaaaactgagcaatggaaagctccgattctaccagagcttgacattgataacatatcccacaaactccatgatgaccctgaaaccattcaaatggcttcaagggactatggccatttaacgcatgaattcccattagctgtgtttcgtccatcttctatagatgacatagtcaccttgataaaatcttcgtacaatagctttgccccttttgacatagctgcgaggggccaaggccactccactcatggacaagccatggctcgtgatgggattgtggtggacatggccagcctaagaaaacaaagaaatggggttgcgattagtgtctctaaggaccctttgatgggtcactatgctgatgttggaggggaacaactctggattgatgtgctacatgccacacttgaatatggacttgcaccagtttcttggactgattatttgtacttgaccgtgggagggacactttctaatgctggaatcagtggccagagcttccgttatggacctcaaatcagcaacgttcatgaaatggatgtcatcactggaaaaggagagttcgtaacatgctcttcacagaagaacttggagttattccacgcggttcttggaggcttgggacaatttggagttatagcaagggcgagaattgctcttgagccagcacccaaaagggttaagtgggtcagactactttatagtgacttttctgcttttaccaaagaccaggaacgattaatctcaatcaatggaaggaaacaaaagaacgcattggattttctggaagggatgctgctaatgaaccaaggccccataaataattggagatcctctttcttccctctatctgaccatcccagaatagcttctttaataactgaacatagcatcctctactgtcttgaagtggctaaatattatgacgaacaaaccgagttaaatgtggacaaggaaattgaagttttgctccaaggactagcctatatccctggatttaattatgagaaaaatgtctcgtacgttgagttcttgaatagggtccgaagtggagagttgaaacttcagtcacaaggactgtgggaagttcctcacccgtggcttaatttgtttataccaaaatctcaaatcttggattttaattcaggagtattcaaagatatagttcttaaaagaaacatctcctctggaccagtcttggtttatcccatgaatagaaacaagtgggacgataggatgtcagcatctataccagacgaggatgttttctacacagttgggtttttgcactcaagtgggtttgatacttggaaggcatatgatgctcaaaacagagaaattttggagttctgtagagatgctggcatcatggtcaagcaatatcttcccaaccacagcacacaagaagattggacaaaccattttggtgctaaatggatgaaattcttagaaagaaaacatcagtttgatccaagaatgattctatcacctgggcaaaaaatcttccacaaaaaattacagccagtgttttaaatattttccctaataaaaaaagtaaaaatgttttgtggtgggactacctcaattagatggctagctagggacaggtgtacatacagtgtgaccaagaccaagatggcttttgtatgctaaagtttgcattgctagctagagaaattaataaggtggggcattaaaggctgcatgagtacttttggcacctattatgctacatattaccttagatacgttaggttgacaaagtcgtactggtaggtacaagaagcacagttgtctctcacactgtctgtcacccaacttgctatatagtgtgtggagaaattcggatcatattgaagatgctgtgagttatagtcctccaatattaaatgtttaacgattctgtctttctcttata

SEQ ID NO: 41 - DNA, 1450 bp, Glycine max
gaaaagtaacgttcagatttgatgatgatggtgttaaggcgataacaactaggaagcaatatttggcaatatatatatatactgagcttgtgataaaaccaattcaatctccgatatcacatatatttcttcagcacttatagtgtggtgagagagaaattaaggaagagctagcttaattgagtgctgctaattctatggctaccctcgttgctccaaaccaaaaatctccggttgaagacgttgaggctctacataaggctttcaaaggatgggggaccgatgagaaaaccgttattgcaatactgggtcatagaaatgttcatcagaggcaacaaatcagaaaggtttatgaggaaatttaccaagaggacctcattaaacgcctggagtctgagctctctggtgactttgagagagctgtgtaccgttggatgctggaacctgcagatcgtgatgctgttttggccaatgttgccatcaagaatggcagcaaaggttaccatgtgattgtggaaattgcttgtgtgctttcagctgacgaggtcttggcagtgaagcgtgcctatcacaaccgttacaagagatctttggaagaagatgtggctactaataccactggtgatattcgccagctgttggttgggttggtgacagcatataggtatgatggtgatgaggtcaatgcaaaattggccaaaactgaagctgatattcttcatgaatctatcaaggagaaaaagggcaaccatgaagaagccatcaggatcctcaccacaaggagcaagacacaacttctggcaactttcaaccgctacagagatgaccatggcgcttccataactaagaaattgttggataatgcatctactgacttccagaaagcattgcacactgctattagatgtatcaatgaccacaaaaagtactatgaaaaggttctgcgcaatgcgataaaaggggttggaactgatgaggatgcactcacccgtgtggtggtctcaagggctgagaaggacctcagggacatcaaagagctttattataagagaaatagtgttcaccttgaggatgcagtggccaaggaaatctcaggggactacaagaaattcatcctcactctgttggggaaggaagattgaagtgattgctctagccaaggacatgtctttagtttgcttatgttggattcgtgtgctgtgatttggttaatttccctatttagagtgtgttttttatgagttctgtatctttgttgtgagagtgtgtacttctgttttaaattactgttgttgatatttgattagtataataaactttaaatatgtaattaaggtctggttttcttataatagtcttttaaaatttcctaatctgatatgtaattagctgaatgatgtgatgtgatgcatataatcgatttcacttaaagaagtaagattttattatt

SEQ ID NO: 42 - DNA, 631 bp, Glycine max
gggcaccatcactagaacatatagaaaaaccaagtaatactagccaaaatatgtctcccaaatacatgtcccttttcctcctagtaatactagggatggcttttctggccactacttcccttgctgatcgtcgttttctttcggtggaagatggtcatgatcaccatcaccaccacccaccaaaaaaacactggcctccaaccacacaaaatgaaccttcaaacgtagaaaacaaggagaacactgaggtggaggatggtcatgatcatcaccatcactacccacctaaaaaacactggcctcctaccacacaaaatgaaccttcgaaagtagaaaacaaggatggtcactataagcctccccacaagaagcatcctccttcgggaaattaattaatctagtgaagctgctacgtactaccatgcatgcactcttgtgaaaatatattagagtatatggtttaatcaattagagtccaatctcttggctcttgaaataaaggcatgcattaatccgatgttattttcttgtattagtgtggttgtttttgctttggtcactttggctttgtatttggttgaaataaagatgcacatgctacatgtatgcaatcatatatgttatcatttcc

SEQ ID NO: 43 - DNA, 351 bp, Glycine max
atggctcctcggtctagcagcaaggacgcccaagaccttttccgcgctctttggtctgcttatgctgcaacccccactaatctcaagatcattgatctctatgtcatctatgccgtattcaccgctttcatccaggttgtttacatggctttggttggatcatttccatttaactccttcctatcaggagtactttcttgtgtaggaactgctgttcttgctgtttgtctcaggatccaagtgaataaagagaataaggaattcaaggatcttgcacctgagcgcgcttttgcggattttgttctctgtaatttggtgcttcatttggtgatcatgaacttccttggttaa

SEQ ID NO: 44 - DNA, 1509 bp, Glycine max
gcaccctcccaaccacctttgatctaggttgttatttttttctctctgaatttaaatgaaatggctcagaaaagacccccaaatagcagggggtactacgttaagatgaaactccttcacaaacatggcagacctcatcatcatcatcagcagcagcaagaaaaaaactgcctctacagatactacaaatgggttctctggctttctctttccctctatttcttcacctcctacctcatcagcaacaacaacaacaacaaccaccactccaaacaaccatctcacgtctcgagagccctcatggaatcaaaccacaccacacctcctcaacaacaagctcttaactctctagggtctttgaagaacttgaaggtgtttgtatatgatcttcctcagaagtacaacacggattggttatcgaacgagaggtgcagcaaacatttgttcgcgtcggaggttgcaattcacagggctttgttgaccagcgaggttcgcacgtttgacccatacgacgctgactttttcttcgtccccgtttatgtgtcttgcaatttcagcaccgtcaacggcttcccggcgattggccacgcgcgctcccttatcgcgtccgctgtcagcctcgtctcctcggagtaccccttctggaaccggagcagaggctccgaccacgtcttcgtcgcctcgcacgattttggctcgtgtttccacaccctggaggacgtggcgatggctgatggcgtgccagagattatgaggaactcgatcgtgttgcagacgtttggcgtagtgtatgatcacccgtgccagagtgtcgagcacgtggtgattccgccgtacgtttcgccggaaagtgtacgggacaccatggagaattttccggtgaacggacggcgagatatttgggctttcttccgagggaaaatggaacttcatcctaagaacgttagtggacgattttacagcaagaaagtgcggacggtgatatggaggaagttcaacggtgatcggcggttttacctgcagaggcaaaggtttgccggttaccagtcagagattgcacgttcggtgttttgtttatgtcctctggggtgggccccatggagtccgaggctggtcgagtccgttgccttgggttgcgtgccagtaatcatagcggacggtatccggttgccgtttatctccgccgtgaagtggccggagatatcaatcacggtggcggagaaagacgtggggaggctggcggagatactcgagcgcgtggcggcgacaaacctcagcaccattcagaggaacctgtgggacccagtgacgaggagtgccctacttttcaacagccaggtccagaaaggggatgccacgtggcagatcctaagggctttaagtgaaaagcttgatagatccttcagaagctcgaggttaaactcggcgcccacttgtgtttacttcttcactcaccggtggtaatgaagatgacatttcaaaaacacgctgcaggacgataataaatag

SEQ ID NO: 45 - DNA, 1206 bp, Glycine max
attaaaataagttcaattcaaatactactgttatcacaattgacattctacacgtctctaacagtaaaaccatataaaaactagctactactacataagttgaagtaatggccttttcttctcactagggcttaagagttaagaagaaagatgcaagggtgctcagacgctccactctccaagaggttagaagacaaagtagcactaatcaccgggggagcaagtggcatcggcgaagccactgcaaggcttttccttcgccacggtgccaaggtcgtcatcgccgacatccaagacaacctcggacactccctttgccaaaacctcaactccggcaacaacatttcctatgttcactgtgatgtcactaacgataacgacgttcaaatagccgtcaaagctgccgtttcacgccacggcaagctcgacatcctcttcagtaacgccggaatcggtggcaattcggactcctccatcatcgcccttgatcctgctgacttgaagagggtttttgaagtcaacgtcttcggcgctttctacgccgccaaacacgccgctgaaataatgattcctagaaagatagggagcattgtgttcacgtctagcgctgtttcggtgactcatccgggttcgccgcacccatacacggcgtcgaagtacgcagtggtgggtctgatgaagaacttgtgcgtggaactggggaagcatggaatcagagttaactgcatttcaccctatgctgtggccactcctctgctgacacgtggaatgggaatggagaaggagatggtagaggaactgtttgcggaggcagggaacttgaagggtgtggttctcaaggaagaggatttggcagaagcagctttgtttcttgctagtgatgagtcaaagtacgtgagtggggtcaacctagttgtggacggaggttacagtgttaacaatactgcttcggctgaagtagctttaggaaagttttctgctgattaagcccaaattgtaagctttcatatttgagttccttcgaccaaaagccaaaaggtgtgttttctagtttgaattaaaataaaataaaaaagtgaaagtgtgtgtaagacaataatacaatatttctgttatttagcaaaaagtgcagttacttttagctggaaaaaaaatccaaatttgtatttctgtatgtgaattaaaatataatataaaatagtatgt

SEQ ID NO: 46 - DNA, 928 bp, Glycine max
ccacttcaattcttctacttaactagcataaaaatcacgtttgcaattctgcgtgttctttccccacccttcattatacgtacatattcatccacccaccctctagctaccaataaaattgttcactttcactaattaattttatagtatggaaattgaagagcaaacgatgaaaatgatgatgaagtcgtcgtctaggttcagacgtatctgtgttttctgtggcaccagccctggcaagaaccccagctaccaactcgctgccatccaactcgccaaacaacttgtggagaggaacatcgacttggtttatggaggagggagcattgggttgatgggtttaatctcacaagttgtgtttgatggtggacgccacgtgttaggggtgattccaacaactcttatgccaagagaggtaacgggggaaagtgtcggagaagtgagagcagtgtcaggcatgcaccaacgcaaggcagaaatggcgagacaagccgatgcatttattgcgctgccaggtggatatggcaccctggaagaactacttgaagtgatcacctgggctcaactaggaatccacgataaacccgtggggttgttgaacgtggatgggtactacaactcgttgctgtcattcatggacaatgctgtagacgaaggtttcattacacccgctgcccgtcacattattgtctctgcccaaactgcccaagatctcatgtgcaagcttgaggaatatgtcccgaagcactgtggcgtggcaccaaagcaaagttgggagatgaaccaacagttataaaaagttaacactgcaaattaccaaattcagatacttgtcgttaaccaagtcgaatctacatattttctctaccccattagaaaatagctttctcaatacaaatcaagccttttttggctgacactgtgtaat

SEQ ID NO: 47 - DNA, 2676 bp, Glycine max
caattccgagtgccaatccaacggtccaagttaattttcaacaaaccccattttccccttagctacctaccaaacaccactgcatgaggacaacaacaacacacaaagttatctctatataaatgcccaaaccaaaccttctttctcctcaggaaatccacaaaactaattaactatctctctctctgtctctacaaaacttagctctgttttttaattattccttgcattctcttgagttcagatggaagcaactaatggccaccaaaatggttcgttttgcttgtccagtactaagggaagtgatccgttaaactggggagcggcggcggaggcgatgaaggggagccacctggacgaggtgaagcgcatggtgtccgagtaccggaagccggtggtccggctcggcggcgagacgctcaccatcgcccaggtggccgccgtggctggacacgaccacggcgtgacggtggagctctcggagtctgcccgagaaggagtgaaggcgagcagtgagtgggtgatgaacagcatgaacaacggaactgacagttacggcgtcaccaccggcttcggtgccacgtcacaccgccgaaccaaacaaggtggtgctctgcagaaagaactcatcaggtttttaaacgcaggaatatttggaaatggaaccgaatcaagccacaccctgccacacactgcaaccagagcagccatgttagtgaggatcaacactcttcttcaagggtactcaggcattagatttgaaatcctagaagccatcaccaagctcctcaacaacaatgttaccccatgtttgccacttcgtggtacaatcacagcttctggagatcttgttccactttcttacattgctggtttgctaactggcagacccaactccaaagctgttggacctaatggagaagtacttaacgctaaggaagcttttgaattggctagcatcaattctgagttctttgaattgcaacccaaggaaggccttgcccttgttaatggcactgctgttggttctggattagcttctatggttctctttgaggctaatatactagctgtgttgtctgaagttctatcagctatttttgcggaagtgatgcaagggaaacctgaatttactgaccatttgacacacaagttaaagcaccatcctggtcaaattgaggctgctgcaattatggagcatattttggatggaagttcctacatgaaagctgctaagaagttgcatgagattgatccattgcaaaagccaaaacaagatcgatatgcccttagaacttcaccacaatggcttggtcctctcattgaagtgattcgtttctcgactaagtcaattgagagagagattaactctgtgaatgacaaccctttgattgatgtctcaaggaacaaggcattacatggtggcaatttccaaggaaccccaattggagtctctatggacaacacgcgtctggctcttgcatctattggcaaactcatgtttgctcaattctctgagcttgtcaatgatttttacaacaatgggttgccttcaaatctcactgctagcagaaatcctagcttggactatgggttcaagggagctgaaattgccatggcttcttactgctctgaactccaatatcttgcaaatccagtaactagccatgtccaaagtgctgagcagcataaccaggatgtgaactctttgggtttaatttcatccagaaagacaaatgaagctatcgagatccttaagctcatgtcttccacattcttgattgcactttgccaagcgattgacttgaggcatttggaggagaatttgaaaaactcggtcaagaacactgtgagccaagtttccaaaaggattcttaccacaggtgtcaatggagaactccatccttcaagattttgtgaaaaggatctgctaaaagtggttgatagggagtacgtattttcctacattgatgacccctgcagtgctacatacccattgatgcaaaaacttaggcaagtgcttgtagatcatgccttggtaaatgcagagaatgagaaggatatgaacacatccatctttcaaaagatagcaaactttgaggaggagttgaagaatttcttgccaaaagaggttgaaagtgcaagggttgcttatgagagtggcaaagctgcaattccgaacaagatccaagaatgcagatcttacccactgtacaagtttgtgagagaggaattagggactgggttgctaactggagagaaggtcaggtcaccaggtgaagagtttgacaaattattcacagcaatgtgccagggcaaaattattgatcctcttctggagtgccttggggagtggaatggagctcctcttccaatctgttgattttactataacttttacaaatattttctttgtacctatgcaagtgcaaccataatcatttggtttgtcaatcctttaacaaatgttcctttaatgtcaaataggaccttgtaatttaatattttaatggaatttcagtagtttgccggagctttggttctattatatactaagtctcattttttgaccctcagtttttatccatcttgcatctttttctatgtctctcctgttttagccatcacatttattactttctttctttcttatatctcccacctgtccattattca

SEQ ID NO: 48 - DNA, 1505 bp, Glycine max
gaaccttttcacacctttcttcatttcacatggaaatcagctccctcgaagactattcttccttcttcttccctgatatggatcccaagcgtcgtggaaacgacaccgctttcaacgacctcctcaattccctcatccacttagacaacaaacaaaccctcatggatcctactcaattccaattcgactcctcccccccaaccaagagacctcgtcgtgccattgaagacatccacaatgtcacggtccaacaacaacaacaacaacaacgccgcctgtgggtcaaggatcgctccaaggactggtgggaccgctgcaaccagccggacttccctgaagaggaattccggcgctacttccgcatgagcaaggccaccttcgacatgatctgccagcatctggactccgccgtcaccaagaaaaacacgatgctccgccaggcgatcccggtgcggcagcgcgtggcggtgtgcatttggcgcctcgccaccggcgaccccctccgcgaggtttccaaacgcttcggcttgggaatctccacgtgtcacaagctcgtgctggaggtctgctccaccataaaaaccgttctgatgcccaagttcctccactggccaaacgaagccgaaatgaaacccatcacccaaaaattcgaatctctctccggaatcccctacgtgggaggctcaatgtacaccactcacattcccatcattgcacccaagtccaacgtcaacgcttacttcaacaagcaccacacagaacgaaaccagaagactagttactctataaccgttcagggcgtggttgattccaaaggtgtctttagtgatgtttgtatcggttggcctggttctctctccgacgatcaagtgttggagaaaagcgcgctgtatcagagagcgactatggggactttgaaggacgtttgggtggtgggaaactctggacaccctcttatggacggtgttttggttccttacacgcatcagaatcttacttggacgcagcacgcgtttaatcagaaggttggggagattcagagcattgctaaggacgctttcgcgaggctcaaagggaggtggtcttgcttgcagaagaggaccgaagtgaagcttgaggacttgcccgtgcttcttggggcgtgctgtgttttgcataatatttgtgagatgagagatgaggaaatggatcctcactggaagtttgagatcttcgatgatgagatggtggcggagaataacgttcgctctaatgctgcagagcaagccagagatcagatagcgcactatttgttgcaccatggtcgtgcagggactaattttttgtagacttctagtttcgattttgtgccatggtcatagtcatgtgttgttgtacttggggtatcagagtttctcattaattcttttggctatggctttattattatagtcacattataataagattttgatgttaatgggtattggtatgtatcaatttgtatagatatctttaatttgccc

SEQ ID NO: 49 - DNA, 1025 bp, Glycine max
aagttagtagtagtgaagaaaatgaaagtccttgttttctttgttgctatagttctagcagcatggcattgccatggctcagaccatgaccatgaccatggccacacctaccaaattttccctctgagaatgaaaactggccatggtggccactacattccggaggtatcatgccaaagttggaggcttggtgtggaagcacacaacgtcattgactggaaaaccgttcctcaagattgcgagggatatattgggaactacatgcttggcgaacaatatagatcagactccaaaatagttaaccaacaagcttatttctatgctaaaaccctcaatatcactgccaaaaccgcatgggtgttcgacatagatgagactacactctctaatctcccttactatgctgaccatggatttggggtggagttatataatgaaacatcgtttaataaatgggttgaccttggcgaggcaccggcactgccagagagtctcaaattgtacaagaaactcttgtctcttggcatcaagattgtattcataacaggaagaccactggatcaaaaggctgtaactgctaccaacttaaacttaaagcttgctggatatcacacatgggaaaagttaattaccaagaatacatctgaataccatggtaagacagcagttacatacaaatctactgagagaaagaagctggaggaaaaaggatacaaaatcattggaaacattggagaccaatggagcgatctattaggaaccaacacaggcgataggacctttaagttgcctgatcccatgtactacattagttgaggtctctagggctcttactagcatgttcctatgaataaaataagatgcatgttgtaataatggagagatcgacttaaaccaaggggcactcgttaattaattgctatgtatgtgactttgtgtgagacaaaaggtcacgtggtaagagactgtacaagcacaacggtgttttatctttcgtggttttaataaatcatgtgtgctataat

SEQ ID NO: 50 - DNA, 1289 bp, Glycine max
atggcaggttcaatttctttgttggagttctctttggtcatttttatccaaatcatgacacactgccatagcagcatcaccacatgcttgcctgagaaacatgctgcactattcatattaggagattcgctatttgataacggaaacaacaattacatcaacaccaccacctcttaccaggcaaactatcctccctatggagaaactttcttcaaatacccttctggcaggttttctgatggacgcatgataccagatgccgttgctgagcttgctaagttgcctatacttccaccgtatctgcatccgggtcacgttgaatatgtctatggtgtcaattttgcttcaggaggggccggtgctctgcgcgaaacatcccagggaatggtgatagacctcaaaactcaggtaagttacttaaaaaatgtaaagaatttatttagtcaaagattcgggcatgcaatagccgaggaaatactgtccaaatctgtctacttgtttaacattggagccaatgactatggttctctactggatccaaactcaaccagtgtacttcttccggttgatcatcaaggatttgtagacattgtaattggaaacctcacagacgcgatcaaagaaatttataatattggtggaaagaaatttggatttcttaatgtgcctccgataggttgttcccctgccataagaattttggtcaataatggaagcacatgctttgaagaattttcagccatagcaagattacacaacaatgcactctcaaaaaggcttcacgagctagagaaacagctcaagggattcaaatattcagtcatggatttttatagtgcattttctcaagtgttcaacaatcctacaaaatatggcttcaaagtagcgagcgttggatgttgtggaagtggaccatatagaggagtggatagttgtggagggaacaaagggataaaagaatatgaattatgtgacaatgtcaatgaacatttgttcttcgactctcatcatcttactgatagagctagtgaatatttcgccgagttgatatggaatgcaaatcgcactgtcacaagtccttacaatctgaagcaactatttgaactttgaatatgttctaccaagcaagatatatctaaagaaagacctattatcatgataaatttgtttccaagttctgtattttggttcactctggtattttgtgttcaataagtgggttgagcttgccaaataaaatggtcttgatgacttgaattcataattattatttttgcactgcaaaaagg

SEQ ID NO: 51 - DNA, 1133 bp, Glycine max
cagtgtgctatagtgagtctacgtgtttgagagaaaggttgagctatagcctaaagggagagacacataaatagtttctactttctattcatagatagatggctggcccaattatgagacctcagattgtgctatttggctcctccataattcaaatgagcttcgacaatggtggttggggtgctattctagctaacttgtacgctaggaaggcggacatcatcttaagaggatactctggttggaattcaaggcgggctttggaggttttggatgaaattttccccaaggatgcttatgtgcaaccatcattggtaattgtgtattttggtggcaatgattctattgatcctcacccatctggccttggtcctcatgtaccccttgaagaatatgttgaaaacatgaggaaaattgctaatcatcttaagagcctctcggaccatattcgcattatatttctcacttctcctccgatcaatgaagaactaatccgcaaaaagctcagtgcaacgcaatcaggaagaaccaatgaatcctgtggagagtatgcagatgggttaatggagctttgtgaggagatgaatatcaaggccattaatctgtggtctgcaattcagacaagagaggattggttagacgttagcttcacggatggagttcatctatcagcagagggaagcaaggtagtggtgaaggaaatattaaaggttctaagagaagtagattggaaacctagtctgcattggatgtcaatgccaactgaatatgcagaagattcaccatattatcctccaagtcctgatggaacaacaaccataaatgtgtctcatattatctcccgaaggtgtttgcagtgggatatatagtactttatgtatttatattgcgtaccatataagaggctcaattatgttgtgtggagtggagctcttggaaaagtgatattttccctaccccatacgttttgggggcgctttaattattctattctagttttcatttgttacatgtacttaaatttgtgttaataaattaaaattccactcaatttacgctttatattctctcttgttttcagctttcaatctcatacacagtaagctcttttattgccaaaaaaaaaaaaaaaa

SEQ ID NO: 52 - DNA, 1770 bp, Glycine max
gtaattggaagagggggatgatgagggtgaactaaaaagctagctagctagtttcatctgggttatgcttatgtctggcacagaacaggtgaccatgggaggattaggactcaatttgactccaattactacatttgctatcattactgtgattgctacggtgctaatatggtggttttggaatgcactcaactgggtgtggctgagacccaagaggatagagaggcgtctcaaggagcagggtatccaaggaaattcctaccgccctttgattggagacatcagagatatggttaagatgattaaggaagccaaatccaaacctatggatcctcactctaatgacattgcaccccgtgtgttgccttacgttgttcacaccatcgctaaatacggtaagagttcatttatgtggcttggtccaacaccaagggtattcatcttggatcctgacaaattcaaagaaatggctactaaggtctatgattttcaaaagcccgacactagtccacttttcaagcttctagcatcagggtttgcaaattatgatggcgacaagtgggctaaacacagaaagatcgtgagtccagcattcaatgtagagaaaatgaagctcttggtaccgatattttgccagagttgcgatgatttgatcagcaaatgggagagtttgttatcttcgtctaatgggtcttgtgagttagatgtgtggccttttgtccaaaacgtgtcaagcgacgttcttgctcgtgcaggctttggaagtagctatcaagaagggaaaaaaatattcgaacttcaaagggaaatgattcagcttacaatgacgctctttaagtttgctttcattccaggttacaggtttctgccaacgcataccaacaggaggatgaaagcaattgacaaagaaatacgagaatcacttatggtaatcatcaaccgaagattaaaagcaatcaaagcaggggagcctactaacaatgacttgttaggcatactcttggaatcaaattacaaggaatctgaaaaaagtagtggtggaggaatgagtttaagggaagtagtggaagaagtgaagctattttacttggcagggcaggaagcaaatgcagaattgctggtctggacattgttgttattaagcaggcatccagattggcaagaaaaggctagggaggaggttttccaagtgttcgggaatgaaaagccagattatgaaaggattggtcaactgaaaattgtgtcgatgattctacaggagagtctcagattatacccaccagtggttatgttcgctcggtatcttcgtaaagatacaaaacttggagagcttacaattcccgcaggagtggagcttgttgtacctgtgtcaatgctacaccaggataaggagttttggggagatgatgccggggaattcaacccagaaagattctcggaaggagtatcaaaggcaacaaaaggcaagctttcctacttgccatttggatggggtcctcgactctgcataggacaaaactttggcttgttagaagcaaaagtagctgtgtcaatgatcctgcaacgtttctccctccatttttccccctcctatgctcatgccccctcctttattatcactcttcagcctgagcgtggggctcatctcattttacgcaaactctagaatgcactttttagtctaaataaggcttcttcatttgaggcgtgtttgcaattttagttttcctattcctgctagtttcttatgtagacgatagatctgaacct

SEQ ID NO: 53 - DNA, 2371 bp, Glycine max
atctcctctctttaaagcttcaaagcctctctcaaaaccttacaacttttgatctcttgtttgtgaacatggcgaccattattctagaaaatgaccctcttaactggagccatgcagcagattcactcaagggtagccactttgaagaagtaaaacgcatggtggcagaataccggaagccacttatctctctgggtggcggggagacactaactatatcacaggtggctgctgttgctgttgccaatgccaaccataacttgcaggctaaggttgatctctcagagtctgcaagggcaggcgttgacgccagctgcgactggatcacccaaaacattaacaaaggcactcccatttatggtgttaccaccggctttggtgctgcctcccacaggcaaactcaacaaggccttgctcttcagaaggaaatggttaggtttttaaactgcgcaatatttggctaccagacagagttatctcacacactgcctaaatcagcaacaagagcagcaatgcttgtgagggttaatacccttcttcaaggctactcaggaatcagatttgaaatcctagaagctatcacgaaactactgaaccacaatgtcacccccatcttgccattacgtggtacagttactgcgtctggtgatctaattcctctgtcctacattgttgcattgctaactggtaggcgaaatagtaaagctgttggaccctctggagagtcacttaatgctaaggaagctttccatttagcaggtctacattctgggttctttgagttgaagcccaaggaaggtcttgccctcgtaaatggcacggccgttgggtctggcgtagcttctactgtgctttttgaggcaaacatattagctttattgtccgaagttctatcagcagtttttgcagaagtcatgcaagggaagccagaatttacacatcatctaatacataagctgaagtatcatcctggtcaaattgaagctgcagctattatggaacacattctagatggaagctcttatgtcaaagatgctaaactgcaacagccagatccattgcagaagccaagaaaagatcgttatgctcttgtaacttctcctcagtggcttggtccacagattgaaatcatccggtattcgaccaaatcaattgaaagggaaataaactcagtaaatgacaatcccttgattgatgtcacaaggaataaggcactgaatggtggtaatttccaaggaaccccaattggagtttcaatggataatgcacgtttagctgttgcttcaattggcaaactcatctttgcccaatttactgagctagtcaatgatttgtataacaatgggttgccatcaaatctttctgctggtagaaacccaagtctggattacggtttcaaggcatctgaagttgccatggctgcttattgttctgaacttcaatatctagcaaatccagtaacgagccatgtgcaaagtgctgagcagcacaaccaagatgtgaactctttgggcttaatttctgctttgaaaactgtcgaagccgttgagatattaaagctcatgtcttcgacttatctggttgcactctgccaagctattgacttgaggcatttggaggaaaatttcaagagtacggtcaagaatactgtaagcagagttgcacagaaaacattaattacagaaggcaaagaagaaattaacccatttcgactttgtgagaaagatttgcttaaagtggtcgatagagagtacgtattttcctacattgatgatccctccaatgttacgtaccccttgatgccaaaactaaagcaagtactttatgagaaagcacacatcagtgccattaatgacaagaatgtgagcttgttgatttttgagaagataggagcttttgaggatgaattgaagtctctcttgccaaaggaagttgaaaatgcaagggtagcttatgagaatggcaatccagcaattccaaacagaataaaggagtgtaggtcatatccactgtacaagtttgtgagggaggagttagagatagggttgctcaccggagaaaagaatctctcacccgatgaggaatttgaaaaagtatatacagccatgtgtcaagcaaagattgttgacccaattctggaatgtcttggagattggaaagggtctcccatcccaatataattaatgtgtaggagtaatctgccgatgtttgacaataactagcaggcaacttcttttttcatgatgtttgttttaggatttgatttgaataactaaatagttaagagaaactagtgtggatttccagtgtacgccttatctatacaactaaatcaataaaaaaaaaacgaatacgtcattcctttagttat

SEQ ID NO: 54 - DNA, 804 bp, Glycine max
actttgatagtttgattagctctaataaggttaaggaccagagagagaaagagaaataagaagaaaaacacaagatgggttattggaagtctaaggttcttcccaagatcaagaaggttttcgagaagaatagcaccaagaaagctgctgctgctgaggccaccaagtcctttgatgagtcaaaggaggaatacaacaaagcctttgaagaaaagaagactgaacttcaaaccaaagttgttgaaatatatgaggcttcatcaactgaaatcaagagtttggttaaagaacccaaggaagctggtttgaagaagaactccacagaagtccagaagttcctagaagagctggttaaaattgatttccctggatcaaaggcggcatctgaagcatcttcaaagtttggaccagccttggcttcaggttcagttttctttgtgtttgagaaggtgtccactttcattgttacagaagaaaaagaagttgaagcccctcctgcagtagaaactaaaacagaagaagaaacaagtagtgttgtcaaagagagggagacagtggttgaagaagaaaaaaaggaagaggaaaaaccacaagcagacgagacaagtgatgagaaaaaagtggaagaaaaacaagctgagactgctgctaaagaggaagagaaacctgctgaaccagcagaaccaccaaagccttgaaaatttcgttctgaacagaagctatatatatgtagctgcttcatgaagatgcagaagatcatatacgtgcattcttagtttgcatatagtccaaaattgtcacata

SEQ ID NO: 55 - DNA, 1230 bp, Glycine max
tacaaatttacattaggataattttgtttgacttgactaatgagagatgtgaatgtgaatatacattttccaaaagaaaaatccttatcagatctgacaaggctgcaagagaatcactataaaaaggtacaaaacatgaagaaagaaatcaatcaattcagattcaactaaaggttgaagatttagcaaaacaatggaagaagtgaagctgattgctacacatcaaagcttcccttgtgccagggtagaatgggctttaaggataaaaggtgttgaatatgagtacttaaaagaagacttagcaaataagagttctttgcttcttcaatctaaccctgtccacaagaaagttccagtgctcctacataataacaagcctatagctgaatcacttgtcatcctggagtacatagatgagacatggaagaagaaccctttgctaccacttgatccatatgagagagcacaggctcgcttctgggctaggtttattgatgagaagtgtgtgttagctgtatggggagctaccgtggcgcaaggagaagagaaagagaaagctgtgggtgctgcactagagtctctggcacttcttgagaaggaaattcaagggaagaagtattttggtggagagaagattggttatcttgatattgcagctggctgcatgtctctttggttcagtgtcctggaagagcttggagagatggagctactcaatgctgagaggttcccttctcttcatgaatggagtcagaacttcttacagacttcacctgtcaaagattgcattccatccagggaaagtgtggttgaatatttcagctttggcatcaactatgtgcgttccttagcagcatccagtaaatcttgaaactgaaaatatacctttaatcaactacatgcatcatttataattgttcacattgtttgtattggaattggagtttggcttcaaatagtgttggttatcttatcatatgtagttgtgtgaaatgtgtaatcagttttctgtgcaatggtggcactactagctatagtgaaattttcagttagctatgctatattgtggtttcatgtgacaatgcaattaatagtagtttattatcggttcctgaggagagaaagaaagaaagttttcttttgttctgtatggctaattcagcagtaaagatagatactgcttaaatagaagaaaaacaaagtacattaatcctttgttcgcctatttga

SEQ ID NO: 56 - DNA, 1036 bp, Glycine max
taatttatgggtggaaattcataagtcaactacatctcatcatttggtcttggttgttgcaggcaacacctcatttcccactttgtccactttagcatagcgatcctctcatcctcctctatataatcactgatctccctatcatcctcttctctccaccatgggcagcatgaaggtgcatcagttcacacgtggattaatctgggagcacgaacctttcctcacacttggctgcaagagattacgccctcttgctcccaagcttcccaacaccaaaactatcactacccctttcgatctcaagagcttcatcaggcccgaaagtggccccagaaaacccgtttcctctgacgacactaagaaggatccaccttcaccccaaggccagattgaaacgcacccaggagggacacggtggaatcctacgcaagaacagataggcatattggagatgttgtacaaaggagggatgcgaactccgaatgctcaacagatagagcagatcactgtccagcttggaaagtacggcaagatcgaagggaagaacgtgttctattggtttcagaatcacaaagcacgcgagagacaaaagcagaagcgcagcagccttgcatcttctcatagtcctcgaactcccacaattcacagtgttgttactttggagacaacaaggggggaagtggtagagagagatcacgaggaagatagtccgtacaagaagaagtgcaggagatgggtatttgactgcttggaagaacaaaacatgtcatcaccttgtgaacaagaggaacatagaactctggagctttttccattgcacccggaaggcagatgaaggggtttgagtttgattgaccatttatctatcatttttcactttgctttagttccgaatcgcagctgattattgaatgaatgtggtttaattaatttgctttacttttcttttttctttgtattgggaaagaagaaagacaaagttgtctctgatctgtactcttccacttaatgctattcctgactttggaaccaaacc

SEQ ID NO: 57 - DNA, 2167 bp, Glycine max
tatatatccacacatcttaagtcatacttccatgtccatgccaaatttagtccaacccattctccccacgctataaaatccccccactccttcttgcttcacacaacatatctttctcttgttccttctccttgtgatcttcttcaaccttcagagtgagaaaacacattcagaaaccacgtttctctcttcttcttcttcttcttctttttcttctctcgaggtcattattaagtattcaccatggaggcaccatctcccaccaaacccaacgacccaacaaaaccttccaacacttccaccacccttagtctggaggccggcccggcccaagcgagcccactccgaaaaatgtttgccgtggcgtccatcgccgccggcatacagttcgggtgggctctccagctctctcttctaaccccctacgttcagcttttaggagtcccccacgcggcggcctccttcatctggctctgcgggcccatttccgggctcgtggtacagcccattgtgggctactacagcgaccactgtacctcccgctttggtcgcaggcgcccctttattctcgggggcgccttagccgtcgccgtcgccgtgttcctcattggttacgcagccgatataggatacgcggcgggcgacgacatatcgaaaacgacccgaccccgagcggtcggggtcttcgtcataggattttggatcctagacgtggccaacaacatgcttcaaggaccctgtcgcgcctttctagcagacctagctgccggcgaccaacgaaaaacaagaattgctaacggttttttctcgtttttcatggccgtgggcaacgtgctaggttacgcagcagggtcctacaaaggtctccacaagatgtttcccttcacggagaccaaggcctgcgacgtgttctgcgcaaatctcaagagctgcttctttttctcaatcctcttgctgctgtttttagcaacggttgctctgctctacgtaaaggacaagcaagtggaagcacgggccttggatgacgccacgcaaccctcgtgcttctttcagcttttcggtgctctgaaagagctcaagagaccgatgtggatgctgatgctggtgacagctgtgaactgggtggggtggttcccttactttttgttcgacaccgattggatggggcgcgaggtgtacggtggtcaagtgggggaggacgcgtacgccaatggagtgcgcgttgggtcgctagggctgatggtaaacgccgtcgttttgggcttcatgtcgctggcggtggagccgttggggaagatggttgggggagtgaagaggctgtgggcgattgtgaactttattctggcaattgggttcggaatgacggtggtgatcaccaaggtggcggagcaccagcgcaagatgaatcccgccgccgtgggtcacccctccgaaggggtggtggttgggtccatggttttctttggggttctcggagttcctcttgcgattactttcagtgttccatttgctctagcatcaatatactgcagcgcttcaggagcaggccaaggtttatctttgggagtcctcaatcttgcaattgtggtgccacagatggtggtgtctacattaagtggtccttgggatgctttgtttggtggtggcaacttgccggctttcatggtgggtgccgcggcggccgcattgagcgccataatggcgattgtcttgctgccaactccaaagccagctgatgaggccaaggcttcaagcatgatggcagggggctttcactagttaatcgagttttgcatgtttcagttttgtcccctgctttaataggaagaaaccaaaaaaagaaagtaataaaagaagaaaatttggatagtcttttaagatttgaattgaagctggtgtgtaattaagtaatagacccaccaagtgctacatttataaccttttgtagcacccactgaacatagtaattgtagagtttggtgtgattgtacataattgcagtggttggaacttggaagacccttcctcttgtataattttagtctgtttcagtgttcagaggaatgtggtgattaaggaggagtcttagttttaagactttatatcaattaatgataataagggaaatgctacttttacaatgttctctcaattacttatttgatag

SEQ ID NO: 58 - DNA, 1039 bp, Glycine max
taaatttctcccaccaacaaaaactaatacacactaataattaccaagattttgctgatataaaaaataattgttattaaaagaaaatagggcacacaaacacgctcctccctctataaatgtagatctaaatctccactgaatatcaaatcgagaaaatggcaagaatatcaaattggtctcttgtcttcctcctcatctctacactactccacatagagcccagcctctcaagcccaagcccaagcccaagcccaagcccgttcataaaatcctcctgcagcagcacccagtacccagccctctgcgtctcatccctctccgtctacgcctccagcatccaacaggaccctcaccaactggttcagacagcactctccctctccctgaaccgcacccaagccaccaaaaccttcgtggctaactgcaacaaattcagaggcctgaaacccagagagcacgctgcactcaaggactgcgccgaggaaatcagcgacagcgttgaccgcctcagccggtcgctgaaggagctcaagctatgtaaggttaagggtgaggacttcacgtggcacattagcaacgtggagacatgggtcagttctgctttgaccgatgagagcacttgcggcgatggattctccgggaaggcgctgaatgggaagatcaaggactctattagggctaggatgctcaatgttgctcaggttactagcaatgctctctcgcttattaaccactatgctgcacaacactagttgggaacaatgatcttggttgcttaatgctttacttttcttcttttatgtgctctatgtaattattatatatgtgagtctttatagatatatggatcttctcatgcttcttaaatgtgaaatatgacgttgtgtttttctatgattatattttcctctctacttctcattgatcgattatggacgaatgccaatcgaaattagtatatcaatgaagtcttctgtttacattaattatgattttgagatttagaaaaatgttaacaagtgctctaaagctttaa

SEQ ID NO: 59 - DNA, 977 bp, Glycine max
catttccctgcacaaagattttttcccagcttgctcatcaagtccaagaaccaacctaccttttgcaatctcaattcaatggcgtcctctctcattgcgaagcgcttcctctcctcttccctcctctccaggtccctccttcgccccgccgcttccgcttcccaccgctctttcaacaccaacgccatgcgccagtatgacaaccgcgccgacgaccacagcaccgacatcgatcgtcactccgaacgctctttccctagcactgcgcgccgcgacgatatcttctcaggtaatgtgttggatccattttttccgactcggagtttgagccaggttctgaacatgatggaccaggtcatggacaatccgttcctctccgcgtcgcgcgggatcggagctggcgctggagtgcgtcgcggatgggacgcgagggagacagaggatgctctgcatctgcgcgtggacatgcctgggctcggcaaggaagacgtgaagatctccgtggagcagaacactctcattatcaaaggtgaaggtgctaaagaaggcgatgaagaagagagcgctcgtcgctacactagcaggattgacttgccggacaagctctacaagattgaccagatcagagctgagatgaagaacggtgtgctcaaggtcgttgtgccgaaaatgaaggaggaagagaggaaagacgtgatcagtgttaaggttgagtagaacaacccaagctgatttggccattttgcatcgacactactctttagttttcttttattaggtggtgatgaatctagttagtagatataacagcttaatagtattgttgctgtgtttggttttgaaacttcaaacgtatctgtaataattgttatatagagttgtgaatgagcttgttgtttagaagtctactgcagtctgaatttgacttgtcaatgaaattcgtgtcattttaaattcgtaaaatcaagcgtttccct

SEQ ID NO: 60 - DNA, 1396 bp, Glycine max
ggctttaaccatgttcattcattacttgttttacttcaattcccacccaccatttcctcaaatcaaaacacctttcactttctttcataccccaaagccgcgctatctatcttctttcctccacctaaccagatcaaccctaatcctatatatcatatcgatcttactacttctcatagcttcaatttgaggcctcacgaatgaataaggatttgggcatttgagagagatcatatggagaatactaagatgatgggggtgaagatacaggactatgcttctaatatggagagtaataattatccattctttgatttctctgaagataagggctctttagggtttatggagctattgggtgcgcaagactatagtcctctccttgatttccctctatcgtcacacgtgtcagggcctcaaacctcttctgttaaggaaccacctgagactaagaaagagtgttccgaggtaactaataacaaccagcaacctgcgactccgaactcttcatccatttcctccgcgtccagtgaggttttctatgatgaacagaataaaactgtagatcaagcacctgaacaccaaaagacaaaggaacagttgaaggctaagaagacaaatcagaagagacagagagaaccgagattcgcgttcatgacgaaaagcgaggtggatcatctggaagatggatacagatggagaaagtacggtcaaaaagctgtgaaaaacagcccctttcccaggagctactatcgttgcaccagtgtttcatgtaatgtgaagaaacgtgtggagcgatcttttagcgacccaagcattgtggtgacaacctacgaaggacaacacacgcatccaagcccagttatgggtcgctccaacaactttggttcggtaattatgtctggatctgctggaaactacatgtcccaatattatcagcagcagcatcaacaagtccacatcgatgcattgtcctctttgggtttcctctcttcttcgtcttcaaggaatgccacttttcctcaagagactgccttgttaagtgactatgggcttcttcaagatgttgtttcttcacatatgttgaaagaagattagatgattacttcattactgtttttatgtgatttcttgttattagtttctgtaggagaatatagatgaggactttgttgctgcactgggatatttaagttttagttttcattctcatcatgatcacacttgtaaaacccacgttatctttttgcacatatactatataataggccttggcatgaaaattattgtaacatgatggattttgatccggttgacgtctcttaattaggatctagtttcctgtttggctgtttccatccaatatattttctatttttaatagatagaaaatgaa

SEQ ID NO: 61 - DNA, 827 bp, Glycine max
atggcgacgttcgaactgtaccgcaggtcgacgatcggaatgtgcctgacggagacgctggacgagatggttcagaacggcactctcagccccgagctcgcgattcaggttctggtccagttcgataagtccatgactgaggctctggaaacacaagttaagagcaaggtctccatcaagggacatctccatacatatagattttgcgacaatgtttggaccttcatcttgcaagatgctttgttcaagaacgaagacagccaagagaatgttggacgggttaaaatagtggcgtgtgattcgaagttgctcacacaataatgtgcccaattctgctaccttgtcaacaaggagaaattgttgtcatctgctggtactatggaaatggtggccagaccatagttacaatggtcttaaattcttgccggaactagcaactactgtatgttattatagatttaaaagccatcagtcttcatatcattaggtaattaatgatgctatattgagaataccgcttgggaattggatgagtctactctcttcttttaagtgtattctgttctattcttctgtgagtaccagtaagtaacctagtttatttttcattggctaactagtaaggttggacaggctatgcatccctaaaatttcttatgcacccagcctccacctataaccaccaaacaccattgttgcaaccacccaatgtcacctccacaccacaccaaacccaccattgtgttgccacccaaccaccacaatcgccatgtgatccacgaccttgtgcgaggtcgcatcactcccacatagtcaccaattgattg

SEQ ID NO: 62 - DNA, 754 bp, Glycine max
gcatggcatttagtaattaataaaccaaccacccattaaaggtgtgggccctccctgagttctccattataaaagattggttggtatgcactcagctcaaaaacaaatctccttcttgaactcaaggagatggctctgggaagaggcagtgcagtggttctactactttgcttcttgctgcttcactctcagatggctcgtgctgccacctacacagttggagattctgggggttggacctttaacactgttgcctggcccaaaggaaagctctttcgggctggtgacacacttgctttcaattatagccctgggactcacaatgtggtggccgtgaacaaggctggatatgatagctgcaagactccaagaggagccaaagtgtataagtcagggacggatcagatcagacttgccaagggacagaactacttcatctgcaattatgttggtcactgcgagtctgggatgaaaattgccatcaacgctgcctgagtttaataatatggttaactacccatacatattgtattgtaatgcaaattgcaccctttagtgggaagttagctcctttaaataatgttaagaaaaaaagtagctatgtgtgctcttctataaatgtcacttatctataaagaataaatgtcggcagtcggtgatctctctctctctccctctctactcctgtgtgtaggtcttacatcagaacttataagtttagtataatacaaacttgcttaggtcactttcattt

SEQ ID NO: 63 - DNA, 676 bp, Glycine max
  misc_feature (589)..(590): n is a, c, g, or t
tatattattttatgtacttaacacttaacacagtacacatgcatatataaagcaacacatttcaaggaaacattctccaaaccatactgatctcccaaaataaactggaagatgattaacttaaagacacacatatatatataagccaaaagcatatatatatatataagacaagtagaaggaatatgctcataattggttgcttatgcttcagccaaagagatggtggtgcttctttccatgagcttcctcatcttgctcctttgcttcctttttctcatgatgttcatggaaggcaaacccaccagatcccactgcagctgctgctgcaacctcctcttctatcttgtgcctgtgagcatgctctgggtctttctctgccttgtgcttctcatgcaaggcgtaagcagcagcagatgcagcacctaactcaccaaggtgctcaagatgcttgtggtgcttctcttccttcttatagtcaacctcatcatcatacccaccaccacttgttttgctgtacccaccatcagtggtagtgtcagggtacacaccaccaccaccagcggcgccatagccaccacttggtttnncgtagccaccatcagtgacgtcagagtanccaccaccaccagcgccatagccaccagaagtctcatcaatgccaccagaataacct

SEQ ID NO: 64 - DNA, 1594 bp, Glycine max
caagcggagatctgatggcgctagagtgggttgtgctagggtacgtcgcggccgcagaggccataatggttattctcctcaccatcccgggcctggaagctctccgaaagggtctgatcgcggtgacccggaaccttctgaagccgttcctctcggtggtgcctttctgcctgttccttttcatggacatatattggaagtacgaaaccaggcctagctgtgagggagattcctgcaccccttccgagcaccttcgccaccagaaatctatcatgaagagccaacgcaacgccctcctcatcgccgccgcactcctcttctactggctcctctactccgtcaccaacctcgtcgtcaggatcgaccacttgaaccagcgtctcgaacgcttgaagaatcgcgattgagatttcacccttgcagataagtcggttaatcaatccctcggatataaacttggtgttttctttcttgtgctaggaaggatctatttctatctcctttttttgtcttttcttttctttctttctaaatcgatgtttgagtgatgacgattattgagtctttttttagtattattactcttgctataactatgctacttgaatttccgccacctagggttccttttggggattttcttattgttgcgctcagggatccaattccgtgcacctcgtcgctcccttgtatcataggaaaacacgatttcgattattgttgagtgttatctgaaatgcctacttacttttcttttacgcattattatgatttcttaattggcgtgagaatgctcgtatgggagtctgttctgttgcttgaaatttgacatgatagtaagataattattaaattttcaacctcaatttgttgattgtcaacaactttggctcattaagtttgacgtgtattgaatccggctatttaattttaacgagttcgagcatcttgttatcaatttctcttgtccagtgcctagagctgtgaaattagggaaagggatctctagcattgcatggttgaaggaacctccctcaaccataaagaatagataacataacaaagaaggggttgatgtaagaatcttttctcttaacgtaactcaaaaccccgttctccaattctattataggttcaaattttactctgatctttgtattctatagaagagccaatgaactatagccccctgttgaatatagtgtaccactcgtttgtctgcctactaaattttcttctagtgttgtattcctgttagttgtaacccgcaatcctttttccgtacactataagctagttaatgttgtctaacttacataatgttaattttaagactctctctgtcagcaattcttttgtacttttgatgaggggtgttaatatagttctgtagactctagaaactataactttggttgaatcttcttttcttgaacttttggagattgggttgtctacttgtcttgggtctcacattatatggttgcctttatttaccaagtgaatatattctaatcctgaaggtgaggcaacaagcagaatcttgctctgttcaagtgctttgtcctgttaaatgtaatggaatgtggatggt

SEQ ID NO: 65 - DNA, 1041 bp, Glycine max
atgactgccaaacaaaccaagcagcattataatagaaagaggacacaaagtgaaagagaagggagctatagccataaggagaaaaactttcaaagaggtgaaagcaaaagagttcttcttttccttttttatatatacttgggtgctaagctgtgtgagttcattgacaagcctgaaatggcaactatgctgacaaaggagcatggtctgaacctcaaggagaccgagctttgcctcggtttgcctggtgggggaggcggcggcggcggcggcggcggcggcggcggtggtgaggtggaaactccaagggccactgggaagagagggttctctgagactgttgatctgaaacttaatcttcattccaaggaagatctgaatgagaatctgaagaatgtctcaaaggagaagaccctccttaaggatcctgccaagccaccggctaaggctcaagtggttggttggccaccagtgaggtcatacaggaagaacatgatggcagtacaaaaggttagcactgaggatgtggcagagaagacaacaagcagcactgctaatcctggggcatttgtcaaggtttccatggatggagcaccttacctgcgcaaggtggacctcacaatgtacaaaagctacaaagagttatctgatgccttggccaaaatgttcagctccttcaccatgggtaactatggggcccaaggaatgatagacttcatgaatgagagcaagttgatggatcttcttaacagctctgagtatgtgccaagctatgaagataaggatggtgactggatgctcgtgggtgatgtcccatgggagatgtttgttgagtcatgcaagcgtctgcgaataatgaagggatcagaagcgattgggcttgcgccaagagcaatggaaaaatgcaaaagcagaagctgaagcgggcctaatacaatgttcaatgaaatcccgtaccaaagtggacctatatatatatatatatatatgaagcacttcagcgagactatggagcggagtgttgttagcttgaatgtgtct

SEQ ID NO: 66 - DNA, 1553 bp, Glycine max
acttccatgcaccaccccattcccaccaactcacgctgcatagcccatcattgagttacacagtttcgtctataaaagtcctaatatacaatacaacaccagcagtgtacgctcacaaaacatctctaaacgcttgtaaagatctccatctcccattgatcctcctgaagtttttgtattcaaccatggctacctatagttggtcaactgagatattttcatttttggttctaacgttgttgtcgacaagcagtcacacaatggttgctggagcacgcaatcttctcgaatcaaccttgtcaaaaccagaagtgccaacacttcccaaacctcatgagttgccaccattgccggaaatccctgaattgcccaagtttgagtcgcctaagatccctgcactcccgaaacctgagctacctaaggtccctgaactgtctaagcctgacatgtctaaagttcctgagttgcctaaagtcccagaaaggcttaaagtacccgagatatctaagatccttgaattgtctaagcccgagttgcctaaaggaccagaattacttaagcctgagttacctagtgtacctaatatccctaaggttcctgaattgcctaaaccagagttgcctgaagtcccaaagttgcctaagcctgagttgccaaaagtaccagaattgcctaaacctgagttacctaaagtacctgagatccctgaattgcctaatctcgagttgcctaaagtaacacaattgcctaagtctaagttaccaaaagtacctgagatccctaaggtccctgaatttcctaagcccgagttgcctaaagttcctgaattgtctaagccagagttaccaaaagcacctgagattcctaaggtccctgaatttcctaagcccgagttgcctaaagttcctgaattgtctaagccagagttaccaaaagcacctgagatccctaaggtccctgaatttcctaagcccgagttgcctaaagttcctgaattgcctaagccagagttaccaaaaatacctgaaatccctaaggtccctgaatttcctaagccagagttgcctaaagttcctgaagtgcctaagccagagttgtcaaaagtaccaaagctacctaagtctgagttacctaaagtacctgagattcctgaattgcctaagtccgagttgcctaaaataccggagattcctaaggtccctgaattcccaaaagttcctaaagcattttcaaccaccaatccttgagtttgaattatgtcttttgcttacttgtcctcaggtcataactggatgtattccagtgcctagttcatgttgtttgatgggtgtgctatgatatatgttaattgtcacttgtgtataatatgttgtagctacttgagtgcaattgagtcggtttactagtattttgaaccatactatagattatgtttagtttgaagttttttaaattgtcaattgcattgatatcattgcatactgatagacatcctctcatgtgttaaatgttgtgaatttaataagattgtaatgtctgagtataattctcta

SEQ ID NO: 67 - DNA, 918 bp, Glycine max
gcggggagggaggggagaagagaaagaaggaaggagacatttataaaaagctctttcccttcccctaaatccctcacactcattccatttttacctttctctctcgctcgcaaacgcaaataataaaaaacaaggttttagccatggcatcgaagcggatcttgaaggaactcaaggatctccaaaaggatcctcctacctcatgcagcgccggtcctgttgctgaagacatgtttcactggcaagcaacaattatgggtcctccagacagtccttatgctggaggtgttttcctagtcactattcatttccctccagattatccctttaagccacccaaggttgcattcaggacgaaggtatttcacccaaatataaatagcaatggaagcatttgccttgacatattgaaggagcagtggagccctgcgctaactatttcaaaggtgttgctctcaatttgttccctgttgacggaccctaatcctgacgatcctttggtccctgaaattgcccacatgtacaagacagacaggaacaagtacgagtcaactgccagaagctggacccagaaatatgccatgggttaagtgaccttgtggtatgtttgagggagagcctttctactctcagtgtttaggtctctcagcccttgtaggaatatttgtcgttttaaatcatgattaaggcccctccagttgaagatgaatgggggaaaacgtttctcctgctgtttgtattgcaaatgttcatggtaatttagaacagtgaaatgttgccccaattaaatgttatgctcaagttttaaccgttctattaagtgccatttcatgtttttcttttgtcatttatgaacttaagccttaatttaagagttgctcatcacctggtaaaaagaccctttgttcaaaaaaaaaa

SEQ ID NO: 68 - DNA, 1953 bp, Glycine max
tcaagcaaagtcgcaaattctcctcctacaccaacaaaaatggcaccttctccacaagaaatcatcttccgatccccactccccgatattcccatccccacacatctcccattgtactcttactgcttccaaaacttgtcaaagttccatgaccgtccatgcctcatcgacggcgacaccggcgagaccctcacctacgccgacgtcgacctcgctgctcgccgcatcgcctccggcctccacaaaatcggcatccgccagggtgacgtcatcatgctcgtcctacgcaactgcccgcagttcgccctcgccttcctcggcgccacccaccgtggcgccgtcgtcaccacagccaaccccttctacacgccggcggagcttgcgaagcaagcgacggccacgaaaaccaggctcgtcataacgcaatccgcgtacgttgagaaaatcaagagtttcgcggacagcagcagcgatgtcatggtgatgtgcattgatgatgatttttcttatgaaaacgacggcgttttgcatttctcaacgctcagtaacgccgacgaaacggaagcccctgccgttaagattaaccctgacgagctcgttgcgcttccgttttcttctggcacgtctgggctccccaagggcgttatgttatcgcataaaaacttggtcaccacgatagcgcagttagttgacggcgaaaacccgcaccaatacactcacagcgaggatgtgctactctgtgtgttgcctatgtttcatatctatgcgctcaattccattttgctctgcgggattcgttccggtgcggccgtgcttattttgcagaagtttgagatcactactctgttggagctcatcgagaagtacaaggtgacggttgcgtcgtttgtgccgcccatcgttttggcgttggttaagagcggagagactcatcgctacgacctgtcgtctattcgcgctgtggtcaccggcgcggcacccttaggaggggaacttcaagaagccgttaaggctaggctaccacacgctacttttggacagggatatgggatgacagaagcaggaccacttgccattagcatggcatttgcaaaagtaccctctaagattaaaccaggtgcatgcggaaccgttgtgagaaacgccgagatgaaaatcgtggatacagaaacgggtgattcacttccaagaaacaaacacggtgaaatttgcataagaggcacaaaggtcatgaaaggatatctaaatgacccagaggctacagagagaactgtagacaaagaaggatggttacacacaggagatattggtttcattgatgatgatgatgaactcttcattgttgatcggttaaaggaattgatcaaatacaaaggattccaagtggctcctgctgagcttgaagcattgttgattgcccacccaaacatttctgatgctgccgttgtaggcatgaaagatgaagctgcaggggaaattccagttgcatttgttgtaaggtcaaatggttctgagatcgccgaggatgaaatcaagaaatacatttcacaacaggtggttttttacaagagaataggtagagttttcttcacggactctattcctaaagcaccctcaggcaaaattctgcgaaaggtattaactgcaagacttaacgaaggtttggtggtggccaattaggtccataattgtgacagaggaaaatcgtggctgttttacttaccgtaccacaggcccttcctgttgtggtttttgttccaattttatatctcgttatcaatatatatatataatatgcaagtattgcatgaatttattcttgtgtaacttataatgttgtatatattggagaccagactctgaattacagcatgaacacattaaaattcagcaagttgttacgatctgtatactaaattagtactaaagtaaaaagaatttaattcatattcc

SEQ ID NO: 69 - DNA, 1204 bp, Glycine max
caccttccagtaaagcaggagtaggataattgttgaacatagatgaaaaggtgattgttatgtctggactgattgaaggacttcctgatgctgttgcaatcaggtgccttgcatgggttcccttctacctccatccaaagttagagcttgtctctcgtgcttggcgagcagttgtccgtggccctgaactatttaaagctcgacaggagcttggttcgtcagaggatttgttatgtgtctgtgcttttgaaccagagaacttatggcagttgtatgaccctctgcgagatctctggattactcttcctgttctaccctcaagaatcaggcacctttcacattttggtgctgtctccactgctggaaagttgtttgtaattggtggtggaagtgatgctgttgatcctttgacgggtgaccaagatggttgttttgcaacaaatgaagtctggtcgtatgaccctgtagtacggcaatggtccccccgtgcagcaatgctcgttccccgttctatgtttgcatgctgtgttatgaatggaaaaatagttgtggccgggggcttcactagctgcagaaagtcaatatctcaagcagaaatgtatgaccctgagaaagatgtttggattccaatgcctgatcttcatcgcacccataattcagcctgttcaggagtggtgattggaggaaaggtgcatgtactgcacaaggatatgtcaacggtgcaagttttagacaatgcaggtcagtggacggttgaggaatatgggtggctccatggtcaaatggcagttatcagggatgccctttatgtaataagctatggattaatcatcaagcaagacaaaaaaatgaggaaggttgtaggttcagcttccgagtttcgaaggagaattggatttgcaatgataggtttaggtgatgaattatatgtgattggaggtgtcattggccctgacaggtggaattgggacatcaagccactgtctgatgttgatgttcttacacttgcgagcgacagaccgacgtggcgccaagcagctccaatgacacggtgtggtggtaccattcttggttgtacactgctgagaatttaggctttcactttcatggcttatgtgatatgtgtgctcctactaaggaatgtgaattccttcaacaggagacaaagaattaggttgttcaatggcttctttgagttgtatgt

SEQ ID NO: 70 - DNA, 2038 bp, Glycine max
ttggttgactccaaaaaaaagaagaaggagaagaaaaactcgtttatgacaatcctcacgtttctgttggtcgcaaaattggaccaacgtacacgtcctaaacacttcctgtagtgtatatataaaaaccgcactcaccctccccgaagtcaccaacaacacttgtcatagaattcacaaccgacaatggcgacaaaacctcgtttggcttatctctcttccacttttcttcttcttcttgttgtaactgtctttacttcggtgtcggcacaagccccagaatccccatcactctacaacaccttccttcaatgcctcaccaaatacacaaataacccctccaacatagtcttcgccaacaccaaccccaagttccccactatcctccaaaactacatccgaaacgcgcgtttcaacacctcctcgacgcgaaaaccattactcatcgtcactccccagcaagaatcacacgtccagggcacagtaatctgcgccaaaagcgttgaaattcaactcaaaatcagaagcggaggccatgattacgagggcatctcgtacatctccgaggaacccttcgtcatcctcgacatgttcaactatcgcagaatcaccgtggacgtaaaaaacgaggttgcagtggtcgaagctggagcaacacttggagaagtttattacagaatctgggagaagagtaaagttcttggctttccagcaggggtgtgtcccactgttggcgtcggtggccatttcagcggcggagggtacggcaacatgttgagaaaatatggcttatccgttgataacgtcatcgacgcgcaaattgttgatgtgaaaggaaatcttctaaacagaaaaacaatgggggaagatctgttctgggctattagaggaggtggtggagcgagttttggtgtcattttgtcgtttactattaaactcgttccggtgccggaaacggttaccgttttccgcgttgagaagactcttgaaacgaatgtcactgccactgaccttgtcgtgcagtggcagcaggtggcgccaaatactgatgatagacttttcatgaggttactgttgcagcctgtgagttcgaaggttgtgaagggaacaagaaccgttagagcctcggttgtggctttgttcctcggaggggctaatgaggttgtgtcgattttggcgaaggagtttcctcttcttgggttaaagaaggagaattgcactgaggtgagttggattgactctgttctgtggtggaacgatgacaatagtttgaagaatggtgacaaacctgaaaccttattggatcggaatttaaacaatgcaggttttctcaaaagaaaatctgattacgtccagaatgctatttccagagatgggttggagtggctatttaaaaggatgattgagttgggaaaaacggggcttgttttcaatccttatggagggaaaatggctgagattccttctgatgcgacgccgtttccgcaccgtaaagggaacttgtataagatccagtactctgtgaactgggatgatccttcacccggtgctgctctgaatttcacgaaccaggctaagaggttgttcagttacatgaccccttttgtgtccaagaatcccagaagtgcgtttttgaattatagggatcttgatattggggttaacagctttggtgagaatagctttcaggaagggttggtttatgggaccaaatacttcaatgataattttcagaggcttgtgaagattaagaccacggttgatcctgaaaactttttcaggaatgaacagagtatccctgttcttcatggcgaagcatagggaatcatacagatacagtgaagaatattgcttagtttttttttttttttccttgtttgacagtaaagaatattgcttagtgctcctgttagttgtggcattttcttttgttcctatagttctatatgaatagttggcccataggccgtgatgtttgtcaattgatttgattattattatatcattgaattttaaagttgaggtatatggtaggttc

SEQ ID NO: 71 - DNA, 1190 bp, Glycine max
ctttgtgcagccgcgttcgctgagtgcgtgtcccctttcatttcattcttatctgataccaatgcctttttggaaaatgctgacctgtgaccctatattcctgagtgtaccctgctagtatatatacctcgcttcttccccttgctttctgaaccaaacaacaaatattcatggctttctttattctctccttcctcttccttcttcttgtttcatctgccactgcttgtgatcgctgcttgtatcaatccaaggcttcctatttctccaaagcttctgctctttcatctggggcatgtgggtatggctctttggcactagacataagtggtggacaccttgcagctggtgtggattctctcttcaaaaatggagcaggttgtggtgcctgctttcagataagatgcaagaacccaactctatgtagcaaagaaggtaccaaagtggtattgactgatcttaatcacaacaatcaaactgattttgtgctaagcagcagagcctttgcgggcatggctcaaaagggtatgggccaacaaatactgaagcttggcattgctgaaattgaatacaagagagtaccttgtgattacaaaaatcagaatttggccgttcgagttgaagaatcaagcaagaagcctgattacttggcaattaaatttttgtatcaagggggacaaacagagatagtagccgttgatgtggctcaggttgggtcttcaaactggagcttcatgagcagaaaccacggggcagtatgggacacaagcagggtaccccaaggcgcattgcaatttaggttagtggtaacggcagggtatgatggcaagtggatttgggcaaagaaggtcctacctgctgattggaaaaatggacttatatatgattctggccttcagattactgacattgcacaagaggcttgttccccctgcgatgatgggacatgattctttgctgaaaaataactcacatatacgcatagatagtaggcatcaattttattaatactatactataggctcacaatgcaacttataataatactaaggaatcatggactagcttaataaatttgaaaagcattcttatttgtaaaccactctccacttcgttactttatattctttctcattgtaagttgtaaccaaccaaaataatatattcttcctttttggagtatatgaatt

SEQ ID NO: 72 - DNA, 1826 bp, Glycine max
ccaggaaccaccacaaaacattctcatcattcacccaacaaaatggctctgcttctgatagtacccatttcactggtaaccctctggctcggttacaccctataccagcggctacgcttcaagctccctcctggcccacggccctggccggtagtcggtaacctctacgacataaaacccgtccggttccggtgcttcgctgagtgggcgcagtcttacggccctatcatatcggtctggttcggttcgaccctaaacgtcatcgtttcaaactcggagttggccaaggaggttctgaaggagcacgatcagcagctggcggaccggcaccggagccggtcggcggcgaagttcagccgcgacgggaaggatctcatttgggccgattatgggccgcactacgtgaaggtgaggaaggtttgcacgctcgagcttttcacgccgaagcgcctcgagtccctcaggcccattagggaggacgaggtcaccaccatggttgagtccgtttacaatcactgcaccaccactggaaatttagggaaagcaatattggtgaggaagcacttggggtctgtggcattcaacaacatcaccaggttggcatttggaaaaagatttgtgaactcagaaggtgttatggatgagcaaggagtagaattcaaggccattgtggaaaatgggttaaagctaggagcatctctagccatggcagaacacatcccttggctgcgctggatgttcccactggaagaaggagcttttgccaagcatggagcccgccgcgaccgactcaccagagccatcatgacggagcacactgaagcacgcaagaaatctggtggtgccaagcaacattttgttgatgccctcctcacattgcaagacaagtatgaccttagtgaagacaccatcattggtctcctttgggatatgatcacagcagggatggacacaactgcaatttcagttgagtgggccatggctgaattgataagaaacccaagggtgcaacaaaaggtccaagaagagctagacagggtaattgggcttgaaagggtgatgactgaagctgacttctcaagcctgccttatctacaatgtgtgatcaaggaagcaatgaggcttcacccaccaaccccactaatgctcccacaccgtgccaatgccaatgtcaaagttggaggctatgatattccaaaagggtccaatgtgcatgtgaatgtgtgggccgtggcccgtgacccggcagtgtggaaggatccgttggagttccgacccgagaggttccttgaggaggatgtggacatgaagggccatgactttaggctacttccattcggggcgggtcgccgggtatgcccgggtgcccaacttggtatcaacttggtaacatccatgttgggccacctcttgcaccatttttgttggaccccacctgaaggaatgaagcctgaggaaattgacatgggagagaatccagggttagtcacatacatgaggactccaatacaagctttggcttctcctaggctcccctcacatttgtacaaacgtgtgcctgctgagatctaatctttctttttcttttccttggattactctttgttgcattaagaaaattgccttgtgtctatgtaactacatttgaaatggcaattcaaggaactaaggaaaaacccatagcgagtttggcttctcctcttcagattttggattgaaattcctgtttaagattaattaaataagttacatgagaagggagtttaccgaaagtaactctaagcatccaatttttggtgaaaggaagctaatggttcaccttatgg

SEQ ID NO: 73 - DNA, 651 bp, Glycine max
atcaaattcacacttataaacatgcccccacattcctcattggcacatcactttattgctccgcgtccttcttgtttcacaagtttccttggaagcggagcgtattaccctctctccctttcttctattttctcattcctgcctctcttctcaacatggccgccaacagatggttgaaacctgaggtatacccactcttcgcttcagttggtgtggctgttggaatctgcggtatgcaacttgttaggaatataaccaccaatcctgaagtcagggtgaccaaacagaacagaactgcaggaattcttgagaattttgcagagggagagaaatattcacaacatagcctgaggaagtatgttcgcggcaagcagcctcagattatgccatccgtcaacaacttcttctctgatccatcaaactaaagcaaatgccaaactttttgttcttgagatttgatttctgggattaattttttgctttccagtattgggaattgagaagtaggacaacgggatttgatttttcagataaagatgtactatgattctgtaatttgttggtggatgacattcaatctgtctaataataaaattctatactttcacatttataaatgatcttcatcagttttctacgatgctgttaata

SEQ ID NO: 74 - DNA, 1510 bp, Glycine max
agggcatttttgtgatttcgatgttataaagagagagagacagacagagaaacagaagaagaaagagagaaggtataagcagccgttttttctgttggtatatgaaggtggtgaaggaagaagaagaagaatggtgacggaccaagatatagcgaaaggggtggagtccctactgcgtcactctgacccaaactccataactacggtaaacggcgtcgttcagcagctggaggccaaactagggttagacctctctcacaaggccagtttcatcagagaccagatcgatcatcttctccggtcacagccacagacatttgctcctcaccctcctccacttcacaaagactattttgcccctcacacccaactgcacttcccaaccacccactttgctccccattttgccctccatgacgagatcaacttcctgcagcacccccaccctcctccaccgcgtaaagtcgagacctttcctcctcaaaatgttgcccctcctcaagtgcccaaagaaagtgtgcaaactggaagcaaaagaagaggtggtgctggtggtctaaacaaagtttgtggtgtttctcctgaacttcaggcggttgttggtgagccggcaatgccaagaactgaaattgtgaggcagctgtgggcatacataaagaaaaacaacctccaagatcctggtaacaaaagaaagataatttgtgatgatgccctgcgtttggtatttgagacagactgcaccgatatgttcaagatgaatcagttgctagctaaacacattatcccacttggtcctacaaaggagtcacaggctaaacgagtgaaggtggatactgaaattaagactgaaagtgctgaacctgctccatctaccgtggcaatatctgaagcgcttgccaaatttttgggcactgagggaagagagatgcaacagtctgaagccataagacttgtttgggagtacatcaagcttcaccatttggaggatcctttaaattcaatggtgatattatgcgatgcaaagcttcaagagctacttggatgtgaaagcatttctgctttaggaataccagagatgttagcacgtcatcatctatttaaacagtctgacacccgttagtgctgatcaattattgccatataagggtatgatctgcacaaaacgctggttatatgtttctgctcgttgctagatcgtataggaactttttacttattcctgtcacttcatttatctctttgttcactgacgtatgtaaactgtatgttgtctgacggttattaggttggttgtttagaatgacaatgtataccttgagctaggcaagaagtatattgtatttaatatatgccctttaggttacccgtgcattttattttatcagttctcaatgtctaacctcaactggctgatcgaattagtgatacttttgtacattgagatacaccaactactctgaaatgcaccttgtttttaccaaattttgtgaaaaatctcttggctttacat

SEQ ID NO: 75 - DNA, 1702 bp, Glycine max
attccaagtagccacagctctcctctcactttcatttctacatccctggctctgttccgtcgatgacgatggcgtttagggtgcgaaaccctgacctccgctttctcattcttctctctctcttgtccatcacttccgccaaggtcttcttcgaggagcgtttcgatgacggatggggaaatcgttgggttaaatcagattggaaaaaagatgagaacctggctggggagtggaaccacacctctggccaatggaatggagacgctaacgacaaaggtattcaaaccagtgaggattacagattctacgctatttcagctcagtaccctgaattcagcaacaaggataagacactagtcttccaattttctgtcaagcatgaacagaagcttgactgcggtggtggctacatgaagttgctcagtggtgatgttgatcagaagaaatttggtggggatactccttacagtattatgtttggcccagatatctgtggttacagtacaaagaaagtacacgctattcttacctacaatgacacaaaccacttgataaagaaggatgttccttgtgagactgaccaacttactcatgtttacacatttatcctccgtccggatgcaacctacagcatcctgattgataatgtggagaagcaaactggtagtctctactctgattgggatcttctccctccaaagacaatcaaagatcccgaggctaaaaaaccagaagattgggatgacaaagaatttattcctgatcccgaggataagaaaccagaggggtatgatgacatcccaaaagagctcccagatcctgaagccaagaagcctgaagattgggatgatgaggaagatggtgagtggacaccccctaccattgccaaccctgattacaagggcccatggaaggcaaagaaaatcaagaaccccaactataaaggaaaatggaaggcaccattgattgataacccagatttcaaggatgaccccgacctatatgttttccccaacttgaagtacgtgggcattgaattgtggcaggtgaaatctggtactttgtttgacaatgtcttaattactgatgatcctgaatatgccaagcaactggctgaagaaacatggggcaagcacaaggatgtaagttttttctattcattgatcttttttggtctaaggtatgctattgaaaggtatgatctccttaatgaaattgtgttcttgcaggttgagaagacagcatttgaagaggctgagaagaagagggaagaggaggaatcaaaggatgacccagttgactctgatgctgatgaggaagaggaagatgctgatgaagctggcaatgactccgatgctgaatcaaagacggaagctggggaagacaccaaggaagagggtgtacatgatgaactctaggaagtttgcaaaacggataaagagtgagcggaagttatattccggatagttatatttcagtcgtagtgttttttgttttccctagataagcagaccgtggaaaactgctagtaaaagctggagcttttgcttcgatcacatttgactatttcactgattagaacaggcaaagatgtctctcttgggttgatctttttatgtttcgatcaattgagtttcccattatgacgcttcctgaaaatctaattaattgcaaatttttataagaaaaatgattgattgat

SEQ ID NO: 76 - DNA, 746 bp, Glycine max
cccttcaccatggctgaacaaaccgagaaagcttttctgaagcaaccaaaagtgtttctcagcacaaagaagactgggaagggaaagagacctggaaaaggtgggaaccgcttttggaagtccattgggcttggatttaagactcccagggaagccatcgaaggaacctatattgacaagaagtgccccttcactggcaatgtttccatccgtggccgtatcttggctggaacatgtcacagtgctaagatgaacaggactattattgttagaaggaattatctccattttatcaagaagtaccagaggtacgagaagaggcactcaaatattcctgctcatatatcacctgccttccgtgtgaaggaaggggatcacgttattattggtcaatgcaggcctctctctaagacagtgaggttcaatgtcttgaaagtgatcccagctggatcttctagtggtgcaaagaaggcttttactggaatgtgagcttcgatttccagtttttattgaagaaaaatgttaccttgttatttagtagatctgatttcttttctggaaggtgacatattaaaaccttatcaattttggattttctttgaggtttcgttcgggctttatgtttaggattttgttatacattatgtttttcgaatactatttgccttttaatttttgaaactatgcagactcttcatttgctttaccagtaatatatttagtttactcgggtttctaaaaaaaaa

SEQ ID NO: 77 - DNA, 1376 bp, Glycine max
gtcagcattagcatccatctctgatctcactcaccattcaccaacctgcttctctgttctcttctttcccttcccaagaagaagaagaagaagatgccttcttcagaatcatcaacaggcttctccgaaaccatttgtgtgaccggcgctggtggcttcatcgcctcttggatggtcaaactcctcttggagaaaggctacactgtccgaggaaccctcagaaacccagatgatcccaagaacgggcacttaaaagagtttgaaggagcttcccagagactaactctgcataaggttgacctccttcatcttgactccgttagatctgttattaacggctgtcatggtgtctttcacactgcttctcccgtcaccgataaccccgaagaaatggtggagcctgcggtgaatggagctaagaatgtgatcatagcagctgcagaggctaaagttagacgcgtggtgttcacctcatccattggtgccgtttacatggaccccaaaaggagcatcgatttggtggttgacgagtcctgttggagtgatttagaattttgcaagaacaccaagaattggtattgctatgggaaggctgtggctgaagaagcggcatgggacacagcaaaagagaaaggggtggacatggttgtagtgaacccagttttggtacttggaccattactgcaacccagcataaatgctagtacaattcacatcctcaagtacctcactggctctgctaagacctatgcaaatgccacacaggcgtatgttcatgttagggacgtggcattggcccacatacttgtttatgagaagccttctgcctctggtagatacatatgtgccgaaagctctctccaccgtggagaattagttgaaattctcgccaagtatttccccgactacccagttcccaccaagtgttcagatgaaaagaatccgagagcaaaaccctacactttttcaaatcaaaaactgaaagatttgggattggaattcaccccagtgagtcagtgtttatatgaagccgtcaagaacctgcaggagaaagggcaccttcctgttcctgcaaggcagcaggaagattcaactactgtgaaaccttaaacgagctatccctttttatctaatttgtcaacaaacaagaagctgcagcttttggcaatctgggctcatcttatattattatagagaatcctacattagaagatttctgtatttggatttcgtaaaaaaaaaaaagacatcgaccaaattgttgccatgtatgaacttgataatattagtggaatttattcctcttgttgagttgaattattctgctcctcaagctcttttatctatcttaaaagaaattgtattctttaatattt

SEQ ID NO: 78 - DNA, 1351 bp, Glycine max
ttacatggttaatcagctaaagtttctttctgagggaccacaaccattcaacttcgtcccatttacacctaataagtgggaaaattttgaagggagagagaaaaagaaaggtttaagaacctctatataaagatagttaggcaagcacaacttctcatcagtatagaccaccttccctctcttctgttaccaatggctccctcatcaaactacatttctttccttttcctcaccacccttttgtttgctcttcaaatcagtgcaagagacagccagttcttcagcaaagtcacccattttgacaaaaacagtgtcaaagagacagaacttcccaacaaagaagcaccagaagtgaacaagccagagcaacaaccacctttcattccggagactgaaaacagctatggcctatatggtcatcatgatgagtataaccaagtcccttccaccaccaccaaccccacttcttaccacccctacaagaccgagtttgagaactccaacaacaagtattacaacaatgatgcctataacacaaggttctccgaaaccggttacaacaacaacaaggattcttatgagggtaaccaatatgagttaagtgacacaaagtacacagaagaagaatacaacaacaacaacaacaacaagtatcacaactcctatcagaacaacaaccagaagtactacaacaacgatgctgccaatggcatatactcctacaacaacaacaacaattacaatgctaacaataacaggtacaatacttacaacaacaacaatgccgttaacgggtacaatggtgagaggcaaggaatgagtgacacaaggtttttggagggtggaaaatactttcatgacgttgccgccgagaagtacaatccaacaaactatggtgactcatcaagggaagtgaacactaacaactggtacaacaacagaggtggcaactacaacgggaaccagaaccagcaggagtttgaagatgaacatgagaacttcgagccatgagcttggatcattggcaaagtaaagagttcactatacttttgagtatccactatgggtgtttaatgtttttattgtgttttagattcaagaaaacacaaagttaaaggatatgttaactatgaagggcttttattttattttttacttagtagtacttctattaagtcttgcttccggattgcgttgtctctgtcatgatctggaaatacctctacgagagcatgagtaatttttgcttaactaataagttgtataatattgtgaagtaagcttgtttcttgctctcatttttatttttcttttgttccctttttggtaagttatatattttttgttcgtt

SEQ ID NO: 79 - DNA, 1273 bp, Glycine max
aaaggaagcctgccaaatttcaaaatggaagcttccgcctaccaaccccataactaccattaaattatttcatcaaatcaaaatgaaaggaattacctatagaatacttgcgctaatttctataaataggcgttttgggcctttgtttacatagcaaatattcagagaattccaactgataaaaagaatattcagagaattccaaagacagagatgactgtcattaaggaagagcaacaaccaaaccaaatagctggccacaaagaacttggtcacaaaagtctccttcagagcgatgcactctatcagtatatacttgaaaccagtgtgtacccaagagagcatgagagcttgaaggagctacgagagttgacggaaaaacacccttggaacctgatggctacaccacctgacgaaggacaacttctaggcatgctccttaagcttatcaatgccaagaacaccatggaaataggcgtcttcactggttactccttgctttccactgcccttgccctcccttctgacggaaagatcttagctatggatgttaaccgggaatattatgaattggggttgcccgtgattgaaaaggctggagtggctcacaagattgacttcagagaaggacccgctcttcctcttcttgacgttctcattaaagacgaaaagaataagggagctttcgatttcatctatgtggatgctgataaggacaattacttgaactaccacaagagggtgattgagcttgtgaagcttgggggattgatcggctacgataacaccctatggaatgggtccgtggtggccccacccgatgctcctctcatggattatgttaagtattatcgcgattttgttatggagctcaacaaagctcttgcacttgattcaagggtcgagatttgccagcttcccgttggtgatgggattaccctgtgccgccgcatcatctgatcattctgctccttccctccccttttacctacttcacctgccacatatggaaaaaacacaaccttctactattttacctaatccttaaataaacataatgtgtgtctgcttggatagttactctgttgttgagttttagtcttctacgcaattatattcataatgtttctcttccttaaaacaaacacatgttttgaatcaaaatcgtataagatatatatatggaatgtgtaagcaattattagctaaattataaatatgctgaataaaagtgtaatcataaattatacttagcgaaacactacattattat

SEQ ID NO: 80 - DNA, 909 bp, Glycine max
gtgcactcttaacatcagccaaatggtttacttggcactgtgctctctgctaacgttggccttatctttggccacaacacatgcggcaaacttcgagatcgtcaacaattgcccctacacggtgtgggccgcggcgagtccgggtggaggccggcgtctggaccgtggccaaacgtggaacctctgggtgaacccgggcactgccatggcccgtatatggggccgcaccgggtgcaactttgatggcagcggccgcggccgctgccaaacgggagactgcacgggcggcctcaattgccaaggctggggggtccctcccaacacacttgcggaattcgcgttgaaccaatttggcaaccaggatttctacgacatctctttggtggacgggttcaacattccgatggacttctaccctctaaacggtgggtgtcacaaaatcagttgcagcgctgatatcaatgggcagtgcccggggccattaagggcacctgggggatgcaacaacccctgcactgtgtttaagacgaatgagtattgctgcaccaatgggcaaggaagctgtgggcccacaaactactcaaggttcttcaaggataggtgccatgattcttatagttaccctcaggatgatccaacaagtacttttacgtgtcccgctggctctaactacaaggtcgtcttctgtccattgggagaacctcatgttactcttcatatgcctgctagtaccgcccaccagtaaataaatgtccttccttgggcctacgaggaaatgaagtttttgctacaacttcatttatatatgatcataatcaataaactcggactttgcttctgtttaattttctgtgtaatgtatgaccaaaattaaatcatcgagtaaaatgacttctgtcaatttgtggg

SEQ ID NO: 81 - DNA, 931 bp, Glycine max
aaataacaacatttctttttataacataaaagtatatataaaaaaaggcaatcattaacgaacatggatgccaccaaggccaagacagttgacaatgcaggcactctcacactccccccaccattatatataacatcccatggtccaagcaataatgcatcctcaaatctctaacaccacatttcaaaatgaagcattgcatctcaaaacattttctaagttgtttcgctttgtttctgttgctagtcgcaacaacctatgccactgttgtccctaccaccacccaaaagccaccaagaagctttgcaaaccaattcctcatcccccagaacgcggcacgtgcggtgctgaggctgcggccattggtgtgggactccaaattagcacactacgcgcaatggtatgccaaccagaggcgcaacgattgtgccctagagcactccaacggaccttacggtgagaacattttctggggcagtggcacggggtggaagccagcacaagctgttagtgcatgggttgaagagcgccaatggtacaattattggcacaattcttgtgccaatggacagatgtgtggacattacactcagattgtgtggagcaccaccagaaaaattggttgcgctagcgttgtttgctctggtggtaagggcaccttcatgacttgcaactatgatcctcctgggaactactatggagaaagaccttactgaaaattaattataagaagtgagacaagtcaataatgttctaataggatcatagtagctacccatgttctccgctggcatattactattgtctgattgccatgtgcccatgtgcatgggtttaattgtggctctgtctcgctagctactctgtatccttgtttttattgcttgtctaatctaatataaagtataaatgtctaatatgcttccttttc

SEQ ID NO: 82 - DNA, 2072 bp, Glycine max
cacgtctctctgaaagctctcccactcgtgccgaaaacagcggtcacgcacgtgccaatcatttatattataccccattacaaacttcctctctccctctgcatcttcaaacacaacacacacacactgtgtttgtgaaactctcacacagacaagtcacaacaacaagtttctgttggtccaatggcatcagcaacaccccttttgaaagatgagctggacatcgtgatcccaacgatcaggaacctggatttcttggagatgtggaggccattctttgagccttaccatctcattattgtgcaagatggagacccttcaaagatcatcaaggtccctgaaggctttgactatgagctctacaaccgcaatgacatcaacaggatccttggccccaaggccaattgcatctccttcaaggactctgcatgccgttgctttggctacatggtctccaagaagaagtacatctacaccattgatgatgactgctttgttgccactgatccatccggacacaagattaatgcacttaagcagcatatagaaaacctgctctgcccatccacaccctactttttcaacaccctctatgaacctttccgagaaggtgcagattttgttcgcggttaccccttcagtctccgggaaggtgtaccaactgcagtttctcatggtctttggctcaacatcccagattacgatgctcctactcagcttgtgaagccacttgagaggaacactaggtatgtggatgctattttgaccataccaaagggcactttgtttcccatgtgtggaatgaacttggcctttgatcgcgatctcataggagcagcaatgtactttggtctcatgggtgatggtcagcctattggacgctacgacgacatgtgggcaggctggtgctgcaaggtaatctgtgatcacttgggattgggaatcaagactggtctgccctatatctatcacagcaaggccagcaacccatttgtgaacttgaggaaagagtacaaaggcatattctggcaagaagacattatcccgttcttccagagcattgttcttcctaaagaagctaccactgttcagaagtgctacattgagcttgccaagcaagtcaaggaaaagctttccaaggtagatccttactttgacaagttggcagatgccatggtcacttggattgaagcttgggatgagcttaaccctgcgggagcatcagtggccaatggcaaagcgtgatagttgtacttatatgaatatcattgcatgaaaacaactgagatatacgataatgatgattgtttaggtaatctgtgatcacttgggattgggaatcaagactggtctgccctatatctatcacagcaaggccagcaacccatttgtgaacttgaggaaagagtacaaaggcatattctggcaagaagacattatcccgttcttccagagcattgttcttcctaaagaagctaccactgttcagaagtgctacattgagcttgccaagcaagtcaaggaaaagctttccaaggtagatccttactttgacaagttggcagatgccatggtcacttggattgaagcttgggatgagcttaaccctgcgggagcatcagtggccaatggcaaagcgtgaatcggtttttggttaactgcataacagccacagaacttggaaatggaactatttatatactgtattttttttccttagtatttatcttcaacttagctgttattctcggcttaaaatttgcagcattgttagctgtaattctgtttaataacttcatcatttgagcttagaaacaataagtacacccgtagggcgttactttctttttattattgtttgatgggtagagatcacatggcagcatttcgttacatgtgaaataatagaaaggttattgatgtatgtcttgtttattactgagtatagatccatagtgatcaatttttaactgatgtgtttggaagtttggatttttctttggtactttatctgccacttgattattgtcctatcatacacttgctatataaataaatattaagtctctttt

SEQ ID NO: 83 - DNA, 913 bp, Glycine max
tgtgcagtggtgatcaaccataaacttattcaccaaagagaaaccaaaaggctcaaaacacaaccatgatcaatcatgctctttgtttcttagtattcctatctgcactccatgcaacacatggagttgaatacactgtcactaacaacgcactctcaactcctggcggcgtggctttccgtgacaaaataggggctgaatacgccaagcgaacacttgactcggccacccaattcatatggaggatctttcagcaaaacaacccttctgacagaaaaaatgtgcagaaggtaagcttattcgttgatgacatggatggtgttgcatatactagcaacaatcaaatccatctcagtgcaagatacgttggaaacaataaaggggacgtgaaaacagagataacaggtgtgctgtaccatgaaatggtgcacgtttggcagtggagtgggaatggtcgtgctcctggtggattaattgaaggtattgcggactatgttaggctgaaggcaaactatgcacctagtcactgggtgaaagcagggcaaggacagaaatgggaccagggttatgatgttactgcacgctttttggactattgtgatagtctcaaaagtgggtttgtggcacaacttaacaagttgatgagaactggttatagtgatcaatacttcgttcagttactgggaaaaccggttgatcagctctggcgagactacaaggccaagtatggcaatattgcctaataacctaagagctttttcatttaaagaataagcgagtatatcgcaatgtttttcaataagattgtatcagctctaagattctgctattacaagttttggtgttttatttaaaataaaaatattctgagtttttaaccataagcatgacaaaattagaaaaacaaacggat

SEQ ID NO: 84 - DNA, 1034 bp, Glycine max
ggcactcaacattttacactcttttcttcttctcgttgttgcttcgctgttgaatcatggatcagaaggtcgatttcagcgctccccattccatgggaaccactatcatcggcgttacttacaacggcggtgtcgttctcggagccgactctcgaaccagcaccggagtgtatgttgccaaccgagcatcggacaaaatcactcagctcactgataatgtatacgtgtgtcgctctggatcggctgctgattctcagattgtctctgattatgtgcgttacttcctccatcaacacacaatacaacttggacaacctgcaacagttaaagttgctgcaaaccttgtccgacttctctcatataacaataagaatttcttagagaccgggttaattgttggtggatgggataaatatgaaggtggacaaatttatggagttcctctgggtggaacaatagtacaacaaccttttgctatcggaggatctggctccagttacttgtatggtttctttgaccaagcctggaaagagggaatgaccaaggatgaagctgaggatttagtaaaaaaggctgtttcactggctattgctcgtgatggtgcaagtggtggggttgtccgaacagtcataataaactcagagggagtgaccaggaatttctaccctggcgaccaacttccattatggcacgaggaaatggagccccacaactccttgctagacattcttggtgccccagagccgatgagcatgtgatgtgaagaagttgccgtgtttcagaaatgtttcctgatagattatatcttgtcttctctcttttatgaactagctaacgatttgtgtagtcaattactttgtttggttggatatttttgatggttgagttatatccttttgttacgtaccatgattaacctttgtttccttcatcatatagctccaaagtaacgtgtactttggtgaattggtcttgcatagaaagcaattatcacaattcattttaaacttgtttttgttcgtggtagtttgtga

SEQ ID NO: 85 - DNA, 946 bp, Glycine max
gtccacactatggtattggggcctacatagaactagagtgaccccagggctacgctagttcttataaatagaggacactcctctgctaaagaaacacacagcaacaaacatcttcacaaactagtaatattattcttcgaatccattttattattcataatgggtattttcacatttgaggatgaaaccacctcccctgtggctcctgctaccctttacaaagctctagttacagatgccgacaacgtcatcccaaaggctgtcgaagccttcaggagtgttgaaaaccttgaggggaacggtggccctggaaccatcaagaagatcactttcgttgaggatggagaaagcaagtttgtgttgcacaaaatagaatcagttgatgaggcaaacttgggatacagctatagcgtagttggtggagttgggttgccagacacagtggagaagatcacattcgaatgcaaattggctgctggcgccaacggagggtctgctgggaagctaactgtcaaataccaaaccaaaggagatgctcagcccaacccagacgacctcaaaattggcaaagtcaagtctgatgctcttttcaaggccgttgaggcctaccttttggccaatcctcattacaactgatccaattcgatcttcagtattcagtgatctgcaaagaccttgttttatattatatacaagagttttcttgccttgtgtggagtgttattcaatcactttgagtgtgtggtgtggcttccaattgatgtaacgagtgtttcctttcttatttcctttttccacagaattgtgagagccagttgatgtctttgtatgtcacttcatcaataaatcaatatgttataataataaaagaaagcatcatttatacttctgcttgttttatgtttactatggtgactttatatagaatttaacccagtttttgagg

SEQ ID NO: 86 - DNA, 1349 bp, Glycine max
agtgagagtcatcacatgaattttactttttttggaaggcaacacatgaattttacttaatagaatacagtagaaaaatgctaaaattaaagtagctaattagaagtcatcataaccatagacaaattgctccatatagggtccaaaatggagtgctcgcaccacactataaataaagcttgaggagcggtgtatacttcacaagttcaaagacacaccattattaaacaatggcctttcactcccatggtcacatattttctttctcctccattttgttttgcatgtttgccatggcatcttctcagctaacttcaaattgctatgaatcaacatgccctcaagctctctctatcatcaaaacggcggtgattggtgcggttgcgaaagagcaccgcatgggagcctccttgctccgtcttcatttccatgactgctttgtcaatggatgtgatgcatctgttctgttggatgacacttcaacctttactggagagaagtcagcggctgcaaatgtgaattctctgagaggttttgaagtaatcgatgacatcaaaactaaagtggaggctgcttgtcccggagttgtttcttgtgcagatattctagctattgctgcccgtgattccgttgttacattgggaggtccatcatggaatgttgggttaggcagaagagattcaaccacggcaagtaaagatgctgccactacggatatcccatctccactaatggatctcagtgcccttatatcttctttttcaaataagggcttcaacaccaaggaaatggtagctctctcaggagctcatacaacagggcaagccaggtgtcagctattcagaggcagggtttacaatgaaagcagcattgagtcaaactttgcaacatcactaaagtctaactgtccaagcactggcggagacagcaacctttccccacttgatgtcaccacaaatgttgtatttgacaatgcttatttcaagaacctcatcaacaaaaaggggcttctacattctgatcagcagttatttaatagtggtggttctacagattctcaggtcactgcctatagcaacgacccttcagctttctatgcagactttgctagtgccatgatcaaaatgggaaaccttagccccttaacagggaaaagtggccaaattcgtactaattgccacaaggttaattgattaattacagaaataatatgagccaaagacacgctcattcagtcacaatacgtatggcctagtttatgtttagtgataataatctaagaatataatattgaagtgtaagctttagtcctctcaaaatagaggggaataaagaaaagtggtgtattg

SEQ ID NO: 87 - DNA, 1815 bp, Glycine max
gtgtttgatacactgtgtatgtgttttctttcagggcctttctttatattataataatcttggaacaatgttaccccagaagcaaggtgaagaaaccataatgtcaaaccttaacgaaacaacgatcgagcgtggtgaagagagagaggaagagaacgtgggtggtggtggtggtggttcacattccagtttgaaaagcattctctggcatggtgggtctgcttatgatgcatggttcagctgtgcctcaaatcaggttgcccaggttctactaacactgccatactctttttctcagctgggaatgctttctggcatcatattccaaatcttctatggaataatgggaagttggactgcttatctgattagcattctgtacatcgagtacaggaccagaaaagagaaagagagtgtcagcttcaaaaaccatgtcattcagtggtttgaagtgctggaaggtttactgggtccatactggaaagccattggcttggccttcaattgcacttttctcctctttggatccgttatccagcttattgcttgtgcaagtaacatatacctcataaatgaccacttggataagaggacctggacttacatattcggagcttgctgtgccacaacagtattcgtaccttcatttcacaactatagaatttggtctttccttggccttggcatgaccacttacacagcttggtacatgaccattgcagccattgctcatggccaggttgaaaatgtgattcacactggcccaaaaaaattggtattgtatttcacgggagccaccaacattctctacactttcggcgggcacgccgtcacagtggaaatcatgcatgcaatgtggaaacctcaaaagttcaaatatatctatctttatgctaccctttatgtattcacactcaccataccatcttctgttgccgtttactgggcctttggtgacgaactcctggaccactccaatgctttctctattcttccccgctctggttggcgcgataccgctgtcatcttaagctcattcatcagttcatcacttttggatttgcttgcacaccattgtattttgtgtgggagaaagtgataaaaatgcagacacaaagagcctatgtttgagggcacttgctaggttgcctgtggtgataccaatatggttttttgctattattttcctttctttggtcctattaactctgctgtgggggctcttctggttaccttcaccgtctatgtcatccctgcttctgctcattgctcacttacaaatctgcttctgcccgacagaatgctgttgagaaattacccttcttcatcccaaattggacaacaatgtatttggtgaatgcatttgtggtggtgtgggttctagtggttggatttggatttggagggtgggctagcatgacaaacttcgtcaaacaggttgacacatttggactctttgccaagtgctaccagtgcccgccaaaactcccagcttccaacaacacaaagctgcatcattgaaaattatatatatgttcttttgcatgatacatatagggacacatcagcatcttcaccgttgtgtatttcccctatcttttgtttaatctccttataaatattgttcatgctgatgttcccttgtgtagcaaggcaacattgttatatttcgctttcgaatcatcgtggttaatcataaacatgcactatagttatagcatatgtagggattgatgatgtttggcaattggaatatagcgacctcaagtatagcctgcatgtttccaataataatgtctttgctctccattataa

SEQ ID NO: 88 - DNA, 857 bp, Glycine max
taccttcaattaagcaaacatgaagcaaaaaatagttatcaagctgcaaatggactgcgacaaatgcagaaacaaagccctaaaaattgcagcagaagtacgaggtgtaactacggtgtccctggaaggggacgacaatgatcgcgtggcggtgagcggtgtgaatgtggacatggtttgcttggccaaccagctgaagaagaagttcagctccgtgacgattctcaccgtggtagacctagttaaagaggaggaggcgaagaagaagaaggacgaggaggagaagaagaagaaggaagaggccgagaagaagaggaaggaagaggaagaaaggttgaagaagatgctgcgttctgttctttgcaaaaagtgtaaaagctccagttgccatggtaagtgtgacactgcatgctgcacaaagtgtgaaagcatccactgtggtggagactgtttcatcgtttgtgttaattgtgatagccccaagtgtgagggtgactgtaagccatgtatcaattgcctcagctctaagtgtgagtgtgagtgtgagccatgccctaagcctccttcaccctgccctaaatggtgcaactgtcataaatgctatgtgccctatcaacaaccttgttattacccttatcctccccaagtagtatgctatgatacatgccctgattctccttgctccatcatgtgatccaccacctggtcttggtgtacaattatcgcatgcatatttgaatttatgacctcatatatacactccaaatcctacctcttggtccacctagtggttctatgaaatcacatggtttaataataagatatttgtatttgatgatatgtttgcaatactgtcc

SEQ ID NO: 89 - DNA, 1038 bp, Glycine max
tctctctttcattctcacttgtactcatctctttttcacaactttttttgtaccttctgatctaatggcagttccagtgattgatttttcaaagctgaatggtgaagaaagaaccaaaactatggcacagattgctaatgggtgtgaagagtggggattttttcagttgatcaaccatggtattccagaggaactccttgagagggtgaagaaggttgcctctgagttctataagctggaaagagaggagaatttcaagaactccacatcagtgaagctactaagcgattcagtcgaaaagaagagcagcgaaatggagcatgtggattgggaggatgttatcactctccttgatgataatgaatggccagaaaaaacaccaggattcagggaaaccatggcagaatatcgagccgagttaaagaaattggcagagaaactgatggaggtgatggatgagaatcttggcttaacaaaaggatacatcaagaaggcactgaatggtggagatggagaaaatgccttctttggcaccaaggtgagccactaccctccatgtccccatccagagctcgtgaagggtctgcgagctcacaccgatgcaggaggcgtaatcctactcttccaagatgacaaggttggtggccttcagatgctgaaagaagggcaatggattgatgtgcagcctttgccaaatgccattgtcatcaacactggtgatcagattgaggtcttgagcaatggtagatacaagagttgctggcacagggttctggccacaccagatgggaacagaagatcaattgcctccttctataatccttcattcaaggccaccatatgtcctgcaccacaacttgtggagaaagaagaccaacaagtagatgaaacttatcctaagtttgtttttggtgactacatgtctgtctatgctgagcagaagttccttcccaaggaaccaagattccaagctgttagggccatgtgagaaaagcaacttgctttcgtatagaaaatgcttttgcgcttccccccact

SEQ ID NO: 90 - DNA, 732 bp, Glycine max
cactcacattcattcattcactcaccccaaaacccaaaaacctttccttttactttttctcaaactgaaacataaaaaatggagggtcttattccttttgtttacaaggctatcatgcaatccaagggcgacaaagagggtcatcctataggatcatggctttgtgagtctccttactcatacatgagacttcctggtgactcgggtaggtttcaaattcaagcaccagctgctgcttcaccctcttccaccaaccctaattcttcttctgctacacaaatcattgtttcctctggtgttcagtccccacatcagtgtttaacacatcgtcgaattgcagcatgaaaatgaacaagtttgtgcctgaagaaaaagccgaaggtgcttaattcaagagttcaagtttgtgctttcaagtgttgggaatcttggttctttgtttcaacttaatttggagttgtaagcgtgtggaaaacaacaaccaaaagggtctcatacgcttattaggtagtggtggtatttcagtatttgtaattacgttgtagatatgggtttctcatgcttaagaaattgttcgtgttttatgaccaaagggtcacttgaactatatgagtttttagacaaaataatgagcaaaatgtaataggtttattgttgtagaataaagcaaataatcaatctgcaattgttcgttgtttattattttctgatttcggactttaattcatcttt

SEQ ID NO: 91 - DNA, 949 bp, Glycine max
attcctcatcaaataaataattaattacggtcaagataaaatgcaaaccaattcaatgcttcatacttataaatagcaatcagcaagctctacataaatcacaattcaatcatcacaagcaattacatatagcacaattgtttaagagtgtattactaaggaaaaacaatgaaagttgtgtacttccttgttgccatcttggctttgacatcctctcttgtctctgcctatgatcctagtcctctgcaagatttctgtgtggcagccaaagaaaaagatggtgtgtttgtgaacgggaaattttgcaaggaccctaagcttgtcaaagctgaagatttcttcagacatgtagaaccggggaagactgacaacccagtaggttcaaacgtgactcaggtgtttgttgatcaactaccgggactaaacacgcttggcatagctttggctcgcatagattttgcaccaaagggtttgaacgctccccacactcaccctcgcggcactgagatccttatagtccttgagggtactctttatgttggatttgtgacttccaatcaagatggaaatcgcctcttcaccaaagtgctgaacaagggtgatgtgtttgtgttcccaattggtctgattcatttccaattgaatgtgggatatggcaatgctgttgccattgctggccttagcagtcaaaatccaggagctatcactattgcaaatgctttgtttaaagctaatccgcctatttcttctgaggttctcaccaaagctttccaggtggacaagactataatcgattatcttcaaaagcaatcttggtacgacaacaataactaatttgggagtatggaggatcatttcgcactgtctagttattggataattgatcactttaattattatttcagtcacgccatacattagacctaataaataaaacctttgtggatta

SEQ ID NO: 92 - DNA, 1423 bp, Glycine max
ccaattaattttcccatgcaatgacaacttgtccgtattctacgtggtaggttaggctacctgccgagacaaattgccttgagacaaattcaatagagaacccttccaagggaccattataaatagagaactttcattaaccgataagccacaccctttcaatcaaacacaaacacttgaagtactaagttagtgtgtttgagcaaattaactatggcttcgttttgttctagattgacaatttgtttggctctgtttgtcctcatatgggggagtgccaatgcacaactttctacaaacttttactaccattcatgtccaaacctcttctcctctgtgaaatccacagtgcaatctgccatatctaaggagacccgcatgggtgcttctctccttcgcttgttcttccacgattgctttgtcaatggatgtgatggttcaattctattggatgacacatcaagcttcaccggagagaagaacgcaaaccccaacaggaactctgctcgtggattcgaggttattgacaacattaaatcagccgtggagaaagtgtgtccaggagttgtttcctgcgcagatatccttgccatcgctgccagagactctgttcagattcttggaggccctacatggaatgttaaacttggaagaagagacgctagaactgctagccaatctgctgctaacaatggcatccctgcacccacttcaaaccttaaccaactcatctcaagatttagcgctcttggactttccaccaaggacttggtcgccttgtccggtggtcacacaattggacaagcaaggtgcacaaacttcagagcccgcatctacaacgagaccaacatagaaaccgcatttgcaaggactaggcagcaaagctgccctagaacatcagggtcaggggacaacaatctggcaccacttgatcttcaaactccaaccagctttgacaactactacttcaagaacctcgttcagaagaagggtctcctccactctgatcagcaactgttcaacggtgggtccaccgactccattgtgcgtggctacagcaccaacccgggcaccttctcctctgatttcgccgccgccatgatcaagatgggagacattagtcctctcactggctccaatggagaaatcaggaagaattgtagaaggattaactaatttaattcagtcttgaatattaagggtcctacacatacgcaagcaatttaattgtgtttaataagttgttaaaacatgttttggttgtattttggattcctagtgtagtttcggtgatcaatgccgtctactttagtgtgttctacttccctttatttttgtttcttttttactttttccttaactatattgtagggaaaaaaaaaatcctttatcaagcatttatcaagaacggagtttgcttttt

SEQ ID NO: 93 - DNA, 1662 bp, Glycine max
cctcgttgccccccgaacatgggaaactgctgcgccacgccgtccacggacgaaacggcgaacaagaagggcaaaaagggaaaaaaggaaaaccccttcgcgatcgactacggtttcaacgccaccgccgcgaacgggtcgaagctcaccgttttgaagtccccgacgggccgcgagatcgaggcccgctacgagctgggccgtgagctgggccggggcgagttcggcataacctacctctgtacggacaaggggaccggagaggagctggcgtgcaagtcgatctcgaagaagaagcttcggacggcgatcgacatcgaggacgtgaggagggaggtggagatcatgcgccacttgccgcagcatgcgaacatcgtgaccttgaaggacacctacgaggatgacaatgccgttcatcttgttatggagctttgtgagggcggggagctcttcgaccgcatcgtggcgcgaggccactacaccgaacgcgccgccgccgccgtcaccaagaccatcgtcgaagtcgttcagatgtgccacaagcaaggtgtgatgcatcgggatctcaagccagaaaactttttgtttgcaaataaaaaggaaactgcggctttgaaggctatagattttgggttgtcggtgttctttaaaccaggggaaaaatttaatgagattgttggaagtccatattacatggctcctgaggtattgaagcgaaattatgggccagaagtggatatctggagtgctggagtaattctatacatcttactttgtggtgttccacctttctgggcagaaactgagcaaggagttgcacaagcaatcatacgatctgttgttgatttcaaaagggatccatggccaaaagtttctgataatgcaaaagacctagtgaagaagatgctagatcctgacccgaagcggcggcttactgcacaggacgttttagatcatccatggttacaaaacgcaaagaaggctcctaatgtttccttaggagaaactgttagagcaaggctcaagcaattttctgtaatgaacaagcttaagaaaagagctttaagggtgattgctgagcatttgacggtagaagaagctgccgggctaaaagagggattccaggttatggatacaaacaacagaggcaagattaacattgatgaattacgagtagggttgcataaacttggtcatcaagttcccgaatcagatgtccaagctcttatggatgctggtgatgtagacggggatgggcacctagattatggagagtttgtagccatttctgttcatctgagaaagatgggaaatgatgagcatcttcgcaaagccttccaattttttgatcagaacaaaagtgagtatattgagattgaggagctgcgtagcgccttatctgatgatcttgacacaaacagtgaagaagttgtcaatgcaattatgcatgatgtggacacagacaaggatggaaggataagttacgacgaattttcgactatgatgaaggctggcacagattggagaaaagcatcaagacagtactcacgagagaggtttgccagtctcagccttacgttgatgagggacgggtcattgcatttaaacaatgaaaaacaatgacggtagatgactactttagaaatgtcaatttttactttg

SEQ ID NO: 94 - DNA, 794 bp, Glycine max
aggttttgctaattaatcttgggtgcaaccaagctaccaacccggttacattaatttcctctacggtttaaccactaagctaaggttacctaagaaatcaaaaccaatatgtgactatataaatccttttgtagtggccagtgtttcttcacggccaccacatgtggttctcatcatttcttagtccttttctaatgcgccactccaaagtacgtcccaatgcaccttctgatgatcaatcatcccaactaacacacaatccgcaacttgatcatggagggtggtctaggcgtggtcgtgaattttgtgaggcaagaagagcgtttttgaacagttaccatttgagtttggaaagaaaaaacaatgttagcttcaaggaaaagttgaagaagtcagtgaaagaggttaatgaagcagcaatgggagttgttttgggcatgcgtcgtggggtgtccaagagaagggttggcatcaaggtttttagggtcaaaatgtcttcacactcgatggttcttgttaccttaagatgttttatcccatggttgaacaaaagtaaggttatataaagattaaaaataatatatacagttctttaggtgttgtttagtatattgttctttgatgaaaattggagttttgtcgattaccaagatgaaactctaatttaaattagaaaatgacaccaataacacctatgaaattgtatctaaattttatccattatataataatcacatatatatcttgtatgtgaacgtattgctatatattaggacaaagaattatcaacatgt

SEQ ID NO: 95 - DNA, 1728 bp, Glycine max
gagcgggtgcaggaaaattaatctccctttcattttctctgcatctgtacatgccgtccttagaggaggagctcttcccttcaaccccaggcaagttcaagatcgagcgggcccaccacatgaaccgccagctctaccgctgcttcgcctccaccagcaccatgttcctgtgggccctctttttaatcgccctcaccgcttcatatctcagcttccaaggcttcgttgattccggcagccgctacctctccgcctcctggggcgggatccagtgggagaagcaggtccgcacctccgcccagatccaccgtcaaggcggcatgtccgtcctcgtcaccggcgctgccggcttcgtcggctcccacgtctccctctcgttaaaacgacgcggagacggcgtcgtcggactcgacaacttcaacgactactacgatccgtctctgaaaaaagcccgcaagtccctcctcgccaagcatgacgtcttcatcgtcgatggcgacctcaacgacgccaagctcttggcgaagctcttcgatgtggtcgctttcacccacgtgatgcacctcgcggcgcaggccggggttcgctacgccatggagaatccccactcctacgtccacagcaacatcgcgggcctcgtcacgcttctcgaggcctgcaaatcggcgaaccctcagcccgcggtcgtttgggcctcgtccagctccgtctacgggctcaacgagaaggttccattttccgaatcggaccagaccgaccggcccgctagcctctacgccgcaaccaaaaaagccggtgaggaaatcacacacacctacaatcacatctacggcttatccatcaccggtttgaggtttttcaccgtgtacggaccctgggggaggcccgacatggcttatttctctttcaccagaaacatcttacaagggaaacctatcaccgtttaccgcggtaaaaaccatgtcgacctggccagggatttcacctacattgatgatattgtcaagggttgcgtcgggtcgttggatacttcggccaagagtaccgggtcgggtgggaagaaacggggacccgctccgtatagaatatttaacctggggaacacgtcgccggttactgttcccactcttgtcagtatattggagcgccatttgaaggtgaaggccaagaggaatattgtggacatgcctggaaacggcgacgttccgtttactcacgcgaatattagttcggcccggagagaactcgggtacaagcccaccaccgatttgcaaaccgggttgaagaagttcgtcaagtggtacctttcctattacggctacaatcacggcaaacctgtaaattaattttattttattttgtttcccccccctttttttttctttttcggaaagggttttttctttttttagtgtggtaggttttaagtcacagattaaaacagacagggaaaaaaaagaagaaagaacctggtcaagaagattagaaggagaagggaaattggggctaaaaggaaggaagaaaagaaaaagaaaaaaagaaaagagaaaagaaaagaagagaaggagaagaatacaattctttacttaggaggccagctaaaacagctgttgttccttcgttgatttcttttcttttttggtattaattggtgtttttttgtattttttttaacattttctttttctgtaaagaaagaaagttccgagttcaaaaaaaaaaaaaaaa

SEQ ID NO: 96 - DNA, 1821 bp, Glycine max
gcagttagcacattcctctttgttatatttacaagagtgttcgtagcttcccccatcatttggggcttgcttaacggacaaaactgcccaactgctatcccgtaaaaccagaaatcacgaagcacttgggtcctacatgttcagtttaatcatgccatttagcccggctttaattcctttataaacaccctccagctacgtactttcctcacacaacacaaatcaaacatggccaccacttttctctctctcctcttcctcctcttctctctcctcaccccgaccctcatttcctcttcccctgtccaagaccctgaatttgtggcccaagaagttcacaggaaaatcaatgcttctgtagctagaaggaatttggggtacttgtcctgcgcaacagggaaccccattgatgattgctggaggtgcgacccaaactgggagaagaaccgccagaggctagcggattgtgcgattggtttcggcaaaaacgccatcggaggaaagaacggaaaaatctacgtggtgacagattccggcgacgacgatccggtaacccccaagccgggaactctccgttatgcagttattcaagacgagcctctctggatcatcttcgcaagagacatggtgatcaagctgaaggaagagctgatcatgaactcattcaaaaccattgatggaagaggtgctagcgtgcacattgcgggtggtccatgcataacgatacagtatgtaacaaacgttataatccatgggattaacattcatgattgtaagcaaggtgggaatgctatggtgcgggactcaccacggcactacgggtggaggaccatatcggacggtgatggcgtgtcgatctttggagggagtcacgtatgggttgaccattgctctttgtctaactgcaatgatgggttgatcgatgccattcatgggtctactgggattaccatctccaacaattacatgactcaccatgacaaggtcatgcttttgggccacagtgattcctacactcaggacaagagcatgcaggtcactattgctttcaaccactttggtgaaggcctagttcaaagaatgccaaggtgtaggcatggatattttcatgtggtgaacaatgactatacccactgggaaatgtatgccatcgggggaagtgctaatcctaccatcaatagccaaggaaacaggtttgttgcacccgatgacagattcagcaaagaggtgacaaagcatgaggatgcaccagagagtgaatggaaaggatggaattggaggtcagaaggggacttgttggtaaacggtgcgtttttcacggcatcgggggctggagcctcctctagctatgcaagagcatctagcttgagtgcaaggccatcttcattggtggggtccataaccactggtgcgggtgcactcagctgtaggaagggttcccgctgctgattatgacccctccattaattccaaaccaaaattatcaaaagggagggaaggggaaacagaatgaccctattaaattaactatggtcatttctttatagttaggatgtatactttcctatcatttccttcctgcattcttttctttagttttttttttttttttaccattgttttccactatttgtcttcttttttttttctttagcttttttacttttgggtttcttctatggtctacttttgtctgattacaacctccgctgtaaacctcatagaaccaaatttgtgaatctggtctgtgagaaagcaagtgcagaagagttgatatatatatatatatatgatgattttacatgtttctatgttgaatat

SEQ ID NO: 97 - DNA, 2205 bp, Glycine max
gatctcaaccacttcactttccaagaggaggtgaggcatcatggctcaaaatggaagtggggatagagtagtggtggacaatggttttgaatccattgatgatgcatacgatcatgaacacaagctgagtcaaaagggtagcaccaaggtcaaggaggaagaggtttctgtggagagggtgttccagcaccttctggtgccatcatggaggaaccaattgacagtgagagcctttgtggtcagctttgcactcagcatactcttcagcttcattgtgatgaagctcaacctcaccactggtattattccttcactcaatgtctctgctggccttctggggttcttctttgtgaagacatggaccaagttcttggagaaatctaacatgttgaggcaacccttcacaaggcaagagaacactgtcatccaaacctgtgttgttgcatcctctggcatagcctttagcggagggtttggaagttatctctttggaatgagtgaagaaatagccaagcaatcctcagaccccagtcattttaaggacccaaaattagggtggataataggttttctctttgtggttagctttctcggcctattctcagttgtacctctacgaaagattatggtcattgacttcaaattgacatatccaagtggtactgcaactgcacatctcatcaacagcttccacactcctcaaggagctaaactagcaaagaagcaagtgaaaatgttgggaaaattcttcagtttgagttttttttggggcttctttcaatggttctacactgctactgaccagtgtggatttcaagccttcccttcattggggcttaaagcatatgaaaacaagttcttttttgattttgctgcaatctatgttggagttggaatgatatgcccatatatcataaatatatcagtgcttcttggaggaattatttcttggggaataatgtggcctctcataaaaaccaaagagggtgattggtatgataagggccttggtgagggcaaccttcatggcatacaaggttatagggtatttatagccattgccttgatactaggagatggtttatataacttcataaaggtgcttactcataccctttggggattgtatcatcaaatccgggaaaaacaaagggagaatgtccttcctgttgcagatcaagactccccctcaaattctcatctatcttatgatgaccagcgccgcacccaactcttccttaaagatcaaattcccacatggtttgcaatttctggttatgttgctattgctgccatctctactgccactctgccacacatcttccccgaactaaaatggtattacataattgtcatctacctaattgctcccaccttagcattttgcaatgcttatggttgtggactaacagattggtcccttgcttccacctatggaaagctagccatctttacaattggagcatgggccggttcatcaaacggtggagttctcgctggcctagcagcctgtggggtgatgatgaacattgtttccacggcctcagacctgatgcaagattttaagactggctaccttacattggcttcgccacgctccatgtttgtgagccaaataattggcacaacaatgggttgcataatttctccttgtgtattttggattttctacaaagcttttcctgaccttgggagaagtacaagtgaatatcctgccccatatgcaattatataccgtaacatggctatattgggggtacaaggttttggccatctaccaaaaaactgcctcttgctttgttacatattctttgctgctgccgtcgcaataaacttgattaaagatttccttggcaagagagggaggttcataccacttccaatggccatggcaatacctttctatatagggccatactttgccattgatatgtgtgttggaagtctgatattgtatgtgtgggagaggattaacaaggctaaggcagatgcttttgcaccagctgtagcttctggtttgatatgtggcgatggaatatggactcttcctgcttcaatacttgctcttgctggagttaagccaccaatttgtatgaagttcttgtcaagagcagcaaatgcgagggtcgatactttattagggaattaaggtcaggtttgaattggatcttgtgaaaagttactgccttgtaaatctgtgaaaca

SEQ ID NO: 98 - protein, 625 aa, Glycine max
MEKLIRMFPLLLVLLLCPNFAFAGHDYGQALSKSLLFFEAQRSGYLPHNQRVTWRAHSGLQDGKASGVDLVGGYYDAGDNVKFGLPMAFTVTMMSWSIIEYGKQMAASGELGHAMEAVKWGTDYFIKAHPQANVLYGEVGDGNTDHYCWQRPEDMTTDRHAYKVDPSNPGSDLAGETAAAMAAASIVFRRSNPAYAGELLRHAYQLFDFADKYRGKYDSSITVAQKYYRSISGYNDELLWAAAWLYQASNNQYYLDYLGRNGDSMGGTGWSMTEFGWDVKYAGVQTLVAKFLMQGKSGHHAPVFERYQQKAETFMCSCLGKSNRNVQKTPGGLIFRQRWNNMQFVTSASFLATVYSDYLASSGRNLRCSSGNVPPAELLSLAKSQVDYLLGDNPRATSYMVGYGSNFPQRVHHRGSSIVSIKVNPSFVSCRGGYATWFSSKRSDPNLLTGALVGGPDAYDDFADERDNYEQTEPATYNNAPLIGILARLGGGHGGYNQLLPVVVPAPKPAVTKPQPTPSPKTTPSPASWSGPISIEQKRTTSWVANGKTYYRYSTVVTNKSNKSLNSLNLSISKLYGPIWGVTKSGDSYTFPSWLSSLSAGKSLEFVYIHSASPADVSVANYVLA

SEQ ID NO: 99 - protein, 183 aa, Glycine max
MEESVLTKRPREEEPLENKDSTYELLESLSKRHRSYNHILSLLESEEDDSTQDLSSLITSLQQEITNCASDSDTLLNQHSLTNTTTTTTTNSNLEDCSSSTTKYSSDMMEEHDDKEGVMRHLLEASDDELGIPNKEDESLDLGEDGFKFNGGDMFSSICDGLLWELEDEAANYYDLLQSQLFL

SEQ ID NO: 100 - protein, 189 aa, Glycine max
MAPATLSLSLLLLFSLSIAVTVRSSDYDYDCVYTAYVRTGSVLKGGTDSKIGLKLYDKYGYYIYIKNLEAWGGLMGKGYDYFERGNLDIFSGRGPCLDGPVCAVNVTSDGSGSHHGWYLNYVQVTSTGPHLSCAQDQYEVEQWLALDTSPYQLWAVRNHCRYSLDRAQPVSERPGSGSGSAFSILNARA

SEQ ID NO: 101 - protein, 142 aa, Glycine max
MGSKTKTFTFEEVAKHNHRKDCWIIVKGKVYDVTPFLDDHPGGDEVLVTATEKDATTDFEDIGHSDSATEMMEKYFVGEVDTNTLPAQVTSSSSVRPPTQAPVYNNQSSGFVVKILQYIVPLLILAFAFGLQYYGKKSKSEN

SEQ ID NO: 102 - protein, 336 aa, Glycine max
MARNLLLVLFAVATLLHGSAAQTRHMVGDATGWIIPAGGAATYTAWASNKTFTVNDTLVFNFATGQHNVAKVTKSAFDACNGGSAVFTLTSGPATVTLNETGEQYYICSVGSHCSAGQKLAINVNRASSTGPSPAPQPRGSGSPPRASPVPTQAPQASSPTPPPRSAPAPAFGPSSEPATFIVGETAGWIVPGNASFYTAWASGKNFRVGDVLVFNYASNTHNVEEVTKANFDACSSASPIATFTTPPARVTLNKSGQHFFICGIPGHCLGGQKLAINVTGSSTATPPSAAAPPTTPSSPSPAGAVTPPPQNSGAASLGVVGVFATLLSVAATFFY

SEQ ID NO: 103 - protein, 204 aa, Glycine max
MGSSKLLGIMAMLFIVLLPMAAKGDNITDFFDKVCEEVECGKGSCVVNTSYPLNFVCECDSGWKRTQDDDDEYATSFLPCVIPECSLNYGCQPAPPPVPEKSFPHNFSAFDPCYWAYCGEGTCTKNRTHTHRCECQPNYYNLLNISVFPCYSECTLGSDCSRLGIKVANSSTDSGSQDSSASIFTGRFHWMVMLLMSTGMVMWS

SEQ ID NO: 104 - protein, 269 aa, Glycine max
MGTSILDALNVRVEGSGDKYLVLAHGFGTDQSAWQRVLPYFTRNYSVILYDLVCAGSVNPDHFDYRRYTTLDAYVDDLLNILDALRVPRCAYVGHSISAMIGMLASIRRPDLFSKLILIGASPRYNKFLNDKDYHGGFEQGEIEQVFSAMEANYEAWVNGFAPLAVGADVPAAVREFSRTLFNMRPDISLFVSRTVFNSDLRGILGLVNVPCCIMQTARDMSVPASVATYMRDHIAGKSTIQWLDTEGHLPHLSAPSYLARQLEIALSQ

SEQ ID NO: 105 - protein, 149 aa, Glycine max
MADQLTDEQISEFKEAFSLFDKDGDGCITTKELGTVMRSLGQNPTEAELQDMINEVDADGNGTIDFPEFLNLMARKMKDTDSEEELKEAFRVFDKDQNGFISAAELRHVMTNLGEKLTDEEVDEMIREADVDGDGQINYEEFVKVMMAK

SEQ ID NO: 106 - protein, 207 aa, Glycine max
MGYWKSKVLPKIKKVFEKNSTKKAAAAEATKSFDESKEEYNKAFEEKKTELQTKVVEIYEASSTEIKSLVKEPKEAGLKKNSTEVQKFLEELVKIDFPGSKAASEASSKFGPALASGSVFFVFEKVSTFIVTEEKEVEAPPAVETKTEEETSSVVKERETVVEEEKKEEEKPQADETSDEKKVEEKQAETAAKEEEKPAEPAEPPKP

SEQ ID NO: 107 - protein, 250 aa, Glycine max
MGKSYPTVSADYQKAVEKAKKKLRGFIAEKRCAPLMLRLAWHSAGTYDVSSKTGGPFGTIKHPSELAHGANNGLDIAVRLLEPLKAEFPILSYADFYQLAGVVAVEVTGGPEVPFHPGREDKPEPPPEGRLPDATKGSDHLRDVFGKAMGLSDRDIVALSGGHTIGAAHKERSGFEGPWTSNPLIFDNSYFKELLSGEKEGLLQLPSDKALLSDPVFRPLVEKYASDEDAFFADYAEAHQKLSELGFAEA

SEQ ID NO: 108 - protein, 502 aa, Glycine max
MDPVSVWGNTPLATVDPEIHDLIEKEKRRQCRGIELIASENFTSFAVIEALGSALTNKYSEGMPGNRYYGGNEYIDQIENLCRSRALQAFHLDAQSWGVNVQPYSGSPANFAAYTAVLNPHDRIMGLDLPSGGHLTHGYYTSGGKKISATSIYFESLPYKVNSTTGYIDYDRLEEKALDFRPKLIICGGSAYPRDWDYKRFREVADKCGALLLCDMAHTSGLVAAQEVNSPFEYCDIVTTTTHKSLRGPRAGMIFYRKGPKPPKKGQPENAVYDFEDKINFAVFPSLQGGPHNHQIGALAVALKQAASPGFKAYAKQVKANAVALGKYLMGKGYSLVTGGTENHLVLWDLRPLGLTGNIYRIGSLPSGFDLLQMSINLTCSLCDCFAGNKVEKLCDLCNITVNKNAVFGDSSALAPGGVRIGAPAMTSRGLVEKDFEQIGEFLHRAVTLTLEIQKEHGKLLKDFNKGLVNNKAIEDLKADVEKFSALFDMPGFLVSEMKYKD

SEQ ID NO: 109 - protein, 224 aa, Glycine max
MAALRKAVFFVIAQCFTFSAYAARFEITNRCTYTVWAASVPVGGGVQLNPGQSWSVDVPAGTKGARVWARTGCNFDGSGRGGCQTGDCGGVLDCKAYGAPPNTLAEYGLNGFNNLDFFDISLVDGFNVPMDFSPTSNGCTRGISCTADINGQCPSELKTQGGCNNPCTVFKTDQYCCNSGSCGPTDYSRFFKQRCPDAYSYPKDDPTSTFTCNGGTDYRVVFCP

SEQ ID NO: 110 - protein, 240 aa, Glycine max
MDRKSLLSLAIIAIVVAAVGGQSPAASPTTSPPAATTTPFASPATAPSKPKSPAPVASPTSSSPPASSPNAATATPPASSPTVASPPSKAAAPAPVATPPAATPPAATPPAATPPAVTPVSSPPAPVPVSSPPAPVPVSSPPALAPTTPAPVVAPSAEVPAPAPKSKKKTKKSKKHTAPAPSPSLLGPPAPPVGAPGSSQDSMSPGPAVSEDESGAETIRCLKKVIGCLALSWATLVLFF

SEQ ID NO: 111 - protein, 653 aa, Glycine max
MAGKGEGPAIGIDLGTTYSCVGVWQHDRVEIIANDQGNRTTPSYVGFTDTERLIGDAAKNQVAMNPINTVFDAKRLIGRRFSDSSVQSDIKLWPFKVIPGAADKPMIVVNYKGEEKQFAAEEISSMVLIKMREIAEAYLGSTVKNAVVTVPAYFNDSQRQATKDAGVIAGLNVMRIINEPTAAAIAYGLDKKATSVGEKNVLIFDLGGGTFDVSLLTIEEGIFEVKATAGDTHLGGEDFDNRMVNHFVQEFKRKNKKDISGNPRALRRLRTACERAKRTLSSTAQTTIEIDSLYEGIDFYSTVTRARFEELNMDLFRKCMEPVEKCLRDAKMDKRSVDDVVLVGGSTRIPKVQQLLQDFFNGKELCKSINPDEAVAYGAAVQAAILSGEGNEKVQDLLLLDVTPLSLGLETAGGVMTVLIPRNTTIPTKKEQVFSTYSDNQPGVLIQVFEGERARTRDNNLLGKFELSGIPPAPRGVPQITVCFDIDANGILNVSAEDKTTGQKNKITITNDKGRLSKEDIEKMVQEAEKYKSEDEEHKKKVEAKNALENYAYNMRNTVKDDKIGEKLDPADKKKIEDAIEQAIQWLDSNQLAEADEFEDKMKELESICNPIIAKMYQGGAGPDVGGAGAAEDEYAAPPSGGSGAGPKIEEVD

SEQ ID NO: 112 - protein, 316 aa, Glycine max
MAGLVVSSQCFLKLLLVVSVFHVSFAARRLNELVQDQSQLLHYHNGPLLYGKIAVNLIWYGNFKPSQKAIITDFVTSLSSPASQSSQPSVATWWKTTEKYYHLSPRKASLSLSLGDQILDETYSLGKSLTGKNLVQLASKGGQRNSINVVLTSADVAVEGCMSRCGTHGSSASHLKKNSKSYKFAYIWVGNSETQCPGQCAWPFHQPIYGPQSPPLVAPNNDVGLDGMVINLASLLAGTATNPFGNGYFQGPAEAPLEAASACPGVYGKGAYPGYAGDLLVDSTTGASYNVKGANGRKYLVPALYDPSTSSCSTPV

SEQ ID NO: 113 - protein, 186 aa, Glycine max
MAKSTFFVCLNLSLLFSLVTATYYSSLTPTLLGFREEKFTHLHFFFHDVVSGPKPSMVFIAEPNGKAKDALPFGTVVAMDDPLTVGPEQDSKLVGKAQGIYTSISQEEMGLMMVMTMAFTNGDFNGSTISVLGRNMIMSEPVREMAIVGGTGAFRFARGYAQARFYSVDFTKGDAIVEYDVFVNHY

SEQ ID NO: 114 - protein, 273 aa, Glycine max
MSVVADNSANNGSHQVVLNVNGDAPKKCDDSANQDCVPLLQKLVAEVVGTYFLIFAGCASVVVNLDKDKVVTQPGISIVWGLTVMVLVYSVGHISGAHFNPAVTIAHATTKRFPLKQVPAYVIAQVVGATLASGTLRLIFNGKNDHFAGTLPSGSDLQSFVVEFIITFYLMFVISGVATDNRAIGELAGLAVGSTVLLNVMFAGPITGASMNPARSLGPAIVHHEYRGIWIYLVSPTLGAVAGTWAYNFIRYTNKPVREITKSASFLKGSEAE

SEQ ID NO: 115 - protein, 539 aa, Glycine max
MGLQIKEPLLFTLVTISLISITKLLHSYFSIPFSPSNLSIAIATLIFVLISYKFSSSSIKHSSTTLPPGPLSVPIFGNWLQVGNDLNHRLLASMSQTYGPVFLLKLGSKNLVVVSDPELATQVLHAQGVEFGSRPRNVVFDIFTGNGQDMVFTVYGDHWRKMRRIMTLPFFTNKVVHNYSNMWEEEMDLVVRDLNVNERVRSEGIVIRRRLQLMLYNIMYRMMFDAKFESQEDPLFIQATRFNSERSRLAQSFEYNYGDFIPLLRPFLRGYLNKCKDLQSRRLAFFNTHYVEKRRQIMAANGEKHKISCAMDHIIDAQMKGEISEENVIYIVENINVAAIETTLWSIEWAVAELVNHPTVQSKIRDEISKVLKGEPVTESNLHELPYLQATVKETLRLHTPIPLLVPHMNLEEAKLGGHTVPKESKVVVNAWWLANNPSWWKNPEEFRPERFLEEECATDAVAGGKVDFRFVPFGVGRRSCPGIILALPILGLVIAKLVKSFQMSAPAGTKIDVSEKGGQFSLHIANHSTVLFHPIKTL

SEQ ID NO: 116 - protein, 472 aa, Glycine max
MVEFGTRMGSIWTVLFCFASVFAIVSAERSIVKMEVPRFNVTSREPQQSFLTKALNFLWQSGESGYQHVWPDMEFGWEIVLGTFVGFCGAAFGSVGGVGGGGIFVPMLSLIIGFDPKSSTAISKCMIMGAAVSTVYYNLKLRHPTLNMPIIDYDLALLIQPMLMLGISIGVVFNVVFPDWIVTILLIVLFLGTSTKAFFKGVETWKKETIMKKEAAKRQESNGSGAVVEYKPLPSGPEKDTKEQEMSIIENVYWKEFGLLVFVWVSFLALQIAKENYTTTCSTLYWVLNLLQVPVSVGVTAYEAAALFSGRRVIASTGEQGKDFTVLQLMIYCVFGVLAGVVGGMLGLGGGFVMGPLFLELGVPPQVSSATATFAMTFSSSMSVIEYYLLKRFPVPYALYFILVATIAAFVGQHIVRKLIILFGRASLIIFILASTIFVSAVSLGGVGIVNMVHKIQNHEYMGFEDLCKYGS

SEQ ID NO: 117 - protein, 313 aa, Glycine max
MATLIAPSNHSPQEDAEALRKAFEGWGTDEKTVIVILGHRTVYQRQQIRRVYEEIFQEDLVKRLESEIKGDFEKAVYRWLLEPADRDAVLANVAIKNGKNYNVIVEIATILSPEELLAVRRAYLNRYKHSLEEDVAAHTSGHLRQLLVGLVTSYRYVGDEINPKLAQTEAEILHDAVKEKKGSYEETIRVLTTRSKTQLVATFNRYREIHGTSISKKLVDEGSDEFQRALYTAIRGINDPIKYYEKVVRNAIKKVGTDEDALTRVVVSRAEKDLKIISEVYYKRNSVLLEHAIAKETSGDYKKFLLTLLGKED

SEQ ID NO: 118 - protein, 581 aa, Glycine max
  MISC_FEATURE (264)..(264): Xaa can be R or M
MEEKMSWTVADAVNYKGFPADRSKTGGWVPAALILGIEIVERLSTMGIAVNLVTYMISIMHLPSSTAANTVTDFMGTSFLLCLLGGFLADSFLGRYKTIGIFASIQTLGTATLAISTKLPGLRPPPCHANSDSCKQANGFQMGILYLSLYLIALGTGGLKSSVSGFGSDQFDEKDEKEKSQMAYFFNRFFFFISFGTLAAVTVLVYLQDEVSRSLAYGICSVSMIIAIIVFLSGTKRYRYKRSLGSPIVHIFQVIAASIKKRKXQLPYNVGSLYEDTPEASRIEHTEQFRFLEKAAIVAEGDFETNVCGSESNPWKLCSLTRVEEVKMMVRLLPVWATTIIFWTIYAQMITFSVEQASTMERNIGSFQIPAGSLTVFFVAAILITLAVYDRLIMPLWKKWNGKPGFTDLQRIAIGLVFSIFGMAAASVCERKRLSAAKSVSGGNQATTLPISVFLLIPQFFLVGSGEAFIYTGQLDFFITRSPKGMKTMSTGLFLTTLSLGFFISSFLVSVVKKVTGTRDGQGWLADNINKGRLDLFYALLTILSFINFVAFAVCALWFKPKKPKQPAMQMGPQQRKSVEG

SEQ ID NO: 119 - protein, 229 aa, Glycine max
MQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGF

SEQ ID NO: 120 - protein, 413 aa, Glycine max
MDPTSIPPPPATTVPFTVEPSNHVTPADNTNTNHPPYDEMIYTAIGALKEKDGSSKRAIGKYMEQVYKDLPPTHSALLTHHLNRLKSAGLLILVKKSYKLPGSDPLPVLQAQKPRGRPPKLKSQPNTELTWPALALNDNPALQSAKRGPGRPKKIAGPVGVSPGPMVPGRRGRPPGTGRSKLPKRPGRPPKPKSVSAISSGLKRRPGRPPKAESNVNVIPFAAPVAPGLPTVQPIVPTASVPNGSPRPRGRPKKIVAGAGAPALSSVGGAPRGRGRPRGVLPLVRPGRPQKLAVGRPKNPARRPVGRPKGSTAAAITAHKAANDDLRRKLEHFQSKVKESLGTLKPYFNHESPVTAIAAIQELEVLSTLDLKAPLRDETHQQPQPQPQVYEQQYPQPQPLLQQFFQPHTSAPS

SEQ ID NO: 121 - protein, 518 aa, Glycine max
MPAVGGINTGGGKEYPGSLTLFVTVTCIVAAMGGLIFGYDIGISGGVTSMDPFLLKFFPSVFRKKNSDKTVNQYCQYDSQTLTMFTSSLYLAALLSSLVAATVTRKFGRKLSMLFGGLLFLVGALINGFAQHVWMLIVGRILLGFGIGFANQSVPLYLSEMAPYKYRGALNIGFQLSITVGILVANVLNYFFAKIKGGWGWRLSLGGAMVPALIITVGSLVLPDTPNSMIERGDREKAKAQLQRIRGIDNVDEEFNDLVAASESSSQVEHPWRNLLQRKYRPHLTMAVLIPFFQQLTGINVIMFYAPVLFSSIGFKDDAALMSAVITGVVNVVATCVSIYGVDKWGRRALFLEGGVQMLICQAVVAAAIGAKFGTDGNPGDLPKWYAIVVVLFICIYVSAFAWSWGPLGWLVPSEIFPLEIRSAAQSINVSVNMLFTFLIAQVFLTMLCHMKFGLFLFFAFFVLIMTFFVYFFLPETKGIPIEEMGQVWQAHPFWSRFVEHDDYGNGVEMGKGAIKEV

SEQ ID NO: 122 - protein, 227 aa, Glycine max
MINFEETELRLGLPGNDSALKGSAAKRGFSETASVDLKLNLSSCINDSASDSPSSVSTEKPKENKTTTAEPPPANDPAKPPAKAQVVGWPPVRSFRKNIVQRNSNEEEAEKSTKNAFVKVSMDGAPYLRKVDIKLYKSYQELSDALAKMFSSFTIEKCGSQGMKDFMNETNGSDYVPTYEDKDGDWMLVGDVPWEMFVESCKRLRIMKGSEAIGLAPRAVEKCKNRS

SEQ ID NO: 123 - protein, 126 aa, Glycine max
MKGQQQLKKSKVVKIDSRKSWEHHITNATNKGYPVMVHFSAYWCMPSIAMNHFFQQLASTYQNVLFLNVDVDEVKEVASKLEIKAIPTFCLMNGGAPVDKIVGANPDELRKRINCFIHQKHSPKSV

SEQ ID NO: 124 - protein, 388 aa, Glycine max
MVSVEEIRQAQRAEGPATVMAIGTATPPNCVDQSTYPDYYFRITNSEHMTELKEKFKRMCDKSMIKKRYMYLNEEILKENPSVCAYMAPSLDARQDMVVMEVPKLGKEAATKAIKEWGQPKSKITHLIFCTTSGVDMPGADYQLTKLLGLRPSVKRYMMYQQGCFAGGTVLRLAKDLAENNKGARVLVVCSEITAVTFRGPTDTHLDSLVGQALFGDGAAAVIVGSDPLPVEKPLFQLVWTAQTILPDSEGAIDGHLREVGLTFHLLKDVPGLISKNIEKALVEAFQPLGISDYNSIFWIAHPGGPAILDQVEAKLGLKPEKMEATRHVLSEYGNMSSACVLFILDQMRKKSIENGLGTTGEGLDWGVLFGFGPGLTVETVVLRSVTV

SEQ ID NO: 125 - protein, 79 aa, Glycine max
MSKVVTLFTLALLLSFNLIHASRPNPSLNVVSSSHEDVAATKEEIDEESCEEGTEECLIRRTLAAHVDYIYTQKHKPKP

SEQ ID NO: 126 - protein, 506 aa, Glycine max
MKELWAQMGSLMATIVFMYTIFERFFPPHLREKLQAYTQKLTNHFNPYIQISFPEFSGERLKKSEAYTAIQTYLSANSSQRAKRLKAEVVNDSQTPLVLSMDDNEEITDEFHGIKLWWSANKVSNNPQRYNPFSYYGSSDEKRFYKLTFHKRHRDIVTMSYIKHVLDEGKDIEMRNRQLKLYTNNPSSGWYGYKQSKWSHIVFEHPATFETLAMDRRKKEDILKDLVKFKKGKDYYAKIGKAWKRGYLLYGPPGTGKSTMIAAIANFMNYDVYDLELTAVKDNTELRKLLIETPSKSITVIEDIDCSLDLTGQRKKKKEENEDEEQKDPMRRNEEESSKSSKVTLSGLLNFIDGIWSACGGERIIVFTTNYVEKLDPALIRRGRMDKHIEMSYCCYDAFKVLAKNYLDVESHHLFGAIGGLLEETDMSPADVAENLMPKSVDEDVEICLHKLIKALEEAKEEKARKKAEEEEEARLKEEKVKEESTQMEEKHKGKTGEDVKENGFH

SEQ ID NO: 127 - protein, 98 aa, Glycine max
MEVEGSSKKMIATQEEMVEARVPLAYRDQCAHLLIPLNKCRQAEFYLPWKCQDQRHSYEKCQYELVMERMLQMQKIREHQQNPNAKQPLIALPKPANA

SEQ ID NO: 128 - protein, 186 aa, Glycine max
MASSLHNYVLAILLLAITQFKTTSAGSHHHLQHLKSLHFSLFQHETINKTGYIIVDGIKGGAGVTQTTTPFGTLFAFQDPLTVAANRSSKLVGIAEGTTVTSSLDGLRSISIAKLTLRLKHHKGSLSIVGVTNNVKPSDLPVVGGTEDFMFVQGYISTSPVDLKGLTVVYKIEFHLYWPPYATQAS

SEQ ID NO: 129 - protein, 312 aa, Glycine max
MESQTQSELPVVDFTNKNLKPGTDAWVSASQVVRGALEDHGGFLALYDKVSLETYDSVYSEMMNFFDLSIETKRRKTTEKPIFSYSGQRPGIPLYESVGIMNPLSFQDCQKYTHVMWPQENHHFCESVNSYAKQLVELDHIVKRMVFESYGLETKKFETLLESTEYVLRGYKYRIPREGESNLGVAPHCDTAFLTILNQKVEGLGVKLKDGKWLEVGASPSLYLVMGGDALMVWSNDRIPACEHRVLMNSKIDRYSMGLLSYAAKIMEPQEELVDEEYPLRYKPFDHYGYLRFFLTEEAIKSDSRIKAYCGI

SEQ ID NO: 130 - protein, 276 aa, Glycine max
MAAQALVSSSSLTFSAEAARQSLGPRSLQSPFGFSRKASFLVKAAATPPVKQGSDRPLWFASKQSLSYLDGSLPGDYGFDPLGLSDPEGTRGFIEPKWLAYGEIINGRYAMLGAVGAIAPEILGKAGLIPQETALPWFRTGVFPPAGTYNYWADSYTLFVFEMALMGFAEHRRFQDWAKPGSMGKQYFLGLEKGLGGSGEPAYPGGPFFNPLGFGKDEKSLKDLKLKEVKNGRLAMLAILGYFVQALVTGVGPYQNLLDHLADPVHNNILTSLKFH

SEQ ID NO: 131 - protein, 172 aa, Glycine max
MAFFILSFLFLLLVSSATACDRCLYQSKASYFSKASALSSGACGYGSLALDISGGHLAAGVDSLFKNGAGCGACFQIRCKNPTLCSKEGTKVVLTDLNHNNQTDFVLSSRAFAGMAQKGMGQQILKLGIAEIEYKRVPCDYKNQNLAVRVEESSKKPDYLAIKFLYQGGQQR

SEQ ID NO: 132 - protein, 403 aa, Glycine max
MGNAFLLQGGDCAESFKEFNANNIRDTFRIILQMSVVMMFGGQMPVIKVGRMAGQFAKPRSDSFEEKNGVKLPSYRGDNINGDSFDEKSRIPDPQRMIRAYCQAAATLNLLRAFATGGYAAMQRVTQWNLDFTDHSEQGDRYRELANRVDEALGFMAAAGLTVDHPIMRTTEFWTSHECLLLPYEQSLTRLDSTSGLYYDCSAHMLWVGERTRQLDGAHVEFLRGVANPLGIKVSDKMDPNELVRLIEILNPQNKPGRITVITRMGAENMRVKLPHLIRAVRRAGQIVTWVSDPMHGNTIKAPCGLKTRPFDFIRAEVRAFFDVHEQEGSHPGGVHLEMTGQNVTECIGGSRTVTFDDLSSRYHTHCDPRLNASQSLELAFIIAERLRKSRIRSQQPLAPLGV

SEQ ID NO: 133 - protein, 365 aa, Glycine max
MGSTGETQITPTHVSDEEANLFAMQLASASVLPMVLKSALELDLLEIIAKAGPGVHLSPSDIASRLPTHNPDAPVMLDRILRLLACYNILSFSLRTLPHGKVERLYGLAPVAKYLVRNEDGVSIAALNLMNQDKILMESWYYLKDAVLEGGIPFNKAYGMTAFEYHGTDPRFNKVFNKGMADHSTITMKKILETYTGFEGLKSLVDVGGGTGAVVNMIVSKYPTIKGINFDLPHVIEDAPSYPGVEHVGGDMFVSVPKADAIFMKWICHDWSDEHCLKFLKNCYEALPDNGKVIVAECILPVAPDSSLATKGVVHIDVIMLAHNPGGKERTEKEFEALAKGSGFQGFQVLCCAFNTYVMEFLKKV

SEQ ID NO: 134 - protein, 319 aa, Glycine max
MEGIEHQTLNVNGINMHIAEKGEGPLILFIHGFPDLWYSWRHQITALASLGYRCVAPDLRGYGDTDVPANPTAYTSLHVVGDLVGLLDAIVGDEEKVFVVGHDWGAMTAWSLSLYRPERIRALVNLSVVFTPRNPKRKPLDTLRAVYGNDYYICRFQEPGEIEAEFAQIGTARVLKEFLTYRNPGPLYLPKGKAFAHPTDSPIALPSWLSEEECDYYASKYDKTGFTGGLNYYRNLDLNWELTASWTGAQVKVPVKFIVGDLDLTYNAPGAKEYIHKGGFKRDVPLLEDVVVLEGAGHFLHQERPDEISNHIYDFFKKF

SEQ ID NO: 135 - protein, 247 aa, Artificial
  Glycine max MnSOD (LOC100101896) with C-terminal 6 x His tag
MAARALLTRKTLATVLRNDAKPIIGVGITAAATHSRGLHVYTLPDLDYDYGALEPAISGDIMQLHHQKHHQTYITNYNKALEQLQDAIAKKDSSAVVKLQGAIKFNGGGHVNHSIFWKNLAPVREGGGEPPKGSLGWAIDTHFGSFEALIQKVNAEGAALQGSGWVWLGLDKELKRLVVETTANQDPLVTKGPNLVPLIGIDVWEHAYYLQYKNVRPDYLKNIWKVINWKYASEVYEKESSHHHHHH

SEQ ID NO: 136 - protein, 202 aa, Glycine max
MGSSKLLGTMAMLFVVLLPMAAKGDNITDFLDKVCEEVECGKGSCVVNTSYPLNFVCECDSGWKRTQDDDDKYAASFLPCVIPECSLNYGCQPAPPPVPEKSFPHNFSAFDTCYWAYCGEGTCTKNRTHTHRCECQPNYYNLLNISVFPCYSECTLGSDCSRLGIKVANSSTDSGSQDSSASIFTGKFHWIVMLLMSTGMVM

SEQ ID NO: 137 - protein, 535 aa, Glycine max
MALNYPFLTYFILLLVTITRLIFTVGKTEQWKAPILPELDIDNISHKLHDDPETIQMASRDYGHLTHEFPLAVFRPSSIDDIVTLIKSSYNSFAPFDIAARGQGHSTHGQAMARDGIVVDMASLRKQRNGVAISVSKDPLMGHYADVGGEQLWIDVLHATLEYGLAPVSWTDYLYLTVGGTLSNAGISGQSFRYGPQISNVHEMDVITGKGEFVTCSSQKNLELFHAVLGGLGQFGVIARARIALEPAPKRVKWVRLLYSDFSAFTKDQERLISINGRKQKNALDFLEGMLLMNQGPINNWRSSFFPLSDHPRIASLITEHSILYCLEVAKYYDEQTELNVDKEIEVLLQGLAYIPGFNYEKNVSYVEFLNRVRSGELKLQSQGLWEVPHPWLNLFIPKSQILDFNSGVFKDIVLKRNISSGPVLVYPMNRNKWDDRMSASIPDEDVFYTVGFLHSSGFDTWKAYDAQNREILEFCRDAGIMVKQYLPNHSTQEDWTNHFGAKWMKFLERKHQFDPRMILSPGQKIFHKKLQPVF

SEQ ID NO: 138 - protein, 314 aa, Glycine max
MATLVAPNQKSPVEDVEALHKAFKGWGTDEKTVIAILGHRNVHQRQQIRKVYEEIYQEDLIKRLESELSGDFERAVYRWMLEPADRDAVLANVAIKNGSKGYHVIVEIACVLSADEVLAVKRAYHNRYKRSLEEDVATNTTGDIRQLLVGLVTAYRYDGDEVNAKLAKTEADILHESIKEKKGNHEEAIRILTTRSKTQLLATFNRYRDDHGASITKKLLDNASTDFQKALHTAIRCINDHKKYYEKVLRNAIKGVGTDEDALTRVVVSRAEKDLRDIKELYYKRNSVHLEDAVAKEISGDYKKFILTLLGKED

SEQ ID NO: 139 - protein, 113 aa, Glycine max
MSPKYMSLFLLVILGMAFLATTSLADRRFLSVEDGHDHHHHHPPKKHWPPTTQNEPSNVENKENTEVEDGHDHHHHYPPKKHWPPTTQNEPSKVENKDGHYKPPHKKHPPSGN

SEQ ID NO: 140 - protein, 116 aa, Glycine max
MAPRSSSKDAQDLFRALWSAYAATPTNLKIIDLYVIYAVFTAFIQVVYMALVGSFPFNSFLSGVLSCVGTAVLAVCLRIQVNKENKEFKDLAPERAFADFVLCNLVLHLVIMNFLG

SEQ ID NO: 141 - protein, 467 aa, Glycine max
MAQKRPPNSRGYYVKMKLLHKHGRPHHHHQQQQEKNCLYRYYKWVLWLSLSLYFFTSYLISNNNNNNHHSKQPSHVSRALMESNHTTPPQQQALNSLGSLKNLKVFVYDLPQKYNTDWLSNERCSKHLFASEVAIHRALLTSEVRTFDPYDADFFFVPVYVSCNFSTVNGFPAIGHARSLIASAVSLVSSEYPFWNRSRGSDHVFVASHDFGSCFHTLEDVAMADGVPEIMRNSIVLQTFGVVYDHPCQSVEHVVIPPYVSPESVRDTMENFPVNGRRDIWAFFRGKMELHPKNVSGRFYSKKVRTVIWRKFNGDRRFYLQRQRFAGYQSEIARSVFCLCPLGWAPWSPRLVESVALGCVPVIIADGIRLPFISAVKWPEISITVAEKDVGRLAEILERVAATNLSTIQRNLWDPVTRSALLFNSQVQKGDATWQILRALSEKLDRSFRSSRLNSAPTCVYFFTHRW

SEQ ID NO: 142 - protein, 278 aa, Glycine max
MQGCSDAPLSKRLEDKVALITGGASGIGEATARLFLRHGAKVVIADIQDNLGHSLCQNLNSGNNISYVHCDVTNDNDVQIAVKAAVSRHGKLDILFSNAGIGGNSDSSIIALDPADLKRVFEVNVFGAFYAAKHAAEIMIPRKIGSIVFTSSAVSVTHPGSPHPYTASKYAVVGLMKNLCVELGKHGIRVNCISPYAVATPLLTRGMGMEKEMVEELFAEAGNLKGVVLKEEDLAEAALFLASDESKYVSGVNLVVDGGYSVNNTASAEVALGKFSAD

SEQ ID NO: 143 - protein, 216 aa, Glycine max
MMEIEEQTMKMMMKSSSRFRRICVFCGTSPGKNPSYQLAAIQLAKQLVERNIDLVYGGGSIGLMGLISQVVFDGGRHVLGVIPTTLMPREVTGESVGEVRAVSGMHQRKAEMARQADAFIALPGGYGTLEELLEVITWAQLGIHDKPVGLLNVDGYYNSLLSFMDNAVDEGFITPAARHIIVSAQTAQDLMCKLEEYVPKHCGVAPKQSWEMNQQL

SEQ ID NO: 144 - protein, 712 aa, Glycine max
MEATNGHQNGSFCLSSTKGSDPLNWGAAAEAMKGSHLDEVKRMVSEYRKPVVRLGGETLTIAQVAAVAGHDHGVTVELSESAREGVKASSEWVMNSMNNGTDSYGVTTGFGATSHRRTKQGGALQKELIRFLNAGIFGNGTESSHTLPHTATRAAMLVRINTLLQGYSGIRFEILEAITKLLNNNVTPCLPLRGTITASGDLVPLSYIAGLLTGRPNSKAVGPNGEVLNAKEAFELASINSEFFELQPKEGLALVNGTAVGSGLASMVLFEANILAVLSEVLSAIFAEVMQGKPEFTDHLTHKLKHHPGQIEAAAIMEHILDGSSYMKAAKKLHEIDPLQKPKQDRYALRTSPQWLGPLIEVIRFSTKSIEREINSVNDNPLIDVSRNKALHGGNFQGTPIGVSMDNTRLALASIGKLMFAQFSELVNDFYNNGLPSNLTASRNPSLDYGFKGAEIAMASYCSELQYLANPVTSHVQSAEQHNQDVNSLGLISSRKTNEAIEILKLMSSTFLIALCQAIDLRHLEENLKNSVKNTVSQVSKRILTTGVNGELHPSRFCEKDLLKVVDREYVFSYIDDPCSATYPLMQKLRQVLVDHALVNAENEKDMNTSIFQKIANFEEELKNFLPKEVESARVAYESGKAAIPNKIQECRSYPLYKFVREELGTGLLTGEKVRSPGEEFDKLFTAMCQGKIIDPLLECLGEWNGAPLPIC

SEQ ID NO: 145 - protein, 432 aa, Glycine max
MEISSLEDYSSFFFPDMDPKRRGNDTAFNDLLNSLIHLDNKQTLMDPTQFQFDSSPPTKRPRRAIEDIHNVTVQQQQQQQRRLWVKDRSKDWWDRCNQPDFPEEEFRRYFRMSKATFDMICQHLDSAVTKKNTMLRQAIPVRQRVAVCIWRLATGDPLREVSKRFGLGISTCHKLVLEVCSTIKTVLMPKFLHWPNEAEMKPITQKFESLSGIPYVGGSMYTTHIPIIAPKSNVNAYFNKHHTERNQKTSYSITVQGVVDSKGVFSDVCIGWPGSLSDDQVLEKSALYQRATMGTLKDVWVVGNSGHPLMDGVLVPYTHQNLTWTQHAFNQKVGEIQSIAKDAFARLKGRWSCLQKRTEVKLEDLPVLLGACCVLHNICEMRDEEMDPHWKFEIFDDEMVAENNVRSNAAEQARDQIAHYLLHHGRAGTNFL

SEQ ID NO: 146 - protein, 264 aa, Glycine max
MKVLVFFVAIVLAAWHCHGSDHDHDHGHTYQIFPLRMKTGHGGHYIPEVSCQSWRLGVEAHNVIDWKTVPQDCEGYIGNYMLGEQYRSDSKIVNQQAYFYAKTLNITAKTAWVFDIDETTLSNLPYYADHGFGVELYNETSFNKWVDLGEAPALPESLKLYKKLLSLGIKIVFITGRPLDQKAVTATNLNLKLAGYHTWEKLITKNTSEYHGKTAVTYKSTERKKLEEKGYKIIGNIGDQWSDLLGTNTGDRTFKLPDPMYYIS

SEQ ID NO: 147 - protein, 369 aa, Glycine max
MAGSISLLEFSLVIFIQIMTHCHSSITTCLPEKHAALFILGDSLFDNGNNNYINTTTSYQANYPPYGETFFKYPSGRFSDGRMIPDAVAELAKLPILPPYLHPGHVEYVYGVNFASGGAGALRETSQGMVIDLKTQVSYLKNVKNLFSQRFGHAIAEEILSKSVYLFNIGANDYGSLLDPNSTSVLLPVDHQGFVDIVIGNLTDAIKEIYNIGGKKFGFLNVPPIGCSPAIRILVNNGSTCFEEFSAIARLHNNALSKRLHELEKQLKGFKYSVMDFYSAFSQVFNNPTKYGFKVASVGCCGSGPYRGVDSCGGNKGIKEYELCDNVNEHLFFDSHHLTDRASEYFAELIWNANRTVTSPYNLKQLFEL

SEQ ID NO: 148 - protein, 256 aa, Glycine max
MAGPIMRPQIVLFGSSIIQMSFDNGGWGAILANLYARKADIILRGYSGWNSRRALEVLDEIFPKDAYVQPSLVIVYFGGNDSIDPHPSGLGPHVPLEEYVENMRKIANHLKSLSDHIRIIFLTSPPINEELIRKKLSATQSGRTNESCGEYADGLMELCEEMNIKAINLWSAIQTREDWLDVSFTDGVHLSAEGSKVVVKEILKVLREVDWKPSLHWMSMPTEYAEDSPYYPPSPDGTTTINVSHIISRRCLQWDI

SEQ ID NO: 149 - protein, 533 aa, Glycine max
MLMSGTEQVTMGGLGLNLTPITTFAIITVIATVLIWWFWNALNWVWLRPKRIERRLKEQGIQGNSYRPLIGDIRDMVKMIKEAKSKPMDPHSNDIAPRVLPYVVHTIAKYGKSSFMWLGPTPRVFILDPDKFKEMATKVYDFQKPDTSPLFKLLASGFANYDGDKWAKHRKIVSPAFNVEKMKLLVPIFCQSCDDLISKWESLLSSSNGSCELDVWPFVQNVSSDVLARAGFGSSYQEGKKIFELQREMIQLTMTLFKFAFIPGYRFLPTHTNRRMKAIDKEIRESLMVIINRRLKAIKAGEPTNNDLLGILLESNYKESEKSSGGGMSLREVVEEVKLFYLAGQEANAELLVWTLLLLSRHPDWQEKAREEVFQVFGNEKPDYERIGQLKIVSMILQESLRLYPPVVMFARYLRKDTKLGELTIPAGVELVVPVSMLHQDKEFWGDDAGEFNPERFSEGVSKATKGKLSYLPFGWGPRLCIGQNFGLLEAKVAVSMILQRFSLHFSPSYAHAPSFIITLQPERGAHLILRKL

SEQ ID NO: 150 - protein, 703 aa, Glycine max
MATIILENDPLNWSHAADSLKGSHFEEVKRMVAEYRKPLISLGGGETLTISQVAAVAVANANHNLQAKVDLSESARAGVDASCDWITQNINKGTPIYGVTTGFGAASHRQTQQGLALQKEMVRFLNCAIFGYQTELSHTLPKSATRAAMLVRVNTLLQGYSGIRFEILEAITKLLNHNVTPILPLRGTVTASGDLIPLSYIVALLTGRRNSKAVGPSGESLNAKEAFHLAGLHSGFFELKPKEGLALVNGTAVGSGVASTVLFEANILALLSEVLSAVFAEVMQGKPEFTHHLIHKLKYHPGQIEAAAIMEHILDGSSYVKDAKLQQPDPLQKPRKDRYALVTSPQWLGPQIEIIRYSTKSIEREINSVNDNPLIDVTRNKALNGGNFQGTPIGVSMDNARLAVASIGKLIFAQFTELVNDLYNNGLPSNLSAGRNPSLDYGFKASEVAMAAYCSELQYLANPVTSHVQSAEQHNQDVNSLGLISALKTVEAVEILKLMSSTYLVALCQAIDLRHLEENFKSTVKNTVSRVAQKTLITEGKEEINPFRLCEKDLLKVVDREYVFSYIDDPSNVTYPLMPKLKQVLYEKAHISAINDKNVSLLIFEKIGAFEDELKSLLPKEVENARVAYENGNPAIPNRIKECRSYPLYKFVREELEIGLLTGEKNLSPDEEFEKVYTAMCQAKIVDPILECLGDWKGSPIPI

SEQ ID NO: 151 - protein, 207 aa, Glycine max; identical to SEQ ID NO: 106

SEQ ID NO: 152 - protein, 225 aa, Glycine max
MEEVKLIATHQSFPCARVEWALRIKGVEYEYLKEDLANKSSLLLQSNPVHKKVPVLLHNNKPIAESLVILEYIDETWKKNPLLPLDPYERAQARFWARFIDEKCVLAVWGATVAQGEEKEKAVGAALESLALLEKEIQGKKYFGGEKIGYLDIAAGCMSLWFSVLEELGEMELLNAERFPSLHEWSQNFLQTSPVKDCIPSRESVVEYFSFGINYVRSLAASSKS

SEQ ID NO: 153 - protein, 224 aa, Glycine max
MGSMKVHQFTRGLIWEHEPFLTLGCKRLRPLAPKLPNTKTITTPFDLKSFIRPESGPRKPVSSDDTKKDPPSPQGQIETHPGGTRWNPTQEQIGILEMLYKGGMRTPNAQQIEQITVQLGKYGKIEGKNVFYWFQNHKARERQKQKRSSLASSHSPRTPTIHSVVTLETTRGEVVERDHEEDSPYKKKCRRWVFDCLEEQNMSSPCEQEEHRTLELFPLHPEGR

SEQ ID NO: 154 - protein, 511 aa, Glycine max
MEAPSPTKPNDPTKPSNTSTTLSLEAGPAQASPLRKMFAVASIAAGIQFGWALQLSLLTPYVQLLGVPHAAASFIWLCGPISGLVVQPIVGYYSDHCTSRFGRRRPFILGGALAVAVAVFLIGYAADIGYAAGDDISKTTRPRAVGVFVIGFWILDVANNMLQGPCRAFLADLAAGDQRKTRIANGFFSFFMAVGNVLGYAAGSYKGLHKMFPFTETKACDVFCANLKSCFFFSILLLLFLATVALLYVKDKQVEARALDDATQPSCFFQLFGALKELKRPMWMLMLVTAVNWVGWFPYFLFDTDWMGREVYGGQVGEDAYANGVRVGSLGLMVNAVVLGFMSLAVEPLGKMVGGVKRLWAIVNFILAIGFGMTVVITKVAEHQRKMNPAAVGHPSEGVVVGSMVFFGVLGVPLAITFSVPFALASIYCSASGAGQGLSLGVLNLAIVVPQMVVSTLSGPWDALFGGGNLPAFMVGAAAAALSAIMAIVLLPTPKPADEAKASSMMAGGFH

SEQ ID NO: 155 - protein, 198 aa, Glycine max
MARISNWSLVFLLISTLLHIEPSLSSPSPSPSPSPFIKSSCSSTQYPALCVSSLSVYASSIQQDPHQLVQTALSLSLNRTQATKTFVANCNKFRGLKPREHAALKDCAEEISDSVDRLSRSLKELKLCKVKGEDFTWHISNVETWVSSALTDESTCGDGFSGKALNGKIKDSIRARMLNVAQVTSNALSLINHYAAQH

SEQ ID NO: 156 - protein, 212 aa, Glycine max
MASSLIAKRFLSSSLLSRSLLRPAASASHRSFNTNAMRQYDNRADDHSTDIDRHSERSFPSTARRDDIFSGNVLDPFFPTRSLSQVLNMMDQVMDNPFLSASRGIGAGAGVRRGWDARETEDALHLRVDMPGLGKEDVKISVEQNTLIIKGEGAKEGDEEESARRYTSRIDLPDKLYKIDQIRAEMKNGVLKVVVPKMKEEERKDVISVKVE

SEQ ID NO: 157 - protein, 287 aa, Glycine max
MENTKMMGVKIQDYASNMESNNYPFFDFSEDKGSLGFMELLGAQDYSPLLDFPLSSHVSGPQTSSVKEPPETKKECSEVTNNNQQPATPNSSSISSASSEVFYDEQNKTVDQAPEHQKTKEQLKAKKTNQKRQREPRFAFMTKSEVDHLEDGYRWRKYGQKAVKNSPFPRSYYRCTSVSCNVKKRVERSFSDPSIVVTTYEGQHTHPSPVMGRSNNFGSVIMSGSAGNYMSQYYQQQHQQVHIDALSSLGFLSSSSSRNATFPQETALLSDYGLLQDVVSSHMLKED

SEQ ID NO: 158 - protein, 106 aa, Glycine max
MATFELYRRSTIGMCLTETLDEMVQNGTLSPELAIQVLVQFDKSMTEALETQVKSKVSIKGHLHTYRFCDNVWTFILQDALFKNEDSQENVGRVKIVACDSKLLTQ

SEQ ID NO: 159 - protein, 121 aa, Glycine max
MALGRGSAVVLLLCFLLLHSQMARAATYTVGDSGGWTFNTVAWPKGKLFRAGDTLAFNYSPGTHNVVAVNKAGYDSCKTPRGAKVYKSGTDQIRLAKGQNYFICNYVGHCESGMKIAINAA

SEQ ID NO: 160 - protein, 116 aa, Glycine max
MLIIGCLCFSQRDGGASFHELPHLAPLLPFSHDVHGRQTHQIPLQLLLQPPLLSCACEHALGLSLPCASHARRKQQQMQHLTHQGAQDACGASLPSYSQPHHHTHHHLFCCTHHQW

SEQ ID NO: 161 - protein, 130 aa, Glycine max
MALEWVVLGYVAAAEAIMVILLTIPGLEALRKGLIAVTRNLLKPFLSVVPFCLFLFMDIYWKYETRPSCEGDSCTPSEHLRHQKSIMKSQRNALLIAAALLFYWLLYSVTNLVVRIDHLNQRLERLKNRD

SEQ ID NO: 162 - protein, 306 aa, Glycine max
MTAKQTKQHYNRKRTQSEREGSYSHKEKNFQRGESKRVLLFLFYIYLGAKLCEFIDKPEMATMLTKEHGLNLKETELCLGLPGGGGGGGGGGGGGGGEVETPRATGKRGFSETVDLKLNLHSKEDLNENLKNVSKEKTLLKDPAKPPAKAQVVGWPPVRSYRKNMMAVQKVSTEDVAEKTTSSTANPGAFVKVSMDGAPYLRKVDLTMYKSYKELSDALAKMFSSFTMGNYGAQGMIDFMNESKLMDLLNSSEYVPSYEDKDGDWMLVGDVPWEMFVESCKRLRIMKGSEAIGLAPRAMEKCKSRS

SEQ ID NO: 163 - protein, 353 aa, Glycine max
MATYSWSTEIFSFLVLTLLSTSSHTMVAGARNLLESTLSKPEVPTLPKPHELPPLPEIPELPKFESPKIPALPKPELPKVPELSKPDMSKVPELPKVPERLKVPEISKILELSKPELPKGPELLKPELPSVPNIPKVPELPKPELPEVPKLPKPELPKVPELPKPELPKVPEIPELPNLELPKVTQLPKSKLPKVPEIPKVPEFPKPELPKVPELSKPELPKAPEIPKVPEFPKPELPKVPELSKPELPKAPEIPKVPEFPKPELPKVPELPKPELPKIPEIPKVPEFPKPELPKVPEVPKPELSKVPKLPKSELPKVPEIPELPKSELPKIPEIPKVPEFPKVPKAFSTTNP

SEQ ID NO: 164 - protein, 148 aa, Glycine max
MASKRILKELKDLQKDPPTSCSAGPVAEDMFHWQATIMGPPDSPYAGGVFLVTIHFPPDYPFKPPKVAFRTKVFHPNINSNGSICLDILKEQWSPALTISKVLLSICSLLTDPNPDDPLVPEIAHMYKTDRNKYESTARSWTQKYAMG

SEQ ID NO: 165 - protein, 566 aa, Glycine max
MAPSPQEIIFRSPLPDIPIPTHLPLYSYCFQNLSKFHDRPCLIDGDTGETLTYADVDLAARRIASGLHKIGIRQGDVIMLVLRNCPQFALAFLGATHRGAVVTTANPFYTPAELAKQATATKTRLVITQSAYVEKIKSFADSSSDVMVMCIDDDFSYENDGVLHFSTLSNADETEAPAVKINPDELVALPFSSGTSGLPKGVMLSHKNLVTTIAQLVDGENPHQYTHSEDVLLCVLPMFHIYALNSILLCGIRSGAAVLILQKFEITTLLELIEKYKVTVASFVPPIVLALVKSGETHRYDLSSIRAVVTGAAPLGGELQEAVKARLPHATFGQGYGMTEAGPLAISMAFAKVPSKIKPGACGTVVRNAEMKIVDTETGDSLPRNKHGEICIRGTKVMKGYLNDPEATERTVDKEGWLHTGDIGFIDDDDELFIVDRLKELIKYKGFQVAPAELEALLIAHPNISDAAVVGMKDEAAGEIPVAFVVRSNGSEIAEDEIKKYISQQVHFTNTIGFFSFLLANEEFKDQFNISIIINNYFFYVNALQKQSHQYMFSYYLSFFFCFTSL

SEQ ID NO: 166 - protein, 344 aa, Glycine max
MSGLIEGLPDAVAIRCLAWVPFYLHPKLELVSRAWRAVVRGPELFKARQELGSSEDLLCVCAFEPENLWQLYDPLRDLWITLPVLPSRIRHLSHFGAVSTAGKLFVIGGGSDAVDPLTGDQDGCFATNEVWSYDPVVRQWSPRAAMLVPRSMFACCVMNGKIVVAGGFTSCRKSISQAEMYDPEKDVWIPMPDLHRTHNSACSGVVIGGKVHVLHKDMSTVQVLDNAGQWTVEEYGWLHGQMAVIRDALYVISYGLIIKQDKKMRKVVGSASEFRRRIGFAMIGLGDELYVIGGVIGPDRWNWDIKPLSDVDVLTLASDRPTWRQAAPMTRCGGTILGCTLLRI

SEQ ID NO: 167 - protein, 544 aa, Glycine max
MATKPRLAYLSSTFLLLLVVTVFTSVSAQAPESPSLYNTFLQCLTKYTNNPSNIVFANTNPKFPTILQNYIRNARFNTSSTRKPLLIVTPQQESHVQGTVICAKSVEIQLKIRSGGHDYEGISYISEEPFVILDMFNYRRITVDVKNEVAVVEAGATLGEVYYRIWEKSKVLGFPAGVCPTVGVGGHFSGGGYGNMLRKYGLSVDNVIDAQIVDVKGNLLNRKTMGEDLFWAIRGGGGASFGVILSFTIKLVPVPETVTVFRVEKTLETNVTATDLVVQWQQVAPNTDDRLFMRLLLQPVSSKVVKGTRTVRASVVALFLGGANEVVSILAKEFPLLGLKKENCTEVSWIDSVLWWNDDNSLKNGDKPETLLDRNLNNAGFLKRKSDYVQNAISRDGLEWLFKRMIELGKTGLVFNPYGGKMAEIPSDATPFPHRKGNLYKIQYSVNWDDPSPGAALNFTNQAKRLFSYMTPFVSKNPRSAFLNYRDLDIGVNSFGENSFQEGLVYGTKYFNDNFQRLVKIKTTVDPENFFRNEQSIPVLHGEA

SEQ ID NO: 168 - protein, 259 aa, Glycine max
MAFFILSFLFLLLVSSATACDRCLYQSKASYFSKASALSSGACGYGSLALDISGGHLAAGVDSLFKNGAGCGACFQIRCKNPTLCSKEGTKVVLTDLNHNNQTDFVLSSRAFAGMAQKGMGQQILKLGIAEIEYKRVPCDYKNQNLAVRVEESSKKPDYLAIKFLYQGGQTEIVAVDVAQVGSSNWSFMSRNHGAVWDTSRVPQGALQFRLVVTAGYDGKWIWAKKVLPADWKNGLIYDSGLQITDIAQEACSPCDDGT

SEQ ID NO: 169 - protein, 509 aa, Glycine max
MALLLIVPISLVTLWLGYTLYQRLRFKLPPGPRPWPVVGNLYDIKPVRFRCFAEWAQSYGPIISVWFGSTLNVIVSNSELAKEVLKEHDQQLADRHRSRSAAKFSRDGKDLIWADYGPHYVKVRKVCTLELFTPKRLESLRPIREDEVTTMVESVYNHCTTTGNLGKAILVRKHLGSVAFNNITRLAFGKRFVNSEGVMDEQGVEFKAIVENGLKLGASLAMAEHIPWLRWMFPLEEGAFAKHGARRDRLTRAIMTEHTEARKKSGGAKQHFVDALLTLQDKYDLSEDTIIGLLWDMITAGMDTTAISVEWAMAELIRNPRVQQKVQEELDRVIGLERVMTEADFSSLPYLQCVIKEAMRLHPPTPLMLPHRANANVKVGGYDIPKGSNVHVNVWAVARDPAVWKDPLEFRPERFLEEDVDMKGHDFRLLPFGAGRRVCPGAQLGINLVTSMLGHLLHHFCWTPPEGMKPEEIDMGENPGLVTYMRTPIQALASPRLPSHLYKRVPAEI

SEQ ID NO: 170 - protein, 89 aa, Glycine max
MAANRWLKPEVYPLFASVGVAVGICGMQLVRNITTNPEVRVTKQNRTAGILENFAEGEKYSQHSLRKYVRGKQPQIMPSVNNFFSDPSN

SEQ ID NO: 171 - protein, 329 aa, Glycine max
MVTDQDIAKGVESLLRHSDPNSITTVNGVVQQLEAKLGLDLSHKASFIRDQIDHLLRSQPQTFAPHPPPLHKDYFAPHTQLHFPTTHFAPHFALHDEINFLQHPHPPPPRKVETFPPQNVAPPQVPKESVQTGSKRRGGAGGLNKVCGVSPELQAVVGEPAMPRTEIVRQLWAYIKKNNLQDPGNKRKIICDDALRLVFETDCTDMFKMNQLLAKHIIPLGPTKESQAKRVKVDTEIKTESAEPAPSTVAISEALAKFLGTEGREMQQSEAIRLVWEYIKLHHLEDPLNSMVILCDAKLQELLGCESISALGIPEMLARHHLFKQSDTR

SEQ ID NO: 172 - protein, 451 aa, Glycine max
MTMAFRVRNPDLRFLILLSLLSITSAKVFFEERFDDGWGNRWVKSDWKKDENLAGEWNHTSGQWNGDANDKGIQTSEDYRFYAISAQYPEFSNKDKTLVFQFSVKHEQKLDCGGGYMKLLSGDVDQKKFGGDTPYSIMFGPDICGYSTKKVHAILTYNDTNHLIKKDVPCETDQLTHVYTFILRPDATYSILIDNVEKQTGSLYSDWDLLPPKTIKDPEAKKPEDWDDKEFIPDPEDKKPEGYDDIPKELPDPEAKKPEDWDDEEDGEWTPPTIANPDYKGPWKAKKIKNPNYKGKWKAPLIDNPDFKDDPDLYVFPNLKYVGIELWQVKSGTLFDNVLITDDPEYAKQLAEETWGKHKDVSFFYSLIFFGLRYAIERYDLLNEIVFLQVEKTAFEEAEKKREEEESKDDPVDSDADEEEEDADEAGNDSDAESKTEAGEDTKEEGVHDEL

SEQ ID NO: 173 - protein, 159 aa, Glycine max
MAEQTEKAFLKQPKVFLSTKKTGKGKRPGKGGNRFWKSIGLGFKTPREAIEGTYIDKKCPFTGNVSIRGRILAGTCHSAKMNRTIIVRRNYLHFIKKYQRYEKRHSNIPAHISPAFRVKEGDHVIIGQCRPLSKTVRFNVLKVIPAGSSSGAKKAFTGM

SEQ ID NO: 174 - protein, 338 aa, Glycine max
MPSSESSTGFSETICVTGAGGFIASWMVKLLLEKGYTVRGTLRNPDDPKNGHLKEFEGASQRLTLHKVDLLHLDSVRSVINGCHGVFHTASPVTDNPEEMVEPAVNGAKNVIIAAAEAKVRRVVFTSSIGAVYMDPKRSIDLVVDESCWSDLEFCKNTKNWYCYGKAVAEEAAWDTAKEKGVDMVVVNPVLVLGPLLQPSINASTIHILKYLTGSAKTYANATQAYVHVRDVALAHILVYEKPSASGRYICAESSLHRGELVEILAKYFPDYPVPTKCSDEKNPRAKPYTFSNQKLKDLGLEFTPVSQCLYEAVKNLQEKGHLPVPARQQEDSTTVKP

SEQ ID NO: 175 - protein, 272 aa, Glycine max
MAPSSNYISFLFLTTLLFALQISARDSQFFSKVTHFDKNSVKETELPNKEAPEVNKPEQQPPFIPETENSYGLYGHHDEYNQVPSTTTNPTSYHPYKTEFENSNNKYYNNDAYNTRFSETGYNNNKDSYEGNQYELSDTKYTEEEYNNNNNNKYHNSYQNNNQKYYNNDAANGIYSYNNNNNYNANNNRYNTYNNNNAVNGYNGERQGMSDTRFLEGGKYFHDVAAEKYNPTNYGDSSREVNTNNWYNNRGGNYNGNQNQQEFEDEHENFEP

SEQ ID NO: 176 - protein, 248 aa, Glycine max
MTVIKEEQQPNQIAGHKELGHKSLLQSDALYQYILETSVYPREHESLKELRELTEKHPWNLMATPPDEGQLLGMLLKLINAKNTMEIGVFTGYSLLSTALALPSDGKILAMDVNREYYELGLPVIEKAGVAHKIDFREGPALPLLDVLIKDEKNKGAFDFIYVDADKDNYLNYHKRVIELVKLGGLIGYDNTLWNGSVVAPPDAPLMDYVKYYRDFVMELNKALALDSRVEICQLPVGDGITLCRRII

SEQ ID NO: 177 - protein, 240 aa, Glycine max
MVYLALCSLLTLALSLATTHAANFEIVNNCPYTVWAAASPGGGRRLDRGQTWNLWVNPGTAMARIWGRTGCNFDGSGRGRCQTGDCTGGLNCQGWGVPPNTLAEFALNQFGNQDFYDISLVDGFNIPMDFYPLNGGCHKISCSADINGQCPGPLRAPGGCNNPCTVFKTNEYCCTNGQGSCGPTNYSRFFKDRCHDSYSYPQDDPTSTFTCPAGSNYKVVFCPLGEPHVTLHMPASTAHQ

SEQ ID NO: 178 - protein, 175 aa, Glycine max
MKHCISKHFLSCFALFLLLVATTYATVVPTTTQKPPRSFANQFLIPQNAARAVLRLRPLVWDSKLAHYAQWYANQRRNDCALEHSNGPYGENIFWGSGTGWKPAQAVSAWVEERQWYNYWHNSCANGQMCGHYTQIVWSTTRKIGCASVVCSGGKGTFMTCNYDPPGNYYGERPY

SEQ ID NO: 179 - protein, 353 aa, Glycine max
MASATPLLKDELDIVIPTIRNLDFLEMWRPFFEPYHLIIVQDGDPSKIIKVPEGFDYELYNRNDINRILGPKANCISFKDSACRCFGYMVSKKKYIYTIDDDCFVATDPSGHKINALKQHIENLLCPSTPYFFNTLYEPFREGADFVRGYPFSLREGVPTAVSHGLWLNIPDYDAPTQLVKPLERNTRYVDAILTIPKGTLFPMCGMNLAFDRDLIGAAMYFGLMGDGQPIGRYDDMWAGWCCKVICDHLGLGIKTGLPYIYHSKASNPFVNLRKEYKGIFWQEDIIPFFQSIVLPKEATTVQKCYIELAKQVKEKLSKVDPYFDKLADAMVTWIEAWDELNPAGASVANGKA

SEQ ID NO: 180 - protein, 225 aa, Glycine max
MINHALCFLVFLSALHATHGVEYTVTNNALSTPGGVAFRDKIGAEYAKRTLDSATQFIWRIFQQNNPSDRKNVQKVSLFVDDMDGVAYTSNNQIHLSARYVGNNKGDVKTEITGVLYHEMVHVWQWSGNGRAPGGLIEGIADYVRLKANYAPSHWVKAGQGQKWDQGYDVTARFLDYCDSLKSGFVAQLNKLMRTGYSDQYFVQLLGKPVDQLWRDYKAKYGNIA

SEQ ID NO: 181 - protein, 233 aa, Glycine max
MDQKVDFSAPHSMGTTIIGVTYNGGVVLGADSRTSTGVYVANRASDKITQLTDNVYVCRSGSAADSQIVSDYVRYFLHQHTIQLGQPATVKVAANLVRLLSYNNKNFLETGLIVGGWDKYEGGQIYGVPLGGTIVQQPFAIGGSGSSYLYGFFDQAWKEGMTKDEAEDLVKKAVSLAIARDGASGGVVRTVIINSEGVTRNFYPGDQLPLWHEEMEPHNSLLDILGAPEPMSM

SEQ ID NO: 182 - protein, 158 aa, Glycine max
MGVFTFEDETTSPVAPATLYKALVTDADNVIPKAVDAFRSVENLEGNGGPGTIKKITFVEDGESKFVLHKIESVDEANLGYSYSVVGGVGLPDTVEKITFECKLAAGANGGSAGKLTVKYQTKGDAQPNPDDLKIGKVKSDALFKAVEAYLLANPHYN

SEQ ID NO: 183 - protein, 320 aa, Glycine max
MAFHSHGHIFSFSSILFCMFAMASSQLTSNCYESTCPQALSIIKTAVIGAVAKEHRMGASLLRLHFHDCFVNGCDASVLLDDTSTFTGEKSAAANVNSLRGFEVIDDIKTKVEAACPGVVSCADILAIAARDSVVTLGGPSWNVGLGRRDSTTASKDAATTDIPSPLMDLSALISSFSNKGFNTKEMVALSGAHTTGQARCQLFRGRVYNESSIESNFATSLKSNCPSTGGDSNLSPLDVTTNVVFDNAYFKNLINKKGLLHSDQQLFNSGGSTDSQVTAYSNDPSAFYADFASAMIKMGNLSPLTGKSGQIRTNCHKVN

SEQ ID NO: 184 - protein, 474 aa, Glycine max
  UNSURE (333)..(334): Xaa can be any naturally occurring amino acid
MSNLNETTIERGEEREEENVGGGGGGSHSSLKSILWHGGSAYDAWFSCASNQVAQVLLTLPYSFSQLGMLSGIIFQIFYGIMGSWTAYLISILYIEYRTRKEKESVSFKNHVIQWFEVLEGLLGPYWKAIGLAFNCTFLLFGSVIQLIACASNIYLINDHLDKRTWTYIFGACCATTVFVPSFHNYRIWSFLGLGMTTYTAWYMTIAAIAHGQVENVIHTGPKKLVLYFTGATNILYTFGGHAVTVEIMHAMWKPQKFKYIYLYATLYVFTLTIPSSVAVYWAFGDELLDHSNAFSILPRSGWRDTAVILSSFISSSLLDLLAHHCILCGRKXXKCRHKEPMFEGTCXVACGDTNMVFCYYFPFFGPINSAVGALLVTFTVYVIPASAHCSLTNLLLPDRMLLRNYPSSSQIGQQCIWXMHLWWCGFXWLDLDLEGGLAXQTSSNRLTHLDSLPSATSARQNSQLPTTQSCIIX

SEQ ID NO: 185 - protein, 224 aa, Glycine max
MKQKIVIKLQMDCDKCRNKALKIAAEVRGVTTVSLEGDDNDRVAVSGVNVDMVCLANQLKKKFSSVTILTVVDLVKEEEAKKKKDEEEKKKKEEAEKKRKEEEERLKKMLRSVLCKKCKSSSCHGKCDTACCTKCESIHCGGDCFIVCVNCDSPKCEGDCKPCINCLSSKCECECEPCPKPPSPCPKWCNCHKCYVPYQQPCYYPYPPQVVCYDTCPDSPCSIM

SEQ ID NO: 186 - protein, 307 aa, Glycine max
MAVPVIDFSKLNGEERTKTMAQIANGCEEWGFFQLINHGIPEELLERVKKVASEFYKLEREENFKNSTSVKLLSDSVEKKSSEMEHVDWEDVITLLDDNEWPEKTPGFRETMAEYRAELKKLAEKLMEVMDENLGLTKGYIKKALNGGDGENAFFGTKVSHYPPCPHPELVKGLRAHTDAGGVILLFQDDKVGGLQMLKEGQWIDVQPLPNAIVINTGDQIEVLSNGRYKSCWHRVLATPDGNRRSIASFYNPSFKATICPAPQLVEKEDQQVDETYPKFVFGDYMSVYAEQKFLPKEPRFQAVRAM

SEQ ID NO: 187 - protein, 88 aa, Glycine max
MEGLIPFVYKAIMQSKGDKEGHPIGSWLCESPYSYMRLPGDSGRFQIQAPAAASPSSTNPNSSSATQIIVSSGVQSPHQCLTHRRIAA

SEQ ID NO: 188 - protein, 221 aa, Glycine max
MKVVYFLVAILALTSSLVSAYDPSPLQDFCVAAKEKDGVFVNGKFCKDPKLVKAEDFFRHVEPGKTDNPVGSNVTQVFVDQLPGLNTLGIALARIDFAPKGLNAPHTHPRGTEILIVLEGTLYVGFVTSNQDGNRLFTKVLNKGDVFVFPIGLIHFQLNVGYGNAVAIAGLSSQNPGAITIANALFKANPPISSEVLTKAFQVDKTIIDYLQKQSWYDNNN

SEQ ID NO: 189 - protein, 320 aa, Glycine max
MASFCSRLTICLALFVLIWGSANAQLSTNFYYHSCPNLFSSVKSTVQSAISKETRMGASLLRLFFHDCFVNGCDGSILLDDTSSFTGEKNANPNRNSARGFEVIDNIKSAVEKVCPGVVSCADILAIAARDSVQILGGPTWNVKLGRRDARTASQSAANNGIPAPTSNLNQLISRFSALGLSTKDLVALSGGHTIGQARCTNFRARIYNETNIETAFARTRQQSCPRTSGSGDNNLAPLDLQTPTSFDNYYFKNLVQKKGLLHSDQQLFNGGSTDSIVRGYSTNPGTFSSDFAAAMIKMGDISPLTGSNGEIRKNCRRIN

SEQ ID NO: 190 - protein, 534 aa, Glycine max
MGNCCATPSTDETANKKGKKGKKENPFAIDYGFNATAANGSKLTVLKSPTGREIEARYELGRELGRGEFGITYLCTDKGTGEELACKSISKKKLRTAIDIEDVRREVEIMRHLPQHANIVTLKDTYEDDNAVHLVMELCEGGELFDRIVARGHYTERAAAAVTKTIVEVVQMCHKQGVMHRDLKPENFLFANKKETAALKAIDFGLSVFFKPGEKFNEIVGSPYYMAPEVLKRNYGPEVDIWSAGVILYILLCGVPPFWAETEQGVAQAIIRSVVDFKRDPWPKVSDNAKDLVKKMLDPDPKRRLTAQDVLDHPWLQNAKKAPNVSLGETVRARLKQFSVMNKLKKRALRVIAEHLTVEEAAGLKEGFQVMDTNNRGKINIDELRVGLHKLGHQVPESDVQALMDAGDVDGDGHLDYGEFVAISVHLRKMGNDEHLRKAFQFFDQNKSEYIEIEELRSALSDDLDTNSEEVVNAIMHDVDTDKDGRISYDEFSTMMKAGTDWRKASRQYSRERFASLSLTLMRDGSLHLNNEKQ

SEQ ID NO: 191 - protein, 134 aa, Glycine max
MWFSSFLSPFLMRHSKVRPNAPSDDQSSQLTHNPQLDHGGWSRRGREFCEARRAFLNSYHLSLERKNNVSFKEKLKKSVKEVNEAAMGVVLGMRRGVSKRRVGIKVFRVKMSSHSMVLVTLRCFIPWLNKSKVI

SEQ ID NO: 192 - protein, 431 aa, Glycine max
MPSLEEELFPSTPGKFKIERAHHMNRQLYRCFASTSTMFLWALFLIALTASYLSFQGFVDSGSRYLSASWGGIQWEKQVRTSAQIHRQGGMSVLVTGAAGFVGSHVSLSLKRRGDGVVGLDNFNDYYDPSLKKARKSLLAKHDVFIVDGDLNDAKLLAKLFDVVAFTHVMHLAAQAGVRYAMENPHSYVHSNIAGLVTLLEACKSANPQPAVVWASSSSVYGLNEKVPFSESDQTDRPASLYAATKKAGEEITHTYNHIYGLSITGLRFFTVYGPWGRPDMAYFSFTRNILQGKPITVYRGKNHVDLARDFTYIDDIVKGCVGSLDTSAKSTGSGGKKRGPAPYRIFNLGNTSPVTVPTLVSILERHLKVKAKRNIVDMPGNGDVPFTHANISSARRELGYKPTTDLQTGLKKFVKWYLSYYGYNHGKPVN

SEQ ID NO: 193 - protein, 406 aa, Glycine max
MATTFLSLLFLLFSLLTPTLISSSPVQDPEFVAQEVHRKINASVARRNLGYLSCATGNPIDDCWRCDPNWEKNRQRLADCAIGFGKNAIGGKNGKIYVVTDSGDDDPVTPKPGTLRYAVIQDEPLWIIFARDMVIKLKEELIMNSFKTIDGRGASVHIAGGPCITIQYVTNVIIHGINIHDCKQGGNAMVRDSPRHYGWRTISDGDGVSIFGGSHVWVDHCSLSNCNDGLIDAIHGSTGITISNNYMTHHDKVMLLGHSDSYTQDKSMQVTIAFNHFGEGLVQRMPRCRHGYFHVVNNDYTHWEMYAIGGSANPTINSQGNRFVAPDDRFSKEVTKHEDAPESEWKGWNWRSEGDLLVNGAFFTASGAGASSSYARASSLSARPSSLVGSITTGAGALSCRKGSRC

SEQ ID NO: 194 - protein, 702 aa, Glycine max
MAQNGSGDRVVVDNGFESIDDAYDHEHKLSQKGSTKVKEEEVSVERVFQHLLVPSWRNQLTVRAFVVSFALSILFSFIVMKLNLTTGIIPSLNVSAGLLGFFFVKTWTKFLEKSNMLRQPFTRQENTVIQTCVVASSGIAFSGGFGSYLFGMSEEIAKQSSDPSHFKDPKLGWIIGFLFVVSFLGLFSVVPLRKIMVIDFKLTYPSGTATAHLINSFHTPQGAKLAKKQVKMLGKFFSLSFFWGFFQWFYTATDQCGFQAFPSLGLKAYENKFFFDFAAIYVGVGMICPYIINISVLLGGIISWGIMWPLIKTKEGDWYDKGLGEGNLHGIQGYRVFIAIALILGDGLYNFIKVLTHTLWGLYHQIREKQRENVLPVADQDSPSNSHLSYDDQRRTQLFLKDQIPTWFAISGYVAIAAISTATLPHIFPELKWYYIIVIYLIAPTLAFCNAYGCGLTDWSLASTYGKLAIFTIGAWAGSSNGGVLAGLAACGVMMNIVSTASDLMQDFKTGYLTLASPRSMFVSQIIGTTMGCIISPCVFWIFYKAFPDLGRSTSEYPAPYAIIYRNMAILGVQGFGHLPKNCLLLCYIFFAAAVAINLIKDFLGKRGRFIPLPMAMAIPFYIGPYFAIDMCVGSLILYVWERINKAKADAFAPAVASGLICGDGIWTLPASILALAGVKPPICMKFLSRAANARVDTLLGN

SEQ ID NO: 195 - DNA, 16 bp, Artificial
  PCR primer sequence
gtaaaacgacggccag

SEQ ID NO: 196 - DNA, 17 bp, Artificial
  PCR primer sequence
caggaaacagctatgac

SEQ ID NO: 197 - DNA, 22 bp, Artificial
  PCR primer sequence
aagaagccctccagcttcaaag

SEQ ID NO: 198 - DNA, 22 bp, Artificial
  PCR primer sequence
atggtgagcaagggcgaggagc

SEQ ID NO: 199 - DNA, 24 bp, Artificial
  PCR primer sequence
tcgtccatgccgagagtgatcccg

SEQ ID NO: 200 - DNA, 19 bp, Artificial
  PCR primer sequence
tcagcctccccgccggatg

SEQ ID NO: 201 - DNA, 24 bp, Artificial
  PCR primer sequence
atgcaaaagacaggattgatcgca

SEQ ID NO: 202 - DNA, 22 bp, Artificial
  qRT-PCR primer sequence
ctaagatgcagaacgaggaagg

SEQ ID NO: 203 - DNA, 22 bp, Artificial
  qRT-PCR primer sequence
gagagcaaaagtggagaaatgg

SEQ ID NO: 204 - DNA, 25 bp, Artificial
  qRT-PCR primer sequence
gcagatgggttaatggagctttgtg

SEQ ID NO: 205 - DNA, 25 bp, Artificial
  qRT-PCR primer sequence
gacatccaatgcagactaggtttcc

SEQ ID NO: 206 - DNA, 25 bp, Artificial
  qRT-PCR primer sequence
cgtggatgggtactacaactcgttg

SEQ ID NO: 207 - DNA, 25 bp, Artificial
  qRT-PCR primer sequence
tggttcatctcccaactttgctttg